SEQUENCE LISTING (210 sequences)

SEQ ID NO: 1 - DNA, 306 bp, Arabidopsis thaliana
atggcgtcgattctgggtgatttgccttcctttgatcctcacaatttcagtcaacatcgtccctccgatccttctaatccctctaagatggttcctaccacctatcgtcctactcacaaccgtacacttccaccaccagatcaagtgataactacagaagtgaaaaacattcttatacgcagcttctatcaacgagctgaagaaaagttaagaccaaagagaccggctacagatcatctggcagcggagcacgtgaacaagcatttccgtgctgcgtcttcttcttcatctactcagggcttataa

SEQ ID NO: 2 - DNA, 517 bp, Arabidopsis thaliana
gtctgaagagaaggaaagatcatcaatcacgattccaatggcgtcgattctgggtgatttgccttcctttgatcctcacaatttcagtcaacatcgtccctccgatccttctaatccctctaagatggttcctaccacctatcgtcctactcacaaccgtacacttccaccaccagatcaagtgataactacagaagtgaaaaacattcttatacgcagcttctatcaacgagctgaagaaaagttaagaccaaagagaccggctacagatcatctggcagcggagcacgtgaacaagcatttccgtgctgcgtcttcttcttcatctactcagggtttataaaaaacttaagttcaagcctataacaatggtcatttgtatgagtacctcttattagtgttttcaatgtagaaaaaaaaagataagagcagttcatgagagaaatgtagtgaaaatgtgtgtgtacataacattaatgtttcttttatttcttgacttaaagttgctcactattca

SEQ ID NO: 3 - DNA, 1406 bp, Arabidopsis thaliana
gtctgaagagaaggaaagatcatcaatcacgattccaatggcgtcgattctgggtgatttgccttcctttgatcctcacaatttcagtcaacatcgtccctccgatccttctaatccctctgttagtttcttcccccaaattcaatttttcaattttacggatctgagttaggctttacttggtcgtattggaaaaaaaatgtgtcctttgttgattcaaagagatgtaattcgaatgtgtatctgggttttgctgcttacttggtcagttccaaaaagttccatcttttcaattatcatcgagtttgctgttggatcttgtgaaaaaccaatacaaattagccatttttgtcagattgattgatcttagaatcataattctgattccatttggccataatttagctgctaagtgacgaagacaagtttcaactagcttgtatcagttaaagattagagttttgatctgttatcgaaggtttgagttttttgttcatgttttcttgcagaagatggttcctaccacctatcgtcctactcacaaccgtacacttccaccaccagatcaaggtgaaacaaaaaatcgtgcttttttgaaaaaccttgcgtgttttttcggctagagattttagaatttcgttacattttatatatagtgtcaaagatttgtcttatgaagttgtgatcttgaacttgctttgattgagtgatttagttactgtctttactatgtatcacttcttagaatctctaggcaaattggtgttaatcagattcaacagtctcgagttttcacagatcatgtcttatgtttttactaatttgtatcttgttcgttatggttgtagtgataactacagaagtgaaaaacattcttatacgcagcttctatcaacgagctgaagaaaaggtgaaacagctctcaactctcattcctcatagtttgtgattctttgattcaaatctccgttgtttccctcgtatatatagttcataacacaggatttcgtaggaaaatagacaaaagaaaacgatatagaacaatcttgaacttcttcagataacaaaactgttgattttggttgtgttatccgaaatcttaatttgttttgtcaaatttgtcaattgcagttaagaccaaagagaccggctacagatcatctggcagcggagcacgtgaacaagcatttccgtgctgcgtcttcttcttcatctactcagggtttataaaaaacttaagttcaagcctataacaatggtcatttgtatgagtacctcttattagtgttttcaatgtagaaaaaaaaagataagagcagttcatgagagaaatgtagtgaaaatgtgtgtgtacataacattaatgtttcttttatttcttgacttaaagttgctcactattcact

SEQ ID NO: 4 - protein, 101 aa, Arabidopsis thaliana
MASILGDLPSFDPHNFSQHRPSDPSNPSKMVPTTYRPTHNRTLPPPDQVITTEVKNILIRSFYQRAEEKLRPKRPATDHLAAEHVNKHFRAASSSSSTQGL

SEQ ID NO: 5 - DNA, 306 bp, Arabidopsis lyrata
atggcgtcgattctgggggatttaccttccttcgatcctcacaatttcagtcaacatcgtccctccgatccttctaatccctctaggatggttcctaccacctatcgtcctactcacaatcgtacacttccaccaccagatcaagtgataactacagaagtgaaaaacatacttatacgcagcttctatcaacgagctgaagaaaagttaagaccaaagagaccggctacagatcatctggcagccgagcacgtgaacaagcatttccgcgccgcgtcttcttcatcttctactcagggcttataa

SEQ ID NO: 6 - DNA, 418 bp, Arabidopsis lyrata
acgattccaatggcgtcgattctgggggatttaccttccttcgatcctcacaatttcagtcaacatcgtccctccgatccttctaatccctctaggatggttcctaccacctatcgtcctactcacaatcgtacacttccaccaccagatcaagtgataactacagaagtgaaaaacatacttatacgcagcttctatcaacgagctgaagaaaagttaagaccaaagagaccggctacagatcatctggcagccgagcacgtgaacaagcatttccgcgccgcgtcttcttcatcttctactcagggcttataaaaaacttaccctggtgagcctatgataatggtcattgtgtatgagttcttattagcattttcaatgtagagaaagaaagaaagaaagataagagcagttcatg

SEQ ID NO: 7 - DNA, 1416 bp, Arabidopsis lyrata
acgattccaatggcgtcgattctgggggatttaccttccttcgatcctcacaatttcagtcaacatcgtccctccgatccttctaatccctctgttagtttcttccccaattctcatcttcgattttttctttcttccaattcttcaactctcggatgaaaatttcagatgtcttctgattctgttttgtctgaattcgtatgagaactctttttatacagatctgagttcggtttactttggtcgtattgaaaaaaatgttgatccaattttgataattcaaattgttcaatgagaaagtaattcgaatgtgtatctaggtttgcttgtttacttgatctgatccaaaagttccatgttttcatcgattttgctgttggatcttgtgagaaacctagacaaaaagtagccatttttgacagatttattgatcttagaatcataattctgttcccaattggttcccattggccttaatttagctgctaagtgactaaggagtatgcaagttttcatttttcttgcaaattccaattagcttgtctcagttaaagattatatttttgatcagttatcgaagatttgagttttgttcatgttttcttgcagaggatggttcctaccacctatcgtcctactcacaatcgtacacttccaccaccagatcaaggtgaaacaaaattgcagttttttatttattttaaatcttgcgtgttcttcggctagagattttagaactttgttacattttgtagtctaaaattggccttttaaagttgtgatcttggctagagactgatcttgaacttgctttgattgagcaatctagttactgtctttaccatggatcacttcttagaatctctaggcaaattgttgttaatcggattcaacagtctccaattttcacgggtcacgtctatgtttttgtttgttttggttgtagtgataactacagaagtgaaaaacatacttatacgcagcttctatcaacgagctgaagaaaaggtaaaacaactctcattcctcatagtttgtgattctttgattcaaatcaccgttgtttccctcgtatatatagttcgtaggaaaatagacaaaagaaaacgatatagaacaaatcttgaacttcttcagataacaaatctgttgatttttggttgtgaattatccaaaatctttatgtttttttttgtcaaatttttcatttgcagttaagaccaaagagaccggctacagatcatctggcagccgagcacgtgaacaagcatttccgcgccgcgtcttcttcatcttctactcagggcttataaaaaacttaccctggtgagcctatgataatggtcattgtgtatgagttcttattagcattttcaatgtagagaaagaaagaaagaaagataagagcagttcatg

SEQ ID NO: 8 - protein, 101 aa, Arabidopsis lyrata
MASILGDLPSFDPHNFSQHRPSDPSNPSRMVPTTYRPTHNRTLPPPDQVITTEVKNILIRSFYQRAEEKLRPKRPATDHLAAEHVNKHFRAASSSSSTQGL

SEQ ID NO: 9 - DNA, 297 bp, Brassica napus
atggggtcgattctgggagatttgccgtccttcgatcctcataatttcagtcaacatcgtccctctgacccttctaatccctctaggatggttccaacaacctatcatccaacccacaaccgtactcttccacctccacatcaagtgataactacggaagtaaagaacatactcatacgcagcttctatcagcgagctgaagataagatgagaccaaagagaccggcttcagaacatctggccggtgagcacggtaacaagcatttccgtgcctcttcatctactcagggtttataa

SEQ ID NO: 10 - DNA, 596 bp, Brassica napus
ggtttctgagccggtccctgaggttaaacacgaggaagcggagaagaaacctagtctccttgagaagcttcaccgaagcgacagctcttctagctcctcaagcgaagaagaaggtgaagatggtgagaagaggaagaagaagaaaaaggataagaagaagattgctactgaaggagaggtgcaaacagaagaggcgaagaaagggtttatggacaagctcaaggagaagcttccaggacacggaaagaaacccgaagatgactcagccgttgcggctgcaccggttgttgctcctcctgtggaggaagcgcatccggctgagaagaaggggatcttggagaagattaaagagaagcttccaggataccactcaaagaccgttgaggaggagaagaaagatgatcactgaaaacatgaatactaatgatgatgagagacatctcgtgttgtttgtgatggatgattatcatctttttcttttgtgctgttgaagtttgttggcttctttatagtttattttgcagtttccctatttttctctttgttgtgtgtttagtgtatggtttcaaggtattttgaagttatgaattccttga

SEQ ID NO: 11 - protein, 98 aa, Brassica napus
MGSILGDLPSFDPHNFSQHRPSDPSNPSRMVPTTYHPTHNRTLPPPHQVITTEVKNILIRSFYQRAEDKMRPKRPASEHLAGEHGNKHFRASSSTQGL

SEQ ID NO: 12 - DNA, 300 bp, Brassica oleracea
atggggtcgattctgggagatttaccgtccttcgatcctcacaatttcagtcaacatcgtccctccgacccttctaatccctctaggatggttccaacaacctatcatccaactcacaaccgtactcttccacctccacatcaagtgataactacggaagtaaagaacatactcatacgcagcttctaccaacgagctgaagataagatgagaccaaagagaccggcttcagaacatctggcgggtgagcacgggaacaagcatttccgtgcttcctcatcatctgctcagggtttataa

SEQ ID NO: 13 - DNA, 396 bp, Brassica oleracea
atggggtcgattctgggagatttaccgtccttcgatcctcacaatttcagtcaacatcgtctatggggtcgattctgggagatttaccgtccttcgatcctcacaatttcagtcaacatcgtuccctccgacccttctaatccctctaggatggttccaacaacctatcatccaactcacaacctccctccgacccttctaatccctctaggatggttccaacaacctatcatccaactcacaacucgtactcttccacctccacatcaagtgataactacggaagtaaagaacatactcatacgcctcgtactcttccacctccacatcaagtgataactacggaagtaaagaacatactcatacgcuagcttctaccaacgagctgaagataag

SEQ ID NO: 14 - protein, 99 aa, Brassica oleracea
MGSILGDLPSFDPHNFSQHRPSDPSNPSRMVPTTYHPTHNRTLPPPHQVITTEVKNILIRSFYQRAEDKMRPKRPASEHLAGEHGNKHFRASSSSAQGL

SEQ ID NO: 15 - DNA, 300 bp, Brassica rapa
atggggtcgattctgggagatttgccgtccttcgatcctcacaatttcagtcaacatcgtccctccgacccttctaatccctctaagatggttccaactacctatcatccaactcacaaccgtactcttccacctccacatcaagtgataactacggaagtaaagaacatactcatacgcagcttctatcagcgagctgaagataagatgagaccaaagaggccggcttcagaacatctggcgggtgagcacggtaacaagcattttcgtgcttcctcatcatctgctccgggtttataa

SEQ ID NO: 16 - DNA, 300 bp, Brassica rapa; identical to SEQ ID NO: 15

SEQ ID NO: 17 - DNA, 2123 bp, Brassica rapa
ccacacatgttcttttggtacgtggataccttctcatgcaacaaatgatatgttatttttgggtggtttgaatgatttgtttaccgatctatagtcatttttttctgtgttttattcttaccatcacaagatcactttctgatacattattaattttaaaactattttgtcctcaaacgcttaatcctgaaatactacttcatctcaaacactcaactttaaaaatttatatgaatatataaccgaaagataagtattttagtgtcaaaatcatttttaagctttttccatgcactaatatatataaaaaaaaccgaaatgttataattttgtgttatagtatagttattcgaacaacaaaaaatttataactaccaataaaatttatagatatatacctttttaactcttaaaaataataaggtttataacaagtcataaactaaaccttaaaaatctaaattgataagatattcatataaaatgtttttttcaggctggttctagagtgagcccagcggaaagcccatataagtctttctattatctctacttcccttgcgattccgagaaggaagatcgcagagctcaaattccaaaatggggtcgattctgggagatttgccgtccttcgatcctcacaatttcagtcaacatcgtccctccgacccttctaatccctctgtaagtccttccctgatttttaactgtaatcattttgaatttggctaaaatcggatctgagaataatatgaattgagacttggtttctaaatctgattcaaatattgatattttcgaattttgtttcaatgagagattggaagaatgttactggggggcttgttttgggttacatattcatctattttttctattggacctttgtgaggaatcaagaaagtaatcgtttttttcttctaaccagattgattgatcttacatgttgttccattctcattttgattgcatgttgttgttaaattaacaatctttttttttgtatgatcttacagaagatggttccaactacctatcatccaactcacaaccgtactcttccacctccacatcaaggtatacccaaaacaaagtctaactactttacatagtttaacttttgtctttattgagtgatcttattatagacaactttgaaggactatctttaccaggaatcactttttttttgttcgtagagagtattgagtcttcatggattatagatagttcattcctagtttgctacggtttatattctcttcttctttttgtgtcagtgataactacggaagtaaagaacatactcatacgcagcttctatcagcgagctgaagataaggtactaaatttctaaaattcaacatgtgcggtatagaacaagtcctcaaactcttttcttttttttttttgttttttgcgaaaagcagatgagaccaaagaggccggcttcagaacatctggcgggtgagcacggtaacaagcattttcgtgcttcctcatcatctgctccgggtttataaaaagcttctcctgcttccaaagcctggctataatggtcgtcacttgtgctactcttcttattagtgttttttttacaaagaatgctttgaatgtagagagaaaagatgagagcagctcactatgtgattgcagggaaaatgttgtatgagttatatgtacataacatttatgtttttatttttaattttaaaatattcgtccaaatcacattgttagcttttgtctcttctagaatgtaaactgaattctttgtttgcttccaacgaattacaagaggaatctgagagtagtggcactcactagccatagtgatatccccgactttttgttcgcatgtttctcctcaccttgtaacgagcacaagtacttgttatacactgcagaccattttccatgatttattttttgttgatgatgtgggtttctacaggagctgtttccttgaagtggatcctaagctagatcccttagttgaagaggatgagcgacactatatctgaattcatttgcaagggaaatgatgaccaggtgattgaattgtggtgtacagaccatggcagagaaagaagaagaagaagacggcctggtgaaatctaatg

SEQ ID NO: 18 - protein, 99 aa, Brassica rapa
MGSILGDLPSFDPHNFSQHRPSDPSNPSKMVPTTYHPTHNRTLPPPHQVITTEVKNILIRSFYQRAEDKMRPKRPASEHLAGEHGNKHFRASSSSAPGL

SEQ ID NO: 19 - DNA, 309 bp, Capsella rubella
atggcgtcgattcttggagatttgccttccttcgatcctcacaatttcagtcaacatcgtccctctgatccttctaatccctctaggatgattcctacaacttatcgtcctacacacaaccgtacacttccaccaccagatcaagtgataactacggaagtgaaaaacatacttatacgcagcttctatcaacgagctgaagagaagttgagaccaaagagaccggcttcagaccatctggcagccgagcatgggaacaagcatttccgcgctgctgcgtcttcttcgtcaactactcagggattataa

SEQ ID NO: 20 - DNA, 503 bp, Capsella rubella
tcttgaactctgaagagaaggaaacattatcacgcacaccacgattccaatggcgtcgattcttggagatttgccttccttcgatcctcacaatttcagtcaacatcgtccctctgatccttctaatccctctaggatgattcctacaacttatcgtcctacacacaaccgtacacttccaccaccagatcaagtgataactacggaagtgaaaaacatacttatacgcagcttctatcaacgagctgaagagaagttgagaccaaagagaccggcttcagaccatctggcagccgagcatgggaacaagcatttccgcgctgctgcgtcttcttcgtcaactactcagggattataaaaaactttccctgcttcaaagccttgtaagttgtaacaatggtcctttgaatgtgttcttattagtgttttcaatgtagagaaaaaagataagagcaattcacgagtggaatgtacataacattgatgtttcttttatattttac

SEQ ID NO: 21 - DNA, 1554 bp, Capsella rubella
tcttgaactctgaagagaaggaaacattatcacgcacaccacgattccaatggcgtcgattcttggagatttgccttccttcgatcctcacaatttcagtcaacatcgtccctctgatccttctaatccctctgttagttctttcttccccctaattctcactctttcatcttcgacttcttttttttttagttccttttcttcgcttcttagatgaaagtgcttagctgttctatgatctgtgttttgtctgaattcgtctgagaactcttcaaactattcaattctacggatctgagttcgttttacttggtcgtattgtgaaaaaaatatgtatcttttgttgatcaaaatttgatattcaaattgtttaaagagtatacattcgaatgtgtgtatttggttttgctggttacttgatctgatccaaaagttcttctttttcattaatcttgtgagaaaccaagaaaagaaaaaaagccatttttggtcttgctgaatgatcttagaatcataattttgattcaattggccttagttagctcctaagttatatgacaagatcttgttatctcatatcaacttactagtatgaaacttttcatttttattttattttgattgagcgaaagattagatttttgatctttaaattaaagatttgagctttgttcatattttcttgcagaggatgattcctacaacttatcgtcctacacacaaccgtacacttccaccaccagatcaaggtgaaacaaagaatcgcggtttttttaaattttgcatgttcttaggctagaagattttcgaattttgtttacattttatagtgtctaaaattggccttttgaagttgtgatcttggctagagactgatatttgaacttgctttgatagagcgatctagctactctgtttaccatgaatcacttcttagatctctaaggcaactgggagttaatcagattacaatagtctcgagttcccacacatctacgcctttactaatgtgtaccttgtttgttttggttgtagtgataactacggaagtgaaaaacatacttatacgcagcttctatcaacgagctgaagagaaggtaaaccaactctcattcgtctttaagttcttgattcttttatccaaatcatttccctcatatagtcttataacacaggatttcttaggaaaacagacaagaacgatatcgatcaaatcttaaacttcttcagataaccaaactgttggttttgattgttttatcctaaatctcagttccttttttttttgtaatatttattatttgcagttgagaccaaagagaccggcttcagaccatctggcagccgagcatgggaacaagcatttccgcgctgctgcgtcttcttcgtcaactactcagggattataaaaaactttccctgcttcaaagccttgtaagttgtaacaatggtcctttgaatgtgttcttattagtgttttcaatgtagagaaaaaagataagagcaattcacgagtggaatgtacataacattgatgtttcttttatattttac

SEQ ID NO: 22 - protein, 102 aa, Capsella rubella
MASILGDLPSFDPHNFSQHRPSDPSNPSRMIPTTYRPTHNRTLPPPDQVITTEVKNILIRSFYQRAEEKLRPKRPASDHLAAEHGNKHFRAAASSSSTTQGL

SEQ ID NO: 23 - DNA, 297 bp, Thellungiella halophile
atggcgtcgattctgggtgatttgccttcctttgatcctcacaatttcagtcaacatcgtccctccgatccttctaatccctctaggatggttccaactacttatcatcctactcacaaccgtacactaccaccaccagatcaagtgataactaccgaagtcaaaaacatacttatacgcagcttctatcaacgagctgaagaaaagttgagaccaaagagaccggcttcagagcatctggcgggtgagcacgggaacaagcatttccgtgcttcatcatctactcagggattataa

SEQ ID NO: 24 - DNA, 559 bp, Thellungiella halophile
gaagaagagaaggaaacatccgcaaattcgaaatggcgtcgattctgggtgatttgccttcctttgatcctcacaatttcagtcaacatcgtccctccgatccttctaatccctctaggatggttccaactacttatcatcctactcacaaccgtacactaccaccaccagatcaagtgataactaccgaagtcaaaaacatacttatacgcagcttctatcaacgagctgaagaaaagttgagaccaaagagaccggcttcagagcatctggcgggtgagcacgggaacaagcatttccgtgcttcatcatctactcagggattataaaagctttcaaccctataatagtcatgttgtatgagtcttttgttagtgttcttattagtgggttgtttacaaagtgaatgcctttaatggtagaaaaaaagataagagcagctcttgtaacaggattgtagagaaaatgtgtatcctgaaacagagtgttcctcatagactttggtggaaacatgctgtttgtattttcctgtacagttacattcaaatattccttttga

SEQ ID NO: 25 - DNA, 1452 bp, Thellungiella halophile
gaagaagagaaggaaacatccgcaaattcgaaatggcgtcgattctgggtgatttgccttcctttgatcctcacaatttcagtcaacatcgtccctccgatccttctaatccctctgtatgttcttccctctattttcagtcaaacactctcattttttcggtgaatttgggtagaatcggatctgagttcgatttaattagttttcctgaaaattgtggattgagtctcagtttccttaatctggaatctgatccaattttgatcatgttggatcctgtgagaaaccatcaaaccaagaaagtagcaatttttttttctcaccagatttgatttctgaatcagaattctgtttccactggccttaattagctgtttaattacttaattctgttatctcatatcagatgaaaagttttcatttttattgcatataacagttgaatttaaaagagcttaaagattaggaattttttttaatatctgttatcgaagattttgagtttttgttcaatgttttttttttttgaaacagaggatggttccaactacttatcatcctactcacaaccgtacactaccaccaccagatcaaggtaaaataaaaaagtcaccttttttttggtcttgccttcttcagtaactacgttatatatagtctacatgtggccttgaacttgtgatgttggccagaaactgatcctgaacttgtattaagtgagttatctgattatgaagaactatctttcacatgattcacttcttagaatagactcggagatttgaactcgtgacaaatgggtgctaatcaaatacaatatagtcttgagttttatggatagctcattgctgattttctagatttcatctctttattccgttgattttttgtggcagtgataactaccgaagtcaaaaacatacttatacgcagcttctatcaacgagctgaagaaaaggtgatagagattcaatcctcaaagtttcttaattttttttatttatttgaatcgttcatcctcctcgtagttcataacaaagctagtttcttaggaagaaatttgttttaccgtgtgagatatagaacaagtgttaaactctgttttttgtgggtcaaattttgtcatttgcagttgagaccaaagagaccggcttcagagcatctggcgggtgagcacgggaacaagcatttccgtgcttcatcatctactcagggattataaaagctttcaaccctataatagtcatgttgtatgagtcttttgttagtgttcttattagtgggttgtttacaaagtgaatgcctttaatggtagaaaaaaagataagagcagctcttgtaacaggattgtagagaaaatgtgtatcctgaaacagagtgttcctcatagactttggtggaaacatgctgtttgtattttcctgtacagttacattcaaatattccttttga

SEQ ID NO: 26 - protein, 98 aa, Thellungiella halophile
MASILGDLPSFDPHNFSQHRPSDPSNPSRMVPTTYHPTHNRTLPPPDQVITTEVKNILIRSFYQRAEEKLRPKRPASEHLAGEHGNKHFRASSSTQGL

SEQ ID NO: 27 - DNA, 285 bp, Glycine max 1
atggattctctgattggtaattggccatcctacgatcctcacaacttcagtcagcttcgaccttccgatccttctagttcttctaaaatgatgccggccacttaccatcctactcacaacaggactcttccagcacctgatcaagtgataagtactgaagccaaaaacatcctcatgagacatatttatcagcattctgagcagaagttgaatccaaaaagagctgcatctgataaccttcttttaccagagcatggatgcaagcaacctagggtttccagctga

SEQ ID NO: 28 - DNA, 1038 bp, Glycine max 1
ggatttttcagcttttcattttgccccaccttctcctttcatctcacaatcataacttgagttgagcacgttcccgagacatctcaaatttcctctgctgagaatttcacaagtttatgagccacaagtgcaaatgggaatgaaatgaagaatggggtttagagttagtcaagacaagtggtgtggtgtcctatgttatgactgcacacatgtgaagtgaagtagagactattcagtccacagcagctgtttctagtgtgtgtgtcattgcattcctcatccttttcctcttttttcacgccttaatttctttctctctttctccctcttcctctctggaatttggagcatcagccagcactctatggattctctgattggtaattggccatcctacgatcctcacaacttcagtcagcttcgaccttccgatccttctagttcttctaaaatgatgccggccacttaccatcctactcacaacaggactcttccagcacctgatcaagttccaacagtgataagtactgaagccaaaaacatcctcatgagacatatttatcagcattctgagcagaagttgaatccaaaaagagctgcatctgataaccttcttttaccagagcatggatgcaagcaacctagggtttccagctgacacttgcccgtttccattgacctttgtgatcgtgagcaagtttccaaaagatcagaacttacaagagtgaacttacaagagtttggtgcttgtttggaactctgaactattttggcagctatatagacttgggagcgtgtgaaaataaaccctgttaatgcccatcaaagtttaccatgaatgaaataatgatatgcttttgttgtttatttttgtgtcatgcttgttgtactctacccacaactgattgtccacaatagttgggaagagataagtgcttgattgaggattttcaaaatctatctatcttttggactcaggagcatattctggggccataatgttctccatctaactacaattatttatatggcgctttt

SEQ ID NO: 29 - DNA, 3509 bp, Glycine max 1
ggatttttcagcttttcattttgccccaccttctcctttcatctcacaatcataacttgagttgagcacgttcccgagacatctcaaatttcctctgctgagaatttcacaaggtaatgtctcacactcacaccgattttttgcacccaactttttgtgctgtgtttagattctgattatttctcacgcttccacatatgcccccattgctgttttccaattggtttctcctaccagtaccaggtggtttattcaaccaatagtggatcttcaatttattggtttcagtgttcttgtatgtgtgtgtgttttcattttgatgaattcatggtaagcctaataatatgtattctttattaattgataattaaagttcagatttttggtgaattttcatacattgggaagatagttttaggtaattttctcattgtttttaaaagggtgttcacttgtcccttttccatgaattgcagtttatgagccacaagtgcaaatgggaatgaaatgaagaatggggtttagagttagtcaagacaagtggtgtggtgtcctatgttatgactgcacacatgtgaagtgaagtagagactattcagtccacagcagctgtttctagtgtgtgtgtcattgcattcctcatccttttcctcttttttcacgccttaatttctttctctctttctccctcttcctctctggaatttggagcatcagccagcactctatggattctctgattggtaattggccatcctacgatcctcacaacttcagtcagcttcgaccttccgatccttctagttcttctgtaagttgctgttgttgttgtctatgaaattgataatcctggtaataattactcattccaagtatgcaaatgtatgctttgttagagcatgtttggttttctgttgcaaaagtatcgtttgaggcaaaattaattttgttaacgaaccaagacccttgcttttgcgttgaatttgcattggaatgtgtgtcaacatttccaactgaaaaccaaacatgcactcaattgttgaaaaatctgattgtgtatgattctgagtttttggtttactactatctatttgcattgatcccggttgaaagattagtgaaggaagggatattagcctctttttcttaatttcccaacccccttgaggacaaaatttggttgttgtccataaatataagaacttcttctacttgcaactttgaatcccactattcaaatttgagaatatttgttaaatttttaagatggtacatggtagagtcagacattagagatgtgtagattagaaaacctgtatttgcaatctttagaattttatgtttgcttcttttttgtcaaaaggtagagtatttgttgacattctaactgttattactacttggatggtcataattcacaatagagtaacagcattgtgcaatttgttgtgttgaagtcttcaccattgatgttatatgcagcattgtctgtgggaacatgtccatttaatctcaataactatactgtttttctttcttcgctattctgcaaagtttctgtaaaggattttaaagtttgaaacttttccagcatgggatgattttctttttccttttttgtttcttgtcattttttactctgaaagtagtactgtataacttggctttctgttcaatattgtaacaacaattatcttcagtgacaaaagtgttttacttttatttctaattagttgaaaaaactagagttatatgtaatttgtcctttacagcatatcagtttccatgatagcctgacacaaatttagttgttttcacagtaccttctgatgaatgtagtcgttcttgtctttacaatagattttctaatttactctattattgctaatctataaaactacaagcaaatacatattgcccatgaaatttgaaaatgaaggagaccattggattcatttgatacctttgctgaagcgtacaagtatgtttacaccatacatacaaattctggttttatgttttttaacaatagataacgtgtttcttctctagttgattacatttgataagaaggaagctcaagtatgagtatgacagatttgatcttggagataaacatactgaatgggtcactgttattccttaccttcttgctaaccaagtcatgaataaaccaagcatatttaaggccaacttctttttatgaaacttgctactcattttgttctaaaatgtattaaggatcacctaatgatatattttcattcacactttgaagatgaaatttttattttaacacagaaaatgatgccggccacttaccatcctactcacaacaggactcttccagcacctgatcaaggtagtcaggataactttcttctactaatgctttgctctgtatttatttgattgataatctgttgaaaaactgtattattcctttggattattcccttcatcatgtttttggaggagtatccaaactggatgcattaaatctattaattttttgtttgccacacacaagtttatagtagagcttctccaatgtatatgaactcaaattgggttcttaactctcacatagtgggtgcctgttgccacataggatttaagaacactcactattttcactccgaagttaaaactcaacttgagttcttaacttattttttagtctcatcttatctctctgtttacacatggaccccattttgacttggaaagttattattttaatggttaagcaacctttttgtagaagggttcttctataataactctagttcattttctccaatgttatttttcaagttgcttaacttttgtttttttaatttcggtagactcaatggtgttttctaattcaaagtttgctaacctgttgaagttccaacagtgataagtactgaagccaaaaacatcctcatgagacatatttatcagcattctgagcagaaggttagtgacttgatggttaaagagcatgtgttttggtgcagttaggtatgcatatgcttgatgctcataaccttttgtgtttttcagttgaatccaaaaagagctgcatctgataaccttcttttaccagagcatggatgcaagcaacctagggtttccagctgacacttgcccgtttccattgacctttgtgatcgtgagcaagtttccaaaagatcagaacttacaagagtgaacttacaagagtttggtgcttgtttggaactctgaactattttggcagctatatagacttgggagcgtgtgaaaataaaccctgttaatgcccatcaaagtttaccatgaatgaaataatgatatgcttttgttgtttatttttgtgtcatgcttgttgtactctacccacaactgattgtccacaatagttgggaagagataagtgcttgattgaggattttcaaaatctatctatcttttggactcaggagcatattctggggccataatgttctccatctaactacaattatttatatggcgctttt

SEQ ID NO: 30 - protein, 94 aa, Glycine max 1
MDSLIGNWPSYDPHNFSQLRPSDPSSSSKMMPATYHPTHNRTLPAPDQVISTEAKNILMRHIYQHSEQKLNPKRAASDNLLLPEHGCKQPRVSS

SEQ ID NO: 31 - DNA, 282 bp, Glycine max 2
atggattctctgcttggtaattggccatcctttgatcctcacaacttcagtcagcttcgaccttccgatccttctagttcttctagaatgacgctacccacttaccatcctacgcacagcaggacccttccagcacctgatcaagtgataagtacagaagccaaaaatatcctcgtgagacacatttatcaacatgctgaggagaagttgaaaccaaaaagagctgcatctgataaccttttgcctgatcatggatgcaagcaacctagggtttctagttga

SEQ ID NO: 32 - DNA, 1183 bp, Glycine max 2
gttgtaagggtggctggaattttcagcttttcattttgcccccaccttttcctttcatttcactatcattacttgagttgagcacgttcccgagacatctcaaatttcctctgctgagaatttcacgagtttatgagccacaaatgcaagtgggaacgaagaatggggtttggagttagtttggacaagtggtgtggtgtggtgtcctatgtaatgagtgcacatatgtgaagtgaaaattcctcatccttttcctcttttttcacgccttaatttctctctctctggaatttggagcaacagccagcactctatggattctctgcttggtaattggccatcctttgatcctcacaacttcagtcagcttcgaccttccgatccttctagttcttctagaatgacgctacccacttaccatcctacgcacagcaggacccttccagcacctgatcaaggtaacacgacgaactttcttatgattcctccagcaaattagccatgctttgtcccgtatttacttgactgatcattttttggaaaaatgtatttttcctttggattattaccttcatcatgtttttggaggaaaatccaaactacatgcattaaacctgttacttaaactgattttctgtctagctcaagtcttgttagggtagactgactggtgttttgtaattgaaagttttgcttgaaaattgttcaaacagtgataagtacagaagccaaaaatatcctcgtgagacacatttatcaacatgctgaggagaaggttagtgactcttgatggttaaacacacatgtgttttggtgcagttaggtttgcacatgcttgatgcccataaccttttgtattttttcagttgaaaccaaaaagagctgcatctgataaccttttgcctgatcatggatgcaagcaacctagggtttctagttgacagtggccatcaaccaactgttgtatgtgattgtgagtaagtttccaaaaatatatccttagaagagtttggtgcttgtttagaatttgaacgattttggccagttatatagacttgggagtgtgtaaacataaacctaataatcagtcaaaatttaaactgaatgaaacaatgacgatttcattgtgtatgtttatgccatatcattaacaactgatcgtgcaagtatcttttgtactcgc

SEQ ID NO: 33 - DNA, 2809 bp, Glycine max 2
gttgtaagggtggctggaattttcagcttttcattttgcccccaccttttcctttcatttcactatcattacttgagttgagcacgttcccgagacatctcaaatttcctctgctgagaatttcacgaggtaatgtctcgcactcaccatttttttgcagccaacttttttactgtgtttggattctgattatttctcacgcttccacatatgcccctattgctgttttccaatttggtttctcctaccagtatcaggtggtttattcaaccaacagtggatcttcaatttgttggtttcagtgttcttgtatgtgtgttttcattttgatgagttcatggtgagcctaataatctgtattctgtattaatttgataattaaagttccaatttttagtgaattttcatacattggaaagatagttttagggaattttctcattgtttttcaaaagggtattattcacttgtcccttttccatgaattgcagtttatgagccacaaatgcaagtgggaacgaagaatggggtttggagttagtttggacaagtggtgtggtgtggtgtcctatgtaatgagtgcacatatgtgaagtgaaaattcctcatccttttcctcttttttcacgccttaatttctctctctctggaatttggagcaacagccagcactctatggattctctgcttggtaattggccatcctttgatcctcacaacttcagtcagcttcgaccttccgatccttctagttcttctgtaagttgttgttgtatatgaaattgataatcctagtaataattactcattccaagtatgcaaatgtatgctttctcaattgttgaaagtctgattgtgcgatgctgagtttttggtttaatgctttctatttgcatcgatcccccgttgaaatatcagtgaaggaagggatatttgccttatttcccaacccccttgaggacaaaatttggttgttaacaaccttcttcttgttgacacacttgcatatttgaattccaccattcaaatctgagaatatttcttcattttttaagatggtaagtggtagagttagactagagaagtgtagaatagaaaacctgtatttgcaatcttcagaattttaagtttgcttcttgttgtcaaaaggtagagtatttgttgacattctaaatgttattgctactacttagaatgatcataattcataatagagtaacagcattgtgctatttgttgtgcccgagattgaattcttcaccattggtgttatatgtagcatgtatgtgggaacatatccatttaatctcaataactttactgttattctttttttatttttcctctttattttgtgcggttctgcaaaagtttccgtaaaggattttaaagtttgaaacttttccagcatggggtggttttctttttcctttctgtctcttttaatttgtgactctgaaaattgtactgcaaaacttgactttgctgttcaatattgtagcaacaattaattcttcagtgacataattgttctacttttatttctaatcagtctccgtgttaccttgacacaaatttagtcgttcttgtcttttccatagatttgctaattgctaatctacaaacttacaagcacatacatattacccatgaaatttagcggttgaaaattgaaaaaatgaaggagatcattggattcatttggtacctttgcttacaccgtgcatacaaattcttgttttatgtttaacaatatgtgatatgtgatatgcttctctaatccattgcatttgataagaaggaagctcaagcatgagtcagctttgatcttggaggttaacacactgaatgagtcacatttattccttacctttttctaatcaagtctgggataaaccaacatatttaaggccaacttctttttatgcaacttctattcttgttctaaagtgcatcatgggtcacctaatgatatataattttataagcgttatgaagatgaaatttctctaatttcatacagagaatgacgctacccacttaccatcctacgcacagcaggacccttccagcacctgatcaaggtaacacgacgaactttcttatgattcctccagcaaattagccatgctttgtcccgtatttacttgactgatcattttttggaaaaatgtatttttcctttggattattaccttcatcatgtttttggaggaaaatccaaactacatgcattaaacctgttacttaaactgattttctgtctagctcaagtcttgttagggtagactgactggtgttttgtaattgaaagttttgcttgaaaattgttcaaacagtgataagtacagaagccaaaaatatcctcgtgagacacatttatcaacatgctgaggagaaggttagtgactcttgatggttaaacacacatgtgttttggtgcagttaggtttgcacatgcttgatgcccataaccttttgtattttttcagttgaaaccaaaaagagctgcatctgataaccttttgcctgatcatggatgcaagcaacctagggtttctagttgacagtggccatcaaccaactgttgtatgtgattgtgagtaagtttccaaaaatatatccttagaagagtttggtgcttgtttagaatttgaacgattttggccagttatatagacttgggagtgtgtaaacataaacctaataatcagtcaaaatttaaactgaatgaaacaatgacgatttcattgtgtatgtttatgccatatcattaacaactgatcgtgcaagtatcttttgtactcgc

SEQ ID NO: 34 - protein, 93 aa, Glycine max 2
MDSLLGNWPSFDPHNFSQLRPSDPSSSSRMTLPTYHPTHSRTLPAPDQVISTEAKNILVRHIYQHAEEKLKPKRAASDNLLPDHGCKQPRVSS

SEQ ID NO: 35 - DNA, 282 bp, Phaseolus vulgaris
atggagtctgtactgggtaattggccgtcctatgaccctcacaacttcagtcagcttcgaccttccgatccttcaagttcttctaaaatggcaccggccacttaccatcctactcacagcaggacccttccaccatctgatcaagtgataagtactgaagccaaaaatatcctcctgagacatatctatcagcatgctgaggagaagttgaaaccaaaaagagcagcacctgataaccttttaccagagcatggatgcaagcaacctagagtttccagctga

SEQ ID NO: 36 - DNA, 994 bp, Phaseolus vulgaris
cggaggtgatgagtagctccaaatgatgatcagttggtaatggtggctgcaattttcagcttttccttttccttttctttcacttctcaaccaaaccataacataacttaacttaactttatcacattcttcatagatctgaaatcccttctcagaatttcacaggtttaccagcatcctgtgcaagtgggaatgaagaattgggtttagagttaggacaaggggtgtggtgtggtatcctatgcaattggtgcacacatgtgatgtgaagttcagtccacaacagctgtttttggattgggttttgtgttgtgtgtcattgtcttcctcatccatttcctctcttttttcacgccttaatctctctctctgaaatttggagcagcaaccgccactctatggagtctgtactgggtaattggccgtcctatgaccctcacaacttcagtcagcttcgaccttccgatccttcaagttcttctaaaatggcaccggccacttaccatcctactcacagcaggacccttccaccatctgatcaagtgataagtactgaagccaaaaatatcctcctgagacatatctatcagcatgctgaggagaagttgaaaccaaaaagagcagcacctgataaccttttaccagagcatggatgcaagcaacctagagtttccagctgacacatgtcattgaccatatgttgcatgtgattgtgaactactttcctatagatatacccttatttttcaagagagtttggtcctagtttcaaattgtgaactatttgccaattatacactggggagttttgtaaatacaaagcctgttattgcccatcaaaatttacagtgaacgatatttttgtgccatgccttattgtgctagacaggtaacaactgattgtccacattagttaggaagagattcgtgctttagttaaagattttcaaaatgcatctgagtcttttggactcaggagtatgcttgtgccata

SEQ ID NO: 37 - DNA, 3323 bp, Phaseolus vulgaris
cggaggtgatgagtagctccaaatgatgatcagttggtaatggtggctgcaattttcagcttttccttttccttttctttcacttctcaaccaaaccataacataacttaacttaactttatcacattcttcatagatctgaaatcccttctcagaatttcacagggtaatttattgtctcacactcaccaatttttctactgtcttccgactcagattatttctaacgcttacacttctcctgttagtgtttttccaatatctgcttcattcaacaaatactggatcttcaatttttttgttttcagtgttcttgtatgtttgtgttttcattttgacgacttcatcagtgagcctgtgtattgattcataatctgatatagttcagagttctggtgaattttatttctcttgcattgggaagatgatgttagggatttttctccttttttctttaattggaattcacttacccctttttcttaatttgcagtttaccagcatcctgtgcaagtgggaatgaagaattgggtttagagttaggacaaggggtgtggtgtggtatcctatgcaattggtgcacacatgtgatgtgaagttcagtccacaacagctgtttttggattgggttttgtgttgtgtgtcattgtcttcctcatccatttcctctcttttttcacgccttaatctctctctctgaaatttggagcagcaaccgccactctatggagtctgtactgggtaattggccgtcctatgaccctcacaacttcagtcagcttcgaccttccgatccttcaagttcttctgtaagttgttgtagtataagaaattgataatcctggtaataactcattttgatgcaaatgtatgctttgtcacttgttgagaaatctgattgtttgtgattctgaatttttggtttactactgtctaatctacaattatttgggttggaaatttagggaagagacaattgtcttgttttttattcttatccctcttctcccttcaggacaaaatttggttgttaaccttcaagataagactttcttctagttaacacacttccatatttgaatcccatcttctaattttgaaactgttcgtcaatgttttaagttggtacatggtagagaagttatagtgtagaagaacatgtatttgcaatcgttacatttttagtttgtttctttttatcgcaagggacagagtatttcttgacattctgattgttattactaattcataataggcttatggcattgtgcaatttgttgttcacctgacgttgaagtcttcaccattgatgttatatgccgctttgtctttggggacatatccatttagtctcaataacttcactgtttatcctttttccccctctacatcttttttgcattctgcaatatattcataaatttgaaagttttaaacttctcaagcatgggatgatttgctgtttcctgttgtttcttgttctttttcactctaaacataatgctgttaaatttggttttcttgttcaatattgtagcaacaataaattcttctatttaaaataactatttcacttttttaaaaatagttagaaaggctagttttatacgtaatttgtcctttatattataagaaatatctttctagctttcaggcatatattagtttccattagcctgacacaaatttagttgcttcaaaataccttttgctgaatgtagtttttcttttcctttccaaagattcttagattcgctaatccataaaagtatgattccactatacgtacatgcatacataattcttgttttatgttaaacaaaggtaagatgtttctctattcaattatatttgataagaaagaagctcaaataagagaatgactggtgtgatatcgaagataaacacacggaatgagctactgttattcatttcctttttcctaaccaagccatggataaacaaagcagttttagggccaacttctttgtgcatacatggattgaagtttgcaacttaagtttgaatgaactttatgttacgaactaagttggtaaaacaactttgtcttttcttccatttcaaactaaatttcaattcagacttgaggcaaaactcagtttacaatcctacaacctgaaaatcaagcaagagattttctcagatttagtttttgaatgcatgttaggggtctctcaactgaaaactaaacgtgccttttttgtacctctggcactcttattttttctgaagtgtactatggatcacttaatcatatattttcatttgctctctgaagatgaaacttctattttcatgcagaaaatggcaccggccacttaccatcctactcacagcaggacccttccaccatctgatcaaggtagtcagtataactttttctacttccgtggcaatgctttgctctgtttttatctgatcgataatccgtttgaaaaatgtattttcctttaatcctgatcttggaggaggagccaaactagatgcattaaacctattactttaacttatttttctctgtttgccacacacaaaaagttttgtgtatatagaaaggtaacaaacatgaaaaacattggttgcaggtttttagtttttctttttttgacagatgtacatccctcattttctgtcactcttgtttttaatttgggtagagttattactgggtgttttgtaactgagggtttgctaacttgctgaaatttgttctgacagtgataagtactgaagccaaaaatatcctcctgagacatatctatcagcatgctgaggagaaggttagtgactcttgaaagttaaatccaatatgttttggtacagttaggtcagcacatggttgtacacattcctaccctccactcatcagcccacagggatctaatagatgtcaatgatctttcatcttttcagttgaaaccaaaaagagcagcacctgataaccttttaccagagcatggatgcaagcaacctagagtttccagctgacacatgtcattgaccatatgttgcatgtgattgtgaactactttcctatagatatacccttatttttcaagagagtttggtcctagtttcaaattgtgaactatttgccaattatacactggggagttttgtaaatacaaagcctgttattgcccatcaaaatttacagtgaacgatatttttgtgccatgccttattgtgctagacaggtaacaactgattgtccacattagttaggaagagattcgtgctttagttaaagattttcaaaatgcatctgagtcttttggactcaggagtatgcttgtgccata

SEQ ID NO: 38 - protein, 93 aa, Phaseolus vulgaris
MESVLGNWPSYDPHNFSQLRPSDPSSSSKMAPATYHPTHSRTLPPSDQVISTEAKNILLRHIYQHAEEKLKPKRAAPDNLLPEHGCKQPRVSS

SEQ ID NO: 39 - DNA, 282 bp, Medicago truncatula
atggattctgtccttggtaatttgccatcttataaccctcacaatttcagtcagattcgaccttcagatccttctagttcttctaaaatgacaataactacttaccatcctactcacgacaggacccttccaccacctgatcaagtgataaacactgaagcaaaaaatattctcctaagacatatttatcagaacgctcgggaaaagttgaaaccaaaaagagctgcagctggtaaccttttaccagaacatggatgcaagcaacctagggtttccacctga

SEQ ID NO: 40 - DNA, 909 bp, Medicago truncatula
ttaacgagttcatgtgtaggatacagttggttttggtgaccaggttaaacgggtcggattcgtgaaagtggatcattgattggagggtaaaccttacttggtcatttcagtccttagtggtctagtgtttttttctcttcttaactcgcggttgtaacagcagtatgaatactcacccctggtaaaaatgatcgtaactacatagctggccgaaagagcaaaagttttcatcttttttctctcatgttgaggaggacaacgttccagagagatctcaataactaattcataattactccactagggtaatattgcctaacgcttattcatgttcatgatttttcaatttttttttctcactcttttgatttgttttgtgcctttgaaattttgatcaaataggtagcaattcatggattctgtccttggtaatttgccatcttataaccctcacaatttcagtcagattcgaccttcagatccttctagttcttctaaaatgacaataactacttaccatcctactcacgacaggacccttccaccacctgatcaagtgataaacactgaagcaaaaaatattctcctaagacatatttatcagaacgctcgggaaaagttgaaaccaaaaagagctgcagctggtaaccttttaccagaacatggatgcaagcaacctagggtttccacctgacagtgttcattgaccaactagtgcatgcagttctcagctactttctcgaatgatatatactcttattttattaccaagattttggtgcttgtttgaaattgtaaactattattagtccgctacatacttggagtgtgtaaatttgacaactcccccaccaatcaatcaatatatacaacaactgaaattatcatgcttttattgtgtatattttt

SEQ ID NO: 41 - DNA, 3361 bp, Medicago truncatula
ttaacgagttcatgtgtaggatacagttggttttggtgaccaggttaaacgggtcggattcgtgaaagtggatcattgattggagggtaaaccttacttggtcatttcagtccttagtggtctagtgtttttttctcttcttaactcgcggttgtaacagcagtatgaatactcacccctggtaaaaatgatcgtaactacatagctggccgaaagagcaaaagttttcatcttttttctctcatgttgaggaggacaacgttccagagagatctcaataactaattcataattactccactagggtaatattgcctaacgcttattcatgttcatgatttttcaatttttttttctcactcttttgatttgttttgtgcctttgaaattttgatcaaataggtagcaattcatggattctgtccttggtaatttgccatcttataaccctcacaatttcagtcagattcgaccttcagatccttctagttcttctgtaagttatcattattttcctttaattttatcaaaatatagttatttgataatcttggtagagcttattcaaccatgcaaatttatattgtattattctgattgtgtatgatcctgtgtttttatattgctaatgctaccctatttatgagtcccaccttaaatgagataaggtctgaacatgggtctatgcaatcctcaccttataagccggtcttgtaggtttatttaatttaatttggaccaattcaaaattataatatggtatcacagcctatgcaaaatccgtcgggttacctgctatcagatcaccactttcaaaccactcggggcttcaagttgtcaaccagcaaggccgggttataatcagtgttaagaattagcattaagctatagtacatgagcttggagctgatgtacctttaccgagggtgtgtttggttctagggtgacaaaaattgattttgactaaattgattttacaaaattgactttggttggaagtgaattgaaggtaaaacgagttatgtttggatacattcattaaaaaaattaatttttatcagtttatgttttggatcagaattgcttttttgtggctttatttgtcaaaaaaattggcaatttattttatcttaccacggtaactagaaatattagcttttaggtagattgattttggggctggattcgattttaaagctataagttaaacataacaatttatttgtcaaatctagtcaaatttgattctgggaggtacaaacatggaaccaaagacaggttaaaatgtactatatgcttaaccaagcaatacatgcatagagagacttgtcatacagcttatcttatcctagtaatacttttctcccactcccttgtgctttccttactctctttacataattggcagagtatttcttcaatttttaagtttatactttgcagagcttgagatgaaaaacttatatttgcaaactttaggaatttaagtttgtttgttttattccatggaggagtatctcgtgacatgttgattggttttccaagttacttggatgaccataacttataatagaataatgatgtggtggtgcaatttgggtcagccctattgttgtatgcagcattgtcttttagaagatatccttttcattggaataacttaaactgtttgtcaaaataagcgtaaaggaattgaatgtttaaaacttttcaaacatggcatgatattctttttttcccttagttactactcatttcattctaaaactgatactgtgaatttaactttcttttccaaattgtagcttagataaattcttcagtaacagaagagttttactgttttttctaattagttggaaaacctagaattatacgtatttgtattttctatcaataacaaaatatctccccggctttcagccatatcagtttccaatgataccctgtcatataatcagttgttttcatattttacaatctgatgattattgttgttattgttttttctatagatttgtttaacatattactagcaaatgacattattatcgatataatgaaccgttagctgttgcaaatcgaggtctttggattcattatatacctctgctaaaggatataaagtatgttcacagaatgtataagtgatcgttgctttatgttaacaaaattattgatggggaagatgctcctctagtcaactggatttgtctaggaagcttaaatataaaaggttcgatcttgaagtgcaaaatactgaatgggtcacattttttccttgccttctttctcaccaagtgaggagcataccaatttcgggagcgtggtgtgtcatgtcgtgtgtgtgttgtttctgattcgtttgtaaagtgaaattcactattttaaaataagtgttttcgggttcaaccgttttttcttattcaacttgttactctttctgttccaaaacatattatggatcgcctaatcatatattttcatttgctttgttgaaacaaaatttctattttcatgcagaaaatgacaataactacttaccatcctactcacgacaggacccttccaccacctgatcaaggtagacaagagctttcttctacttctgttgtaatgttctgctattagttgattgataatctatttgaaaaattgtatctttcctctggattattcttttcccctatatcactatttggaggaaaagaattgaaaacagaaaatgtttccacaaaccaaatgtaccattacaattttaactgtggttgcagtttttcttctttacgaagctgtatgctgcaaattttctctcagcgttattcttttgattgatttgggatactgtgattgagggttttctaacttgtggaaaattcttttgacagtgataaacactgaagcaaaaaatattctcctaagacatatttatcagaacgctcgggaaaaggttagttttgaaagtttgttttagcacaggtaaggtaggtatgcacatgaatgtgcaatactcatacatcaccatatagtggcccaactgaactgctgcatatgtccatttttcattgcagttgaaaccaaaaagagctgcagctggtaaccttttaccagaacatggatgcaagcaacctagggtttccacctgacagtgttcattgaccaactagtgcatgcagttctcagctactttctcgaatgatatatactcttattttattaccaagattttggtgcttgtttgaaattgtaaactattattagtccgctacatacttggagtgtgtaaatttgacaactcccccaccaatcaatcaatatatacaacaactgaaattatcatgcttttattgtgtatattttt

SEQ ID NO: 42 - protein, 93 aa, Medicago truncatula
MDSVLGNLPSYNPHNFSQIRPSDPSSSSKMTITTYHPTHDRTLPPPDQVINTEAKNILLRHIYQNAREKLKPKRAAAGNLLPEHGCKQPRVST

SEQ ID NO: 43 - DNA, 282 bp, Arachis hypogaea
atggattctgtccttggtaattggccatcttatgatcctcacaactttagccagcttcgaacttcagatccttctagatcttctaaaatggcaccggccacttaccattctattcacaacagggacgtaccaccagccgatcaagtgataaataccgaacacaaaaatatccttctaagagaaatctaccggcgtgcagaggagaagttgacacccaaaagagctgcatccgataacctcataccggagcatggatccaaacaaccaagggtttcaacgtga

SEQ ID NO: 44 - DNA, 513 bp, Arachis hypogaea
caaaagtttgttgcagcctctgcctcatggattctgtccttggtaattggccatcttatgatcctcacaactttagccagcttcgaacttcagatccttctagatcttctaaaatggcaccggccacttaccattctattcacaacagggacgtaccaccagccgatcaagtgataaataccgaacacaaaaatatccttctaagagaaatctaccggcgtgcagaggagaagttgacacccaaaagagctgcatccgataacctcataccggagcatggatccaaacaaccaagggtttcaacgtgaaaatttttctttgaccaacaaatgatgaatatggtttgtgaacaactctttcagaagtcagatatgcccttatgtaacaaagaagactttggcatgtttggtattgtaaactatcttttcaagtatagagttggttagccccagcataattattcagtgaatgaagtgagatagtgattatgaatttcattgtagattttgtgct

SEQ ID NO: 45 - protein, 93 aa, Arachis hypogaea
MDSVLGNWPSYDPHNFSQLRTSDPSRSSKMAPATYHSIHNRDVPPADQVINTEHKNILLREIYRRAEEKLTPKRAASDNLIPEHGSKQPRVST

SEQ ID NO: 46 - DNA, 285 bp, Populus trichocarpa
atggggtctttgcttggtgactggccttcatttgaccctcataactttagccaacttcgaccttctgatccttctaatccttctaaaatgactcctgccacctatcatcctactcatagccggactcttcccccacctgatcaagtgataactactgaagcaaaaaatattctgctgcgaaatttctatgagcgagctgaagagaagttgagacaaaagagagctgcctctgaacatctaatgccagagcatggatgcaagcaggctagggcttctacctcataa

SEQ ID NO: 47 - DNA, 285 bp, Populus trichocarpa; identical to SEQ ID NO: 46

SEQ ID NO: 48 - DNA, 1516 bp, Populus trichocarpa
ttcaaatgcattaattccacttgtgagtttaatatggattggaatcaatatcaaagtctatccatatggtcaaaaaaaattagccaggataaaaagaaaagcataactaatgatctcactcggtggtcatatgatgacccggtgaatctgttgtttgtacacaactctacttgcattgctcaacagtttagaacaataagcaagccctgctcagtcaaccaattggcacactgggcagtgatgtggtttgtatttacaacgagagtcgacatgccagactattaagtaacatacgaaagcttattatgaggtagaagccctagcctgcttgcatccatgctctggcattagatgttcagaggcagctctcttttgtctcaactgcaacaagaacgggaatgacaagctaaaataagtacgcgtccaatgggtgcacaaatagaagagaaagatcaaatatgtgaaaacattttaatttaccaaccttctcttcagctcgctcatagaaatttcgcagcagaatattttttgcttcagtagttatcactgccaaaggagtttaagccagattaaccgcagattatgaaatcaccttcagataaaatgaagaacaaaaaactgaattacaataacaaaatgcaggaagttcagctgatcaaacttgcataagcatgtcataatcaattgcacatcatcccaagcttttcagaatgcccaggcaatatccatgatgatacaaaagaaggcccatggaaattcttcgccatcccaagtggttaatcatggtcaaagaccaactcatggagcaagaggcatgcagattagagatagtgaaactgctccatccagcactgaaacatgtaaaattcaacatcgattgcagaaaacccccccgactttaggccagatgctttgtcactcacatccaaagtaaactaaagctccttgttgttttattaggatgttataatgagattgcaccagttttatccagaatgtcgtattgatttctcccaacttcatgcacaccatggactttgaaagggcggccccaagcctttacaagaatctatgccggttttgctcatgcatgattgttggattcaatctccccacctccccatcttttatattctacatttttgcctttcaagaatctttcttaatgaagtcacaaaatattaatatcttgatgaacaggaatcggaagatagatcaaagagcaaaacgctagcatctaagctcatcaaacatttacattcataacaagagagacattatgataacatgcacctttgaataacatgtccagataaagatgtcaaatttggcagccatatttaaaggtcatcccatgtcttagaaaacaaaatatgccaggtattccctatcttcacccaaacagaaaatttggggagaagaaactgcaagcagatagaaagttctgttctttaccttgatcaggtgggggaagagtccggctatgagtaggatgataggtggcaggagtcatttt

SEQ ID NO: 49 - protein, 94 aa, Populus trichocarpa
MGSLLGDWPSFDPHNFSQLRPSDPSNPSKMTPATYHPTHSRTLPPPDQVITTEAKNILLRNFYERAEEKLRQKRAASEHLMPEHGCKQARASTS

SEQ ID NO: 50 - DNA, 285 bp, Populus tremula
atggggtctttgcttggtgactggccttcatttgaccctcataactttagccaacttcgaccttctgatccttctaatccttctaaaatgactcctgccacctaccatcctactcatagccggactcttcccccacctgatcaagtgataactactgaagcaaaaaatattctgctgcgaaatttctatgagcgagctgaagagaagttgagacaaaagagagctgcctctgaacatctaatgccagagcatggatgcaagcaggctagggcttctacctcataa

SEQ ID NO: 51 - DNA, 500 bp, Populus tremula
gattgtatggacttataaagactaagaaatttatcatgccaacctgcggaggttggttctagaatcagaccattgttgtctctcataatctctctatctcgcattctaatggggtctttgcttggtgactggccttcatttgaccctcataactttagccaacttcgaccttctgatccttctaatccttctaaaatgactcctgccacctaccatcctactcatagccggactcttcccccacctgatcaagtgataactactgaagcaaaaaatattctgctgcgaaatttctatgagcgagctgaagagaagttgagacaaaagagagctgcctctgaacatctaatgccagagcatggatgcaagcaggctagggcttctacctcataataagctttcgtatgttacttaatagtctggcatgtcgactctcattgtaaatacaaaccacatcactgcccagtgtgccaattggttgactgagcagggcttgctta

SEQ ID NO: 52 - protein, 94 aa, Populus tremula; identical to SEQ ID NO: 49

SEQ ID NO: 53 - DNA, 288 bp, Linum usitatissimum
atggggtctatgcttggtgacttgccttcatttgacccccacaacttcagccaacttagaccctccgatccttccaatccgtccaaaatgactcctgcaacctatcatccaacacacagtcgtactcttccaccacctgatcaggttatggctactgaaacgaagaatatccttttaagaaacttctacaagcgcgctgaagagaagatgagaccgaagcgagctgcaccagagagccttataccggatcatggtggcaagcaggcgaggccttctacctcaagctaa

SEQ ID NO: 54 - DNA, 288 bp, Linum usitatissimum; identical to SEQ ID NO: 53

SEQ ID NO: 55 - DNA, 2260 bp, Linum usitatissimum
atggggtctatgcttggtgacttgccttcatttgacccccacaacttcagccaacttagaccctccgatccttccaatccgtccgtaagctcacactttttcccccatctttcatttaccgccccaaccttttctttttccttgaattgtgttgaatcttgagagttgattcggttcgcatctgaagttctgattttttctttgatttagtctcaatttttcctatccgagtgctacatatgaccctaatatgggttgagaactgatgaattttatgtttttttaatccaatgagtttatcggttgtttccccctttcttcttgaggtttggtatccgtcttttgtcgaatgctagggctaatattcgaattagattccatctattagacttttcagtgaaatttgcttgattcttcaattgaatactaggaacgagtcaaatttttgcgatgcagaatgtaatgctattctcaatttgcctaatgagtttaatctgcctattattatcgctgtttatattcctggatctttttgttcgaattagggctctcgttggcaagttattactgtaactgcttctccagaagttccatagaattaattgcatccataatttgataggacgagttgatttaagaaaaattaagtttttgacagtcaacaaaggattgattgtgatactgaaatgcagaatgtaggtaggctaattaggacttaggaatgataaaagttcgcttcttcatgtaaatgaagagatgtgcatcaaggataaaggccatcttaatactcgtaggttgagtcttttttaagaataatgcagttgctgatgcggcaagaaaaagttaagctagttgaagaagaaaggagtcagaaatgtatagatatttccttccctttgagtatttctcttctctgtaactgcttattgcttcaaagttttggcccttgttctaataatcacaacctttctataggaacatctaatggctagtgcttgataattccatgtatatacctcttactgacatgcttgtattttggttaccatttctgttggtttaagtcgtcaaatggcatagattcaatagggcactcataaacaagtaactacaatgcatactagagtaggtgtctgaatgcattgcaagcttccaacatttggttgttcaggaactctggaattgttccactagcatgttcaatgcaagatattcatgttcagtgttaaactgtgtaagtgaggagagtttaattcattgtacgcatgccttgggggtgtcagtacgaggatttaagctcaatttatcaagtctgtcattaaactagtacttcagcagggacttgacctgtcaataatctgtcatatgaatgctatcatgttcgtcagttttaaggaagagaaactctttaaatgtgcttatcatgaagttattggattcggttttatttattctgacattttcctacgatcatttgcagaaaatgactcctgcaacctatcatccaacacacagtcgtactcttccaccacctgatcagggtaatgactagctttcctttataatttgtgtttcctgccttaaaagtttccaccttttgtagaaagaacgtggtgtttcaacacttgactactcaagggatagtaattgttaattacttttttaactgtgtttatcgggcatggggacatttgaagttcttatagtttaaaacatcttattgcaactcaaaatccttcaacttccataagtaatagctaacctagagaactcaaggtctctaacccagttgaagcggttcgatttaatctgcatacattcttttccatcgctatcggtttctgtttcacattagaagtgagtctctccgattaagtcctcgaatctctggcctagctagttggccgcgcttaggaaaagtctgctacttttaaatctgtgtcgtaaagcaagcttgattagctgagcccattcaaggtctctagttcatgttccttgttctggctcttttgcagttatggctactgaaacgaagaatatccttttaagaaacttctacaagcgcgctgaagagaaggtttgagtcactcattggcatcgcattgttagccttggtttcatatgtacattattatttgcaacgatgtgtatgtcactgagcttatttgtgtttttccagatgagaccgaagcgagctgcaccagagagccttataccggatcatggtggcaagcaggcgaggccttctacctcaagctaa

SEQ ID NO: 56 - protein, 95 aa, Linum usitatissimum
MGSMLGDLPSFDPHNFSQLRPSDPSNPSKMTPATYHPTHSRTLPPPDQVMATETKNILLRNFYKRAEEKMRPKRAAPESLIPDHGGKQARPSTSS

SEQ ID NO: 57 - DNA, 288 bp, Ricinus communis
atgagctctctgctgggtgactggccgtcttttgaccctcacaactttacccaacttagaccgactgatccttctaatccttctgtaatgactcctgctacttatcatccaactcatagccggactcttccaccacccgatcaagtgataactactgaagccaaaaatatccttctgagaaacttctatgagcgagctgaagagaagttgagaacaaaaagagctgcctctgaaaatctaataccggagcatggatgcaagcagcctagggcttctacctcatgctaa

SEQ ID NO: 58 - DNA, 201 bp, Ricinus communis
atgactcctgctacttatcatccaactcatagccggactcttccaccacccgatcaagtgataactactgaagccaaaaatatccttctgagaaacttctatgagcgagctgaagagaagttgagaacaaaaagagctgcctctgaaaatctaataccggagcatggatgcaagcagcctagggcttctacctcatgctaa

SEQ ID NO: 59 - DNA, 1458 bp, Ricinus communis
ttagcatgaggtagaagccctaggctgcttgcatccatgctccggtattagattttcagaggcagctctttttgttctcaactgcatagagaaaaaataccaatatgaagctagaagtatgtgtagcaatcagataaagcaaatgctatatctgaattatgtgacattgtgtatcattaaccaaccttctcttcagctcgctcatagaagtttctcagaaggatatttttggcttcagtagttatcactgccaattttcaaacaaatgaatcacaaaattttcttttgatcattcaataccataaactgtattataatatcagaaaaacagaagctaggaaagttcagctgattaaacttgcttaaaatttaaaatctagacaccaaccctgtagtacaagcttttcaaaatgtccctaaaatatcaacagtgaaaaacactaattaagacccataaacattttatgcaatttctggctgaaatcggggatggcaagcaacttatggaggcaaaaggcaagcagtatcagatgtatgtgaatccaacattgatctcaaagtccatccctctagcatcaaatatatgtgaatccaacattgatctcaaagtccatccccctagcactaaatatatgtgaatccaagggctctatccagcaatgaatatgtgtgaacccaacattgatttcaaaattatccaccccctagcataaaactgaatgcatgtcactcatatttaaatgttagacaatagcccttaccaattcatggatatagaataatcacaaagtgacattgggtcatcggaataatttcctgatcccaagtcccactctcacatcaatttaatttaggaaaagcattccatatgtagttagttaacacttaacagcattttttctttactctttcaaacttgaatagcttggatcatttttcagcagtacctaggtcttgaggtcttgaggagttagactagactctagaggagctcaacatcatgtcctcacgcatggttgtcagactcaatctctctactctgatcatgcaaatatattcttgtttatcctagatttttgcattttagtacactttcttagtccaaggaggtttttctttcccattctttattctgttgtactagagcattttgttttgaacaacttaactcttatccaagaaaataataggaagagcagaagcatctccgaatagcatgagtaagcttaagaaagaagttaacaggtagatgaaattgcaaaggctggcatttagcagagacaagcatacgtcacacacaacaccagaaacaaaacacatttacacactcatgtatgattaaatcacaggataacaactagttccagcattcaaagaatcacacaccaactaattcttaccttgatcgggtggtggaagagtccggctatgagttggatgataagtagcaggagtcat

SEQ ID NO: 60 - protein, 95 aa, Ricinus communis
MSSLLGDWPSFDPHNFTQLRPTDPSNPSVMTPATYHPTHSRTLPPPDQVITTEAKNILLRNFYERAEEKLRTKRAASENLIPEHGCKQPRASTSC

SEQ ID NO: 61 - DNA, 285 bp, Theobroma cacao
atggggtctatgctcggtgacctgccgtcgtttgacccccataacttcagccaacttcgtccctccgatccttctaatccttctaaaatgacacctgccacctaccgccctactcatagccggactcttccaccacctgaccaagttataactactgaggccaaaaatatacttataagaaatttctatcagcgtgctgaggagaagttgagaccaaagagagcagccactgaacatctaataccagagcatggatgcaagcaacctagggcttctacctcatag

SEQ ID NO: 62 - DNA, 1495 bp, Theobroma cacao
atcatccagcactagtacgaaaaaggctgagtctagaatcggggcaggcattgttgtggtttctctcccattttctcaattgtcccaatctctctccggagattttctgggtgcagaaaccagcatattctttttccccaatggggtctatgctcggtgacctgccgtcgtttgacccccataacttcagccaacttcgtccctccgatccttctaatccttctaaaatgacacctgccacctaccgccctactcatagccggactcttccaccacctgaccaaggtattgaactgatatttttctccttgtttttacttgtgaaacaatattcccgaggaaatataagatattattggccttataaactgtctgcaatggtaccttctaggatgttgaatgttgacttctgtttgagagcagcaagtgctggaaattatgtggagatgtctgaattggaactggatatgatgtcatttttctgtaaaaatggtattgccttgacaaatgggcttcaaataattgcaaaacccacccccactacgatctccaacaagtccattttgttgcctaatcctcgtatcataaccgccaggcatcataataacatcctacaatcagcatcatcatcatcatcttcttcagcttttactcttacagcttcaatttcaccgggtgctacttcggttgcagtcgatggacccaccacctccacgaaaccttccaagtctttgccgtttagagtgggccatggcttcgaccttcatcgtttggagcctggctaccctttgatcattggtgggattgatattcctcatgatagaggctgcgaggctcattcggatggagatgtgctgcttcattgtgttgtggatgcaatactgggagctttagggcttcctgatatagggcagatatttcctgactctgatcccaagtggaaaggagctccatcttctgtctttatcaaagaagctgtgagactcatgcatgaagtaggctatgagattggaaacttagatgccaccttaattcttcaaagaccaaaattaagtccacacaaggaggctatcaaagccaacttgtctgagctgctgggagccgacccatctgttgtcaatcttaaagcaaagactcatgagaaggtcgacagtcttggtgaaaatcgaagtattgcagcccatactgtggtcctactgatgaggaagtaaatataggtctcggatatcagtctcgagtatggaaattgtatggcataccatgagcattagttgtaaaactgccataaattatggcattgctaagtatgaaagcttgatgtgtttggttggaccacaatgttagagttgtgttttcaacattttaccaaaacgacttgaacaacaacgatgtgagttaacgagtgaacctacatctacaacacggtaccgtgtgagtcaaatctgtcggacctttattgcggaattaattcgggaaacaaattttttttttgaaa

SEQ ID NO: 63 - DNA, 10274 bp, Theobroma cacao
atcatccagcactagtacgaaaaaggctgagtctagaatcggggcaggcattgttgtggtttctctcccattttctcaattgtcccaatctctctccggagattttctgggtgcagaaaccagcatattctttttccccaatggggtctatgctcggtgacctgccgtcgtttgacccccataacttcagccaacttcgtccctccgatccttctaatccttctgtaagtatcccagaatcctttttaaacccaaccccattaacaaaaatacatgaaaatatgaattcttttctggtatgatctgaataagttgttcgtttcaactgttctgatactgcaatgaaacccacatgcggttttagatgagtagtgaagaactgttagatttttatgattaggtttcaaggtttacccagatatgatgttggtggattcttttctcaaagcgcttttctgaatttggaccttaaaaaatgctgtagtccatataatcagttagtcgtcagaagctttttggataaagttcgtttgtggttacaaatgaatatcttgcttttgtatttataggggttaaatgattctcggaatttctactccggtgttattacacgtttagtgcttttgttgtttgcattgcaagatacatttaacactagtatgtttattaatttgaatgaatggaatgtaatatggtgatgttaccatgtgaagcatctaagttatgcaaataggacttgttattatctgtttgctaaaatgacaaagatctttatacagcacaagcattagcgtggaaatgctttcttgtatgggaatggcagtttccttaaaattgtagggtaactattcatgagcttgtgattttgaccactgcatgctactgtcagcttctaaatctaccaagattttaagttcatgctctagaagcttttcagatcttcctgtgtacttggtagtttaagttcttaagactggttcttaacaatgatcaagaagttctatatttcagatcttcctgtgtacttggtagttgaaagtttgtttctatgtgggtattgagagaattggtttgagaatttgctgttttcttgataaaaaattaatcctgtccatgacatggacatgctattgacagaacgcatgaaattgagcttttgcattctaacttgggcaccgttttactggtgattcgtattgagataatgattgtgttgatgacatttgggcagtcgcttgaaaatattgaaatgtcaggagaaaagagagaaggtgaagggaaagcccaaaaggaacgagagatattagatcaccctttttctttcctttatcttcttcttttacttggtctacctcctgatcattttcttgaaattctcactaaattctagttttgtttagatcttgaatttgtatagggtaatataattgccgaaaagagttcagtaaggcagggtttcacctggtaaagaagctgcatggtgaattttgaattgctgtcctctagcacatggtgcactacaggatatcaatttcctttcatagcatgcatacatgtaggcttatatgaatttatgtatctatttttgtttcaatagtttatagtggttccctgcctaactgttataaggtttccgattaagtaagagttgttgaaatgtccaaagggtatagaacatttttcaccagactttctatcctttttagctattttagcatgtgagatgctatgctaatggatggagttcatatggagctcctttatgttttcaaatgaagaatttatgacaaattagtctttttgctttcagattgataggttggtcttttaacaaaattacacttgtctttttgctttcagattgataggttggtcttttaacaaattacacttacttgtatggtttgttactttgcttgaatattttaggagcataaaatgcttctccttacttttcagtcatgttaagaattgattgcactttaacttttgtacttaatcttctctttttggctagaaaatgacacctgccacctaccgccctactcatagccggactcttccaccacctgaccaaggtattgaactgatatttttctccttgtttttacttgtgaaacaatattcccgaggaaatataagatattattggccttataaactgtctgcaatggtaacatgataacctttggttggctgattctttcagactttgtctacatggtgataatatattttaaaaattcatgtcatatactgtgataattatttggctaggtaccttctaggatgttgaatgttgacttctgtttgagagcagcaagtgctggaaattatgtggagatgtctgaattggaactggatatgatgtcatttttctgtaaaaatggtattgccttgacaaatgggcttcaaataattggtattggactgagaacttgatttttacatggcactctatgcttgctaccactgctacttgcttacattttttttgtcatctgtctctgaaatggtggaattgctttaccttgtttctttagttctttgtttccttctgcagtaatctaaatgtcgtagagtactcatctggaattgttcatcttctctcagtttcattttgatatcgacgaagaatggagatttctaattggaaaccagtgattgattgcgtacttgattgaaattgctgtttgaagtttatcaattggaatgaaattgttaagttgtatgcgtgttctaaattgcctgtgctacaaacacaaaggcactactaaatgagattgcattctggtgcagtgccacagctcattggcttaaattcttgatttcttatccttgtttttttaattgacctgtgaacattttctttttgtgtaactgacctctcaaacactttcaacagttataactactgaggccaaaaatatacttataagaaatttctatcagcgtgctgaggagaaggttagtaaattgtttatgatttttcatgttataacactgcattcaaataatttcagtcactgttacaaattcaaggacacatgcatgcaccttgagagtgggtgtctgggttcatttagtttctttttgcttttctcattgcagttgagaccaaagagagcagccactgaacatctaataccagagcatggatgcaagcaacctagggcttctacctcatagtcgtgatgagattttcttgggttcctttgatgctcatgtaaatgtatattctcatataaatgtgttgtagaaactgtgcggcagttgttgtaaaacgggacaccacacatgtacattttgtgtgtagaaatcaaacttatagctgggttatcccaataacaaggagtgttctgtttagttttacatgttccgtttggtatttcattccatgtggcgtggctaaattgctgaaaagctaaatccatgtatgctcattttatatgatgtgcacagttgcatctctaagtaattacaggtttgaatatattgcttgggctcaaggagctgcatattttaatataatatccatgctcctgttgcttatgaagttagtttatgcctgtttcaattacataaattgaaggttttccctgtggggttacagaagataagagtagtttgggaatcaactgagattcaaaacaacaggtagaggtacggtagtgtgttgcatcacttcttaacagaatattccatgaggtttaagacttaaagatctgatgaccattcatgtttcacggagttaagggtcgttttctgtctagggtctcccaaatttggcaacattaggcctctcaaagtcttgagtttggcaccaattagcccacacgcaatatcctgatgaaaaaacgtttccgaacagttacttgagaatgtgggacaaaatcatatattgcagtggctgtgtttgcacttgaatgaatttcggtaaacacattggaaatgggccattcaccggagttgcacccagcggttgacaaatggaaagtagacaacgaatgatattatttggttcctgcaattaaatacctacctttaaaataaaaattaaggaaaaaaaaaggaaacaaattactgccacaaataccaaaaaagagaaaaaaaaaaaaaaaggaaaaggaaaaacgaagggaacccacgaaacagagtcaacaaagaaactgaaactactccacaaagaaacagagccttttctctgccaaagcaaagatttcaacagctatggccactcacttttacagttgttctccaattccagcaaaacccacccccactacgatctccaacaagtccattttgttgcctaatcctcgtatcataaccgccaggcatcataataacatcctacaatcagcatcatcatcatcatcttcttcagcttttactcttacagcttcaatttcaccgggtgctacttcggttgcagtcgatggacccaccacctccacgaaaccttccaagtctttgccgtttagagtgggccatggcttcgaccttcatcgtttggagcctggctaccctttgatcattggtgggattgatattcctcatgatagaggctgcgaggctcattcggatggtatgttcacattccaatcgtccaattttgctttatttttgtttttcccaactttaatatccattttttatatcttttagtttcttgtatttggatagttttaatttatcaaagttcttttttttaaaccccgtatatgttttctcggaatctgtatctcttggttgtattatctgtttaaattatccacaatttgcttttgatgctaactatatggggattaacgtgttgcaggagatgtgctgcttcattgtgttgtggatgcaatactgggagctttagggcttcctgatatagggcagatatttcctgactctgatcccaagtggaaaggagctccatcttctgtctttatcaaagaagctgtgagtaattttgggtaattttgagaaattcgttttttttttttgggtattttattttcagtgtatgctttgactggttcactgttggataccttaaaaacagagttcataacacacagacacacacaaaagaaaaaaaagctttatttgccatactatgaaacttctacttaaaatctggcctcaacttggacggattgtgttaggttgggagaagggtcctatattagaacactagtactctatgaactcttgttgaagagaaagtaaacgaaaaggcaacacagagctgaattatgcaattatttattttgggtgcatagttaaggaaggaaaatattaatcattctgaaagtcataggctccaataatgtactttgacacaacatttaagtataaaaatcttatgaaatatcctttttagccttgttgataagaaaaataaaataaaaaggaattccttgagtggattggacagactacacagtcagtttttgtagtcaacccatatatgatatagcaccacttattgatctcttgaggggcatggctggttaatttctatctgcttagaaacaaaaactgagtctgctagttcatagcatttggtaatttgtattagatgcattagcagaacaaatcaatagagggctgcctgtattgaggagtggagaagtgagtatgatactgctgcatcagagcaaaactctgattgtctgacactgatagtgactttccaaccacaatgcaatgaaccttgtactttatgtgatacaatgaatctaacagtgcaatgaatctttcttcagaacagcttaacttgtgtatggatggaaattgtattaagttctttattgtcgactgtacttactgataagggcatgtgcatgtatataactgaagaaaattctgtataaccttgtattctgcctagcctgcaaagtaatactttgtgcagatgcaccttgtaaacatgatgtttgacttaaaagttttaggtaaaataagttaataaaagttgatgcttttaagagcaatgtatcgggtatgatattgcttcacttttgaattctgtaaaatctctgatgctttttggacagtgacttcaccaaccacaattcaatgaacctttcttcagaacagcctaatttgtttgtgattggaaatagaatcaactttttattattgactgaactattgcatattcatgtatgtgcatgaaaaccctgtataaccttaaatttgtatttatccatcttccaaactgatgctatatgctgatgccagtctaggctgtaaagtaaaaagctatattacatcttatgccatatgcttcatctagaagttctcatgtagttgtccctttgtatagtatagtggaggatccttgctgaacctgcatttgcatgtatagtgaattcagtcagagaagagactaaagccctgttgggagcacaggctagtgtggtgggattcccccccaaaatggcactggacccatatggctctcactcctaaatagcctctaaatcaataatgtgttctcctaatgcctctgcccttccacctcttttaatgctactttcttcacaaggattgattgtttggcattctaaaagttttaacttaatgtgaggttgccaaccttaaattggtaggatagatctatcttgagttgacctagaactggcataaggaagaatgattaaccgcaaactacaaagctggggaactgtaatgatctgagtccaagcaccactggcccaacaactttttgagcctaaacctcgtagttcttaaaagtttaagctgaagttactcttagtgattgacttagactcttatataggttttaacaatcacatttgcatctaatgtgggattgggtatcactatctcactcactcaaatccttgatgtcctcatcaaggccacacatcacaatcacaatcacaatcaaagcccactctacaggatcaaacatctcaccagcacaatgtgaaagtccacaccaaacaaacacatgttctaataccaattataatgttctgggtctaagcaccaccagcttaacaacttctcagacttaaaccatgttctattaccatttattcttttcataggttatttccatggtgtgttaaataaagtagctaatacatgtaagttggtactagtaattgacttagacccttctataaggcttaataatcacattcacatatgatgtgataggagcaaatgacaacatgatcactttgaaatgtggatgtggatgtggatgacccacaaactgatttgattaaggaaactgatatttttactttctactttatactgaattcaaatcatagaaattagtgataaataactaaatatttatatcagttgaagaacttcctgactaagtttgtgcagaccaatatgctaagcatgatcaaacagaaataatagagtattatgtaaaaaatgaaatgagtataaattgtttgtgttggaatcctttttatcctcatgcattatctaccctgctcgtgttagtcatgctaggctttcattgtttatatcttccattctaggaatataggattatgtctccaattttagctgttcatacaatttaaattaatcatactcataccctgcagcatggtttttttcaggtgagactcatgcatgaagtaggctatgagattggaaacttagatgccaccttaattcttcaaagaccaaaattaagtccacacaaggaggctatcaaagccaacttgtctgagctgctgggagccgacccatctgttgtcaatcttaaagcaaagactcatgagaaggtcgacagtcttggtgaaaatcgaagtattgcagcccatactgtggtcctactgatgaggaagtaaatataggtctcggatatcagtctcgagtatggaaattgtatggcataccatgagcattagttgtaaaactgccataaattatggcattgctaagtatgaaagcttgatgtgtttggttggaccacaatgttagagttgtgttttcaacattttaccaaaacgacttgaacaacaacgatggtaagttttgacgaggctacggtttcccgattggtcattagtctatgacgtttgtcaaaggctcaaaacatgaagtagaatacagaccaaagtcaaattaagcgtttatatctatgttctaaacagtttcccaacttcaatgttgaattcctagtttctgctgcattaaagtgactatggctgcgttcacgtgttcaactttggtgaattcaccggtttcctgattgaatttgaattgttctcaagttccacattaaaccatcatatgcgtgactaccatgaatttcacatgactattgattgacgtttggtgtgttttctcttttcaaatattgtttgctgcatgcgcccaacacgaaaactacgaaggaaatatgaattgtatttttggaaacttttttgttgttttgttggacaaggaatgagaatgttcccctcaccctcaggtcaaagtagaagccgattttaaaactctctgaccattgccaattaatcccatttttctgatgattttcccgatatgattgtcgccgtatgttcatgtttatggagtgtcatccactaagaataacccagaaaaagtccctcaagaccatttcagaggcggaattcagactctttgtaactggtttttcaaatagccaaagtgttatcgtaccagattaagaattttcaacgctacagggagcaggaatccaatctgttgacttcctgaaaagattaattacaagctttcaactcaaactgaaagagaaagttaggcaagtgggtcgatttggtcgcctatgaaggcttcaaactgcacatctctgatcaatctcttaccctgagaatctcaggaggaatgaccccccactcgccggcctcccctgagactgacaaagcttgtaaccaagtagcaacttgtagaagccaccaaggatttttttgtcttcttctagttccaaaacttgtcatatgttccctggagaaaaggctgaatgtctccaaactcaatattttgtgtaactttctgccgatgtcacacgattaattgttctgtactttatcatgttgaatactactgggtattcaatagcggcagtttggagaacaaagtaaatttttttaaaaaagttaaagaggaattccttaacaaggaagggagtattaaatgcatgaagaaggtaacgttgcagtcaaaggttgatagaggaagtaagctaattaacccagctgacaaagtaattaattagctggtaatacagaaaatcttgatcaaatcttcccttgaaagatgaaggcatcaacggagatattaaaaggattagcttagctttgtgtaatccaactgtgctcaaagggacaacaagaaataaaaaaatggggataggcacaataaagaattgaatgcttatgtcagatcagacccggccaattatgccaccatcgctcgttgccctttctcggcactcttttgagtaaacactattaatatagcatttataaaatggggccatagagagttgaattattctatagaaagggccatagcagataagccagattgggaccttctactgaattgatccaggcacacagcaaaatttgacacatattaaaaggtttctcttcgctgtttcaagtcctcccaatctgaagtcggtcttcaattttttacaattaatttattgtcggtgattacggattttccattttaaatacttagtaaagcaatggaatgcctcctgatgtcgctatgctttctgcaaatactctaatcctgggttacaaccaatcctctagaagtgtccactccactaatcttgattccacttttcaaagtcattgaactaatcagatgacttcaccttaatcattgtttaagcagacactaagcaagagggtccaaagggttgggagaacagcaccaatgacttgcaatagttgtctgcaccagccctgcctctgatcacctgagatggctcgtgctccatgtttaacatatgagaatggagctcagtaaaatgcccacatgggcgaggcaagccgaaagctgaatctcttcattgacaaagatgattacccttgaccttactatacaaagtactagtatgataatcacatgggacggttctgtagttcactttgtgacttgcagtgagttaacgagtgaacctacatctacaacacggtaccgtgtgagtcaaatctgtcggacctttattgcggaattaattcgggaaacaaattttttttttgaaa

SEQ ID NO: 64 - protein, 94 aa, Theobroma cacao
MGSMLGDLPSFDPHNFSQLRPSDPSNPSKMTPATYRPTHSRTLPPPDQVITTEAKNILIRNFYQRAEEKLRPKRAATEHLIPEHGCKQPRASTS

SEQ ID NO: 65 - DNA, 288 bp, Manihot esculenta
atggggtctatgctcggtgactggccttcctttgaccctcataactttagccaacttagacctactgatccttccaatccctcgaaaatgacacctgctacttatcatcctactcacagccggactcttccgccccctgatcaagtgataactactgaagccaaaaatattcttctgaggaacttctatgagcgggcggaagagaagttgagaccaaagagagctgcctctgaaaatctaataccagagcatggttgcaagcagcctagggcctctacttcatgctaa

SEQ ID NO: 66 - DNA, 207 bp, Manihot esculenta
atggggtctatgctcggtgactggccttcctttgaccctcataactttagccaacttagacctactgatccttccaatccctcgaaaatgacacctgctacttatcatcctactcacagccggactcttccgccccctgatcaagtgataactactgaagccaaaaatattcttctgaggaacttctatgagcgggcggaagagaag

SEQ ID NO: 67 - DNA, 3411 bp, Manihot esculenta
atggggtctatgctcggtgactggccttcctttgaccctcataactttagccaacttagacctactgatccttccaatccctcggtacgtatgctccctatctatccttttaactcctcaaatcattttaagttgtatatatatgttttttttgtttaatgatccgtctagtttatcttaattctttgctgagttttgtgcctcctagtggttcagataaggttttggcttgagtttaacagctatcaaataaaatttagattttggcgattctttctaatcccatttttagattgctttctgggtttacttagctgagctatgttaatggggcctgattaggaaaaattggtcctattattatagacacgttaaatttctggatttatgtagttttttttttttcattattagattgggttttcctgcgggagatgcaagttgaaaagatttttggctgttcaaaatgtagtattcctattcaacttttttgttaattttggttagattggcatttgcagcacacggattatgagacatgtagattgtgaattcatgagatgggacaatgtcttttgtggtaaagctaataatgttacggctttgaaaatcagatcttagattgtgggaaattacctcgttgttaggtccaagcagctgtaatgaggacttatctaaattcaactgcaatttgaaattgtgagtgtatctaatggcaatttgaacattaatgatatgaatatcaaatgttgcatgtgagcataagctgcattttattcaaaataatgtaccagatccagcgagattgagatgcttttatttgtgaatgtgttggtttcaaaaagctggtgtcgctaagactaagctaccaactccttaaagcaagaaacatcttctgcattctgtatgctcacctgcatgagtagacttttacattccaattattcattagctaaatacattagttgtttgactgtgagttaatttttttttctgatgttattatttattgtcaaatgaattatgcatgcctcattttttcttagattcaacctcgcattgattatggcccatagccaactagatttgcttgataaatttggcaccattatatctttaaaggtaagtatgtaaatgaagtggaatagaaaggtcttcgccctactatttctttgcttccctgatccttcaccttcttactgcgtctgctgttcagttgtatacattcccaaaacaatttctttgctttattctctcaagtgaaatagaaacacttctggccaatatatgaagcattagctcttttaccatgcaaaccgatggtccatcaactatgaagttcgaaaatttgacatgtcccatagttaatgaattttgtagattatttgtgtagataatcgggtaaatccttttggaatgtgaattctcatacatattgttgattttgggaaacaagctagggctcattttgcagcttctccagctgaggcaaaatatttggtttgatactgtaatagcaactatattaagttttgaaataaccagaaaaaaaaaaaaaagaaaaggcaacaaatagaggagtcaagcctgcgtatataagaggaacaggacgaatatcacagtactctatttacctgttgatatacctacgctaagatgaaattctttttttaaaccatctaagcatataggcacttgtataaacgtttgcttgtttgttattaactttgtgatattgttctctgtgcgctttgatggttacacttgtgatattgttctccatgtgctttgatggttacacttctggattggtgcctgccattagaattggtcttattaattcggaaatctctaaagcaagtgattgtgtcctatatgatatcctctagtgcttacaagttctgggatgaggctgctgtgctatggcagtttgactgttaaaatttccatttcaagatataaagaaaatatggtttgtgaaacatgtgtgctggttagtgatgaaccttaaatatacgactcaatacattgtcccatctttccagattgataatgtttcattgtttgtcaactttaggtctacttgaaatgcttctcatcacattttcaatgattcttattctgagaatggaatgaacttctattcttatttagagttataattatcttttatgcttttctgtagaaaatgacacctgctacttatcatcctactcacagccggactcttccgccccctgatcaaggtaataaacaagactttccaattttcatcttcaggaacttgttttccataaatataaggaaaatttgtttgccatgtaatgccatttgaatatttttgcaagatattttttttttcgggtagtgatattgcatgagtttgtatttctgttgtatctctttcactgtatgaaaatacgttataatatattaaatgccaacgattgcaacgccatgtatgatctatctattaaattctttgcatcaagtcatatttacaaatttatgcagatgtcgcagtctcttttgtttgaaaacattgtgaagctactgtagcattaagcttgttcttggtatctagaaatacttctcttgattttcttagaaatgagtggaatagaaacacttccttgagctaagaatgcgtgctaaaatggaaaatctagcaaatataagcataaaatcacacagggtgagtaggaagattgaactagattcccatgcgcgagggaaaaaatgttgtgatgagattactatattgaaagaattatttttcttagttggttagcattttaagtgacttgagcctacttgaaaaatcaagaaaccaacattgattaatctttttagttcaatgataaaggttgagttgagctcaagctcgacttggatttaaacagccaaacttgaacattgtgatgcttgaccaaagacccaggagttgtgaccagttcacaagctacgtgatatgtgtgaagcaaccttggatgttatataaccacttaaatgaacagatgttttcatttaatgctcgggagatattttaaggtaaatgtgggattcagatccattactggacggaccagtttggctccctgtaatctgcatgccttttccctctatgagttgcttatcaatgttaattgcaatctggacactataaagacctttgttggcttttgtcactgtgcatattgtaaggtcatactcaaaagcttgagctacagaggttgtgtttagatttcaaaaacttgagcaagtttggtggttgcacttttgattttgtaatgcagtttttctcgtatatggtgacgatttcctcatttgtctgaaatcatcttgccagtgataactactgaagccaaaaatattcttctgaggaacttctatgagcgggcggaagagaag

SEQ ID NO: 68 - protein, 95 aa, Manihot esculenta
MGSMLGDWPSFDPHNFSQLRPTDPSNPSKMTPATYHPTHSRTLPPPDQVITTEAKNILLRNFYERAEEKLRPKRAASENLIPEHGCKQPRASTSC

SEQ ID NO: 69 - DNA, 287 bp, Hevea brasiliensis
atggggtctatgctcggtgactggccttcctttgaccctcacaactttagccaacttagacccactgatccttccaatccatcgaaaatgactcctgctacttatcatcctactcacaaccgtactcttccaccacctgatcaagtgataactactgaagccaaaaatattcttctgagaaacttctatgagcgagctgaagagaagttaagaccaaagagagctgcctccgaaaatctaataccagagcatggttgcaagcagcctagggcttctacttcatgcta

SEQ ID NO: 70 - DNA, 539 bp, Hevea brasiliensis
ctatcgctcttcctcttctgatatctttctctgtctagatttggccaccgaaaccccgcattccatggggtctatgctcggtgactggccttcctttgaccctcacaactttagccaacttagacccactgatccttccaatccatcgaaaatgactcctgctacttatcatcctactcacaaccgtactcttccaccacctgatcaagtgataactactgaagccaaaaatattcttctgagaaacttctatgagcgagctgaagagaagttaagaccaaagagagctgcctccgaaaatctaataccagagcatggttgcaagcagcctagggcttctacttcatgctaagctttgtttactgttggaagtacaacatgccggttgtcaatgtaaatacaagtcaagtcatgtcatgtcatgccaaatgtgtcaatttgtgtgaaagggatttgcttgcgcaatgctctgaactttagagccatacatgtagatatgtgtgtagaagtgagatttacagctgagtaatcaaatataa

SEQ ID NO: 71 - protein, 95 aa, Hevea brasiliensis
MGSMLGDWPSFDPHNFSQLRPTDPSNPSKMTPATYHPTHNRTLPPPDQVITTEAKNILLRNFYERAEEKLRPKRAASENLIPEHGCKQPRASTSC

SEQ ID NO: 72 - DNA, 288 bp, Gossypium raimondii 1
atgatggggtctatgctcggtgacctgccgtcatttgacccccacaacttcagccaacttcgtccctccgatccttctaatccttctaaagtggtacctaccacctaccgccccacacatagccggacttctccacctcctgatcaagttataactaccgaagccaagaatatacttattagaaatttttaccagcgtgcagaggagaagttgagaccgaagagagctgctactgaacacccaacaccggaacatggatgcaagcaacctagggcatccaccacatga

SEQ ID NO: 73 - DNA, 758 bp, Gossypium raimondii 1
gtttattattaattaaataaattatagaagaattttgaagtccccagcattagcggggatccatgctagttatataagcatcaatttacccattaatgatccagcttcagcacaaagaaggctgattctagaaccgagtcagccattgtcctttttttctctctcttggccgggttctctttgtaatctccggtgattttttgggtgcaagccacaaaaccagcaattttttcttcttttccgatgatggggtctatgctcggtgacctgccgtcatttgacccccacaacttcagccaacttcgtccctccgatccttctaatccttctaaagtggtacctaccacctaccgccccacacatagccggacttctccacctcctgatcaagttataactaccgaagccaagaatatacttattagaaatttttaccagcgtgcagaggagaagttgagaccgaagagagctgctactgaacacccaacaccggaacatggatgcaagcaacctagggcatccaccacatggtcataatgagattttcttttggtttttcaatgctcatctaaatgtatcttctcataccaaatgtgtgttgtagaatctgtgaggaaagttgctttgcacattgttgttaatcaggatgccatgcttgtgcattgtatgtgtacaaattaaactcataggttaatcaataacaagaagtgttgttatgtttctgcattctcttctgggtattattgattcttgtcggc

SEQ ID NO: 74 - DNA, 3456 bp, Gossypium raimondii
gtttattattaattaaataaattatagaagaattttgaagtccccagcattagcggggatccatgctagttatataagcatcaatttacccattaatgatccagcttcagcacaaagaaggctgattctagaaccgagtcagccattgtcctttttttctctctcttggccgggttctctttgtaatctccggtgattttttgggtgcaagccacaaaaccagcaattttttcttcttttccgatgatggggtctatgctcggtgacctgccgtcatttgacccccacaacttcagccaacttcgtccctccgatccttctaatccttctgtaagtaacctcataatcccttttaacctaaccccattttccataacatgtgaattttgtttctgggtttgaattgcaatgaattaatccccacatgcagtttagataaatagtgaaaaaccttttaaatttttatgtttatgtttctcggtttaccctgatatgatgttattgttatctgttttttttttttgtgaaaagtatttattattcatgaatttcggaccttaattgtcagaatctttttggataaagttgtttgtggttacaaatgactaggggtttaactttaaatgtttcatagaattccaactctgttattcctacactttgttttttttaatggtttgcattgcaagatatattatttattatttagatgttgattaatctaattgattgttgtgtaatgcttctatatgaagcatgtaaataatgctaatacttgttattatttgtttgctaaagttatagttatatgttcacacaaggatcatgcttcttcttttttttttgcatgggatgatgatggattttcctttaaaacttcaagttcatgctttcgaagctttcttatcttgctgtgtatgtggtagtttttgttcttagagctggttcttaacaatggtaacaaagacaagttctatgattaagacacaaatgagaatttgtttctatgtgggtatacatatgcatagacaattagatatttagacatattggtatttatgtattggtttttttgttccagtggtttacagtggttccttgcctatctgtttaaccatttttgagtaagcttagactggttgaaaggtcgaaatggtttaaaaaaaaaaaaaaaacaattttcaccttcccttctatctttttgagctatttcagcatgtgagatgctataatgttcatggagttcatattgagctcctctatgttttaatctgaacagttatattatgataaatgagtctttttgctctcattggttagcctttaactaaaattacagacttcccttgtatggattttattttgcttgaacattttcggggcataatttgcttgaatttttttttgctagaaagtggtacctaccacctaccgccccacacatagccggacttctccacctcctgatcaaggtatcgaacaaatattctctttctcctttttttcactacgaaaacaatattctattctgagttaaagtaagatactattggcgctttaaactgtttgtggtgataatatggtaactttggttggttaaattactttcaggctttatccacaaagcatgccattgccatattacatgataattattagtctgggtttccttctaggatgttgaatgttgacttatgtttgagaccatccagtgctggaaaattacattgatatgtctgaattggaacttgatatgaaataatttttctgaaaaccttgtgttgtgttctccctttgcatcctttttttctttctctcagatttcttgattggtagtgatttggttcatctgtctaactttggttgacactgagcagcaaagtgttttggatatttcaggttcaaatcacaacaaattggtattgaagtaaattattagaaatcaattggtgttgtctttcgtaatagcttttggtaatgctagcattagcaagagctttggtgtgagaatgtcattgaagatggatgaaaaattcagtgttagaaaagtagtcttattacggttcgtttcttttttcaagttttttttattggtaaaactcagtttatcagtctaatttctaagtggattacttttagcagcttctgcttcaatgtgttttggatatttttggtctgaatcccagaaaactggggtcgagcaaattcttggaaattgattggctttcaatctagcactagggaagagtttgatgcgggatgatgtcgaatatggatgtcaactaatttgatggtagaaaagattttagcctttgtcatgtaaaagtagagttgttcttatctccttctcaatagccattgttgtgtaagatcaactcttacacgccaaacgttacaatattttttactatcaaatttgttaacatccatctttgatgtcatccttttacccaaacacttgctaatcttaagattatcgaaagccatgacgaaatgattgttattgcacccgaactcttgctagtcttaagattatggataagctcacaccacaaaatgcctcaaccggattcccaagtgtttctcattagctaatgatagtacacaactaaaacctgacccaagagacaatttacgaaacttaaaatcttatcttttcattcttttcattttcactctctcatccctttgccaaaaccaagcatcagtgcacccctgccatctttaattgtcaacacccaacctcatgtcccaattgccaacttgtcttacactttgcaaacttctttagttgaaaatttcggctagtttcaataaatataaaggtactagtgattgaaattgcattatgatgcaaaatggcacagctcattctctaaatcctcgagtttttaagttgaatacgatttttagttcacctttgaacagcttttttattggagactgacttctgaagcacttttttacagttataactaccgaagccaagaatatacttattagaaatttttaccagcgtgcagaggagaaggtcagtaattcatttatgattttccatagtcatagtttgaaactcatagacacatgcataaactttgagagtgagcgtgttgatacttcattttgattcttactgccgttatcattgcagttgagaccgaagagagctgctactgaacacccaacaccggaacatggatgcaagcaacctagggcatccaccacatggtcataatgagattttcttttggtttttcaatgctcatctaaatgtatcttctcataccaaatgtgtgttgtagaatctgtgaggaaagttgctttgcacattgttgttaatcaggatgccatgcttgtgcattgtatgtgtacaaattaaactcataggttaatcaataacaagaagtgttgttatgtttctgcattctcttctgggtattattgattcttgtcggc

SEQ ID NO: 75 - protein, 95 aa, Gossypium raimondii 1
MMGSMLGDLPSFDPHNFSQLRPSDPSNPSKVVPTTYRPTHSRTSPPPDQVITTEAKNILIRNFYQRAEEKLRPKRAATEHPTPEHGCKQPRASTT

SEQ ID NO: 76 - DNA, 285 bp, Gossypium raimondii 2
atggggtctatgctcggtaacctcccgtcctttgacccccacaacttcagccaacttcgtccctccgatccttctaatccttctaaaatggttccttccacctaccgtcccactcatagccggactcttccaccacctgatcaagttatagctactgaggccaaaaatatacttattagaaatatctaccagcgtgctgaggagaaattgagatcgaaacgtgctgccacagaacatctaataccagagcatggatgcaagcaaacaaggccttccacctcttag

SEQ ID NO: 77 - DNA, 684 bp, Gossypium raimondii 2
taagccacatagctctttaaatacacatcaaattacggattactcatgaagcaatagcccaacacagggctgattctagaaccaggtcaggcattatggtggtttctctctcatcttgtcaatcatatgcatcccaatctctctgacattttaggtgcaggccagaaaccatcgtttcattcttccccaatggggtctatgctcggtaacctcccgtcctttgacccccacaacttcagccaacttcgtccctccgatccttctaatccttctaaaatggttccttccacctaccgtcccactcatagccggactcttccaccacctgatcaagttatagctactgaggccaaaaatatacttattagaaatatctaccagcgtgctgaggagaaggttagtagtattgagatcgaaacgtgctgccacagaacatctaataccagagcatggatgcaagcaaacaaggccttccacctcttagttgtaactcttcgtttttttctttgaggctggtgtaaatgtatcttctcatatcaaatgtgttgtaaactgtgaaaaaaagttgcttacccactgttgtagactgggacaccatacatacgtgtatattttgtgtataaatcaaacttataaatggttatcattataatttttatggcgaccactgttatttgtagttca

SEQ ID NO: 78 - DNA, 3382 bp, Gossypium raimondii 2
taagccacatagctctttaaatacacatcaaattacggattactcatgaagcaatagcccaacacagggctgattctagaaccaggtcaggcattatggtggtttctctctcatcttgtcaatcatatgcatcccaatctctctgacattttaggtgcaggccagaaaccatcgtttcattcttccccaatggggtctatgctcggtaacctcccgtcctttgacccccacaacttcagccaacttcgtccctccgatccttctaatccttctgtaagtatccttgtcatcctttttcaacccaactccagtttccaaaatacatcccatatgtattttatttctgggttttacctgaataacttgtttattttagctgttctgatattgcatcaaaaccccatatgcagtttacatgagtagtaaacttttgttacattttatgatttggtgggtttacccagatatgaactacggatttctttttatgagtaaatttcctaaattcttaccttaatgctattgcccatattatcggttacttctcagaacctttatggataaagttgtttgtggtttcaaataaatatatttgcttcccgatctatattggctttaaatgattcatgtaattccaactctggtgttattacacattagtcattttattgtttgcattgcaagatgcatttattattaggatgttgattaaactgatggaatcaagtttaatatggtaaactttctatgcaaagaatctaagtaatgctaatagtaaaacttttttttttttgctaaagtaacaaagatctttgccaacaacctcttggcctaatggcaagagtattaggttgtgaggcatgagagcttgggttccatcccaagcaaccccatccccaacccaattataaaaaaaaaagaaaaaagaaaaagaagtgacaaagatctttatacaacaccagtataaacttggaaatgctttcttgcactagataataataggagtttccttaagatggaagtctctattctttgagagcttgagattttctccagtgtttcagtttaaaactactaagactttaagttcatgctttagaagctttccagatattgatgtgtagttggtagttttagttcttacaagttaagaccggatcttaacagttatcatgtagttctttgttttatttaagatactagctgagaatttgtgtctatgtggccatatgcataggtatacttgtagacaataatttatttatatgtatctatatttgtttccatagtttgcagtgattccttgcttctgttataatgtttccggttaagatcataacagttgaaaggtccaaatggtgttgttatgactgtgaatttggaattcagtcacggatgatggatgagaaaattcctcaagctcatcacagggagcctaaccttagaacttgttacagagaaaacccagatctcttttcagggttttgataaaaattcgacttcagttttattattccattcttctcagttttattacataattaggattttttttttggaaaggaaagaaatcaattgaaataaaatctcaatcgagatacctgaggataaactgaaataacatattaaaatcataattgagaaacctgagccttaattagagaacccaaattgatggcctatcctaacataggtttggtccacagagcatccacaataggcgtacaacaattttctcctgcttcttatccttttaagctattttagtatgtgagatataagctattctacggagttcatcttgaactcctctatgtttttaaatgaacaacttatgacaaagtagtctttttgctgtcatggattggtctttaacaaaattacactggcttcacttgtatggtttgttactttgttcaaacattttagatgcataacacacttcttatttttaagtcatgttaaagagttgattgcactaatattttatattctaacattttctttttggctagaaaatggttccttccacctaccgtcccactcatagccggactcttccaccacctgatcaaggtatcgaacggatattctttctccatgtttttacttttgaaacaatagatcattttgagttaaagagagatattcttggcactttaaactttttgtgatgaaaatatggtaacctttagttgtgaatactttcaggctctacccatatggtgataaattgataatatattttcataatgtgtgtcgtatagctctggcaactttttggctaggttactttcagggatgttgaatgttaagttgatgtttgagagcagaagtgctggaaaagtatgctgagatgtctgaattgaacttattaatacatgatttagttttgttggaaaaatggtattttcttcacaaatgggcttcaaataaatagctatggattgagaaattgatctttatgtagcacttaatggatgccattgctactatttgtcacacttttttttttttataatcttcctctgaaatggtggaattgatttatcttgtttgtttagttctttgtttccttcagtaatctatacgccatagattgattatttgaaattcttcatcttctctcacttttgctttgattgatatcaacaaagaatagaattttttttatcagaaaaatcattagttgctttacatgattgaaattgtttttgaagttttaacaaccagaatgaacgtgtttgaggttcttatccgtacacgtgttatgctactgatataaaggcaccagtggttggaatttcattctgttgcaaaatgccacagctcattgacttaaattcttgactttctttatatttttttcccatatatgattgacctgtcgaatactttttaacagttatagctactgaggccaaaaatatacttattagaaatatctaccagcgtgctgaggagaaggttagtagtagtaggttttccttcttttgatgttataatactgcatatacaaataatttaatcatcgtaaatagaaatacacaggcacatgcatgcacagataattcatctagtttcttactgctttcccattgtagttgagatcgaaacgtgctgccacagaacatctaataccagagcatggatgcaagcaaacaaggccttccacctcttagttgtaactcttcgtttttttctttgaggctggtgtaaatgtatcttctcatatcaaatgtgttgtaaactgtgaaaaaaagttgcttacccactgttgtagactgggacaccatacatacgtgtatattttgtgtataaatcaaacttataaatggttatcattataatttttatggcgaccactgttatttgtagttca

SEQ ID NO: 79 - protein, 94 aa, Gossypium raimondii 2
MGSMLGNLPSFDPHNFSQLRPSDPSNPSKMVPSTYRPTHSRTLPPPDQVIATEAKNILIRNIYQRAEEKLRSKRAATEHLIPEHGCKQTRPSTS

SEQ ID NO: 80 - DNA, 288 bp, Vitis vinifera
atggggtctacattgggcgactggccttcgttcgaccctcacaatttcagccagcttcggccctccgatccttcaaatccatcaaagatgatccctgccacgtatcatcctactcacgatcggacccttccaccacctgatcaagtgatatccactgaaaccaaaaacatccttcttagacatttctaccagcgcgctgaagagaagttgagaccaaagagagctgcctcagaacacctgacaccagagcatggatgcaagcaacccagagcttctgcctcagactga

SEQ ID NO: 81 - DNA, 635 bp, Vitis vinifera
cgggactggaaagaatggcgccaaaacgacgtcgtttgttgtatttgcaaccgttcgcgataactcctgcgtagaatccagacgactgcgaacatcaggtgcctctgtcatccggctctctctcatggggtctacattgggcgactggccttcgttcgaccctcacaatttcagccagcttcggccctccgatccttcaaatccatcaaagatgatccctgccacgtatcatcctactcacgatcggacccttccaccacctgatcaagtgatatccactgaaaccaaaaacatccttcttagacatttctaccagcgcgctgaagagaagttgagaccaaagagagctgcctcagaacacctgacaccagagcatggatgcaagcaacccagagcttctgcctcagactgagcttttctccattgggaagtcaaatatcgtcttcagcttgtatataactatatatgtattcccatactcaaatgtgtaaactgaaagaagacttgctttatcattatcgcaaaaaatgcttagccacaggctagtagatgttgggtgtaaaaatcagattaagatatagctggattattcccatcccagacagtgaaattatgaaattgtctttcttctcata

SEQ ID NO: 82 - DNA, 4495 bp, Vitis vinifera
cgggactggaaagaatggcgccaaaacgacgtcgtttgttgtatttgcaaccgttcgcgataactcctgcgtagaatccagacgactgcgaacatcaggtgcctctgtcatccggctctctctcatggggtctacattgggcgactggccttcgttcgaccctcacaatttcagccagcttcggccctccgatccttcaaatccatcagtatgctttggctttatctaaattttattcatttatttatttattttgggtttctcttcactcgatttgattgtgtgcacagatgcatttcatcattcttcttccatagttgcatcttaggttttctgggtgcccctggctgagttcattagaatttcaggggcttgttgaattcaaaatatgtataacctttcgtttctgaatttggaccctaatctgttgtatagcactgatcaatcaagcactgtgtgtggaaaatgttctggtttgagagttcttaagtcaagtagtagtattagactattatcctttctgattatggctgaagctattgactctcttggtacgtggaaataagatttgggaatgggaaatctatccatttcccgtttgttaccgtgattggatttcttattagaaattagaaatggggataggaacatggaaaccaaaacccacccttttagaattttgattcctctcttcaacatggcaatctgattcacttcgattccgatttatacttccattcctattcccaagtctcatttttggtctcacctgatggcaacaaaactgatctttctagattttgtttgctcatgttattgtttaatctttcagcttattgatcaacatttttctcctttcaagaatccattttaggcccttattcctaactgtttaatgatgagtccaacatatcttttcctctacttttccatgtactaaatgcttatgcttgtagaaaatgaacactcgttagcatgaattataatttaaccaaggcatatgttcatttattaatttagacttgacaatgcactggaaatctctgaattggtttgaagctgttaaggggttccaatagcttataaaactattgaagattgaaaaaaggtctattgatgtatgctaatgttcaaatagatttgttcaggactagattatgttataatttttagtgaatttgtctgaatatctgctctttatttgtttacccttgttgtttattcagtctagcccattttctgacatggtgtaagggtaattgtttctgagacacatgccaacccagtttaagctctgtttccctgctaatgggagagttggactacaacatagcaggttgggtcaggttaaagatttaaccgagggtcaatcttatgcataaaggctcagtcattggctaagctcaacctgtgctccagcagtagttgcttaacctgggttgaacccaagtaattttttatcttgagtgatgtgggtcagggttaggtgaggtttggttgggttaaattaggcttttggttgccaaagaaaggtcaaatgcaggttgtgctttttcaaaataactaaaggagaagaggagtgtgtattaacaaaatttagacggcagcattagcaatgtggtgatggtgatcagagatggcatgatgcaaaattggtgatggggaagattggtgggtctgtggtttcccacatgatactttgagagaaaatgatattgatttgagagtttatcagcaagggttatttgctttattttcttaagtcacaaactctattgaaaaccccaaatgaaacaacgaatgcacaagaggtcagattcaggttactcaacacctcagacccaatccaccaccaaagtaaaattgttttggagattttcctgcccatgcagctcccatggatcaggttgggtgggctcaaatctgcctaagttggagctctatatgaggggactatttctttgaagacaaaatgaaaagtaagcattcattgaatgtaagcaagcaatcattattgtgaaacgtactttatagcgcattcattgtgaattatagttgtggctacttgcttgtggttttcactctcttattctcttggaaaacaaggaccaggggaatgaggggtgagtttccttgcaagtactaaggggtgaaatgcactaattatttgtagttatttggatatatgttattaggcttagatttgattacaatttagctagtgattattttggaatattttctttttgtttctgtttaagatacatactgaatttaacccgtgtttatttttgtggaaattaattccaccttttctacaatctgtcaaagatatttcccccaattgcaataaagtgcattgtctttatttttcttaagatgcatttatgttttgtatcagaagatgatccctgccacgtatcatcctactcacgatcggacccttccaccacctgatcaaggtaatggactccttagtctttcatttttggattttttttcttttttttcatttgtttgtttgatttatttatttactttttgggaaggtgtggttggtatatccacatattgataatcattgtaataaatccatatagaaactggttgatgctactggatctcgtctaattatttggtgatgttattgtgaatattttgtttttaacatgtcttttgactggtatgccttttttgcttcttgagaatatttaactagagggacagatgtttgccccaattcaacaccattatgttgcaaaatgaaacaattttaacagtgttaatccatgaagttatatgggcatccacttttctttatggggaaaccctagataaataggcttcagaatcacatttagattggaaacatacatttatttgtagtgcctgtagttaaataaaattgggaggtttctccccacttacactgaatttgaatccaacatccttcttacagaaacctatagtctcctagtgatatgatatccttgatgtgtactccagaaagcacaagttcaaaaaaaaaagggggggaaagacttgtatgtgaacttcagatcttaaacttgatgccagaggattgaggtaataggggaattcaaatcctaaatgcatgaccatctcatgatgtgcatgatgtgggtgctttactcatttcttgcttcataagtagtcataacatgacaggtacacttgtggtaaccttggtcactgaggctcaagccttaaggttaaccatcaagggcctcaaagcaattctttgtgatgctggcctggaggcataggggtgtccaccacattgattgtcctttatagttgttgttcaatgttctttgataagtttcaggtgagtctctttaaactattactctttttatcttcagggttgtcattgcacttccatcaaacagtatttcaaagtacagatggtctttcagtgaaaatttcaactatgatttaaaaaaaaggtcctaatgccctgtagttgtgcaactgacatcttattactttaagaagattctcaaataaaggttctaaatttgctgcactttggggtttgaaatctgatttcaataacagtgaaagaaaggcgtaattgcagcatttttgtatttgaaacctattcaatgaaaggtgatcatgttggggtgcaataatgcccactcttaggcctgggataaatctcccaaatgatggtgatgttgaataccataaaaggcttgacctatctcattggataccaattggttttcaaatgagatggtcagagcctgattcaagaatttgtataagagccaatgtcataggtttggctcatgggagcctctttttgggttacacaaatgaggctaaataccataaaaggtttgtcctacattcactggacattaattggttttcaaatgagatgattggagtccggtccaagaaactaagtaactctacttcctgtaatttggatgtttgcttcataaagtttgattctacctagccactttagtttcatcttgcatctcacccatctaaatcctcatggcagtgatatccactgaaaccaaaaacatccttcttagacatttctaccagcgcgctgaagagaaggttagaattcagttccttatgttgaatcaataagcacacatagcagaacctagtttttttagcaactcatttccttcctacctgcagttgagaccaaagagagctgcctcagaacacctgacaccagagcatggatgcaagcaacccagagcttctgcctcagactgagcttttctccattgggaagtcaaatatcgtcttcagcttgtatataactatatatgtattcccatactcaaatgtgtaaactgaaagaagacttgctttatcattatcgcaaaaaatgcttagccacaggctagtagatgttgggtgtaaaaatcagattaagatatagctggattattcccatcccagacagtgaaattatgaaattgtctttcttctcata

SEQ ID NO: 83 - protein, 95 aa, Vitis vinifera
MGSTLGDWPSFDPHNFSQLRPSDPSNPSKMIPATYHPTHDRTLPPPDQVISTETKNILLRHFYQRAEEKLRPKRAASEHLTPEHGCKQPRASASD

SEQ ID NO: 84 - DNA, 294 bp, Malus domestica
atggggtctttgttcggtgactggccgtcgtacaaccctcacaacttcagccagctccgaccatccgatccttcaaacccttctaaaatgacacctgcaacctactatcctactcacaaccggactcttccgccacctgatcaagtgataactaatgaagccaagaatatccttttgaggcacatgtatcagcattctgaagagaagttgagacaaaagcgggcagcgccagaaaaactctcaccggagcctgtatgcaagcaacagaggtattctgtctcagatactgcctaa

SEQ ID NO: 85 - DNA, 429 bp, Malus domestica
atggggtctttgttcggtgactggccgtcgtacaaccctcacaacttcagccagctccgaccatccgatccttcaaacccttctcaatgtcattgtaaatttgtaatgctgaagagtgctggctgctttgctgttggacctgcttttggtcacggccccagattaggatggaatgttcattgctcaagtaatatttatagcctttcatgggtccctaggaaaatgacacctgcaacctactatcctactcacaaccggactcttccgccacctgatcaagtgataactaatgaagccaagaatatccttttgaggcacatgtatcagcattctgaagagaagttgagacaaaagcgggcagcgccagaaaaactctcaccggagcctgtatgcaagcaacagaggtattctgtctcagatactgcctaa

SEQ ID NO: 86 - DNA, 2588 bp, Malus domestica
atggggtctttgttcggtgactggccgtcgtacaaccctcacaacttcagccagctccgaccatccgatccttcaaacccttctgtgagttttcactttctgaaattctgaatcaaacccctttttccaccttcttattcaagctgaatagtcgtgcaaagattttcgtttttgttgaagttcttgatttttctgaattgggtggttcttaattcagttaaaggtgaggaattgtggtttttctgtctgcatcaagtttatttctgggacctgtttgaatttcataggaaatttggagtaatttttgtaattagttataactaggagatttttgtgcagattttacttctaagtttatggtaatcgtaattgatgttgcagcaatgtcattgtaaatttgtaatggtaaagtgtgtgtagcttaatataaatatagaatttcaggtcataaatttaccacttttgttgaatttcacaatcctgaaatcggcttaatgatatttggttaagatggtgcgactgtttggaattggatcatcgtcttgaattatgctatatgtttgattttgtgatcaatgaacaataagctgttgttgcttgtgatttgtaatatttggatctaaattacgttcgatatttctgttaatcacttcactgatctctaaaagtttcgtgctttgttattgcattgatcttatataagtgtaattatcaagattggtgctttgttccagttacgatatactgtactgtattgctcaattctcatagagttcgtaagaattttgaaatcagaagcataattcatagctatgcaatatctcataatttttaacattcggattgaagttacatggttaatttaccattttcatgaagctgattcactggctttcttagttaaggtagaaatactagcatttaaagagcgtcctaaattagttttgccttcctgaatccgagctatagagaatatccgcataacctgtcagatataaggtgtttttcttgtcttctgattggcgggttcttgtcaaaaccaacccaaaaccaaaaccaatcggtaatgggtggagagacctgattcaagtttaaactgtgagatgtgtggttactatattctcatcattgccattcacgtatagtggaactaagaccaacacgtgtggttagcaatgttagggcacatgaagtgtgtgtgaacaaagacacggaatgaattgtccactgccatgtttaattttcaacataaacttctggtgcttgagctttttccggaaatggttgatatgtgctatagctgaattttggatttgtttcaaaccaactgagctggcattgtgggccactaggacgaaatcactgaaaacgttacaatgttacaaacatagaaatatagtaagtcggagaaaattacacgtgctctttacatctcatactctaattatagagagaagcacgatttttggtaaaaggttgcttcgtgatgattctgagtcatacgttcatacattctggtgggtatcagccattgtttggcttgagttagtgttgatgtagtgaaacgagcctatctaatgaaaattatctctctgttccaaccaccagactagtatatatttgagcttatattctgctaatagaattataaacaatttaattacagctgaagagtgctggctgctttgctgttggacctgcttttggtcacggccccagattaggatggaatgttcattgctcaagtaatatttatagcctttcatgggtccctagggtactcgtagatgcatcttcaaaaactgaagggatataatcaatctaattattgctttcttgttcttttagcagccaaaagaaaaaaggaaaacgagaagatgttatgagttatgctatgatgtaaagtgatcaaagtcaaatgcattcttttcttctttttaagtaatgcttccatttttgtctgtagaaaatgacacctgcaacctactatcctactcacaaccggactcttccgccacctgatcaaggtattaatgaatttgtatttccttagtgtttcattctggattgttattcttttctattatttccctatttttcccatttctgttgattaattttcctttccgtgttgtattattgtgactctttgagaaaggtccattctagttagttaagctttttaaaataaatcctgaaaactgcaagttgatctgttctggacgtggttttgactctagatattcgttaggcaatgtgaaatcgtgtagtttgacaacgtgaaattgaccctatcaaaacccctttgcagtgataactaatgaagccaagaatatccttttgaggcacatgtatcagcattctgaagagaaggtacttgatcagcattacatccgcacttgacgcccggtttcccccttttcacatgaatgcttacatacacatgattgagagagtgagagaggctttcttctagtttttcatggactttttcattgcagttgagacaaaagcgggcagcgccagaaaaactctcaccggagcctgtatgcaagcaacagaggtattctgtctcagatactgcctaa

SEQ ID NO: 87 - protein, 97 aa, Malus domestica
MGSLFGDWPSYNPHNFSQLRPSDPSNPSKMTPATYYPTHNRTLPPPDQVITNEAKNILLRHMYQHSEEKLRQKRAAPEKLSPEPVCKQQRYSVSDTA

SEQ ID NO: 88 - DNA, 230 bp, Prunus persica
ccgatccttccactccttctaaaatgacacctgctacctatcatccaactcacagccggacccttcccccacctgatcaagtgataaccaccgaaaccaaaaatattcttttgaggcacatgtatcagaatgatgaagagaagttgagacaaaagcgagctgcatcagaacatcttttaccagagcatggatccaagcaacttagggcttctgtctcagataatgcataa

SEQ ID NO: 89 - DNA, 2747 bp, Prunus persica
ttttgcattatctgagacagaagccctaagttgcttggatccatgctctggtaaaagatgttctgatgcagctcgcttttgtctcaactgcaataaacagtccatgaaaaaactaggagaaagcctctctctctctctctcaatgtatgtccgcagttgtgtgcaatggggaaaagggggcataaagtactcatgtaatacttatactcaccttctcttcatcattctgatacatgtgcctcaaaagaatatttttggtttcggtggttatcactgcaaagggttcagacaggttaatttcattttgtgaaacagcacgattagcaaattgctaatcaatctcaagggtaaaaactacacccataacagtcaactttcagttgtcaggattaatttcaaagcttaactggaatggcctttcttaaagaatcatgagttgttgatacaggaactaagccaatgggtaaggatccagaatgaaacaatgaggagttataaagttttcattattaccttgatcaggtgggggaagggtccggctgtgagttggatgataggtagcaggtgtcattttctacagataaatggaagcataattttaaacaaagaaagagaatgcgtttaattctgatctatttgcatcataaacctaactcatgacatgttctttttgtttcctttttaaaatttgtggttgttgaaagaacaagcaaataacaaattttgcattagttaaatatgttcaaattctgaagatgcatccgtgagcgcccgagcgacgtacaacaatctataatctggatttgtgaccggaaataggtccaacaatgaagcacccagaactctacagctgtaaatgataactttataaaattattagcagaatataagctaaactgcagaatagtctggtggttttggaacagagaaacattttccatcagataggcctgtttcaccacatcaacactaatgaagctaaatgatggttcatacacaccagaatgtatgtttcagacacatcactaagcaaccttctaccaggaatcctgattctctctacaagttggctagacttaaaagttttgctcttcggttcatctcatagcactgagtcctactgtaacacagaatcttcaccttaactttgagaaatgctattattcttaagtattgggaagtaatgtataaatagaatttataagtagaaaacaatatttttcggacttcctcactaaatttctatgttgaaaacatcaagcaatatgtcctagtggcccacaatgtcagcccagttggtttgaaacaaatccaagatttagttacctagcatgcatcaactatttttggaaacacttagggttttcttaatgtataaagtctaagccactctgcatattttcattggttttggatggactaacatggcaccaaagtgcacagttggtcatgtgtcagacccgttttggtcgtacgtgcttcaagtcacctaagataattaatgcatgtgttatactaaacgcgaaagagaatgcagaggatatagaaacttgaaacttaatatatacgaatcccaggcctctccacccaattactaattgtgggatgcttgaactcaagagtttctaaacaacttacaaactagaagaaaaagtaaacaccttatatctggcaggtatttatggggattttctctatagcccatatttaggaatgataaacataattcggacgctgtttaaatactagaacttctaccttagcttagaaacggactaaatgagcttcatgcatatggtaaattaatcatgcagtatccatgatcccatatcattccaatatatgatatttcaatccaactgctataaaatctgaagttaaaaataatgcatctgatttcaaatttcttgtgaactctacaagactagagcactatatgttagctgcagaacaaagcatcaatcctaataaatataagaccaacgtaataacaaagcatgaaactttcggagatccgggaagtgaaacaatgcgcgtagtttaagtgaaagactacaaatcagagacatcaaagcacaaacaacagcagcatattgttccctgatccaaatatcaaacgaatagcatttcttatgaattcatatgagatatggaagaaaacaaatgaaaatatacaagaagatgttccaattcaaaccacactcatttgcacacgatttattacaatgaaagatttatcatttcaatgacattgctgcaacatcatttaccataaaatataaataaaataaaaagaagcaaaatctgcactaaaatctatctttttggatgtacaaacaaaaatcatttcaaactttccataaaattctttcaaactttccataaaattcaaaccgggtctcgaaattttttgatgaaaataacaggaagcccttaccttgagctgaacagtatcataaaaggcagaatctttttttcccacaacaactcagttcagaacaatcaacaacttcagcaatttcaccataacagatgtacaaaaccaaacaaaaagaaagcaaagaacttcacaggtaaagaaaaatcaaagctttgcatgtttatatacagcttaaaagaaaaagaatggcaaattgggtttgattgagatgctgaaaactcacagaaggagtggaaggatcggcgggccggagctggctgaagttgtgagggtcatatgacggccagtcaccgaacaaagaacccat

SEQ ID NO: 90 - protein, 97 aa, Prunus persica
MGSLFGDWPSYDPHNFSQLRPADPSTPSKMTPATYHPTHSRTLPPPDQVITTETKNILLRHMYQNDEEKLRQKRAASEHLLPEHGSKQLRASVSDNA

SEQ ID NO: 91 - DNA, 297 bp, Fragaria vesca
atgggttctttgttcggcaactggccctcatatgaccctcacaacttcagccagctccgaccctcggatcccactactccttctaaaatgactcctacaacctatcatgctacccacaaccggacccttccgccacccgatcaagtgataactactgaatccaagaacattcttctgaggcacatgtatcagcagcatgctgaagagaagttgagacaaaagcgagctgcatcagaaaaccttttaccagagcatggatcaaagcaacttaagggttctgtctcagataagtcctaa

SEQ ID NO: 92 - DNA, 853 bp, Fragaria vesca
ggtgggacaagaaagaattagaacaggatcgtaggctctatataaaatggcacacatggattgattcatagataccaactctgtgcataattcagggtttgtctctagaaaccaacaggccattctctctgtttccgatttggtttgctgcatttcatttcatgggttctttgttcggcaactggccctcatatgaccctcacaacttcagccagctccgaccctcggatcccactactccttctaaaatgactcctacaacctatcatgctacccacaaccggacccttccgccacccgatcaagtgataactactgaatccaagaacattcttctgaggcacatgtatcagcagcatgctgaagagaagttgagacaaaagcgagctgcatcagaaaaccttttaccagagcatggatcaaagcaacttaagggttctgtctcagataagtcctaacaagcaaaactgcctttatcacttccaactgctcatttgttctcacatggatactggaagttcagcattcccatcagtgtgaatattagtgtcacaggcaaaagatgtgtagactgtaccctgtcgtagatagaaggggtatttgattgcacttagttgtaaaagttgcttcactagacatgtagacttgcgtgtacgaattagattacagctttaaacaaataaaatgaatagttacaaggtttgcttgtgttctggttctatatgtctttacaaatgttagttccatgctcatttaaatcgaatgaagaacatgcttcccccaaaattgcttgtatcacgtgactgcgggtttggaaaatacataaaactgataaaagacagcatatgtcaac

SEQ ID NO: 93 - DNA, 2606 bp, Fragaria vesca
ggtgggacaagaaagaattagaacaggatcgtaggctctatataaaatggcacacatggattgattcatagataccaactctgtgcataattcagggtttgtctctagaaaccaacaggccattctctctgtttccgatttggtttgctgcatttcatttcatgggttctttgttcggcaactggccctcatatgaccctcacaacttcagccagctccgaccctcggatcccactactccttctgtaagtcttcactctcccaataaccaatctttgatttgatttgatttcttgtcaaagttttctgctttaatcgtcttgtttaataagatgtagtgtttgttgccaagttctgtttgttttgctctttctgaaccaagttgtgtgaaaagaaggttgctttttgttgtaatcttattcagttctagatgagggcttctgggtatgtgcattaagaaacttttgaggcccagtttgaactgtatcagaattatgggttctggtagtaactataatcttggttcttgtcaagaatagtgtaagtaaatacagaattctagcatcccaagaacttatcagttcttgaattgtcactagattagcttaatccatatattacagtcccttatgttgctcgagttagtcagaatttatcagatggattttctgtttgagcttttgatcattgaacaatgtgttgatcttagttatggcttactgtgattgttaacaatcatgtcaattagatcatcattcctccgtaaagtttcattcttttttactatattgatacaattaaaaatgtttccagccaaatgaagctttgttcttcagttaacatatagtgttgtattcaaatcttagagttcacaagaaatttgcaaacagatgcatcagttacaactaagtgcagtttgatatattttggcatttggacctcttctacatggcaatgatgagtgattatagcttctactgatgaatattccatatacatgaagctcatttaataccttgctaagttacagagggagaactagtatttgtaaagatgcgtattaggtttttccttcctatatatggctgttatagaaaatatctccagaattgtctgccagatagaatgcgtttaataaggttttgttgtatttagtaagatattttgcctttccttatacctagaagacaacttagcaacatttatgtttaaggtgaaggttatgtttcttgtatgactgattgctaagaaaagaatgaattgaaactgctaagagtttatacctaccttgtaaagagaaacaggattctcagtgaataggttgcttaacaatactctgagacacatgttagtatgggtatcaaccatcattcagcttggctaattggcttagtgttgatgaggtgaaaaagtcatatctgattccatttaccagaatacatattatatctcatatatactaataattacagctgcagcatattggctacttattctgatcacgaatctagattagaagggaaattgaagtattaattatagcttgccataggtctctcatcaaaatttgaaaatgatgtgaatcaatgcttgcatgtcatgatttatggctatgattgcaaagtgattggaagtaaatgctttttttctttcttaaaatcatgtctccatctgtctgtagaaaatgactcctacaacctatcatgctacccacaaccggacccttccgccacccgatcaaggtaatgaacaaatatttctatcccttagcttcaataatagcttcagacctaaaaaataagctttaataatagccttttgtttgacgtcacatttactgttgtgagcatttggttcctgcatcattattcaggatctttaagaacgatcccatcaggggtgttgttttacaattgtaaacttacctgattggaacccctttgcagtgataactactgaatccaagaacattcttctgaggcacatgtatcagcagcatgctgaagagaaggtactaatccctttatgcctcctttttccgactgcttacatttgctgaagtgcacacaactgagattagtaagagagaagctttattctagttttcatgacttcttgctgcagttgagacaaaagcgagctgcatcagaaaaccttttaccagagcatggatcaaagcaacttaagggttctgtctcagataagtcctaacaagcaaaactgcctttatcacttccaactgctcatttgttctcacatggatactggaagttcagcattcccatcagtgtgaatattagtgtcacaggcaaaagatgtgtagactgtaccctgtcgtagatagaaggggtatttgattgcacttagttgtaaaagttgcttcactagacatgtagacttgcgtgtacgaattagattacagctttaaacaaataaaatgaatagttacaaggtttgcttgtgttctggttctatatgtctttacaaatgttagttccatgctcatttaaatcgaatgaagaacatgcttcccccaaaattgcttgtatcacgtgactgcgggtttggaaaatacataaaactgataaaagacagcatatgtcaac

SEQ ID NO: 94 - protein, 98 aa, Fragaria vesca
MGSLFGNWPSYDPHNFSQLRPSDPTTPSKMTPTTYHATHNRTLPPPDQVITTESKNILLRHMYQQHAEEKLRQKRAASENLLPEHGSKQLKGSVSDKS

SEQ ID NO: 95 - DNA, 288 bp, Citrus clementine
atgggctctatgctcggcgactggccctcttttgaccctcacaacttcagccaacttcgtccctccgatccctctaatccgtctaaacttacacctgccacctatcgtcctactcacagccgtactcttccaccacctgaccaagtgattactactgaagccaaaaatattctcatgagaaatttctatcagcgagctgaggataagttgagaccaaaaagagctgcctcagagcatctaattccagagcatggatgtaagcaacttagggcttctacgtcaaactga

SEQ ID NO: 96 - DNA, 789 bp, Citrus clementine
ggctaagctaagtctagaatcgtgcggggcattgtgctcgtgggcgctctctctctctctctttctctgtgtctgtctgtctgtctgtctgtctgtctgtctgtctggtggtggctcttgaaattagattagggtgcataaaccggcatttgcaatgggctctatgctcggcgactggccctcttttgaccctcacaacttcagccaacttcgtccctccgatccctctaatccgtctaaacttacacctgccacctatcgtcctactcacagccgtactcttccaccacctgaccaagtgattactactgaagccaaaaatattctcatgagaaatttctatcagcgagctgaggataagttgagaccaaaaagagctgcctcagagcatctaattccagagcatggatgtaagcaacttagggcttctacgtcaaactgagatggacgcatgcaactaggcttccaccttacataagttttcctgctttacccaggaacccaactgttactaaatttccatgggtgtgtgtgtgtgtgtgtgtatctcgtaatggtgtcatatatattgtaatctgttgagttcagatatgtacattttttgtgtactaataatatttgcttgggtgatcccttttacaaggttccgggatgatcagttaatactttgcactccttcctgtgctggtatcattttatgtgaatgactgatgcaggccttcacatcacatgcacatttaattgcatgaggctagtgtgtttatatatgggtttgctgcatttgatttt

SEQ ID NO: 97 - DNA, 2925 bp, Citrus clementine
ggctaagctaagtctagaatcgtgcggggcattgtgctcgtgggcgctctctctctctctctttctctgtgtctgtctgtctgtctgtctgtctgtctgtctgtctggtggtggctcttgaaattagattagggtgcataaaccggcatttgcaatgggctctatgctcggcgactggccctcttttgaccctcacaacttcagccaacttcgtccctccgatccctctaatccgtctgtacgtacttcactatacccattttttttgtccttaatgattaattttcttatcaatcagaaataagcaaaatactacagagctgatcctgataagattttctggagcttgtgcagtgaaataaattattatctttttctagagtcggttctgggtatttctcatgtgaattattagagtctattaaagatttaaaaaagaaaaaaaaagagcacatcatttgggtagcttatgcttcctgttgtgcattaaagaaaaaagtgccattttaaaagtctggtagtgtagatattgttgtggtgtttgtttttcattttgatgcatcctagcttgggatgtctggttcgatcaaatctagtgtaatcacagaaatgttggttgatgatgactttaactgcagcatttctagtaggttaattgtggtgtctactttgtgactatggtgttaaatgctattgatgtatgcttagtttttctatgaagttgtggaggtgtaacatatcagattgatctttcacttagaagctttggcggccgctaaccagaagttgtcagagtctcctacctaagaggcattgtgtatatttcaacatgcttacccaagcaaacttagtgcatgtttgggattgtcatggattttcaaacaatcacttatttgagaatctacttgccaattgtgcatgttactagtcgtttaagttgttggaaacaaattgctgcattgatacctacgttagaatctttttaattatgctttgcctatattattttaatttagttaggcaagatttttggttgtagcccttgtcaagagagccgatatcttgattgagaatttgctcaaggatgatactgacagaaatggtagctggatagcttattgtgataattaatcatcagagatgttgaatcatggcaagattgaagagagagatttgtaaatgatgacattaacttctttaacctacttttggctcttggaagtgacataattggtattgaaataatggacacactaatgaagtatgactctgaggtcgtgaacatgtagagaacttatatgtaggagcaaaatcaggaagagggatagagtgagttaatgatcgagtattttattggcgcaccctcttgttctgtcctctattttatgttcttctgcctcttgattgcttcttgttttatcttcagtttgatccgagtttcaatgaagaaatggccacggtaactaaataacttaagacactcttgtttggaaagttgttattgcgttgcattagattatatgatacatttgattcaacatgaattttgatatctttactcgctaacatatatattaatatatttaattggcttaaagattactgttgttaaatcaatgacccatcaaataaggtgaaccaagttctctcatgtataatttcctttttttgtgtctgtctagaaacttacacctgccacctatcgtcctactcacagccgtactcttccaccacctgaccaaggtattgacacaatctttgtatctccttattgcatcaaaagcttcttgccagaagggttattcctggctttttgaatcatctgctgtatcatatacatagtaatctttaaattgattttgtgacaattcctctcttcacgtggtgtatattttcatgatacgatctttcaatctgttaaacttgttttgctaggtttggtttcatggatgatgttgaaatttttatgtttgtgtatatcaatgccagcacttccatacatgctgaatccattaactatctttttagaaaatattgatggtgtacaactatatatggataagccagtttggtttttagaaaagactgaaaaattagggtagagactctctattataacagagagatcatgattgcttgggtagtaaaagaattctttatttaaatcttgaccaagatgcgattattaatgggattgtagttgtgcgtttgtctgttgtcaatgtgtacattgtatgtaattctggtagaacttacttttgttatagcttcccatgctgtttttgtttcgccacataattactatggagacaaatagacaatgaacactgtttttggcagtgattactactgaagccaaaaatattctcatgagaaatttctatcagcgagctgaggataaggttagtatcatttaagatgtatcttgtgccagtacatgtgtagcaagagagtgaatttacaaacacttctttaacttcttcctctcttttggcagttgagaccaaaaagagctgcctcagagcatctaattccagagcatggatgtaagcaacttagggcttctacgtcaaactgagatggacgcatgcaactaggcttccaccttacataagttttcctgctttacccaggaacccaactgttactaaatttccatgggtgtgtgtgtgtgtgtgtgtatctcgtaatggtgtcatatatattgtaatctgttgagttcagatatgtacattttttgtgtactaataatatttgcttgggtgatcccttttacaaggttccgggatgatcagttaatactttgcactccttcctgtgctggtatcattttatgtgaatgactgatgcaggccttcacatcacatgcacatttaattgcatgaggctagtgtgtttatatatgggtttgctgcatttgatttt

SEQ ID NO: 98 - protein, 95 aa, Citrus clementine
MGSMLGDWPSFDPHNFSQLRPSDPSNPSKLTPATYRPTHSRTLPPPDQVITTEAKNILMRNFYQRAEDKLRPKRAASEHLIPEHGCKQLRASTSN

SEQ ID NO: 99 - DNA, 288 bp, Citrus sinensis
atgggctctatgctcggcgactggccctcttttgaccctcacaacttcagccaacttcgtccctccgatccctctaatccgtctaaacttacacctgccacctatcgtcctactcacagccgtactcttccaccacctgaccaagtgattactactgaagccaaaaatattctcatgagaaatttctatcagcgagctgaggataagttgagaccaaaaagagctgcctcagagcatctaataccagagcatggatgtaagcaacttagggcttctacgtcaaactga

SEQ ID NO: 100 - DNA, 610 bp, Citrus clementine
tgcggggcattgtgctcgtgggcgctctctcactctctctttctctgtgtctgtctgtctgtctgtctgtctgtctgtctggtggtggctcttgaaattagattagggtgcataaaccggcatttgcaatgggctctatgctcggcgactggccctcttttgaccctcacaacttcagccaacttcgtccctccgatccctctaatccgtctaaacttacacctgccacctatcgccctactcacagccgtactcttccaccacctgaccaagtgattactactgaagccaaaaatattctcatgagaaatttctatcagcgagctgaggataagttgagaccaaaaagagctgcctcagagcatctaataccagagcatggatgtaagcaacttagggcttctacgtcaaactgagatggacacacgcaactaggcttccaccttacataagttttcctgctttacccaggaacccaactgttactaaatttccatgggtgtgtgtgtgtgtgtatctcgtaatggtgtcatatatattgtaatctgttgagttcagatatgtacattttttgtgtactaataatatttgcttgggtgatcccttttac

SEQ ID NO: 101 - DNA, 2744 bp, Citrus clementine
tgcggggcattgtgctcgtgggcgctctctcactctctctttctctgtgtctgtctgtctgtctgtctgtctgtctgtctggtggtggctcttgaaattagattagggtgcataaaccggcatttgcaatgggctctatgctcggcgactggccctcttttgaccctcacaacttcagccaacttcgtccctccgatccctctaatccgtctgtacgtacttcactatacccattttttttccttaatgattaattttctcatcaatcagaaatgagcaaaatactacacagctgatcctgataagattttctggagcttgtgcagtgaaataaattattatctttttctagagtcggttctgggcatttctcatgtgaattattagagtctatttaagatttaaaaaagaaaaaaaaagagcacatcatttgggtagcttatgcttcctgttgtgcattaaaaaaaaaaaagtgccattttaaaagtctggtagtgtagatattgttgtggtgtttttttttcattttgatgcatcctagcttgggatgtctggttcgatcaaatctagtgtaatcacagaaatgttggttgatgatgactttaactgcagcatttctagtaggttaattgtggtgtctactttgtgactatggtgttaaatgctattgatgtatgcttagtttttctatgaagttgtggaggtgtaacatatcagattgatctttcacttagaagctttggcggccgctaaccagaagttgtcagagtctcctacctaagaggcattgtgtatatttcaacatgcttacccaagcaaacttagtgcatgtttgggattgtcatggattttcaaacaatcacttatttaagaatctacttgtcaattgtgcatgttactcgtcgtttaagttgttggaaacaaattgctgcattgatagctacgttagaatctttttaattatgctttgcctgtattattttaatttagttaggcaagatttttggttgtagcccttgtcaagagagccgatatcttgattgagaatttgctcaaggatgatactgacagaaatggtagctggatagcttattgtgataattaatcatcagagatgttgaatcatggcaagattgaagagagagatttgtaaatgatgacattaacttctttaacctacttttggctcttggaagtgacataattggtattgaaataatggacacactgatgaagtatgactctgaggtcgtgaacatgtagagaactaatatgtaggaacaaaatcaggaagggatagagtgagttaatgatcgagtatttgattggcgcaccctcttgttctgtcctctattttatgttcttctgcctcttgattgcttcttgttttatcttcagtttgatccgagtttcaatgaagaaatggccacggtaactaaataacttaagatactcttgtttggaaagttgttattgcgttgcattagattatatgatacatttgattcaacatgaattttgatatctttactcgctaacatatatattaatatatttaattggcttaaagattactgttgttaaatcaatgacccatcaaataaggtgaaccaagttctctcatgtataatttcctttttttgtgtctgtctagaaacttacacctgccacctatcgccctactcacagccgtactcttccaccacctgaccaaggtattgacacaatctttgtatctccttattgcatcaaaagcttcttgccagaagggttattcctggctttttgaatcatctgctgtatcatatacatagtaatctttaaattgattttgtgacaattcctctcttcacgtggtgtatattttcatgatacgatctttcaatctgttaaacttgttttgctaggtttggtctcatggatgatgttgaaatttttatgtttgtgtatatcaatgccagcactttcatacatgctgaatccattaactatctttttagaaaatattgatggtgtacaactatatatggataagccagtttggtttttagaaaagactgaaaaattagggtagagactctctattataacagagagatcatgattgcttgggtagtaaaagaattatttatttaaatcttgacccagatgcgattattaatgggattctagatgtgcgtttgtctgttgtcaatgtgtacattgtatgtaattctggtagaacttacttttgttatagcttcccatgctgtttttgtttcgccacataattactatggagacaaatagacaatgaacactgtttttggcagtgattactactgaagccaaaaatattctcatgagaaatttctatcagcgagctgaggataaggttagtattatttaagatgtatcttgtgccggtacatgtgtagcaagagagtgaatttacaaacacttctttaacttcttcctctcttttggcagttgagaccaaaaagagctgcctcagagcatctaataccagagcatggatgtaagcaacttagggcttctacgtcaaactgagatggacacacgcaactaggcttccaccttacataagttttcctgctttacccaggaacccaactgttactaaatttccatgggtgtgtgtgtgtgtgtatctcgtaatggtgtcatatatattgtaatctgttgagttcagatatgtacattttttgtgtactaataatatttgcttgggtgatcccttttac

SEQ ID NO: 102 - protein, 95 aa, Citrus sinensis; identical to SEQ ID NO: 98

SEQ ID NO: 103 - DNA, 297 bp, Cucumis sativus
atggggtctatgctcggtgacctgccgtcatatgaccctcacaacttcagccaactccgaccctctgatccttcaactccttctaagatgattcctacaacctatcatccaacccacagtaggacccttcccccaccagatcaagttataaatactgaggccaaaaatatacttatacgacacatttatcagcatacagaagaaaagtcaagaacaaagagacctgcagccgagcatcccatgcccgagcacggaagcaagcaaccaagagcatctactaccaacacttcaaattga

SEQ ID NO: 104 - DNA, 737 bp, Cucumis sativus
agttgtaaatccaatggcgatgtgattcctaatgattcccttctagaaaaaccacttcttcttcctttttcttcttcatcttcttcttctcctctgtagatttcgaacaatcaacatatattcagcagcattttcatggggtctatgctcggtgacctgccgtcatatgaccctcacaacttcagccaactccgaccctctgatccttcaactccttctaagatgattcctgcaacctatcatccaacccacagtaggacccttcccccaccagatcaagttataaatactgaggccaaaaatatacttatacgacacatttatcagcatacagaagaaaagtcaagaacaaagagacctgcagccgagcatcccatgcccgagcacggaagcaagcaaccaagagcatctactaccaacacttcaaattgagcttgggaggacattttcctccaacaaattaaagctattcatgttgtgatagatgactcctgtataatgagagtgaattgcctgctcactgaagaagaaacggctcggccagacatttacatgttgtatatagattttactccttgtaagattaccctaaactcaaccacatcaaattgttgtcaaaatcataaaactcagttgaagaattgtaactatatgcgtgtgcttccaaacaatattattggaggcctccttcctataaagcaaaagatcctcaccttgttctttttcctggtttggat

SEQ ID NO: 105 - DNA, 2585 bp, Cucumis sativus
agttgtaaatccaatggcgatgtgattcctaatgattcccttctagaaaaaccacttcttcttcctttttcttcttcatcttcttcttctcctctgtagatttcgaacaatcaacatatattcagcagcattttcatggggtctatgctcggtgacctgccgtcatatgaccctcacaacttcagccaactccgaccctctgatccttcaactccttctgtaagttctcaataatggcctaatcatcataccctttctttctcttcttctaattcatttctctatttctttaaacccttcactccttttcttgatcttgggtgtttccctccgttttgcatgattctttgtttcgtttctattaatgggaagttgctcgacttgtggtagggtgtgaattgtgatggggttctgattaaatttaacctcctcttgatcttctttgctctttcgttgttggggctcagctaatttttggttgggattattggcattattgatctgttttttgttgttgtcttatttggagattcccattgcgtgatacttggaaaatcttgaattttggcatgtgggttctttgtttaggcatgtttgtagatgtgggttacaattaagttccgcatttgacgtgtttgaatgttctatgaattttccaaatgtttctcgtaggtagaagttgaactttgtttacccccccacttccccaccctgttggaacaaatgaatcagatggtgtttttgttttaattttcactctctctaggtattataatcttgtgaagtcaatcctctgaaagaaacggttccgttaacccagtcgcaaagaattcgctatatcatgatacgaaggggacagaaatctgaaggaacaatagcactgagttaccgatcttccggagaaacgattctatttaattctcatatgtagcccatcttcaagtttcggaattctacgattaggatccctagtgcttttaccctttgaatggagcagcctgcaaccaatttaagctatctcttcctttgaatataataaaactcattgggccaaaatgaaaagcccttgaattgcagggttatgaatttcttctatactgcgaatagttatgctatgctcatgctttctctgatttcttgagtggtttacactttgctttcaagggtttaaacttaaacgtcccaattaaatacttgatataaagtttgaagttattcaaacagtactcctttgagccactttagtatttttttgcctttgtccttctgccctgaatggaaatatttagataatgcagaaggattaacaactctaagaaacattagtgagaactgagaagctgttgactgaagagattctaaaagcatctcaactaaagctaccagatgtggtatcccttttggatataaagaaaggaagcaaatagtttatagtttctacattaaagtaaggtcaaatcaaattctccttgtaattgtgtgtacaaacttgtgcatgctttggatttatctcccatctttgaattgatctcaaatccgacacctcatgggtggccatcccttgttaatttgcggtagattgagtgaacatgtgagtggaagagctgctgtctggatcaactaaaatgctttcattcaaagcttaatctgcgctataatgcaacatattgtttactgtatctgctagctgcaagtaaagtagaacaagaagatgaattaactatttttcaatgtaaggaataagttgatgttggaattacaatgctaagttggttttatttttatgtagaagatgattcctgcaacctatcatccaacccacagtaggacccttcccccaccagatcaaggtaagcaagaaaagttgtttcttcactactctaccgataactttgtatcttgctcggataacagtattctttagtcagtagttttccactgttggctttagtttccatctttcttctgctttttactcaaaaaagaactcctcactgatttttactgcagttataaatactgaggccaaaaatatacttatacgacacatttatcagcatacagaagaaaaggttagtaaaagaaatctgttatgctttgatctgaaatacaatccatacatgtagtaagctaccttgtgagaccactcacccattccctgtggacttcccctggtctttgtaatggcagtcaagaacaaagagacctgcagccgagcatcccatgcccgagcacggaagcaagcaaccaagagcatctactaccaacacttcaaattgagcttgggaggacattttcctccaacaaattaaagctattcatgttgtgatagatgactcctgtataatgagagtgaattgcctgctcactgaagaagaaacggctcggccagacatttacatgttgtatatagattttactccttgtaagattaccctaaactcaaccacatcaaattgttgtcaaaatcataaaactcagttgaagaattgtaactatatgcgtgtgcttccaaacaatattattggaggcctccttcctataaagcaaaagatcctcaccttgttctttttcctggtttggat

SEQ ID NO: 106 - protein, 98 aa, Cucumis sativus
MGSMLGDLPSYDPHNFSQLRPSDPSTPSKMIPTTYHPTHSRTLPPPDQVINTEAKNILIRHIYQHTEEKSRTKRPAAEHPMPEHGSKQPRASTTNTSN

SEQ ID NO: 107 - DNA, 294 bp, Cucumis melo
atggggtctatgctcggtgacctgccgtcatatgaccctcacaacttcagccaactccgaccttctgatccttcaactccttctatgattcctgcgacctatcatccaacccacagtaggacccttcccccaccagatcaagttataaatactgaggccaaaaatatacttatacgacacatttatcagcatacagaagaaaagtcaagaacaaagagacctgcagccgagcatcccatgcccgagcacggaagcaagcaaccaagagcatctactaccaacacttcaaattga

SEQ ID NO: 108 - DNA, 714 bp, Cucumis melo
aatggcgatgtgattcctaatgatacccttctagaaaaaccacttcttcttcctttttttcttcttcatcttcttcttttcctctgtagatttccaacaatcgtcttatattcagcagcattttcatggggtctatgctcggtgacctgccgtcatatgaccctcacaacttcagccaactccgaccttctgatccttcaactccttctatgattcctgcgacctatcatccaacccacagtaggacccttcccccaccagatcaagttataaatactgaggccaaaaatatacttatacgacacatttatcagcatacagaagaaaagtcaagaacaaagagacctgcagccgagcatcccatgcccgagcacggaagcaagcaaccaagagcatctactaccaacacttcaaattgagcttaggaggacatttccttccaacaaagttaaagctattcatgttgtgatagatgagtcctgtataatgagagtgaattgcttgctcactgaagaagaaacggctcggcccgacatttacatgttgtatatagattttacttcttgtaagattgccctaaactcaaccacatcaaattgttgtgaaaatcataaaactcagttgaagaattgtaactatatgcgtgtgcttccaaacaatattattggaggcctccttcctataaagcaaaagatcctcaccttgttctttttc

SEQ ID NO: 109 - protein, 97 aa, Cucumis melo
MGSMLGDLPSYDPHNFSQLRPSDPSTPSMIPATYHPTHSRTLPPPDQVINTEAKNILIRHIYQHTEEKSRTKRPAAEHPMPEHGSKQPRASTTNTSN

SEQ ID NO: 110 - DNA, 294 bp, Castanopsis sieboldii
atgggttctctctttggtgactggccgtcatttgaccctcacaacttcagccaactccgaccctccgatccttctagtccttctagaatgacacctgcaacctatcatcctactcacagccgcacgcttccaccacctgatcaagtgatcactactgacgccaaaaacattctcttaaggcacatctatcaacgtactgaagagaaggatctgagaccgaagagagctgcgccagaacatcttgtacctgagcatggatgcaagcaacctagggcatcttccagttcctgctga

SEQ ID NO: 111 - DNA, 589 bp, Castanopsis sieboldii
cattgtgctctctttctctctccccctagattttttgtgccgaaagaaaccagcattttatgggttctctctttggtgactggccgtcatttgaccctcacaacttcagccaactccgaccctccgatccttctagtccttctagaatgacacctgcaacctatcatcctactcacagccgcacgcttccaccacctgatcaagtgatcactactgacgccaaaaacattctcttaaggcacatctatcaacgtactgaagagaaggatctgagaccgaagagagctgcgccagaacatcttgtacctgagcatggatgcaagcaacctagggcatcttccagttcctgctgagcccttatcttgttatatggaaccccaaaatagttaattcgtgtaaatgtttttgtcatgccaaatatgcgtgagtttcttgtgggttgaaaaggggttttattttgcttgatcattgctgtaagcagcttaaccagaagtgtagattttgtgtgtataattcataaatactatagagttgggtgatccctattacagtttacatggatgatgaaatgaaagtaatagatattatt

SEQ ID NO: 112 - protein, 97 aa, Castanopsis sieboldii
MGSLFGDWPSFDPHNFSQLRPSDPSSPSRMTPATYHPTHSRTLPPPDQVITTDAKNILLRHIYQRTEEKDLRPKRAAPEHLVPEHGCKQPRASSSSC

SEQ ID NO: 113 - DNA, 306 bp, Actinidia setosa
atgggttctttgctcggggactggccttccttcgaccctcacaacttcagccaactccgaccctccgatccttcaaatccttcaaaaatgacgcctgtcacttatcatcctactcatgatcggaccattccaccccctaatcaagtgatttcttccgaagccaaaaatatacttctgcggcatttctatcagcgtgccgaggacaagctgagaccaaagagagctgcgtcggaacttctgacacccgaacacggaggcaagcatcccagggcctcggcttctgcttcaaaagcgcctccctgctga

SEQ ID NO: 114 - DNA, 617 bp, Actinidia setosa
ccccaaaccactccattgttctcttcctttatctcgattcttccattgaaatcgcagcttccaatccatgggttctttgctcggggactggccttccttcgaccctcacaacttcagccaactccgaccctccgatccttcaaatccttcaaaaatgacgcctgtcacttatcatcctactcatgatcggaccattccaccccctaatcaagtgatttcttccgaagccaaaaatatacttctgcggcatttctatcagcgtgccgaggacaagctgagaccaaagagagctgcgtcggaacttctgacacccgaacacggaggcaagcatcccagggcctcggcttctgcttcaaaagcgcctccctgctgagctttcctgctattgcttgaagaatatctcaagagtcaagttctattgaatgtcattgtgaatattcccatcatcatattaccaatttgtgttttccgcaattataaagggtatttctgtgctcattgtacattttgcatgtataaactccagttgttcaccttccccttttcaagtgctgatgtagaatctagtctcatcgcatgcttctcccctttgcctgtgttgggcattacatagtcgt

SEQ ID NO: 115 - protein, 101 aa, Actinidia setosa
MGSLLGDWPSFDPHNFSQLRPSDPSNPSKMTPVTYHPTHDRTIPPPNQVISSEAKNILLRHFYQRAEDKLRPKRAASELLTPEHGGKHPRASASASKAPPC

SEQ ID NO: 116 - DNA, 282 bp, Solanum tuberosum
atggggtcaatgtttggtgaatggccctcaattgaccctcacaatttcagccagcttcgcccttctgatccctcaactccttctagaatgacacccgtgacttatcgccctactcatgataggactcttcctccaccaaatcaagttattagttcagaagccaaaaatatacttctgagacacctagagcagcgtgctgaagagaagttgagaccaaagcgagctgcggctgaaaatctggcacccgagcatgggtcgaagcatcttaaggtatccaactga

SEQ ID NO: 117 - DNA, 659 bp, Solanum tuberosum
cacaatatatatatttgtgctctctctttaaagagtggcattgttctctggattcttcccattttgggtgctatggggtcaatgtttggtgaatggccctcaattgaccctcacaatttcagccagcttcgcccttctgatccctcaactccttctagaatgacacccgtgacttatcgccctactcatgataggactcttcctccaccaaatcaagttattagttcagaagccaaaaatatacttctgagacacctagagcagcgtgctgaagagaagttgagaccaaagcgagctgcggctgaaaatctggcacccgagcatgggtcgaagcatcttaaggtatccaactgagatgcttttctttttggtgctacccccgggcgggaagaagatgaggtaatgcgaaacaggacgatacacaacttggttttagaagagtaactaacttctaaataggttaaaatcttctggttttctgcatatttctgtaaatattgctgtaatgatgcagatgcatgttgttgtaaaactatgaagagcttgtttatcactagtcatatagcaaatgagatgtacactagagaaaatgttgttggatgagatttctctgcatgagtattgataaatgtttcatgctgagggtttatcggaaacaa

SEQ ID NO: 118 - DNA, 5156 bp, Solanum tuberosum
cacaatatatatatttgtgctctctctttaaagagtggcattgttctctggattcttcccattttgggtgctatggggtcaatgtttggtgaatggccctcaattgaccctcacaatttcagccagcttcgcccttctgatccctcaactccttctgtaagaacccttttcattttttttcaatttttttttatataaaacttaaatctttgattttttttaacacccttttccccattcaatctgttttttgaattctactgtgtttttagctgatttatgttcgacccatttttgtctgatagcaaaaaatgcattcttgggataattttagctgatttgtgttcctgaatggagcagaatgaaatccagctaatttgggactgaaatgttgttgattattttgttgacccatttttgctatgtttgttttatgcaaaaaaaaaaaaaaaagtgttggaacaattttgtgggtgtatttgaaaaattcttgatcttttcaagttagagtttttatttggagaaactttgatttgtgttaagagatcaattgtttagtatttgaaatgaaatgggtccgaacggatcagaatgaatatgtatatatagggagagaattcatatagctgaagtgttgaacctagctaatttgggattgagatgtattacgtttccctctgttttaatttgtttgtcttactttccttttcagtttgtttaaaaaagaatgtctctttacttttttgtcagctctttaatttcaactttcaagattagaaggcattttggtatattctacgtatgttgagtttaagtctactttactttcgtaagctccgtgtcaagtcaaaattagacaaacaaattgaaaaagagggagtattagttaattgactgtcgttttttgcatgcttatgtggtgttgtgttactaaagctagcataaaatactctgatttaggcttaaggctgatatttcagtttgagaatgttttctgatgtaatgatgtttgatttgcaaatgggctatagggttaccataatggtgcctaatctataaaagagtgatatttaagctatatgtaatttaatgcggattttgtataagtgtattctagttttggtgcattaacttctatcagatagacggcatatacatatatcaatgcgtgtggagatatcatattacctcaagcaataatgtggatcattatttatcggaaaaatgaaacaacaagaagaattgagtacaatctatgtagctgtcttcttgatggtttggtggtgtctaggtttaatttgtcgcttctgtgagacttaattgcatgctatgctcagctttgtgtaaacaatccctctttgctagtgtagactcacaccacgtggcatttgctgccataatacattgtacactagcttaatgataattgcgtattagattgttatctattatgacattggttataagcatgcactgcttatgttactgttatttcagttactcttacttctgaacgcatattgataaatatgtaggagaggaatcagaaaacaataatattctaagacttcgtagaaaagatatataatacttgtttcaatgtatttttactatctgttgcttcatttttctttgtatcttgtcatctttttcttccctccccggacaccacttgtgggattccattgggtatgttgttgttgttgttgtagggtataacatgttagtatgaaatgtcttactctcaagagaagcggaaaaaagaagccttatcggctcttgtattaccataacaacattttcattaactgatacaacttttttccttttttgagttccttcttaccttttctcattgtgcttcttggctccttgaatatgatcccattgatccttcggctttcaagcctcaagaagcaattgaagacctcatcttcgtcccccgttatgtcgaccaatcgtttacctcgctttaatgtagttcttttgatccacgctgaagctaccacctttaatcctttatctgtggtggctaatttaattgcttggagagaagtgagaaggttgattttcttcttgtggtgacagtctttttctcagtggtgccatgctttgcttctatttacagcattcactttaatttctctagaaccgtgtatgttacaacactaggtttatgatttaccttgtgagcttttgatgtagaagagctgcaaattatttgaagcatctattatccacaataaactttttctatattattgttgtgtatacagtctggtctccctatttccttgccctttcccccctttctggtagttaaaacttaaaaacaaaaataatgaaggtaaatatagtggagtgtttgaagataaagttacttggataactcatacattgaggctcattatgtctccaaaggagttgtcgtgaagctgaatttgtgaagttacttactacttactataatggtttcctataacaatcggcttgagggactctcttcacgggaagtcatgcctgggcaagggtagtactaggatgggtgacccccaaggaagtccttgtgttgcatcgttgctttgaatggcatgataagtgaagtgaactaacatttgtaatctgcatatgcacttccaaatctctttaaaaattattatagcattacataagtgttccatgatgaagttatttgttggtaaaccttttgtactttgacttttagtagctgaaacaatctctctgtttcttttttttccagtttcgaggatactagtttttttttttttccacttatctaatcctgtgttctttctttttctagagaatgacacccgtgacttatcgccctactcatgataggactcttcctccaccaaatcaaggtaaatcaagaacatgattctttttgtttcttacacatcttttgatatcataatttacatttatcttttagttgctcttaccctctacctttatcaggtatttggattatatacatttgactagaatccttgagccaagggtttgtcagaaatagactctctaccttccaaggtaggggtagggtctctatgcacactaccctcctcagaccccacttatgggattacattgggcatgttgttgttgtatatatttgactatactaacctcccacccttcctccctaagtggatggtgaatggaataactttagctcagctgctgttattggttattcttatgagttggtccttagaattaagccagtcatcctcttgaggatttgaacttcagtttattcactttagaagttgtttcaacctcaaatgattgtgtcgcaaaagcattgggcccggatcaataaggttgccaactggcaatgagttgtttatgagtagcagaatcaagcacccgtaattactagttttgaatttttcgctccggcttttgtgaagataaacagtctaaaaagttctctgtggatgctagtgttgaggctttcatcgaaacaaagagtgagacctgtctggtgcttttttctatgttagtcaggtgaaagtaaatagattagtgtattaagtaaggcactgctaaagcctctattcccgttttttgagcgataaaactcttagcttccagcaagctaggtgcataagctttttttactacataaaagattccaagaaatgcaggcagggtagagctcggcagagggttcgagaatagggtagggtagggtccttaagattcaataagaaagagtgtgcctttaaagcaaataatgcagcccttttctttcctcatatggctatatttaattcaaaactcctgtaaatgtgtgtgtttacaagtgacaatttgtcatgttcatctcctgttatgaaattgtggtttaattctttcggataatattataattatcttgccccactaccacatctctatactcgactaagaatgatttcaattgcactaggataaactacttttcaatagctttaattgcatcatcttatgtaagatcctattaccgaaaaggaaaaaagggaattctctttggttgtaacatttggtaaatatagtatcaatagccatctaaagtacattcatgttattggtattaaacttttatgaggatgcacatattcgtgagccgagggtctatcgaaaacaacttctctacctttgaaggtatgggtaagttgcatacacaccaccctccccagacctaatcatgaaaatctaatgtaatgtgcacgtttgtttgtcggtttactttctcatgcttcatctcaatcttttctttttttctttattgaatctcgattaatcagataagggaagtatactagatatgaattgcgtgagcttaaaacttttgcacttaaggtatgcttatatctaactcaacatatactaatgacctgcatttgaatttccagttattagttcagaagccaaaaatatacttctgagacacctagagcagcgtgctgaagagaaggtcagtattcattttgatcgacatttattccttcttttgatggatatttggcaatttgtaattgtgttatattgtgaggattcatcttaaagtgactagttttacaatggctatcaacctattgcaatgtttctcctttatccagtcttgtgaccgacaatatgagcaaactcacacataatcaatgaggattcgtataaccaaccctagcttccttgggattgaggtgttgttgttgtgaaatgtgttctatccctttacggttaaaatgcttttttgtgttgtagttgagaccaaagcgagctgcggctgaaaatctggcacccgagcatgggtcgaagcatcttaaggtatccaactgagatgcttttctttttggtgctacccccgggcgggaagaagatgaggtaatgcgaaacaggacgatacacaacttggttttagaagagtaactaacttctaaataggttaaaatcttctggttttctgcatatttctgtaaatattgctgtaatgatgcagatgcatgttgttgtaaaactatgaagagcttgtttatcactagtcatatagcaaatgagatgtacactagagaaaatgttgttggatgagatttctctgcatgagtattgataaatgtttcatgctgagggtttatcggaaacaa

SEQ ID NO: 119 - protein, 93 aa, Solanum tuberosum
MGSMFGEWPSIDPHNFSQLRPSDPSTPSRMTPVTYRPTHDRTLPPPNQVISSEAKNILLRHLEQRAEEKLRPKRAAAENLAPEHGSKHLKVSN

SEQ ID NO: 120 - DNA, 282 bp, Solanum lycopersicum
atggggtcaatgtttggtgaatggccttcaattgaccctcataatttcagccagcttcgcccttctgatccctcaactccttctagaatgacaccggtgacttatcgccctactcatgacaggactcttcctccaccaaatcaagtaattagttcagaagccaaaagtatacttctgagacacctagagcagcgtgccgaagagaagttgagaccaaagcgagctgcggctgaaaatctggcacccgagcatggatcgaagcatcttaaggtatccaactga

SEQ ID NO: 121 - DNA, 728 bp, Solanum lycopersicum
aacccacaatatatatatttgtgttttctctttagagagtggcattgttctctggattcttcccattttgggtgttatggggtcaatgtttggtgaatggccttcaattgaccctcataatttcagccagcttcgcccttctgatccctcaactccttctagaatgacaccggtgacttatcgccctactcatgacaggactcttcctccaccaaatcaagtaattagttcagaagccaaaagtatacttctgagacacctagagcagcgtgccgaagagaagttgagaccaaagcgagctgcggctgaaaatctggcacccgagcatggatcgaagcatcttaaggtatccaactgagatgcttttcttttcggtgctacccctgggcgggaagaagatgaggtaatgcggttgcaaacaggacgatacacaacttggttttagaagattaactaactcttctaaataggttaaaatcttctggtttttctgcatatttctgtaaatatgactgtaatgatgcagatacatgttgttgtaaaactatgaagagcttgtttgtcaagtagtcatatagcaaatgagatgtacactagataaaatgttgttagatgagtattgataaatgtttccctccgaggatttatcggaaacaacctctgtgatccgtcctctgtgaccctagaaaatgatattttatcataatgatcaaactttttaacattga

SEQ ID NO: 122 - DNA, 5269 bp, Solanum lycopersicum
aacccacaatatatatatttgtgttttctctttagagagtggcattgttctctggattcttcccattttgggtgttatggggtcaatgtttggtgaatggccttcaattgaccctcataatttcagccagcttcgcccttctgatccctcaactccttctgtaagaacccttttcattttttttctattttttttttcatataaaacttcaatctttgattttttctgaacacccttttccctattcaatctgtttttttgaattttaatgtgttttttagctgatttatgttcgacccatttttgtctgatagcaaaaagatgcattcttggacagttttagctgatttcatttgtgttcctgaatggagcagaatgaaatccagctggtactatgtgttaattctccgttttaagaacgtctcttttttcttttgtcagctctttaatttcaactttctactgacatgtctaaggccacaagattaaaaaagagcaattttggtactatgtctgttaatttaagaccacaagtttcaaaagtttactttactttcttaaactccgtagaggcaaacaaattgaaacggagagagtagtttattgattgtcattttttgcgtgtttatgtggtgttatgttacttaagctctctgatttaagcttaagccttatgtttcagtttgagaatgttttctgatgtaatgatgtttgctttgtaaatgagatatagggttacggtaatggtgcataatctatagaagaatgatatttatgcttttaagtaaattcaacgtggatttggattaagtatattctagttttggtgcattaacttctatgggatagatggcatatacatatatcaatgcctgtagagatctaatgttatctcaagcaacaatgtggatcattattaatcagaaaaatgaaacaacgagaagaattgaatagaatctatgtagttgtcttattgatggtttggcatagtctaaatttgtctcttgtgtgagacttaattgcacgctatgctcagctttgtataaacgatcttctttgatggtgtagagtgtagactcacaccacttgacatttactgccatattacattgcactctagtttaatgataaccacatacagattgttatctattaaggcattgattgtaagtatgcactgcttatgttactgttatatttcagttattcttctgaatgcagattgataaatatgtaggggaggaacagaaaaccatgttatcctaaaactttaaagaaaagatacataatacttgtttcaatgtgtttttactatctgttgcttctttttgctttgtatcttgatattttactgtcatttttttctccctccccagataccacttgtgagattccattgggatgttgttgttgttgtggggtatatcatgttagtatgaaatgtcgtactcttaagagaggcggaaaaaagaagccttatcagaacttctattaccatgacaattttcattaactgatgcagcttttttccttttttgagttccttcttaccttttcttcttgtgctgcttggctccttgaatatgatctgcccattgatccttcagctttcaagcctcaaaaagcacttgacgacctcatcttcatcccccgttagttaaccaattgtttaccatgctttaatgcatttcttttgatccacgtcgaagctgccacctttttcgtggtggctaatttaattgcttgaacagaagtgagaaggtcaattttcttcttgtggtgacggtctttttctcagtagtgccatgctttgcttctttttacaccattcactttaattcctctagaatcgtgtatgttacagcactaggtttatgatttaccttgtgagctttagatgtagaagagctgcaaattatttgaagcatctattatccacaatgaactttttctgattattgttgtgtatgcagtctggtctcccttttcgttgcccttttttcccctttctggtagttatcacaaaaataaggaaggtaaatatagttgagtatttgaagataaagttacttagataactcatacattgaagatcattatgtctccaaaggagttgtcatgaagctgaatttgtgaagttccttactacttactataatggtttcctataataatcggcttctgggactctcttctctggaagtcatgcctgggcaagagtagcaccataatgattgacccccaggaagtcctcgtgttgcataattgctttgaatggcatgataagtaaagtgaattaacatttgaaacctgcatatgcacctcaaaatctctttaagaattattagagttacataagtgttacatgatgaagttatttgttgaacttttgtactttcgctttcagtagctgatttttgtccagttttgaggatactagttattttatttatccacttatccaattctgtgttctttctatttatagagaatgacaccggtgacttatcgccctactcatgacaggactcttcctccaccaaatcaaggtaaatcaagaaaatgatttttttttttgttccttagacgtctttgatatcataatttacatttatctattggttgctcttaccctctacctttatcaggttttcggattatatagatttgactagaatccttgagccgaggttttgtcagaaatacctctctaccttccaaggtaggggtaaggtctctatacacactaccctcctcaaacctcacttgtttaatatggataaaaataataataccaataatagaaaacaatactcctatattttttggttaataaataaaaatggttgaaataataaagatagtgattggacattatttggacaagatttacctaatctacttttcaaaattaaaatctttattcaaagagacacaagactaattattcattaaggcaagggatttaatattggatctctaaagtcttaaataactaaatcaaggtgatattgatccattttaatcatgattaattacaactgctcaacgcttaaacatccaacaatgtgtcagagtgatacgtataaagtattgttggatgttattgtccaacaatactttatatttgggcatgttattgttgtatatatttgactagaatcctacccttcctccctaagtgaatggtgaatggaataactttagctcagctgctgttattggttattcttatgagttggtctttagaattaagccagtcatctcttgaggatttgaacttcagtttattcactttaaaagttgtttcaacctcatatcattttgtgtcgcaaaagcattgggcatggatcaataagaatgtcaattggcaatgagttgtatatgggtagcagaatcaagcacccctaattactagttttggatttcaagcacccctaattactagttttggaccttctccctctgcttctggttttggtgaaggtaaaatagtctaagaagttctccgagtatgtctgtgttgaggctttcatcgaaacaaagagtgaggcctgcctggtgttttttctatgttagtcaggtgaaaggattggttcactataaatagtttagtgtattaagtaaggcactactaaagcccccattcccctttttgggcgataaaactcttagcttccagcaagctaggtgcataagctttttttattacataaaagattccaaacaatgcaggcagggtagagctcggcagagggttcaagaattgggtagggtagggcccttaagattcaataagaaagaatatgcctttcaagaaaataatgcagctattacctttcctcatatggctatatttaatttacaactcctgtaaatgtgtgtgtttacaagttacagttctcatgttcatctccattcccttttagaaatcttggtttagttctatcggatgatattataattatcttgccgcgttaccacatttctatactcaactaagattgatttcacatgcactaggataaactacttttcaatagctttaagatgcatcatctttttttatttagacatggctagtaactatatgtaagatcctgttaccgaaaaggaaaaaaagggatttctcttaggttaaacaattggtaaatatagtatcatagccatctaaagtacattcatgttattggttaaaaaacttttatgaggatatgcacatattcgtgagccaagagtctattggaaacagccactctaccttcacaaggtatggtaaggttgcgtatacaccaccctccccagacctaatcatcaaaatctaatggaatgcgcaagtttgtttgtcagtttactatctcatgcttcatcgtcaatcttttcttttaattgaatctcaattaatcagataagaggagaaatatattggatatgaattgcatgtgcttaaagcttttgcacttaagctatgctatttctgattcaacatatactaatgacctgcatctgaatttccagtaattagttcagaagccaaaagtatacttctgagacacctagagcagcgtgccgaagagaaggtcagtgttcgttttgatcgacatttattccttctttcgatgaatatttggcaatttgtaattgtgttatattgtgaggattcatcttaaagtgactagttttacaatggctatcaacctattgcaattcttctcctttatccagtcttgtgaccgacaatatgagcaaactcacacataatcaattgaggattgaggcgttgttgctatccctttacggttaaaatgcttttctgtgttgcagttgagaccaaagcgagctgcggctgaaaatctggcacccgagcatggatcgaagcatcttaaggtatccaactgagatgcttttcttttcggtgctacccctgggcgggaagaagatgaggtaatgcggttgcaaacaggacgatacacaacttggttttagaagattaactaactcttctaaataggttaaaatcttctggtttttctgcatatttctgtaaatatgactgtaatgatgcagatacatgttgttgtaaaactatgaagagcttgtttgtcaagtagtcatatagcaaatgagatgtacactagataaaatgttgttagatgagtattgataaatgtttccctccgaggatttatcggaaacaacctctgtgatccgtcctctgtgaccctagaaaatgatattttatcataatgatcaaactttttaacattga

SEQ ID NO: 123 - protein, 93 aa, Solanum lycopersicum
MGSMFGEWPSIDPHNFSQLRPSDPSTPSRMTPVTYRPTHDRTLPPPNQVISSEAKSILLRHLEQRAEEKLRPKRAAAENLAPEHGSKHLKVSN

SEQ ID NO: 124 - DNA, 282 bp, Nicotiana tabacum
atggggtcaatgctaggtgattggccttcttttgaccctcacaatttcagccagcttcgccctttcgatccctccaccccttctagaatgacacccgtgacttatcgtcctactcatgataggactcttccgccaccaaatcaagttattagttcagaagccaaaaatatacttctgagacacttagagcagcgtgctgaagagaagttgagaccgaaacgtgctgcgactgaaaatcttacaccagagcatggatctaagcatcttaaggcatccatctga

SEQ ID NO: 125 - DNA, 589 bp, Nicotiana tabacum
ggctttgccaacatcaatattttgtccaacccaccatatattttgcagcttctatttacctccggtgtctaaaacagtggcattattctctcgattcttcccgttaataattcaatggggtcaatgctaggtgattggccttcttttgaccctcacaatttcagccagcttcgccctttcgatccctccaccccttctagaatgacacccgtgacttatcgtcctactcatgataggactcttccgccaccaaatcaagttattagttcagaagccaaaaatatacttctgagacacttagagcagcgtgctgaagagaagttgagaccgaaacgtgctgcgactgaaaatcttacaccagagcatggatctaagcatcttaaggcatccatctgagttgcttctctttttgtgctactcctggggcgggaagaagatgagaaaatgccaagtgtgacagtttcaagtcggatggtacacaacttggttttgagaaatgacttctaaataggtttgacgtcttcgggttttcttcatatttctgtaaatattgttttaatggcagagatgcatgttgttgtaaaattga

SEQ ID NO: 126 - protein, 93 aa, Nicotiana tabacum
MGSMLGDWPSFDPHNFSQLRPFDPSTPSRMTPVTYRPTHDRTLPPPNQVISSEAKNILLRHLEQRAEEKLRPKRAATENLTPEHGSKHLKASI

SEQ ID NO: 127 - DNA, 300 bp, Eucaliptus grandis
atgggttctatcctgggcgacttgccgtcgttcgatcctcacaacttcagccatttcaggccctccgatccctccaacccttccaaaatgacgccaacaacctatcatcccacccacagcaggactattccaccacctgatcaagtgataactactgaatccaaaaatattctgataagaaatttctatcggcgtgctgaagaaaagatgagaccaaaacgggctgcctctgaatttcttgcacaagaaccaggatgcaagcaaccaagggcttccatgaccacctcagataccccataa

SEQ ID NO: 128 - DNA, 830 bp, Eucaliptus grandis
ggagttttcggtggcattaaggcttcatgttttcacgacggattatttctttcgtccatagatttgtgtctatacttcggagcgtctcgtgtcgggggagtattaatgagctttcgtcgtaaggtcagacacgaccgtcctgtcctgtttccaggcaactccagcaccagcagcgaggctgattctagaatttaaggccatcgtctctctctctctctctctctggattcgaggggggaacactgtgcagaggttctgcattcactctttcatgggttctatcctgggcgacttgccgtcgttcgatcctcacaacttcagccatttcaggccctccgatccctccaacccttccaaaatgacgccaacaacctatcatcccacccacagcaggactattccaccacctgatcaagtgataactactgaatccaaaaatattctgataagaaatttctatcggcgtgctgaagaaaagatgagaccaaaacgggctgcctctgaatttcttgcacaagaaccaggatgcaagcaaccaagggcttccatgaccacctcagataccccataatgagcttctgcatcggggtttgcgacatgagaagttcagcagtctgcactcattgagtgtatatatactgctgtaatatcagactggtctgtaggcttggcatctgcctattttaatggtatgtggttgctgaccttgtgtctgttatttgctgatgctggttggtttctgtgaagaaagcgccttttgggggatgcacagcctctcccaccgtgtacattggaaataatcaatccttgattttcaccatctcaataaa

SEQ ID NO: 129 - DNA, 3218 bp, Eucaliptus grandis
ggagttttcggtggcattaaggcttcatgttttcacgacggattatttctttcgtccatagatttgtgtctatacttcggagcgtctcgtgtcgggggagtattaatgagctttcgtcgtaaggtcagacacgaccgtcctgtcctgtttccaggcaactccagcaccagcagcgaggctgattctagaatttaaggccatcgtctctctctctctctctctctggattcgaggggggaacactgtgcagaggttctgcattcactctttcatgggttctatcctgggcgacttgccgtcgttcgatcctcacaacttcagccatttcaggccctccgatccctccaacccttccgtgagttccccgtctttctcttggccctccgccttgttccctttttattttttgggtggcgtgtgcttcgtttagtttcctcaatttctgctgctgctaccgatgcgtgtgattctttttcttgtcgctgtccttagtctaatttttcgtggtggaagatgatgaagaatttgcataggagaagtgaggtttcctcagcttccatgtccgaactgaggggtttttagcagcagagatcatttgatggagctgggttcacccgttttgcggcttaactggggcaaaatcacaactttgttccggctgaatgagttcttggcttatatcttaattcttctagttgattttagccgggtctaggattcggactgattgggacggtaatgcttgttcttagagatagtgtttttatacattttggacgcattgagttcctcaatttctgctaccgatgagaatgattcttttcttgtctctgttgttagtcatttttttggtggtgaaagatgatgaggaattgcataggaaaagtgaagtgtcctcggcttccatgtctgaactgaggggtatttatcagtagagatcgtttgatagagttgggttctgcacattttgtggcttaaatggggcaaaatcacaactttattcaagctgaatgagttcttagctgatgtcttaattcttctagttgatttcagccagggctaggattcggactggttgggacagcaatgcttgttcttagagaagtgtctttatacatattcgacacattgagttcctcaatttctgctattgatgcgtgcgattctttcattgtctctgttcttagtctatttttcagtggccaaagatgatgaagaattgcataggagaagtgaagtgtcctcggcttccatgtccagactgaggggtttttatcagtagaggtcatttgatagagttggcttctgcgcgttttgtggcttgaatgggggcaaaatcacaagtttattccatctgaattagttcttagcttatgtcttgattcttctagtcgattctcgctggggaccagttgggacagcgatgcttgttcttaaaaaagtgtttttttatacttcttcgacgcattgagaatatgttgccttgccatggcttaccacatcttatctatcttgtgatgagatttttctggtcttcttggctctgccctgctcaagtgctagttctaaaacctgagaaacactgggaattagggcggtcacgagaagtctttagcacaaatttggctgctcatgtcatgtcctttgtaacagagccataagctttaagccaacgagccattgtagtttggccagatgggcacacagtgctgcatctttgtgactggtttggaacatggtgttttatagtttgtagggatgatttgatcttgaggagatgtagagtaacttctcctttgaaattccggcaaattagttgtacttgtgcagaattcctgaagtactttaagaaactataatcacataacaagtttctggtcttttgaataagtttccttcttggtttagagaattcttatggtagttcttttcaagattaggaggttatatcaggacatcaactgatctaagctacacctatcaaaattaaagggccatacattgtgttcaaatttaattttaagttattaatgaccggacggaaccaagaaataacacctcatgtgcagtttgctgttggcaattcagctgtccaacaaatgacacagcttgtgtctggtgattggtgaataaagtgagctctcttcaattccttctaggtcgaggaagctagatttgattaaatcaccttgtggatgacaaagatctctttgctttataactgatgtctattgcaggaacgctgttttgttactttttgattggaacaagaaatagcatggacccgagataagatgatttgaacatagctttacttacaattttcaaatgttatatgtagaaaatgacgccaacaacctatcatcccacccacagcaggactattccaccacctgatcaaggtaataatgaattagatatgcatctccagcactttttgctcattgatttaatgtcgatgttgagctctttagctgattgagaagagaatgtcacttttatgtggaagataagcattttttccatttcagtttttggagatgtggttctcatcctcttattgcgatgaccctgacttggttgcaactccttccagtgataactactgaatccaaaaatattctgataagaaatttctatcggcgtgctgaagaaaaggttagtcattcatatgaaaccagaattttgataacctacatagcacgtgcctctctcatgcatcctttgcatttcgcatccatagactacgcatgctacgtggatgaatgttcgcatgcatttgcactcttcattggtggatgtctgccttacacacacaaaaacacacagatgctttccattgctgattagtttttgtcaatatcagatgagaccaaaacgggctgcctctgaatttcttgcacaagaaccaggatgcaagcaaccaagggcttccatgaccacctcagataccccataatgagcttctgcatcggggtttgcgacatgagaagttcagcagtctgcactcattgagtgtatatatactgctgtaatatcagactggtctgtaggcttggcatctgcctattttaatggtatgtggttgctgaccttgtgtctgttatttgctgatgctggttggtttctgtgaagaaagcgccttttgggggatgcacagcctctcccaccgtgtacattggaaataatcaatccttgattttcaccatctcaataaa

SEQ ID NO: 130 - protein, 99 aa, Eucaliptus grandis
MGSILGDLPSFDPHNFSHFRPSDPSNPSKMTPTTYHPTHSRTIPPPDQVITTESKNILIRNFYRRAEEKMRPKRAASEFLAQEPGCKQPRASMTTSDTP

SEQ ID NO: 131 - DNA, 299 bp, Lactuca serviola
atggggtcgtggattgttggtaattggccttccttcgacccccacaacttcagccaacttcgccccaacgatccgtctgctccttccaagaagacaccaattacatatcatccaactcatgaacgaactcttccaccacctgaccaagtaatatcttcggatgccaaaaacatacttctgaggcaattctatgagcgtggtgatgaaaagttgagaccaaagagagctgcccctgagaatctggcacccgagcaagaatgcaagcatccaagaggttcttcttcagatcctctatcatg

SEQ ID NO: 132 - DNA, 554 bp, Lactuca serviola
cgggggagccactgttccaaaaatgtgtcaagtatccctaaacaatcaatttcctcatctgtccatctccagaaactgcgattatcggggctgagaaatctgtgggactgtaatcgattttaatggggtcgtggattgttggtaattggccttccttcgacccccacaacttcagccaacttcgccccaacgatccgtctgctccttccaagaagacaccaattacatatcatccaactcatgaacgaactcttccaccacctgaccaagtaatatcttcggatgccaaaaacatacttctgaggcaattctatgagcgtggtgatgaaaagttgagaccaaagagagctgcccctgagaatctggcacccgagcaagaatgcaagcatccaagaggttcttcttcagatcctctatcatgatggattcatgagtaatcgagtatgcatgcataaatcataatgcattgcacattgatgtaaatattatttggttgtcgatgctatatgtgtgttgtatgtttttgggaagctatgagataaagccaattgtta

SEQ ID NO: 133 - protein, 99 aa, Lactuca serviola
MGSWIVGNWPSFDPHNFSQLRPNDPSAPSKKTPITYHPTHERTLPPPDQVISSDAKNILLRQFYERGDEKLRPKRAAPENLAPEQECKHPRGSSSDPLS

SEQ ID NO: 134 - DNA, 312 bp, Helianthus exilis
atggggtcgtcgtgggatgttggtaattggccttctttcgacccccacaacttcagccaacttcgccccaacgatccttccgccccttccaagaagacaccaattacttatcatccaactcatgaacggactcttccaccccccgaccaagtaatatcttcggaagccaaaaacatattgctgaggcaattctatcagcgtggtgatgagaagttgagaccaaagagagctgctcccgagaatctttcaccggagcaagaatgcaagcaccctagagcttcatttgcttcatcttccgagcctccaaaatga

SEQ ID NO: 135 - DNA, 590 bp, Helianthus exilis
gaattcgtatgcgtatgcacatcatcaatctatcttccgatctgctgctgctgctgcgaatctaataatcggggctgaatggggtcgtcgtgggatgttggtaattggccttctttcgacccccacaacttcagccaacttcgccccaacgatccttccgccccttccaagaagacaccaattacttatcatccaactcatgaacggactcttccaccccccgaccaagtaatatcttcggaagccaaaaacatattgctgaggcaattctatcagcgtggtgatgagaagttgagaccaaagagagctgctcccgagaatctttcaccggagcaagaatgcaagcaccctagagcttcatttgcttcatcttccgagcctccaaaatgaggcatactcacctttctgcacaatgatgtaaatagtcttcactgctgagtgttgataccatatgttgtgtgtgttttagtgaggtatgatacgagtgaatcgtttgcatcttggtgtgtactttcagctatacagacttgtacatttctatatttataaacaggcagataactaaatatgcaaaacaacatccttggcat

SEQ ID NO: 136 - protein, 103 aa, Helianthus exilis
MGSSWDVGNWPSFDPHNFSQLRPNDPSAPSKKTPITYHPTHERTLPPPDQVISSEAKNILLRQFYQRGDEKLRPKRAAPENLSPEQECKHPRASFASSSEPPK

SEQ ID NO: 137 - DNA, 312 bp, Helianthus annuus; identical to SEQ ID NO: 134

SEQ ID NO: 138 - DNA, 553 bp, Helianthus annuus
cgtatgcacatcatcaatccatcttccgatctgctgcgattatatcaccggggctgaatggggtcgtcgtgggatgttggtaattggccttctttcgacccccacaacttcagccaacttcgccccaacgatccttctgccccttccaagaagacaccaattacttatcatccaactcatgaacggactcttccaccccccgaccaagtaatatcttcggaagccaaaaacatattgctgaggcaattctatcagcgcggtgatgagaagttgagaccaaagagagctgcccccgagaatctttcaccggagcaagaatgcaagcaccctagagcttcattcgcttcatcttccgagcctccaaaatgaggcatactcacctttctgcacaatgatgtaaatagtcttcactgtcgagtgttgataccatatgttgtgtgtgtgttttagtaaggtatgatacgagtgaatcgtttgcatcttggtgtgtactttcagctatacagacttgtacatttctatatttataaacaggcagataactaaatatgac

SEQ ID NO: 139 - protein, 103 aa, Helianthus annuus; identical to SEQ ID NO: 136

SEQ ID NO: 140 - DNA, 318 bp, Zea mays 1
atggggagccctctgggcgggtggccgtcatacaacccgcacaacttcagccagttggtccctgccgacccctccgcgcagccctcgaatgtcacaccagccacttatgttgcgacccacaggacagatccgccacccaatcaagtgataaccacggaggccaggaacatcctgctgaggcacttgtaccagaaatccgaggagaagctgaggccaaagagagctgcggcggacaacctcgctccggagaacaacaacaagaagcagcccaggggacctgtgggcgacgtcgggggccagtcgagcgcaagaagctga

SEQ ID NO: 141 - DNA, 965 bp, Zea mays 1
ctttttccccgaaaccaaaacagaaaaaaagtaaagtcctgctggcagctgtcaaccacccgtggtcccgtggaagagaagagagcatcgccggacccggggacggcgcgccgagaaggaacaaaagaagacggcggcggggcggagatggggagccctctgggcgggtggccgtcatacaacccgcacaacttcagccagttggtccctgccgacccctccgcgcagccctcgaatgtcacaccagccacttatgttgcgacccacaggacagatccgccacccaatcaagggcgtgttatttgtgagcaactggacaattcaaaacatctgaatgggtacttcagccacagacttctggtgaggtgcagtgataacagcagagatatcccaatttgtatagcagataaattgataaccacggaggccaggaacatcctgctgaggcacttgtaccagaaatccgaggagaagctgaggccaaagagagctgcggcggacaacctcgctccggagaacaacaacaagaagcagcccaggggacctgtgggcgacgtcgggggccagtcgagcgcaagaagctgaagacgcacagctggtggccgtcctcccctgcttctcatctatcggtgtcatgcagcctgcatctctcactcacagctgagctggtagctggtggtggttgccctcccctcccctgtgcgtcctcttcgcctctcacgtctcgtatgtacgtatggtatgaccaggagagctagtttgcatacaatggatatactggatgtgcatagccacctgagacgagacgagacgggactggacgaggtcggtgcgtgccatttcacacggcactaccgcactagtctgtgcggcagcctctctctctctctctctctctctctctctctctctctctctctctctctcatcctctgcaatgcaaaaatatggatgcgcccatgctg

SEQ ID NO: 142 - DNA, 4133 bp, Zea mays 1
ctttttccccgaaaccaaaacagaaaaaaagtaaagtcctgctggcagctgtcaaccacccgtggtcccgtggaagagaagagagcatcgccggacccggggacggcgcgccgagaaggaacaaaagaagacggcggcggggcggagatggggagccctctgggcgggtggccgtcatacaacccgcacaacttcagccagttggtccctgccgacccctccgcgcagccctcggtcggtcagccgtcagcaacttgccttcctggcgatctggcctctagtatcatgatgtactgctaggctccgtacttcctgctaagcttacacaaccatggatatgtctaattgaccgtgctcggctgacctgtttcttttctctgctgtctggtgtcgtcgcgagaaaaaaaaaactctctttttttatcccgcagtatcactttcgggcaggaggcagtaatcggtgcccgtattcaggggcggacccaagtagggtcgagtgagggcaatcgctcgtttctttgttctaaatactgaatctagattttcgctataaggttctctactcagtcatattctgtcttgggtccgccccagtgacggatctacaccccgggccattcggtccgtggcccggggtttgatccatgtagctatatatatgtctctatttaatatggtataagatatttaaaataaaatgaagagaagataatttggtagatttggtctgggtcatagaaaaattctggatctggtccgcgtctacccgtattgttagtttttgctggaattttggtatgtatggatggagaaatggggtctcaccgtttgattttagttcaactgccaaagacctgttgaatttgaggggactgtctggcgaatttccaaacgcatggtctggttttctcatggtcatgtctaccctgggcagattcagttgatgtggtactgatgaactaactgtagttcagttcatgggctgctaatgctacccgctaccggttgatttttataacgtcagaaattcatgctagcaattgacattatgaatgatatatccatttcacctggtggtaatgttagttctttttcctttccctgtgtgtttctcatcagaatgtcacaccagccacttatgttgcgacccacaggacagatccgccacccaatcaaggtaaaccctttccatgtcctaagccaatgatgttctgctgcatccatcagcaaatttgtcccatctgatttcctagttttgcatttgcacgtaattcatatcgtacaattcctttcacattaagccaagaatcccgctattttgtttatcgtagtgttttttacattacattgcaaaataggtttcatgagctgtttcgtctgaactgacgttcttcagttagatgactctctttgcttcaatggagcatgattagccataagcttttgtgcatggggttgaaatgtagaacgtgtctgccagtcacagatggtggtatcagccatagcgacagactgacagaagctctgaataccttatcctacacaaatgacgcctctgcaactcttctgtgtttagtgtttagctgaacccagcagctctgaacttcctcgttgttgctagtaatcagaattcagaagttaatactccctctgtttcaaaatataatttgttttagactaaacatacattcataaattaacctatgaatgtggtttgtatgtatgtctacattcattatttttcattcgaacgtggacagaaaaaaaagagggctaaaaagaaatatattttgggacagatggagtatattttgggacagatggagtagatatgatcgataactcagcagttgctgtcttagccatgtactccaatacaataaatacacaacgttgcaagtactaacagttccaggctaccagctttgactatgcgattccatataacattctttctttgttgcaaaatccttcagcaattatagtgttatgccttcaaagaacgcagctgggaaacattgcctgttgtatttaggaagttctagattctgaaggtcagctttcttattttactgaaagtctgaaacacactgactattaacacattaatattgattcatctagtcacatcaaatggtaaattgatttgtgacactaatccagattaatgcatagtaagtattcacctcgaatacagtagatcaacagaggtgagattataccatagcctataatctctcttgatatctcagcatttggcatggcttatacatattatggctgagttgttttgtgccttttgtaccgtttttgtctggaatatgcaggaggactgcatatcgttgcattcatagaataaagagaggggaaagacccccccccccgtacaacacacccaagccacccaaagatctcaccaggaaaaaaacagcaagcagacgaccgacccacacagatctacctaaaccggcagacccagagggcggatagattttttggccccagccatagaacaaaacagcacctcatccttttgttccgttgtgtaggttccttaggagatttgcagtgttttacatacttaatctccaaaacactttcttatagcacgagaacgaggaagaaaagtttgggttaattactgctttatggatcagggatgctggcgttcaaaatcaacccagacaccagttaaatgcatccatcataataatagaccttaagagtggattctctggactttttcagtaaagttcggagcctttcgatcatatgaaaatgctatccacatggagctctagaactgagatgaccttgagagaagttaggttaatttactactgaatgatagcctagtaatcacaccgactaggattttttggcctgaacactctagttgtcagtttctgtaacatatgtcgctctttgctgcggtcattctctggtcccggacaatttaccaggttaagtgaaaccaccggaagcccttattgaattcgtgccttttggcgcggctgattccacatcccgtcggtggaaaatatagtcgtgtgctgcctaccaactgcataaaaaggtcccgaaagaaacaaacaggctatgattgtgcgtttatatggagctcatgacatattttcagggcgtgttatttgtgagcaactggacaattcaaaacatctgaatgggtacttcagccacagacttctggtgaggtgcagtgataacagcagagatatcccaatttgtatagcagataaatgtactgaacaaaccgtgggcattcttttaactatatacatgcatgacaattcttctctctctctctctctctctctctctctctctcttccaaatttccttgtacctatcataccactgcattattttatatatgtatataggtccatagttcacagtacttgaggatcaatgactgcttcctacagactactgcggactgaatcgcgccctggaattgcagtgataaccacggaggccaggaacatcctgctgaggcacttgtaccagaaatccgaggagaaggtgagcagctactgctactgctagtaagacttcactatcacgcacggctacataaaaccacatcaccgataaaggttaaaaccctgtcctgaactgtagctgaggccaaagagagctgcggcggacaacctcgctccggagaacaacaacaagaagcagcccaggggacctgtgggcgacgtcgggggccagtcgagcgcaagaagctgaagacgcacagctggtggccgtcctcccctgcttctcatctatcggtgtcatgcagcctgcatctctcactcacagctgagctggtagctggtggtggttgccctcccctcccctgtgcgtcctcttcgcctctcacgtctcgtatgtacgtatggtatgaccaggagagctagtttgcatacaatggatatactggatgtgcatagccacctgagacgagacgagacgggactggacgaggtcggtgcgtgccatttcacacggcactaccgcactagtctgtgcggcagcctctctctctctctctctctctctctctctctctctctctctctctctctcatcctctgcaatgcaaaaatatggatgcgcccatgctg

SEQ ID NO: 143 - protein, 105 aa, Zea mays 1
MGSPLGGWPSYNPHNFSQLVPADPSAQPSNVTPATYVATHRTDPPPNQVITTEARNILLRHLYQKSEEKLRPKRAAADNLAPENNNKKQPRGPVGDVGGQSSARS

SEQ ID NO: 144 - DNA, 318 bp, Zea mays 2; identical to SEQ ID NO: 140

SEQ ID NO: 145 - DNA, 772 bp, Zea mays 2
gtaaccatccttttcccagaccaaaacagaagaaaggaaagtgctactgggcattggctactggcctactgccaactacccgtggggtcccgtggaagagaagagagcatcgccggagttggggtcggcgcgccgacgaggaacaaaagaagacggcggtggggcggagatggggagtcctctgggcgggtggccgtcttacaacccgcacaacttcagccagctcgtcccggccgacccatccgcacagccctcgaatgtcacaccagccacttatgttgcgacccacaggactgatccgccacccaatcaagtgataacaacggagtcgaggaacatcctgctgaggcacttctaccagaaatccgagaagctgaggcccaagagacctgccccggacaacctcgctccggagaacaacaacagcaacaacaagcagcccaggggaccggtcggcgacgtcggtgggcagtcgtcgagcgcgagaagctgaagccacagctggtggccgtccactcctccccctgcctgcttgtttctcatctctgggtttcgtcatgcagaggcagaggcagatgcagccctgtgttgtcctcttcgctcctcacgtctgtacgtacgacccaaagagctactaacctaatctgaagttagggttacatacatggatatcggatagatgggtgtacataagcacctgagagcagtgttgtgttatcctaaatgctaaacggtaaacagtcgttcaacatcctctgtgtttagcgtataa

SEQ ID NO: 146 - DNA, 5516 bp, Zea mays 2
gtaaccatccttttcccagaccaaaacagaagaaaggaaagtgctactgggcattggctactggcctactgccaactacccgtggggtcccgtggaagagaagagagcatcgccggagttggggtcggcgcgccgacgaggaacaaaagaagacggcggtggggcggagatggggagtcctctgggcgggtggccgtcttacaacccgcacaacttcagccagctcgtcccggccgacccatccgcacagccctcggtcagcaacttgcccttcctggcgatctggcctctaatatcatgctgtgctgcttggctccgtacttcctgatgagcttacacaagcttgagtatgtttaattggccgtgctcatctgcgcttggcttatttcttttctctggtttatggcgtcgttgaggaaaatatctttaaaaaaaaatccgtagtatcgttttcgggcaggaggcagtaatcggtgattcggtgcccgtgttgttagtttttgcttggaattttagtatgaatggctagagaaatggagcctcgctgtttgattttagttcaattgccagagacctgttcaatttgaggggactgtcggcccaatgtccaaatatctggtctggttttctcatggtcatgtcatgtctacctgagtatattcagctgatgtagtgctgatggactaacggtagttcagtatttcagttcatgggatgctaatgctaccagttaatttttataacgtcagaaattcgtgctagcaacttgtattatgagtggtatattcatttaacatggtgctaatgttggttcttttttctttccctgtgcatttctcatcagaatgtcacaccagccacttatgttgcgacccacaggactgatccgccacccaatcaaggtaacccctttccatgtccttaagcggagccaacgatattctgctccacccatcagcgagtttgccccatctgattttctaggtatcatttgcataaaatccatgccgtgcaattgcttattcgcattaagctaagaatcccttttttattattctagtgctttgcgtgttacattgcaaaataggtttcctgggctgtttcatctaaggcaacgactgctatgcaagcagtccttctttgcaagcgtgcaagcaaatcatctgatccattagattacgattcaaccacagatacaaccatgatttgttaggatttttttataaagattattgagctggtggtggaagggtttaagtgttaaatgtgacctacggttggatcacgatctaatggatcagatggtttgcttgcacgtttgcacagaaggactgcttgcatagcagtcgatgccttcatctaaaccgacgttcttcaggtagatgactctctttgccttcaacggagcattattagccataagcttttgtgcatggggttgaaatgtagaacgtgtctgcaagtcacagattgtggtatcagccatagtgacagaagctctgaataccgtatcctacataaactgtgcctctgcaactcttctgtgtttagtgtttaactgaacccagtagctctaaacttcctcgttgttgtcagtaaccagaactcaaaagctaatagatatggtagataattcagcagttgctgtctttagacatgtactccagacaataaatgcataaccttccttgctggcaaatttaccagttccaagagctaacagttccaggctaccagctttgactatatggttcttataaccttctctctttgttgcaaaatcctgtactaattctagcctatgccttcaaaaccacaactagaaaacatggtctgctgtattcagaaaattttagattctgaaagctagctttcttattttactgaaagtcttccgaaacactgaatatttaatcacagcctaaaattcgctcccatggggagtcgaacccaggacctgaggagcgttactcagaccacctaaccaactcggctagatcccctttcgcttttcaatgcctctctaaccttagattcttgtatcacacgcacaaagtacctattggtatcatcaaaagagtcacaactaaaaggtcgtgtcctcatcctccccgtaaccccattgaccagtttgtcaaaatactcttgtcatatatatctggtctcatctgccttcactaagatgtgctctattttatccttaatgcacttaactttgttgaagtctcttgtctttctctcacggaccctagccatcctatatatgtcttactcacaagttggtaaaggttatcgtatgccctattctttgccacacttacaagtcgttttgcggtcttccttgctagcttatacttctctatgttgtccacactcttgacatggtacaaacgtttgtagcattatttattctccttaatagccctttggttttcctcattccaacttcaagcgtctttagcttcacctccactctctttggttgctccacacacctctgaggccaccttccgaatacaggttaccatcttctccaatatgttgtttctgtcatcttcttgctttcaagtgtcctctttgatggctctttccttggagactcctgatgtctcttatttcagtttttaccactttgttctcacaatcttggtttgtttatccatatgtgcacacacctaaaaaatggaagaccgcaaccacaaacttatgttggtagacatcacactcctttggtatcaccttgcagtctaagcatgctcgtttatcctctcttcttgtgaggataaagtcaatctgactagtgttctgggcttttggccactattttaggtcaataaatgggattctctcttcttaaagaaactagtggttatctgtcgagaatctttcaacagttaatttgtgtggtggtgtttgcgagatcaaagaactaaaagtaaaagcacaagagacaatatttagactggttcgggctctcgtcgtgagataatacctcacgtccagtattatggtggctatgcttacgatctgttgtgctctgccctcttggggcgcctcgcccctccttttatagttggaggggtgcggttacaagaaaacgtgtggtcgtactagacaaggactcggacctaaaagtcctcggttacaaggatccctatcatcgctataatggcttcccttataaatcgggtattaccgttacaatgaagatatgacccatatatcgtacaaaccctacaatcttccttatttgctcgaccacgtagactttatcccgacatgtggattaggttctcctgaaagtctgagaccgagtccaagtcctagactgagtccgagttgggcccacaagccaacacctttaggatcaacgtacctatggtaccccttgtatatattgtttacactatcaataggtcaaaagctactacaaagtttaagatttcctctccctcttggtttgtactagtatacctaaaacctccacctacatgttcattgtgatctcctatgcaaagctcctcactaatatgtagagctctaaacatgccatcgaagtcttcctaccactctcatcgtcgtctacttgagaagtatactcgctaattacatttaagaccaaatcatccatggtaagcttgactaagataattgtatttccttcccttttcacatccatcacaccgttctagatgctcttatcaattaaaactccctacttcatttctatttgcagctatccatatgtaccaaagcttaaaaccggtattgtccacccccttcgtcttctgacccttccatttagtcttatggacacacaagatatttacatgtctcttagtcattatctcaactaactcctttatagctcattacctgtaagcgaccctatattccagctgcctaaaaagattatagtttgttccatccactagctttcttcccctttgcacctacgatgatgtgaagacccttacatatttttttactatatctgggcacttatcataatcttcctttgccatggtttgggacctgctatattgagacaacataggcggattttatatacttaatctccaaaagactttgaatcttagaacctgagaaagaggaagaaaagtttgagttaattggttctttatgggtcagggatgttggtgttcaaaatgaacccaaggaccagataatgcatccatcaaagagtggagcatctaaggcttcgtcataatgatcacactgactagaaattttgcttgagcactctggttgtcgatctccgtaacatatgtcgctgtttgttgcggtcgtttccctggtcctgaacaatttaccaggttaggcgaagccacaggaagtccttattgaatttgtgccttttggcacagtcgattccacatcctgtcggtggaaatattattgtgtgtgctgctgcctaccatgtgccagctgcataaaaggccccgaaaggaacaaactctgattgtgccttttatctgcggctgatgacatgtttttggaagatgttatttgtgaacaattgagtatatccaaaacatctgaagggatacttaggggctgtttggttcgtggctaaatgtgccacactttgtctaagattagtcgttcgaattgaagaactaaccttaggcagaaaagttagttaaagtgtggcaagttagctatcaaaccaaacagacctttaatcctggactaccgccggcacttgagccacagacttctggcgaattgcagggatatcccaatttgtagcagaaaaatgaactgaacagatcgttgggtccttcaactatatccctgaaaattctctctcttttaaattttcttgtacctatcagttatcacgccactgcattgttttgtttatataggcccgtagttcacagtagttcatgctcaataactggttcctaccaattactgtgggggcactaatccgttcctgtggaattgcagtgataacaacggagtcgaggaacatcctgctgaggcacttctaccagaaatccgagaaggtgagctggtactgctagcaagtaccatgaaaccagatgaccgaaacgaatctaagcttgaaatcctgtcctgaactgtagctgaggcccaagagacctgccccggacaacctcgctccggagaacaacaacagcaacaacaagcagcccaggggaccggtcggcgacgtcggtgggcagtcgtcgagcgcgagaagctgaagccacagctggtggccgtccactcctccccctgcctgcttgtttctcatctctgggtttcgtcatgcagaggcagaggcagatgcagccctgtgttgtcctcttcgctcctcacgtctgtacgtacgacccaaagagctactaacctaatctgaagttagggttacatacatggatatcggatagatgggtgtacataagcacctgagagcagtgttgtgttatcctaaatgctaaacggtaaacagtcgttcaacatcctctgtgtttagcgtataa

SEQ ID NO: 147 - protein, 105 aa, Zea mays 2; identical to SEQ ID NO: 143

SEQ ID NO: 148 - DNA, 312 bp, Brachypodium distachyon
atgggaagcccactgggcggctggccgtgctacagcccgcagaacttcagccagctcgccccggccgacccctccgcgcagccatcgaatatcacaccagccacttacatagcgtcacataggacagatccacctcccaatcaagtaattacaaccgatcccaagaacatcctgctgaggcatttttaccaacagtcagagagcaaggtgaggcagaagagagctgcgccggacaatctcgcccggcataacgacaagcagccgaggggccccttcgccaacggtggaagcctggcgagcacaagaagctga

SEQ ID NO: 149 - DNA, 696 bp, Brachypodium distachyon
atgggaagcccactgggcggctggccgtgctacagcccgcagaacttcagccagctcgccccggccgacccctccgcgcagccatcgaatatcacaccagccacttacatagcgtcacataggacagatccacctcccaatcaagtaattacaaccgatcccaagaacatcctgctgaggcatttttaccaacagtcagagagcaaggtgaggcagaagagagctgcgccggacaatctcgcccggcataacgacaagcagccgaggggccccttcgccaacggtggaagcctggcgagcacaagaagctgaggatcagagctggtggttctcctgatctcctctgcattgcagtgtcctttgctgccgcatgccacactgcagccctcatgccataagcgttgccagtctctcatttaacatggtacgcgtgatcaaaacaacggggagcctttacatgcaggcaatgtactgatgtacatagacaggcatattcttggtcttattctcacccactcggtgcggttggttatctttgacagggcactataattgcagactttttctgtagataatgtgcccacaccaccaccatgggacgacgctccccccaacttgtacttttggtgaaataatttatcatcatcatcatcatcatctcattgcctctgtaattgatctatgtacactttagat

SEQ ID NO: 150 - DNA, 3771 bp, Brachypodium distachyon
atgggaagcccactgggcggctggccgtgctacagcccgcagaacttcagccagctcgccccggccgacccctccgcgcagccatcggtcagtcagcgcctccttttcgctgttgcgctaccacatatcatacttgtcgtggatcccatgtgtgcttgaattggagactactccatcgattccacaagttgtattatgcttgcgcaaatttgtttgtgcctagaaggagaggaatttcctcgcgacagaaaggagagggttcgtgtctgctgtacctgatgcagtaacaggagtcgctcctactgtgcttctttgatgaaattgtagtacacataggtggctaaatactgtagtttttacattgaagtcctgattgccgaactattggatcctattcaagtattcagagggctttggcacaatgactaagcatttaggtttaggttttagcttctctcatttgttagtacattgagtatattcatttggtgtagtcctaactattaacgggtttattgcggttgctatttttatgcatactacaggtcagatattcagttgccatggtgctaatgatactagttattcctgtgttttctgtaatgccttgtaggcttatagtcagaaaataaaaggcagaacacaaaaataaaaataaaaatatatcatgctagcaacttatactgtgggcaatagtagatatattatttggtgacctggtgctaatgttaattctcatttctttgtgtttctcgccagaatatcacaccagccacttacatagcgtcacataggacagatccacctcccaatcaaggtaactccatcccccgcttcttctaccaatgtctctggtctgtgctgcttctgcttctcccttttacaaatctagtctgactgatttcctcgttaggcctttgtgcataacttaatgtaattgctccttcgcattacgtcaaattatctttacgatggtatttcacagggcaaaaaatatgtcccataaactgtttgatcaactaagccttatgtttagccatgagaatccgtaaaccatattatgttctcaataatgtagaagtagacgcgccttggttctgctaggccaagcgccataggatttgggagggcgttcctaaattctgcaattttatgatgttgattagcaacagtactgtctatgatgtgggatctgtgctggagatattatgctgccacagcccacatgtccaaaggtgatgtggacaagtgtttagattcccaaccatcaccgtgccacattaactgcagtattgtatcgaaggtagtattttctgcatatcctaacaaatggtaacaatcttatgttgcccatagtgacttaacaccatgttctacactaatagtggcttccgccttcctcactcgaacttctgattcttttaaacacagttaacattggtaaggcggtaattgctatggataatgattcaactagacctgttttcaacttaactactcgctttgctgacccaacagcagctctctatatttcatctactgtatttttgtcgtgcaaatgtccaggtactagccttttcccaatgttaaatatatctttacaaaatctcactttgtgaaaaatatttgttctatcatgcatttcaagattaggaaaacatgatcccaaattttactgagacacataggctaacagcacatttgtaaggtcgaaacaactacatagttactgttcatctcaattgcaatagtgcaacagagttgagagacatgtcacatatggccatcgatgttggtgatcaaacttccttgcaaacactcaaatgcttgtaacagttaaatgtctctgggagaatcccttgtggagatgcagaggtgttttctgttatgggtgatgcattcaagtgtagaccagttagagcattaaatattccttcacaatcgttcctttttccatttccttgactatctcagggtttggcatgtagtatgtaggttatgtacttaatggcccatggctatattgccggacctttcatactttaatgtaggtacgcagggatgtccacatgatctttgcactgatatatcgtctaagtctcccaagttaactgttctctcggagcatgaggaagaggaagacaaggtgtagttaaaaaatacttaaatgtgtcagcagggatgtcaaccttgaaagtagaaatggtaggatcttcaaggttcccttcaagttgaattgtggcgtactcccaatgaaaattccttcaacgtggagtgcttgtagtgagaggtcgttgagaggaattgcttgggttgagttgaaggggcaatcggttgcctatgatgaatcaacaaaactggttggatattaggctcagaggtttcgtagagcacaccatggtactttctatgacatttcatgggccatttaccggggtctttttttctggggcctaatcaatttatgatgccaggctgaactgcctaagtctctgtctgctttcaagaaaagatatatttacaatacatcaaactaagatatttttaggcagagaaatatgctgacgaaccgaacactctaacgacattatgtgcctttttagggtgccaccagcatatttgtgaaggagatatttgttaacaaactgagaaatatgcaagaacaaaaatcatttaagtagacacttaaccctagactgtagttccgagtttctggcatatccttcaaagatgttctttttggtatattgtagggatcgagcaggtcctctattgccagcttctttcaatatttataataactaattatgtttctggtagaaacactcgccaaacaaattgctaatggaactaaccgccagttatatgttttgcatatcttttgaatgcattagtttatacatatgttcagagtagctcagactcaatggctgccccttgttttcttcttcttttttgccttttcgttaatttatattcgttggaggcactcatccactctcaccgtaattgttgcaaatcttctatgtccattttcttatgctctatgaaaaccacctttgcggtgtctcgactgtttatgctgataatctgtcccctggaaactgcagtaattacaaccgatcccaagaacatcctgctgaggcatttttaccaacagtcagagagcaaggtgagctaagccacccaagacactgatgaagaacagatagattaaaaataccgtcgaataataaaatcttaatctcaacattatatatttcttcgtatcttcatccataggtgaggcagaagagagctgcgccggacaatctcgcccggcataacgacaagcagccgaggggccccttcgccaacggtggaagcctggcgagcacaagaagctgaggatcagagctggtggttctcctgatctcctctgcattgcagtgtcctttgctgccgcatgccacactgcagccctcatgccataagcgttgccagtctctcatttaacatggtacgcgtgatcaaaacaacggggagcctttacatgcaggcaatgtactgatgtacatagacaggcatattcttggtcttattctcacccactcggtgcggttggttatctttgacagggcactataattgcagactttttctgtagataatgtgcccacaccaccaccatgggacgacgctccccccaacttgtacttttggtgaaataatttatcatcatcatcatcatcatctcattgcctctgtaattgatctatgtacactttagat

SEQ ID NO: 151 - protein, 103 aa, Brachypodium distachyon
MGSPLGGWPCYSPQNFSQLAPADPSAQPSNITPATYIASHRTDPPPNQVITTDPKNILLRHFYQQSESKVRQKRAAPDNLARHNDKQPRGPFANGGSLASTRS

SEQ ID NO: 152 - DNA, 324 bp, Oryza sativa ssp. Japonica
atggagagctccctgggcggctggccgtcctacaacccgcaaaacttcagccaggtcgtccccgccgacccctccgcgcagcccttgaatgtcgtaccagccacttacattgcaacacacaggacggatccacctcccggtcaagtgataacaacagaccccaagaacatcctgttgaggcatttttatcaaaaatccgaggaaaagttgaggccaaagagagctgcaccagacaacctgaccccacagaacaacggcaaacaaccaagaggccctctctctgatggtggtggtagccaggcaactgcaagtggtagaagctaa

SEQ ID NO: 153 - DNA, 727 bp, Oryza sativa ssp. Japonica
ggaaaagggtggagaaaccaagagggggcgtcgccggagtcggagtcggagacgtcacggcgagctccgcggcggcgatggagagctccctgggcggctggccgtcctacaacccgcaaaacttcagccaggtcgtccccgccgacccctccgcgcagcccttgaatgtcgtaccagccacttacattgcaacacacaggacggatccacctcccggtcaagtgataacaacagaccccaagaacatcctgttgaggcatttttatcaaaaatccgaggaaaagttgaggccaaagagagctgcaccagacaacctgaccccacagaacaacggcaaacaaccaagaggccctctctctgatggtggtggtagccaggcaactgcaagtggtagaagctaaaacgcagctgttgttctctccggcatctcttgtgctgctgaactgacagcatgcatgtcatactacctgtatgtatgtgtgtgtgcttgttcaggcatatgcttactagtagtgtcatcatctctcttgtgtgattgatcaaaagagctccccatgcatgtacatacaccctcatcctcagtgtcagtgcggcacctttgatacggaactagcactattgcagtcttttatgccgacactagcacaacttgatgaaaccatttttccctacataattgcctcagcgtcagctttccaaaggctgaaagtgatcattgcctctctta

SEQ ID NO: 154 - DNA, 3425 bp, Oryza sativa ssp. Japonica
ggaaaagggtggagaaaccaagagggggcgtcgccggagtcggagtcggagacgtcacggcgagctccgcggcggcgatggagagctccctgggcggctggccgtcctacaacccgcaaaacttcagccaggtcgtccccgccgacccctccgcgcagcccttggtcggtcagcccctctgtgactctgtcccccctcactcgcgatcgatctctgtgcttagctgtcgtgatctcatgcctccgaccgccaagtacaattcagaattaggttgattgtgttgatgatgccatgaattgtgtgcctgatccctcacgccggagctttacctctagtcagtcctattgtgtgtcagagcgtgtgtctgaaattccagtgcgcatgattaacaaaaggtggttagtactatttgtttctaagtcccaatgccaaatagttggatcataatatctcatttgggaggtatttgtgaatcatggattatacatttaggatttggtttctctcatggccaacgccctttaaggtttcagttgtggtgatgaattactagtcggtttttgctccgaatgtcattccaggcagccacttgcattgtaatttgcaaagcatatattcagtttgtcatatttgtgtttctcgtcagagtatcaagctggtaacttattctctggagtccggacatgttgcattggtgcgaaactgccaatacttatccctattttttttgtgtttcagaatgtcgtaccagccacttacattgcaacacacaggacggatccacctcccggtcaaggtatcttgctcccttgtttcttttaccaatgctgatttccctgtgctgctcctcacttctgagtgtgtggttcctaaacgttcaaatgtgtatagttgaatgtgactgctcattcacattgcatcaaattccttttacagtagtactctacatggcatagcagatcctctgagttgtttggtctgagccaaaaagtgactatcttgcctttcatggctggcatatttaactccaatcatctatactgttttcagtaatgcagcctctttagtatcttctgagtgatagatccatgaatagattctgttggactgttagatatgtcttcaaagtcaatattttttgcattccctaggactgtagttttggcttcttgatatgtaactcacatacattatggctaatttgcccagcttactttcaagtagttcccttacgacctttgttctggtacattttttatagcttcaaagcaaacttttctcttggagtatgactttatgagggagagcagaaaaggttgagagtcagagagccagggcctagccggcaaactggaaagaccaactaaacctgatcttacaccttcatagttgagtccccttgaattgatttgataagttaaatgcttttataattttagcaatcaaattttggaataacacatgctagagcaacatctatttggactagaggttggagtttcctaatgatgaatcaacataatttagtattttcgcctcagagactttgctctgacccactatgtgtacctattacaattttgtatgagcattctgttaagtcctttttttcctcctagcctaatcttttggcaggccaggccgaaccaccaaacatcctggttgctttcaggcctgtccactgaactatctccagatttcaggtacaggtattcagtaaaaaattgattgcatgctgcttaccatctccacaaaagaaaaatgaaagaaaactataggcaagctgggcaatttattagtaaggagcaaatgaactggccaaccaaagcaacctctcatattagtcatcttcctctagtgctgccaacatagttttattgaagtgttctgtttatgccatattgccaatatgatttggatggttacatttatagaacacagtttttcttctcgactcattgcccgtgtgcctgttggcagaggccaaagatgtaaaccatttccattatctaaaaaaaaagaacattgtttttagatattctatgtaaacattgtacttcattggtccgttgcaggcatggagctagttccttagtttccagtttattcaggggctgttcagattgatgccattttcaaccatatcattttttggcaaagttgccaaaaatgtgcctacatttggtttgttgccaaattttggtaaatacataagaaatcctgccaaaattttggtaatattgtcaacttgctaaaattttaggtaaggtttattttggcaacaatctgaacagcccctccgtatttttgctgcctattccctcttgaatttcctgtgcatgggatattgcttctgatagtggtgaattcttgagtgctttagttcattagagcaagtttaatagtatagcccactgctaactccaattcatctatagccaatctaatagccaattcatacaatagttgcttactatactattaatatatggtcacacctgtcatacatacattgcgtcttagagtccgcgatgcagctggctacagatctatagcccgctgctcttctctctcatcctttctctcattaaaatatgtttacagctggctaatagcctgctattgtacctgctcttagatattttcgtagtcgatcagactcgatagttgctcgctctctttacctcgttttggttctttctgggctctcgtccattcctaccagaactaccccaactattccaagttttcttttttactgtacagaaatcacctcttttttttttgttgctattttcactatttccctgaccgtttgtgtctggaatcgcagtgataacaacagaccccaagaacatcctgttgaggcatttttatcaaaaatccgaggaaaaggtgcgctgctaagagcctaagactctcacaaaggttacataaatcagtatggaacatctatttatcaacgctttatcttgactgtagttgaggccaaagagagctgcaccagacaacctgaccccacagaacaacggcaaacaaccaagaggccctctctctgatggtggtggtagccaggcaactgcaagtggtagaagctaaaacgcagctgttgttctctccggcatctcttgtgctgctgaactgacagcatgcatgtcatactacctgtatgtatgtgtgtgtgcttgttcaggcatatgcttactagtagtgtcatcatctctcttgtgtgattgatcaaaagagctccccatgcatgtacatacaccctcatcctcagtgtcagtgcggcacctttgatacggaactagcactattgcagtcttttatgccgacactagcacaacttgatgaaaccatttttccctacataattgcctcagcgtcagctttccaaaggctgaaagtgatcattgcctctctta

SEQ ID NO: 155 - protein, 107 aa, Oryza sativa ssp. Japonica
MESSLGGWPSYNPQNFSQVVPADPSAQPLNVVPATYIATHRTDPPPGQVITTDPKNILLRHFYQKSEEKLRPKRAAPDNLTPQNNGKQPRGPLSDGGGSQATASGRS

SEQ ID NO: 156 - DNA, 324 bp, Oryza sativa ssp. Indica; identical to SEQ ID NO: 152

SEQ ID NO: 157 - DNA, 601 bp, Oryza sativa ssp. Indica
tccgggtcaccacgcgtcgcggacgcgtggggggggcgtcgccggagtcggagtcggagacgtcacggcgagctccgcggcggcgatggagagctccctgggcggctggccgtcctacaacccgcaaaacttcagccaggtcgtccccgccgacccctccgcgcagcccttgaatgtcgtaccagccacttacattgcaacacacaggacggatccacctcccggtcaagtgataacaacagaccccaagaacatcctgttgaggcatttttatcaaaaatccgaggaaaagttgaggccaaagagagctgcaccagacaacctgaccccacagaacaacggcaaacaaccaagaggccctctctctgatggtggtggtagccaggcaactgcaagtggtagaagctaaaacgcagctgttgttctctccggcatctcttgtgctgctgaactgacagcatgcatgtcatactacctgtatgtatgtgtgtgtgcttgttcaggcatatgcttactagtagtgtcatcatctctcttgtgtgattgatcaaaagagctccccatgcatgtacatacaccctcatcctcagtgtcagtgcgg

SEQ ID NO: 158 - DNA, 777 bp, Oryza sativa ssp. Indica
atggacgtcagccaagccaccgaagagcaacttccttcacacggccagcaccagagctccttggaagagactgcaacatgtcatcattgccgagcgtcgccgcacccccatcagcaagcagcttcgacttcattagcagaaacaaccgaagagcgtgttacccaccacaagaaagaagcggacccaagaaggcgaaggcctcaccgaacaaaagcttaccttgatataccactccatttggacaaattgtggagaatccgaactctcccacgactgtcctccacgagtccatggtcgctggaaaagggtggagaaaccaagaaaagagggggcgtcgccggagtcggagtcggaaacgtcacggcgagctccgcggcggcgatggagagctccctgggcggctggccgtcctacaacccgcaaaacttcagccaggtcgtccccgccgacccctccgcgcagcccttggtcgaatgtcgtaccagccacttacattgcaacacacaggacgggtccacctcccggtcaaggccaggccgaaccaccagacatcctggttgctttcaggcctgtccactgaaatatctccagatttcagtgataacaacagaccccaagaacatcctgttgaggcatttttatcaaaaatccgaggaaaagttgaggccaaagagagctgcaccagacaacctgaccccacagaacaacggcaaacaaccaagaggccctctctctgatggtggtggtagccaggcaactgcaagtggtagaagctaa

SEQ ID NO: 159 - protein, 107 aa, Oryza sativa ssp. Indica; identical to SEQ ID NO: 155

SEQ ID NO: 160 - DNA, 309 bp, Hordeum vulgare
atgggaagcctgctgggcggctggccgagccacaaccctcagaacttcagccagctcgtcccggccgacccctccgcccagcccacgaatatcacaccaacaacttacattgcaacacataggacagatccacctccaaatcaagtgatcacgacggagcccaggaacatcctgctgaggcatttctaccagaactctgagaacaagccgcggccgaagagggccgccccggagagcgttgccctgcgcaacggcaagcaggcgaggagcctcgccgacggcggaagccagtcgagcacgagaagctaa

SEQ ID NO: 161 - DNA, 412 bp, Hordeum vulgare
gcacgaggaccaaccgtcggcaaaaaaagggcagagcttggccggagcgagagacggcgcagacgtcgcgggcggcggcagcggcgatgggaagcctgctgggcggctggccgagccacaaccctcagaacttcagccagctcgtcccggccgacccctccgcccagcccacgaatatcacaccaacaacttacattgcaacacataggacagatccacctccaaatcaagtgatcacgacggagcccaggaacatcctgctgaggcatttctaccagaactctgagaacaagccgcggccgaagagggccgccccggagagcgttgccctgcgcaacggcaagcaggcgaggagcctcgccgacggcggaagccagtcgagcacgagaagctaaacaagcaggcgaggagc

SEQ ID NO: 162 - DNA, 3540 bp, Hordeum vulgare
tcggcaaaaaaagggcagagcttggccggagcgagagacggcgcagacgtcgcgggcggcggcagcggcgatgggaagcctgctgggcggctggccgagccacaaccctcagaacttcagccagctcgtcccggccgacccctccgcccagcccacggtcggtcagccccccttcccttccctcccctccctctccgtggcagttccgtggtctcttcgtcctgtcctgccccgtaccacactgctagggtatgctccagtcggagggcgcctgattccacaggtttcaggtgccatgcatgcttgcttgcttgcacaagtgcggagttcatcggtgcccaaaaggggagcggggtctgctgcctgagccaggaattaggagttactcgcactgtgcgtgtgtgcgtctccactggaattctggtatagatggccaatgattgtagctttctgttgactcaagcccaactgtcgagcgattgggtcgtattcagagggatcttgcacagtgaataagcatttaggttttggttttggttttggttgtggtggttgttgctatgtgtactgcaggtcatatgttcagttgatctggtgttatgtatgctagttttcctgtgtttcttgtcagaatgactagaattcagaaatagaaaaggcaggccgaaagaaagaaaaaaaaaactatcatgctagcacttacaatgttgcaagtgtattggtatttggttgacatggcgctaatgctgatcctcattgtttgtgtttgtttctcaccagaatatcacaccaacaacttacattgcagcacataggacagatccacctccaaatcaaggtaaactcttcccatatttattcaaccaatgtctctttgctgcttatacgttctgcgaatttaacctgcctggcttcccctttgctttttgcgcgtgattgaatgtacgcccctctgcactgcgtcaaattctgtattccccatggcaaaataggtttcaggaactgattgatttgaactaaaaactgtataggtagatgggtatcttttcattcaggagggcatcatatttagccatgagagtccatattatgttcccaataatgtcgaaagggcatgggttgattctgctaggccaaacaccatatcatttcgcaggatgttcctaaattataatttggctccttttttttatgatgttattttggcaatggtactggcgatgtcatgggattctgtactcaagaaattatggtgccacatgtccagatgtgatatgtactcaaccacttctaattccccattgacatgctactttaatctccataatatattgtatccaaggtgctatgttctgcatgtcctacacaaatggtggcaatcttatttgccaacagtgactggacacataatacgctaataatagtggtctcctcctgccctcacttgagcccctggtctgtaagaatcacggtttgtgccggtaagtggtgattaccagcaaatagctatggatgataactcggttacacctgtttaaacactgtataagactgaactgctcacctcgattacgtagcagtagttcgatttatttcatctcctacagcttttcattcgaatgtccagttattggaccgtgtcctatattaggtcttcacaaaatatcaaaatatcactttgaaaatattgtctattccattgagacattccaagattacgaaagcatgctccccaattattctgagtcacattagttaacggcacattagtaaggttcagacagctaaatactttagtttcatctaaaatacagtagcttaatgagattgtgattcatgtcattggccatcgttgttggtaattgaacttcttcagcaaactctgaagttcttgcaacagttaaatgtcactgatttacatgatttgggtgatttggaaggaacggactgtcgtgtctttcataacaaggccctgcagcctttggatgtggccgtaataattcaagaggaggtagtgcagtggccatgggcatatgccttctcccatgataccaggcatagttgcaattcccttgctttggcttctttgctaccctagcgtgtttgggttttgccttttgccctttattatatattttgtatattacttgttttaatttcaatctatatgaatgggaagagcacctcccttttccaaaattagtcgtacatttcattataggtctgcaggttgcactgttacaaattcttaatccacaagttagtttttctcttgggagcatgaggaagaggaggacatagcagtgatgtcagacttacattttttacaccttaaaggagattctttaatgttcccttcaagttgaattcttagagaactcatactgagagtgccttcgacaaggagatatagcagcactcggtccttgagaggaattggttgagttgagtgaaagaggcaagcagttgcctaatgattaatcatgaatataggtgggttctcggttcataggcttatggcccattgtctggtgcctaataaatttattaggctaggctgaactgccaaaaatcctggcacatcggcccagctggcggtcagtgaaatttttattgcatactcctaacgaactctacaaagaaaggttaatttcaataataagaactatatgattgatttcctttaaaaaatcgacataatatttttaagagcagagtgcactgacagagcaagcacctgctacattatgtgccatttttcggcgttgcgagcataaattgtgaagaagttatcagttgctaacattgagaagtagacaagaactaaaatcatctgaatagacccttaacactgaacttgcaagtttcagagctgtgccacagattttggttagctgaaatgcactgaacactcgatttggttcattgcagggatcgagttgcttccttttattgccagcttgtgtcaacatttcgtgatatacacattttcttatgaaattcctgtgtggcaaactattcttccggcagatagattggctaaacaagctgctaatgaaacggactgctagttatgtgttgacagaacctccttttctaaaaaaaagttatgtgttgagtgtgtcggtgttgcgtgcctcgttgagtgattagttttgcatatatgtatgttccaagtagataagcctctggctgctcccgtcgagttaatctgtactccgctgacaatctgagccctgcgactgcagtgatcacgacggagcccaggaacatcctgctgaggcatttctaccagaactctgagaacaaggtgagctaccagcactctgacgaagaagagtaggtagaccggcaaccgttgttctcaacgccgtatactcatgtctgcagccgcggccgaagagggccgccccggagagcgttgccctgcgcaacggcaagcaggcgaggagcctcgccgacggcggaagccagtcgagcacgagaagctaaacaagcaggcgaggagccaccgctgcagtcacttttctgctttgtgtg

SEQ ID NO: 163 - protein, 102 aa, Hordeum vulgare
MGSLLGGWPSHNPQNFSQLVPADPSAQPTNITPTTYIATHRTDPPPNQVITTEPRNILLRHFYQNSENKPRPKRAAPESVALRNGKQARSLADGGSQSSTRS

SEQ ID NO: 164 - DNA, 309 bp, Triticum aestivum
atgggaagcccgctgggcggctggccgagccacaacccgcacaacttcagccagctcgtcccggctgacccctccgcccagcccacgaatgtcacaccaacaacttacattgcagcacataggacagatccacctccaaatcaagtgatcacgacggagcccaggaacatcctgttgaggcatttctaccagaactcggagaacaagccgaggccgaagagggccgccccggagagtgcctccgtgcgcaacggcaagcaggcgaggagccccgccgaggacggaagccagtcgagcacgagaagctga

SEQ ID NO: 165 - DNA, 531 bp, Triticum aestivum
ccacgcgtccgcaactgtcggcaaagagagcctggccggagcgagagacggcacacacatcgcggtcgcggacggcggcagcggcgatgggaagcccgctgggcggctggccgagccacaacccgcacaacttcagccagctcgtcccggctgacccctccgcccagcccacgaatgtcacaccaacaacttacattgcagcacataggacagatccacctccaaatcaagtgatcacgacggagcccaggaacatcctgttgaggcatttctaccagaactcggagaacaagccgaggccgaagagggccgccccggagagtgcctccgtgcgcaacggcaagcaggcgaggagccccgccgaggacggaagccagtcgagcacgagaagctgaaccagggctggtgttcttgtcttgcgccgccgccgtggcatctctgaatctctgaatctcctcatatgtttgattttgagaaaggtgttgcacgcatgcacgcatgcactgcacatacatgtgggccaggcctagt

SEQ ID NO: 166 - protein, 102 aa, Triticum aestivum
MGSPLGGWPSHNPHNFSQLVPADPSAQPTNVTPTTYIAAHRTDPPPNQVITTEPRNILLRHFYQNSENKPRPKRAAPESASVRNGKQARSPAEDGSQSSTRS

SEQ ID NO: 167 - DNA, 309 bp, Triticum turgidum; identical to SEQ ID NO: 164

SEQ ID NO: 168 - DNA, 610 bp, Triticum turgidum
gaattcggcacgaggggacggcggcagcggcgatgggaagcccgctgggcggctggccgagccacaacccgcacaacttcagccagctcgtcccggctgacccctccgcccagcccacgaatgtcacaccaacaacttacattgcagcacataggacagatccacctccaaatcaagtgatcacgacggagcccaggaacatcctgttgaggcatttctaccagaactcggagaacaagccgaggccgaagagggccgccccggagagtgcctccgtgcgcaacggcaagcaggcgaggagccccgccgaggacggaagccagtcgagcacgagaagctgaaccagggctggtgttcttgtcttgcgccgccgccgtggcatctctgaatctctgaatctcctcatatgtttgattttgagaaaggtgtttgcacgcatgcacgcatgcactgcacatacatgttggccaggcctagtgcggggtgacaccggcatggcaccactgcagtctctttctcctttgtgtagataataataatgtggccgcaccagcacaccaacatgtactactgtagtagcgcttttgtataatatttggatgatttccct

SEQ ID NO: 169 - protein, 102 aa, Triticum turgidum; identical to SEQ ID NO: 166

SEQ ID NO: 170 - DNA, 315 bp, Sorghum bicolor
atggggagccccctgggcgggtggccgtcgtacaacccgcacaacttcagccagctcgtccctgccgacccctccgcgcagccctcgaatgtcacaccagccacttatgttgcgacccacaggacagacccgccacccaatcaagtgataacaacggaggccaggaacatcctgctgaggcacttctaccagaaatctgaggagaagctgaggccaaagagagctgctccggacaacctcgctccggagaacaacaacaagcagcccaggggacctgtgggcgacgtcgggggccagtcaagcgcaagaggctga

SEQ ID NO: 171 - DNA, 950 bp, Sorghum bicolor
tgtaaccatcactctttttccccgaaaccaaaacgaaaaaaaaaagaaagtgctgctggctgctgccaaccacccgtggtcccatgaagagagcatcgccggagtcggggacggtgcgccgagaaggaacaaaagaagacggcggcggggcggagatggggagccccctgggcgggtggccgtcgtacaacccgcacaacttcagccagctcgtccctgccgacccctccgcgcagccctcgaatgtcacaccagccacttatgttgcgacccacaggacagacccgccacccaatcaagtgataacaacggaggccaggaacatcctgctgaggcacttctaccagaaatctgaggagaagctgaggccaaagagagctgctccggacaacctcgctccggagaacaacaacaagcagcccaggggacctgtgggcgacgtcgggggccagtcaagcgcaagaggctgaagccacacagctggtgctggtgcccgtcctcccctgcctctcatctctcggtgtcatgcagatgcagcctgcatctctcgctcacatgtcacagctggtggttgtttctcccctgtgcgtcctcttcgcctctcacgtatgtacgtatgacccaaagagctgaggtatacatacctggatggttggatggatgtacataaccacctgagacgagacaaagctcggtgcgtgccatttcacatggcactaggtgtgctgcagcctctccttttcatcctctacaatgcaaaaatatggatgtgcccatgctgctatgctagctagccctactccccctgtgctttggatcgtgcaccgcgtcagcagctttttgaaaggctggtggtgatgattgcactctgaaaatccccgtcttctgctgtcagattatactatacgctgctgccgtgcagctgctgctgcgccagccaggggcagcca

SEQ ID NO: 172 - DNA, 3571 bp, Sorghum bicolor
tgtaaccatcactctttttccccgaaaccaaaacgaaaaaaaaaagaaagtgctgctggctgctgccaaccacccgtggtcccatgaagagagcatcgccggagtcggggacggtgcgccgagaaggaacaaaagaagacggcggcggggcggagatggggagccccctgggcgggtggccgtcgtacaacccgcacaacttcagccagctcgtccctgccgacccctccgcgcagccctcggtcggtcagcaacttgcccttcctggcgatctggcctctagtatcatgctgtaatgctaggctccgtacttcctgacgagcttagataagctcgaatatgtttaattgaccgggttcatctgcgcttggcctgtttcttttctctggtttcaggtgccgtcgagaaaaaaaaatctctcttttttttaatcccgtagtatcactttcgggcaggagacagtaatcggtgccggtattgttagttttggctgaaattttggtatggatggctggagaaatggggtctcactgtttgattttagttcagctgccaaagacctgttcaatttgaggggactgtctggccaatttctgaacatctggtctggttttctcatggtcatgtctaccctgggtagattcagttgatgtggtactgatgggctaatggtagttcagttcatggttgctaatgctaccggttgattttctacaacgtcagaaattcgtgctgtcaacttatattatgaattatatatgtccatttcacctggtgctaatgttagttcttttttctttccatgcgtgtttgtcatcagaatgtcacaccagccacttatgttgcgacccacaggacagacccgccacccaatcaaggtaacccctttccatgtccttaagccaatggtattctgctgcatgtaattgatgccgtacaattgcttattcgcaataagccaacaagccccttttttttgtttattgtagtgctttgtatgttaattgcaaaataggtttcatgagctgtttcgtctgaactgacattcttcaggtagatgactctctttgcttcaatggagcatgattagccataagctcctgtgcatgtgtagaatgtgtctgcaagtcacagatggtggtatcagccacagtgaacagaagctctgaacgccttaatccttatcctacacaaacgacgccctctgtagctttgctgtgtttacctgaacccaggtgctctgaacctcgatcttgttgctggtaatctgaactcagaatagtagatatggtagataattagcagtcgctgtcttagccatatactccaatacaatacaatacattaccttccttgctggctaatttaccagttgcaagtactaacagtaccaggctaccagctttgattatgcgatttcatataattttctttctctgttgcaaaatcttatagcaattctagcgttaagccttaaaaaaacacaactgggaaacattgcctgttgtattttggaaattttagattcttaaggctagctttcttattttactgaaagtctgaaacacactgacaactatcaacaaattaatattgattcatctagtcacagcaaatggtaaatttgtttttgacggtaattcagactactgcatagttagtgttcacctcgaatacagtagtccagcagagttgagatttataccatagctgatcatcgctctagatatctcaacatttggcatgctaatggccttctgtggcttatactattctatgcctgagttggtttgcctttgttccatcgtgtaggttgcttgggagatttacactgtttttccttcaaatattttcttagagcatgagaaagcggaagaaaagtttgagttaattggtgctttatggatcagggatgttggtgttcaaaatgaacccatagactagttaaatgcatccatcataatataccttaagagtggagtctctaagacttcagtaaagttggagccttccatttgaaaatgcaatccacagagttctagaactgggatgaccttgagagaagttaggttaatttactactaaatggtagcctaatggtcacattgactaggaatttggcttgaacactctggttgtcaatttgtataacaatatgtcgctctttgttgtggtcattctctggtcctgaacaatttaccaggttaggcgaatccacaagaggtccttattgaattcgtgcctttttggcacagctgattccacatcctgtcggtgaaaatataattatgtgctgtctaccagctgcataaaaggtcccaaaaggaacaagctatgattgtgccttcatctgggggctaatgacatatttttcggagatgttatttgtgaacaatcgaacaatccataacattttaaaggatacttaatcctgaactattgccagcacttcaaccacagacttctggtgaattgcagtgataacaggctaacagcagagatatcccaatttgtggcagataaattactaacaaatcatgggcattctttaactacatgcctgacaattctctccttttagtttccttttacttatcatactgctgcattattttatatatatgtccatagttcacactaatttaggctcaataactgcttcctaccatatcagtgtatttactttcaattcttgtggggacactgatatgttcccgctaaaattgtcacaaaccccccaattcctttctcaactttgctgcatgaaaaccaaccttgttatatttttacctcttactgcggactgaatcgcaccctggaattgcagtgataacaacggaggccaggaacatcctgctgaggcacttctaccagaaatctgaggagaaggtgagctgctactgctagtaagacttcaccatcaaggctacataaaaccacatcactatagaatctaagcttgaaatcctatcctgaactgtagctgaggccaaagagagctgctccggacaacctcgctccggagaacaacaacaagcagcccaggggacctgtgggcgacgtcgggggccagtcaagcgcaagaggctgaagccacacagctggtgctggtgcccgtcctcccctgcctctcatctctcggtgtcatgcagatgcagcctgcatctctcgctcacatgtcacagctggtggttgtttctcccctgtgcgtcctcttcgcctctcacgtatgtacgtatgacccaaagagctgaggtatacatacctggatggttggatggatgtacataaccacctgagacgagacaaagctcggtgcgtgccatttcacatggcactaggtgtgctgcagcctctccttttcatcctctacaatgcaaaaatatggatgtgcccatgctgctatgctagctagccctactccccctgtgctttggatcgtgcaccgcgtcagcagctttttgaaaggctggtggtgatgattgcactctgaaaatccccgtcttctgctgtcagattatactatacgctgctgccgtgcagctgctgctgcgccagccaggggcagcca

SEQ ID NO: 173 - protein, 104 aa, Sorghum bicolor
MGSPLGGWPSYNPHNFSQLVPADPSAQPSNVTPATYVATHRTDPPPNQVITTEARNILLRHFYQKSEEKLRPKRAAPDNLAPENNNKQPRGPVGDVGGQSSARG

SEQ ID NO: 174 - DNA, 315 bp, Setaria italica
atggggagccctctcggtgggtggccgtcgtacaatccgcgcaacttcagccagctcgtcccggccgacccctcctctcagccctcgaatgtcacaccagccacttacattgcaactcacaggacagatccgcctcccaatcaagtgataacaacagagcccaggaacatcctgttgaggcacttctaccagaaatccgaggagaagctgaggccaaagagagcagctcctgacaatctcgctccagagaacaacaacaaacagcccaggggccctgtcgccgatgttggaagccagtcaaacgcaagaagctga

SEQ ID NO: 175 - DNA, 980 bp, Setaria italica
atggcctgttcggtagtgctggctgctgcgctgctgctgctacagtcagcgttcaacacagcgagtggctggctcgctgggctgctgcagctgccgcagccggccgaaaaaagtgcagccgaatatctgccaaggaacacgcacaggtccttcacggatcttgtttttcggttttacaggcaagtaggcaaccatcgcccgttctttgaccccgtcggagttcagatgatcgtggccgtggccgttcagcgatcaggagctggaagacgatgtgaggggagcttcgccggagttagagacggcgcggcgattccggctcaacaaaccaccaggggaacaagaggggcggcggcgtggagatggggagccctctcggtgggtggccgtcgtacaatccgcgcaacttcagccagctcgtcccggccgacccctcctctcagccctcggtcgaatgtcacaccagccacttacattgcaactcacaggacagatccgcctcccaatcaagtgataacaacagagcccaggaacatcctgttgaggcacttctaccagaaatccgaggagaagctgaggccaaagagagcagctcctgacaatctcgctccagagaacaacaacaaacagcccaggggccctgtcgccgatgttggaagccagtcaaacgcaagaagctgaatacagctggtgcttgtcctcccctgcgtctctcaatgccgtgtgcaacctgcatgctgcatgccagctgaagccctggtcctcttgatccaaagagctacgctcattacatgcatgaatgtacataacaacctcccccctttccctccaacattggtttgttatttgttagcgactggtggctgcattttagtgacagattttagtaaagaaaaaggatggttcggcatgaaaagatagccgcttttctcttgcttatgcaatactccgtacaatttagtaaaatatagacactatttgta

SEQ ID NO: 176 - DNA, 3998 bp, Setaria italica
atggcctgttcggtagtgctggctgctgcgctgctgctgctacagtcagcgttcaacacagcgagtggctggctcgctgggctgctgcagctgccgcagccggccgaaaaaagtgcagccgaatatctgccaaggaacacgcacaggtccttcacggatcttgtttttcggttttacaggcaagtaggcaaccatcgcccgttctttgaccccgtcggagttcagatgatcgtggccgtggccgttcagcgatcaggagcgtgggcctgctagtccaagttgggccacgaccacgcactgacgagcgtatggccggtctgggccagatagcgctatgggccgcaacaagattctttttttctcccaaaaagggaggtggaaaaaaaaagaaaacgaaaagtgctaaccaccagtggaagacgatgtgaggggagcttcgccggagttagagacggcgcggcgattccggctcaacaaaccaccaggggaacaagaggggcggcggcgtggagatggggagccctctcggtgggtggccgtcgtacaatccgcgcaacttcagccagctcgtcccggccgacccctcctctcagccctcggtcggtcagcacttccccctctttggcgatctcgtctccaatacaccgcactgactctctccatagttcctgatgatcttgcataagcttgaatatttagttaggaggtattggttggtgcttggtcagtgacatctgtggactcttgtatccacaataaaaaacttccttttgtactgctttcaggcagggagcatgaatcaatggtcgtatggttcgattctgctgaaaccacagtatggatggtttgagaaagagggtatcgatgtttgtttttagttcatctgccagagacccggctcagtttcagtgaattttctgcatactgtccaaacagttaggtcttggttttgtcatggctgccctgataagattcagttgatgttgtacttatcgattgatggtggttctttttgtgtttctaatcgtactccatcaccagcagctcatatggcacatatatatatccatttagagtggccctaatgctattatttagtttttgtattcttgactgagcttgatgctgacaagtgagaacttatactatgaaagatatattcggttgacctcatgctgactgttttttcccctttccatgtgtatctcatcagaatgtcacaccagccacttacattgcaactcacaggacagatccgcctcccaatcaaggtaacacctttccatgttctcgaaccaacgttcgtgtgctgctcccttaaatgtatcctattttatttgctcattgtccacttgcatgcaattgaggccatgtaattactgttcataacttcatattatgccgacaatttcttttctacgtattgtcgtactatgtaagctccgtgttgcaaaataggtctcctgaactgtttggtctgaactgaaagttctgtaggtagatggctctcttatctttcaggaacggagcatgtttctccaaacattttgcgcctcagggtagagtgctgcaagtcgcaaagagtgataccagccataatgactgaactactgaacaccttatcctacacaaatgatgacctctgctacgtttccccaacctatagtccaaaagttcctcgctgccactagtatcagcagctgaatgctatgattgttttttcaatagtagctgtcttaaccatggcgcagtatagcactacttgcctcacttgccaatttaccagctgcaactgtcagtagtaccaggctttatgtgatttattaaatgctgtttgtttcttgcaaaaagcccaggtactgccccttctcaccaaagtttacattcaaacttaattaggaaacctttttttgttacattaggaaattctgagaactgattgatactcgcttggtacagtccccgttcgaggaggccacccacctaggcttgaaactgggtgcttgcaaaatgtttgtgtgtgtacctctgctttcatcgagtttcccggtcagccacagttttgcactcccgttcttgaaacaaagcatgggggatttcatcttcccatggtcaagttttttttaagtaggaaattccaatttatgaaggtatgtttcttagttgtactaaaacacattggctaacagcatattagtatttctcttgggtttgatctccatatgagtggctgggtttttatgctggctcgccaagcctatcacaacccccctcctcctttatccgggctatgttgaaacaacacaggctgacaggcagagttcttggtatgtccatgtgtagcttatctatattgctgccaagttggtttgccttctgttcagacatctagatccctcagaaccattgccctgatctatatggtaagtgcccgaaagaatattgatattggagcatgaggaagatgaataaaggttgggtaagtagcgttcaaaggcgagggtggagcaagggtgttggtgtttaagatggaacaacatgccagttaaacgccattggagccgtccaggtgaaatgccatccacatagagtgctagaactgcgatatccttgagagtagtagtaaactaatggtcaaactgattaggttgttggcttaaacactctggtttgtcaatttctattacatctcaccctaaacatttaccaggcgacacaaaaccagaagaagtccttattgcatccaggccttttggctcagccatgtcctctgcatgttggtgaaaacttgatggtgcactgctgaccaagtgtaccaaaaaaaaagatcaaagatagaaacaaactattctttttaaggagcaaaaggtactgatagataaaagcatactaggtgcccattcggccattcctggggctggtgaggcgatgaaatattattggagaagttttgtgtgaacaatttaaatcatttggagggatacttaactctgaactattttattaagtaccatgcctagatgttacagtcatggagttgattccttaaaagttgttggcagattctatttctctgtctatttcattgtggaagttgtatgtggccaagttaatttccaagttttggcagatacactcaataaaaaaatcgttggtgtttctttaaccaatatatctgccagttatgtgtgttttctgtttatgtgactgcattcgtttatagatatgctacacttcatatttgatcaggctgaccaagtgctttctattatttcattttattgaattttaattcctctgaggcactcatgaacttcctctaaaattgtcatagattgcccaattcctttctcttctccactgcaccaaaaccatctacctggtgttgtatttttctgcttcctgaattcagtaattgatgaatcgtgccctgggattgcagtgataacaacagagcccaggaacatcctgttgaggcacttctaccagaaatccgaggagaaggtaagctgttccatcaaggctgcatatacgccacatcacaatggaatctaatcatctatgcttaaatcctgtcctggactctagctgaggccaaagagagcagctcctgacaatctcgctccagagaacaacaacaaacagcccaggggccctgtcgccgatgttggaagccagtcaaacgcaagaagctgaatacagctggtgcttgtcctcccctgcgtctctcaatgccgtgtgcaacctgcatgctgcatgccagctgaagccctggtcctcttgatccaaagagctacgctcattacatgcatgaatgtacataacaacctcccccctttccctccaacattggtttgttatttgttagcgactggtggctgcattttagtgacagattttagtaaagaaaaaggatggttcggcatgaaaagatagccgcttttctcttgcttatgcaatactccgtacaatttagtaaaatatagacactatttgta

SEQ ID NO: 177 - protein, 104 aa, Setaria italica
MGSPLGGWPSYNPRNFSQLVPADPSSQPSNVTPATYIATHRTDPPPNQVITTEPRNILLRHFYQKSEEKLRPKRAAPDNLAPENNNKQPRGPVADVGSQSNARS

SEQ ID NO: 178 - DNA, 315 bp, Panicum virgatum
atggggagcccactcggcgggtggccgtcgtacaacccgcacaacttcagccagctcgtcccggccgacccctccgctcagccctcgaatgtcacaccagccacttacattgcagctcacaggacagatccacctcccaatcaagtgataacaacagagcccaggaacatcctgctgaggcacttctatcagaaatctgaggagaagctgaggccaaagagagcagctccagacaatctcgctccggagaacaacaacaaacagcccaggggtcccgtcgccgatgttggaagccagtcaaacgctagaagctga

SEQ ID NO: 179 - DNA, 958 bp, Panicum virgatum
aaggaaagcgctaaccaccagcggcagacgaagtgaggggagcatcgccggacgccggagtcagagacggcgcggcgattccggctcaacgaaccaccaggggaacaagacgggcggtggcggcgcggagatggggagcccactcggcgggtggccgtcgtacaacccgcacaacttcagccagctcgtcccggccgacccctccgctcagccctcgaatgtcacaccagccacttacattgcagctcacaggacagatccacctcccaatcaagtgataacaacagagcccaggaacatcctgctgaggcacttctatcagaaatctgaggagaagctgaggccaaagagagcagctccagacaatctcgctccggagaacaacaacaaacagcccaggggtccatggaatacaaaaccgctcgataatcgcgattatcggtgaaatttaccgttaccgatgttgactgatatcggttttcaattgatttttcgatggatttcgatccaaatttcaaaaattcaaagaaatttataactagtgtggaaaaaattctataaaaaactagagcctctctatagtctagaatgatgtcacatattaaaaacaaccaccgtttgtttagacaaaaaaatgtttccaatactaaagcctgataattgatgcaaatccatcgataatcaatgcaaatcagttgatattcaacaattttggttgattttctatttcctttcaccaacttgaccaaatatgcatggggtatttactatattgttgtatattatgctacaaatggatggttatactgataatttccaatgtagattagtgttaaatattagtggtgggaagaaagacttcaatgttgacttgttgttaaatcagttaggatacaataggcttcaatgttgactataatatgtatgcttatactaaaaaaaactatgtctaactggt

SEQ ID NO: 180 - DNA, 5458 bp, Panicum virgatum
aaggaaagcgctaaccaccagcggcagacgaagtgaggggagcatcgccggacgccggagtcagagacggcgcggcgattccggctcaacgaaccaccaggggaacaagacgggcggtggcggcgcggagatggggagcccactcggcgggtggccgtcgtacaacccgcacaacttcagccagctcgtcccggccgacccctccgctcagccctcggtcggtcagcacttcccctctttggcgatctcgtctctaatatacagtaattgactgtctccatacttcctgatgatgctgcataagcttgaataggttagctaggacgtattagtgggtgcttggcctgtgctgtgacaactgcggcctcttgttatcttgtatctgcaataaaaacttctttagtactgtactgcctttatgcagccaggaagcaggatgatcgtattgttcgattctgctgaagtcgcggtatggatggctggaaaaggagggtataaatgtttgtttttagctcatcagccagatactcggctcaattttagtgaattttctgcatgatgtccaaaaattattaggtcttggtttctcatggctgccctgacaagattcagttgatgtagtgttgtcctaatcgattaatgctagttcttttagtgtttctgatcacactgtaccgccagcggctcacatggcaaagcacatatatatccatttagagtgactctaatgttattaggtagtttttatgttcttaacagaacttcatgctgacaagtgataatttaggctatgaaagatatactctgttgacctcatgctgatgctgatggtatgtttcctttccttgtgtatcccatcagaatgtcacaccagccacttacattgcagctcacaggacagatccacctcccaatcaaggtaactccttcccatgttcttgaaaaaatgttctgtgtgctgctacctgctgtaaatgtatcttattttattttctcactgtgcattttcccgcaattgagatcatgcaattacttgttcgcaataagccgagaatttcttttctgtctattttagtactatgtaagctcaatattgcaaagtagatcttgtgaacccgtttggcaaaagttcttagcatgtttctccataagattctttgctcatggtatattgtgtctgcaagtcacagaggtgatatattagccatgatgactgaactactgaacaccttatcctacacaaatgatggtctcccctctgctatgtctccccaatctatagaccataattttccttgctgccactggtaatcagcagctgaaagctatgattgattgttggctgtcttaaccatgtgcagtataatactaattgtcttactgcccatttacctgctgtaagtgtcagtagtaccaggtactgcccctttttcaatatcaaagttttaccaggtaatgcatgcagtgcaatttttctttgatctacatggacaacaattcaatttgctaaatactgcacatatagtactgattccaaaatctgaggatgctactgatctctcacattatagacctatcagcttgacaagtagtattccaaaattattctcaaagctgcttgcactcagattggccaagagtttggacacactaatctcaaggaatcaaagtgcttttattcgaaggagtatccatgataacttcttatacacacaaaatctcattcgagctctacataaagatggcaggccctccctttttattaagctggacattgcaaaggcttttgacactgtgcgatggaattatctgatggaggtgttagagaaacttgggtttggtcacaaatggaggggctggatttctttactgctatcaactgccacttcctcggtcttagtcaatggagcacaaactccaaaatttaagcacatgatcaggttaaggcagggagaccctttgtctccaatgcttttcatcctggcacttgaacctttgcaacacttgctggctttagaagaagcttcgggcaacctatcaccaatacacacaaatatggcaacgttaagaataagtttatttgccgatgatgctgcagtttttctaaacccagtgaaagaagagattgatgtgatcaaagaggtatttcaggcatttggaaatgcttctggactgaaggtgaacttaagtaaaagtgctatctatcctattagatgtgagggcattgatcttgaagaagtactgcagaatttcccatgccaaataaaagccttcccctgcaagtacctgggactaccagtgagtacaaggtgtctaagaagaattgaggtgcaacctttatttgacaaaattgcagctaggctgccagcatggaaggggaagcttttgaatagagcaggctggttgactttggtaaagtctgtactcgccgcagtgccaatttatttcctcacggtgtttcctcttaagaaatgggccttaaagaaaattgatagactgagaagagcctttctttggagaggaactgaggaggcccgtggtgattactgcttggtcaattggaagaaggtaatgctaccaaaggagatgggagggctcgagtattggatctaagttgttttgggagagctctaagattgcgttggttgtggtacgcttggacagagcctgacagaccttgggtgggatcggcaccaccatgtgatgaggtggataaacaacttttcagagcaagcacaattgttcagttgggggatggtaacaaagcttctttctggaaatgtagctggttaaatggaagggcccctagggacattgcacctgggctgtttaagttggcttggagaaagaatagaactgtaagagaagacatcataaatcagcaatggacaagggggctctgtagaatggattcagttgagttaatgtcacagtttgtggttctttgggatgcagtacagcaggttcagttgacggataggccggatgagatagtctggagatggacagctaatggggcttatacttcaaagtctgcttatcttgctcaactcaagggaactttttgtacatttgatgcccaatcaatctggcatgcacatgctgaagggaaacaccgcttctttgcttggcttctagtgcaaagcaaaatattaacggccgacaagctggtcgctaggaattggctgtgtgacactaattgtgctttgtgtgaccaagttcatgaaacagctgcacatctgtttgcattgctcttatgctaagcaggtctggctcgcgatgagcaactggacatcaggcgccatacacatactggcggttcaagacgagggggtcgaggattggtggaacagaagcttagcgttgctaccggtggcacagaaacgctcagttgcggccatcttgatgtacacttgctggaatttgtggaaagaaaggaacaggagagtgtttgaccaaaaatgtttgcagccacatgaagttgtccagctgatcaaggaagaagtcaacctgagaagggtggcttgtggcacacccatggtgttctagttggttttcatgtttagaggattcttgtttagaggagggttaatgtttttatgtaaattaaactcttattgaactcgcttgcttccttcttaaatgcatcggcagcgctcctgccaaactttcaaaaaaaaaagttttaccttaaaaaactaattaggaaaccttctctgttacattagggaattccaaaaagcaatcatacttgctttctacagtctccttcgaggaggtcacccacctagcctcaaacctgggtgcttgcaaaatgtgtgtacctctctgagaactgaaagaacaagtttcctggtcagccacggccgggtcctccccttcttgaaacaaagccagggggaattcatcttgccatggtcaagttctttctaataactttgcattaggaaattccaatttatgaaggcatgcttcatagttttactgaaacatattggctaacagcacattagtatttctcttgggtagctcggtttcatctccatatgaaaccacaagaaatccttgttgcattcaggccttttggcccagtcatgtcctccgtgtgttggtgaaaacttgatagtgcgctgctgaccaagtgtaccaaaagacaaacgaacgaaagaaagaaagaaacaagctattcttgttaaggagcgagaggaggtggtagaagaaaagcatgtgccttattctggggctgatgaggcaatgagatactattggattagtttttatgtgaacaattcaaatcatttggaggcatacttgaatctgaactatacctcagacttcaggcacaaacttctggtggtgaatatttattaaataccatgcctagatgttacaggcatggagttgaatccttaaaagctgttgacagattctatttctgctgtctactttccttaaggaagttgtatgcggacatgtttatttccaagttttagcagatacattcaatgaataattcgttggtgttttgttaaccaatatatcttcttttcattatgtgagtgcattcgtctatagatatgctacactcatgttagatcagactcaagaagcgctttatataaaagtcatccatgttgtatttttactgcttccttaattcattgattgacaaatcgtgccattggaattgcagtgataacaacagagcccaggaacatcctgctgaggcacttctatcagaaatctgaggagaaggtaagctgttccatcaaggctgtacagatcacatgactatggaatctaaccatctataccttaatcctgtcctgaactttagctgaggccaaagagagcagctccagacaatctcgctccggagaacaacaacaaacagcccaggggtccatggaatacaaaaccgctcgataatcgcgattatcggtgaaatttaccgttaccgatgttgactgatatcggttttcaattgatttttcgatggatttcgatccaaatttcaaaaattcaaagaaatttataactagtgtggaaaaaattctataaaaaactagagcctctctatagtctagaatgatgtcacatattaaaaacaaccaccgtttgtttagacaaaaaaatgtttccaatactaaagcctgataattgatgcaaatccatcgataatcaatgcaaatcagttgatattcaacaattttggttgattttctatttcctttcaccaacttgaccaaatatgcatggggtatttactatattgttgtatattatgctacaaatggatggttatactgataatttccaatgtagattagtgttaaatattagtggtgggaagaaagacttcaatgttgacttgttgttaaatcagttaggatacaataggcttcaatgttgactataatatgtatgcttatactaaaaaaaactatgtctaactggt

SEQ ID NO: 181 - protein, 104 aa, Panicum virgatum
MGSPLGGWPSYNPHNFSQLVPADPSAQPSNVTPATYIAAHRTDPPPNQVITTEPRNILLRHFYQKSEEKLRPKRAAPDNLAPENNNKQPRGPVADVGSQSNARS

SEQ ID NO: 182 - DNA, 315 bp, Phyllostachys edulis
atggggagccccctgggtgactggccgtcctacaacccgcacaacttcagccagctcgtcccggccgacccctccgcccagccctcgaatgtcacaccagccacgtacattgcgacgcataggacagatccacctcccaatcaagtgataacaactgactctaggaacatcctgttgaggcatttttatcaaaaatccgaggagaagttgaggccaaagagagccgcaccggacaatcttaccctgcagaacaattgcaaacagccaaggggccctgttgccgatggtggaagccagtcaagtagtagaagctaa

SEQ ID NO: 183 - DNA, 658 bp, Phyllostachys edulis
gaagaggaagaagaagaagaagaagaaggaagcatcggcggtggcgtcgcggcgatggggagccccctgggtgactggccgtcctacaacccgcacaacttcagccagctcgtcccggccgacccctccgcccagccctcgaatgtcacaccagccacgtacattgcgacgcataggacagatccacctcccaatcaagtgataacaactgactctaggaacatcctgttgaggcatttttatcaaaaatccgaggagaagttgaggccaaagagagccgcaccggacaatcttaccctgcagaacaattgcaaacagccaaggggccctgttgccgatggtggaagccagtcaagtagtagaagctaaatcaccgccagtgttctcctctcctgcatctcttacggtcgttgcggctgctgctgatgcatgtcatgctacctgtgtggctgtgtgcttgttcaagcatgcgaagccctctcatttctcatgtattatcaaaagagcttggatgcatgtacatacccttcagcgagcccctcagtgcggtacctttcacatggcactactgcagtctcttctgaatataatgtgcccacactagccaacttgtgcttttgattgaaacaaaaccatggctccataattgcgttgcttc

SEQ ID NO: 184 - protein, 104 aa, Phyllostachys edulis
MGSPLGDWPSYNPHNFSQLVPADPSAQPSNVTPATYIATHRTDPPPNQVITTDSRNILLRHFYQKSEEKLRPKRAAPDNLTLQNNCKQPRGPVADGGSQSSSRS

SEQ ID NO: 185 - DNA, 276 bp, Picea glauca
atggggtcattgcttggagattggccctcctataatccgcacaatttcagtcagttgaggccgtcggatccctcgcatccctcgcaattgacaccggtcacttactatcctactcataatagaacagcacccccagcacaccaagtaatttcaactgaggctacaaatatccttttaaggcagttttatcagcgagcagaagagaagttgaaggcaaagaggccggcctctgatgctcttgtacaagaacacatgaacaagcaccccaagagctga

SEQ ID NO: 186 - DNA, 645 bp, Picea glauca
aagacacatggatcggttctgcacatgcagccgcgaggatctgcgtccaggcagtggctggagacggcccctccacctgttattcgcgtcaagaaacggactctccctgcgcagaaactggagaccatagcagaagaatcctgctgtttcgaagaccctgaaagcatcgagcctgattccccgtcacagacacgggcgtcagctttgagatttgggcagagcggctacgaaatcatcgagcccgattccccgtcacagacacgggcgtcagcgttgagatttgggcagagcggttatgaaagcttcgagcccgatttcccgtcacagatacgggcgtcggcgttgagatctgggtaatgacgggttctgtttttctgctgtattggttgagtgggttgccgtcaagtgacgattctagactgacggggggttaagcgtgtttcgggctcaaatgggtttttttattttatgtaatttgtcagaaattttctccatcggcgatcgtatggatcaagatggcagttatctcctcgtgtacagtggaattttctgttgtcaatctcatgtacataatttggaattttctgttgtcaatctcatgtacataattcgtggatatagtggaatcggaattttctgtacgtc

SEQ ID NO: 187 - protein, 91 aa, Picea glauca
MGSLLGDWPSYNPHNFSQLRPSDPSHPSQLTPVTYYPTHNRTAPPAHQVISTEATNILLRQFYQRAEEKLKAKRPASDALVQEHMNKHPKS

SEQ ID NO: 188 - DNA, 294 bp, Selaginella moellendorffii
atgggttccttgctgggcgatcttccttcgtacaacccgcacaatttcagccagttgagaccatcggatccttctcatcgctcccaactcacaccgctcacttatcacgctactcacgaccggacgatgcctccggcggatcaagtcatctccactgaagctaccaacattttgctgaggcacttctatcaaaaagccgatcacaagctcaagttgaagcgctcggccaccgattcgcctctcggggatcacaagcgtcccaagagcacaacttgcgctccagagaagagatga

SEQ ID NO: 189 - DNA, 732 bp, Selaginella moellendorffii
ggctcttttccatgtcataggaggaggagagaagggacattcttttagctgcggggttgcgatcgatcgagcgagagggaatcggtgtgcgccttaaaatcctggtcgctctatcggatagaagcgagcgatcgtgtcgcttgcgctcgaagggtagggtttttggttctcccagagtgtaggtagggctttgcaatgccgctgcgcctcctcctctagaagcgcgcagatctatcgtcttcgtcgagtagcaacgcaaagcgaaaaaagaggttttcttttcgcgaggatcacaatgggttccttgctgggcgatcttccttcgtacaacccgcacaatttcagccagttgagaccatcggatccttctcatcgctcccaactcacaccgctcacttatcacgctactcacgaccggacgatgcctccggcggatcaagtcatctccactgaagctaccaacattttgctgaggcacttctatcaaaaagccgatcacaagctcaagttgaagcgctcggccaccgattcgcctctcggggatcacaagcgtcccaagagcacaacttgcgctccagagaagagatgatcgcgagttctccctgtacttaacaagcccgcgatggaaaaaaaaacagaggttggctacacaggtttgatgagcagaatccattttctcgatctctaagcttgtgaatatctagatcgacaatggtaactttcttttagaaa

SEQ ID NO: 190 - DNA, 1018 bp, Selaginella moellendorffii
ggctcttttccatgtcataggaggaggagagaagggacattcttttagctgcggggttgcgatcgatcgagcgagagggaatcggtgtgcgccttaaaatcctggtcgctctatcggatagaagcgagcgatcgtgtcgcttgcgctcgaagggtagggtttttggttctcccagagtgtaggtagggctttgcaatgccgctgcgcctcctcctctagaagcgcgcagatctatcgtcttcgtcgaggtatgtggagtaatctctccttgttcttcccctcttctcattagctcttttcattcatcagtagcaacgcaaagcgaaaaaagaggttttcttttcgcgaggatcacaatgggttccttgctgggcgatcttccttcgtacaacccgcacaatttcagccagttgagaccatcggatccttctcatcgctccgtaagagatcgacgagcattttctcttcggtttttcttctcttcgtgttttcttcgttgttcttgcttgactgaccaccatttcttttttttttttctttttttttttgcagcaactcacaccgctcacttatcacgctactcacgaccggacgatgcctccggcggatcaaggtaaccatcaccatagcttcgcgaatttgagctaactttgctttctttgcagtcatctccactgaagctaccaacattttgctgaggcacttctatcaaaaagccgatcacaaggtaagttcttcccgatcaatgctatgattcattcatcactcactcgagtgtatgcaagcagctcaagttgaagcgctcggccaccgattcgcctctcggggatcacaagcgtcccaagagcacaacttgcgctccagagaagagatgatcgcgagttctccctgtacttaacaagcccgcgatggaaaaaaaaacagaggttggctacacaggtttgatgagcagaatccattttctcgatctctaagcttgtgaatatctagatcgacaatggtaactttcttttagaaa

SEQ ID NO: 191 - protein, 97 aa, Selaginella moellendorffii
MGSLLGDLPSYNPHNFSQLRPSDPSHRSQLTPLTYHATHDRTMPPADQVISTEATNILLRHFYQKADHKLKLKRSATDSPLGDHKRPKSTTCAPEKR

SEQ ID NO: 192 - protein, 113 aa, Arabidopsis thaliana
MGSSSLMLGDWPSFDPHNFSQLRPSDPSSNPSKMTPATYHPTHSRTLPPPDQVITTEAKNILLRHFYQRAEEKLRPKRAASENLLAPEHGCKQPRGPVASSTSDTQSSASGRS

SEQ ID NO: 193 - protein, 13 aa, Arabidopsis thaliana
PHNFSQLRPSDPS

SEQ ID NO: 194 - protein, 15 aa, Arabidopsis thaliana
RTLPPPDQVITTEAK

SEQ ID NO: 195 - protein, 5 aa, Arabidopsis thaliana
NILLR

SEQ ID NO: 196 - protein, 8 aa, Arabidopsis thaliana
KLRPKRAA

SEQ ID NO: 197 - DNA, 54 bp, Artificial Sequence
  AtDDA1 primer
ggggaccactttgtacaagaaagctgggtagaatagtgagcaactttaagtcga

SEQ ID NO: 198 - DNA, 59 bp, Artificial Sequence
  AtDDA1 primer
ggggaccactttgtacaagaaagctgggtataagccctgagtagatgaagaagaagacg

SEQ ID NO: 199 - DNA, 22 bp, Artificial Sequence
  AtDDA1 primer
ctgggttttgctgcttacttgg

SEQ ID NO: 200 - DNA, 22 bp, Artificial Sequence
  AtDDA1 primer
tcctacgaaatcctgtgttatg

SEQ ID NO: 201 - DNA, 20 bp, Artificial Sequence
  AtDDA1 primer
ccctccgatccttctaatcc

SEQ ID NO: 202 - DNA, 24 bp, Artificial Sequence
  ACT8 primer
gctgcgtataagaatgtttttcac

SEQ ID NO: 203 - DNA, 20 bp, Artificial Sequence
  ACT8 primer
ggtactggaatggttaaggc

SEQ ID NO: 204 - DNA, 20 bp, Artificial Sequence
  AtDDA1 primer
gtccaacacaataccggttg

SEQ ID NO: 205 - DNA, 22 bp, Artificial Sequence
  AtPYL8 primer
ctatgacgtcccggactatgca

SEQ ID NO: 206 - DNA, 21 bp, Artificial Sequence
  AtDDA1 primer
ggtgaagagagatgattgaag

SEQ ID NO: 207 - DNA, 24 bp, Artificial Sequence
  GFP primer
tcgtccctccgatccttctaatcc

SEQ ID NO: 208 - DNA, 20 bp, Artificial Sequence
  ACT8 primer
cttgccgtaggtggcatcgc

SEQ ID NO: 209 - DNA, 20 bp, Artificial Sequence
  ACT8 primer
ggtactggaatggttaaggc

SEQ ID NO: 210 - DNA, 20 bp, Artificial Sequence
  AtDDA1 primer
gtccaacacaataccggttg

(19) United States
(12) Patent Application Publication
(10) Pub. No.: US 2016/0102316 A1
(43) Pub. Date: Apr. 14, 2016

(54) STRESS TOLERANT PLANTS

(71) Applicant: CONSEJO SUPERIOR DE INVESTIGACIONES CIENTIFICAS(CSIC), Madrid (ES)

(72) Inventors: Vicente Rubio Munoz, Madrid (ES); Elisa Iniesto Sanchez, Madrid (ES); Maria Luisa Irigoyen Miguel, Madrid (ES)

(21) Appl. No.: 14/894,109

(22) PCT Filed: May 29, 2014
(86) PCT No.: PCT/EP2014/061214

§ 371 Date: Nov. 25, 2015

(30) Foreign Application Priority Data
May 29, 2013 (EP) 13382197.5

(51) Int. Cl. C12N 15/82; A01H 1/06

(52) U.S. Cl. CPC C12N 15/8202; C12N 15/8261; A01H 1/06

(57) ABSTRACT

The invention relates to transgenic plants and methods for modulating abscisic acid (ABA) perception and signal transduction in plants. The plants fmd use in increasing yield in plants, particularly under abiotic stress.

### FIELD OF THE INVENTION

The invention relates to transgenic plants with improved phenotypic traits, including enhanced growth under stress conditions. The improved traits are conferred by altered ABA receptor signalling. Also within the scope of the invention are related methods, uses, isolated nucleic acids and vector constructs.

### INTRODUCTION

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits. A trait of particular economic interest is growth, in that it is a determinant of eventual crop yield.

Plants adapt to changing environmental conditions by modifying their growth. Plant growth and development is a complex process involves the integration of many environmental and endogenous signals that, together with the intrinsic genetic program, determine plant form. Factors that are involved in this process include several growth regulators collectively called the plant hormones or phytohormones. This group includes auxin, cytokinin, the gibberellins (GAs), abscisic acid (ABA), ethylene, the brassinosteroids (BRs), and jasmonic acid (JA), each of which acts at low concentrations to regulate many aspects of plant growth and development. Abiotic and biotic stress can negatively impact on plant growth leading to significant losses in agriculture. Even moderate stress can have significant impact on plant growth and thus yield of agriculturally important crop plants. Therefore, finding a way to improve growth, in particular under stress conditions, is of great economic interest.

ABA has a central role in the control of seed germination and the regulation of responses to abiotic stresses, such as drought, high salinity and low temperatures (Chinnusamy et al., 2008; Hauser et al., 2011; Hirayama and Shinozaki, 2010). Plants respond to ABA in many ways, including closing stomata under drought stress, maintaining seed dormancy and inhibiting vegetative growth. For example, mutants with reduced ABA content or displaying insensitivity to ABA are more tolerant to salt stress during germination. ABA inhibits vegetative growth under stress conditions, in particular under drought conditions, when it accumulates to help plant survival through inhibition of other processes, including, stomata opening and plant growth. Thus, stress tolerance comes at the price of reduced growth and thus reduced yield. This has a particular impact on agriculture in temperate climates where limited water availability rarely causes plant death, but restricts biomass and seed yield. Moderate water stress, that is suboptimal availability of water for growth, can occur during intermittent intervals of days or weeks between irrigation events and may limit leaf growth, light interception, photosynthesis and hence yield potential. Leaf growth inhibition by water stress is particularly undesirable during early establishment. ABA signaling is mediated by the PYR/PYL/RCAR family of ABA receptors, which allow direct ABA-dependent inhibition of clade A phosphatases type-2C (PP2Cs), for instance ABI1, HAB1, HAB2, PP2CA, which are key negative regulators of the pathway (Rubio et al., 2009; Saez et al., 2006). Inhibition of PP2Cs leads to activation of sucrose non-fermenting 1-related subfamily 2 (SnRK2) kinases, which, in turn, regulate transcriptional response to ABA by phosphorylating specific protein targets, including ABFs/AREBs transcription factors.

The CDD complex is conserved in humans where it has been termed DDD-E2 since it contains, in addition to DDB1 and DET1, a canonical E2 Ub conjugase (highly homologous to UEV COP10) and a small protein with no obvious motifs called DET1-, DDB1-Associated 1 (DDA1; (Pick et al., 2007)). Functional characterization of hDDA1 showed it acts as a positive regulator of multiple CRL4s, although the molecular basis of this activity remains completely unknown (Olma et al., 2009).

There is a need for methods for making plants with increased yield, in particular under moderate stress conditions. In other words, whilst plant research in making stress tolerant plants is often directed at identifying plants that show increased stress tolerance under severe conditions that will lead to death of a wild type plant, these plants do not perform well under moderate stress conditions and often show growth reduction which leads to unnecessary yield loss. The invention is aimed at addressing this need by providing transgenic plants and methods for manipulating stress response based on the findings that DDA1 binds ABA receptor PYL8 in vivo and facilitates its proteasomal degradation when overexpressed in plants, and that overexpression of DDA1 mitigates the detrimental effects of ABA on plant growth and germination.

### SUMMARY OF THE INVENTION

The invention is directed to methods for modulating plant response to ABA. In certain embodiments, crop yield is maintained by ablating the detrimental effects of ABA on plant and seed development. In particular, the invention comprises compositions and methods for abolishing, disrupting or delaying ABA signaling or function. The compositions and methods are useful for abolishing, disrupting or delaying ABA function or effect in a tissue-preferred and/or developmentally-preferred manner to insulate vegetative and/or reproductive tissue from stress and adverse environmental conditions. This may advantageously alter the developmental time frame of certain tissues so as to minimize effects of abiotic stress. For example, the timing of certain aspects of endosperm development may be altered to avoid negative impacts of abiotic stress.

In a first aspect, the invention relates to a transgenic plant with an altered response to abscisic acid (ABA) wherein said plant expresses a nucleic acid construct comprising a DDA1 nucleic acid, preferably a plant DDA1 nucleic acid. In a second aspect, the invention relates to a product derived from a plant as defined herein. In another aspect, the invention relates to a vector comprising a DDA1 nucleic acid or a nucleic acid construct comprising a DDA1 nucleic acid, preferably a plant DDA1 nucleic acid. In another aspect, the invention relates to a host cell comprising a vector according to the invention. In another aspect, the invention relates to a method for altering or reducing a plant response to ABA, said method comprising introducing into said plant and expressing a DDA1 nucleic acid or a nucleic acid construct comprising a DDA1 nucleic acid, wherein said DDA1 nucleic acid is preferably a plant DDA1 nucleic acid. In another aspect, the invention relates to a method for modulating the interaction of a PYL receptor, for example PYL8, with ABA said method comprising introducing into said plant and expressing a DDA1 nucleic acid or a nucleic acid construct comprising a DDA1 nucleic acid, wherein said DDA1 nucleic acid is preferably a plant DDA1 nucleic acid. In another aspect, the invention relates to a method for reducing seed dormancy said method comprising introducing into said plant and expressing in said planta DDA1 nucleic acid or a nucleic acid construct comprising a DDA1 nucleic acid, wherein said DDA1 nucleic acid is preferably a plant DDA1 nucleic acid.

In another aspect, the invention relates to a method for increasing yield and/or growth of a plant under stress conditions said method comprising introducing into said plant and expressing a DDA1 nucleic acid or a nucleic acid construct comprising a DDA1 nucleic acid, wherein said DDA1 nucleic acid is preferably a plant DDA1 nucleic acid. In another aspect, the invention relates to a method for mitigating the impacts of stress conditions on plant growth and yield said method comprising introducing into said plant and expressing a DDA1 nucleic acid or acid or a nucleic acid construct comprising a DDA1 nucleic acid, wherein said DDA1 nucleic acid is preferably a plant DDA1 nucleic acid.

In another aspect, the invention relates to a method for producing a transgenic plant with improved yield/growth under stress conditions said method comprising introducing into said plant and expressing a DDA1 nucleic acid or a nucleic acid construct comprising a DDA1 nucleic acid, wherein said DDA1 nucleic acid is preferably a plant DDA1 nucleic acid. In another aspect, the invention relates to a use of a DDA1 nucleic acid or a nucleic acid construct comprising a DDA1 nucleic acid, preferably a plant DDA1 nucleic acid, in altering or reducing a plant response to ABA, improving yield/growth under stress conditions and altering a plants' stress response. In another aspect, the invention relates to a method for increasing expression of a DDA1 nucleic acid in a plant, preferably a plant DDA1 nucleic acid compared to a control plant.

The term DDA1 nucleic acid as used herein designates any DDA1 nucleic acid from any organism. Preferred organisms are plants. According to the various aspects of the invention, the DDA1 nucleic acid may be AtDDA1, a functional variant or a homolog/ortholog thereof or a functional variant of such homolog/ortholog.

According to the various aspects of the invention, the stress is preferably water shortage, for example drought conditions, or salinity.

In another aspect, the invention relates to a plant with increased expression of an endogenous DDA1 nucleic acid wherein said endogenous DDA1 promoter carries a mutation introduced by mutagenesis or genome editing which results in increased expression of the DDA1 gene. In another aspect, the invention relates to plant with increased stability of the endogenous DDA1 protein wherein said endogenous DDA1 nucleic acid carries a mutation introduced by mutagenesis or genome editing which results in increased a DDA1 protein with increased stability.

In another aspect, the invention relates to a method for overexpressing a DDA1 plant nucleic acid, producing plants, a method for mitigating the impacts of stress conditions on plant growth and yield and a method for producing plants with improved yield/growth under stress conditions comprising the steps of mutagenising a plant population, identifying and selecting a plant with an improved yield/growth under stress conditions and identifying a variant DDA1 promoter or gene sequence. In another aspect, the invention relates to a method for increasing expressing of a DDA1 plant nucleic acid, a method for mitigating the impacts of stress conditions on plant growth and yield and a method for producing a plant with improved yield/growth under stress conditions comprising the steps of altering the DDA1 promoter sequence using genome editing and identifying and selecting plants with an improved yield/growth under stress conditions. In another aspect, the invention relates to a method for increasing stability of a DDA1 plant polypeptide, a method for mitigating the impacts of stress conditions on plant growth and yield and a method for producing a plant with improved yield/growth under stress conditions comprising the steps of altering the endogenous DDA1 nucleic acid sequence using genome editing resulting in a mutant protein with increased stability and identifying and selecting plants with an improved yield/growth under stress conditions.

The invention is further described in the following non-limiting figures.

### FIGURES

FIG. 1. DDA1 associates with the CDD complex in planta.

(A) Gel filtration fractions obtained in the purification of the CDD complex from cauliflower were separated on a 15% SDS-PAGE gel and subjected to silver staining (upper panel) or to immunoblot analysis (4 lower panels). Antibodies used in each case are shown on the right side. The position of specific protein bands was determined according to data reported by Yanagawa et al., 2004.

(B) Isolation of DDA1-associated proteins by Tandem Affinity Purification (TAP) techniques. DDA1-TAP fusion was expressed and purified from transgenic cell cultures. Specific bands obtained were excised, trypsine-digested and analyzed by mass spectrometry.

(C) Proteins identified in (B) are listed. Accession numbers and names of proteins co-purified with DDA1, together with the number of positive identifications in two independent TAP experiments are shown.

(D-E) DDA1 interacts with DDB1 proteins in yeast two hybrid assays. DDA1 interaction with CDD complex components DDB1a and DDB1b (D), and DET1 and COP10 (E) was assessed. Growth of yeast transformed with the indicated constructs on selective plates is shown. Selective media contained different concentrations of 3-amino-1,2,4-triazole (3AT; ranging 0.5-10 mM). Previously reported DET1-DDB1a interaction was used as positive control. Empty vectors were used as negative controls.

(F) DDA1 interacts with the BPA domain in DDB1a. Interaction of DDA1 with a series of DDB1a deletion constructs, containing different domain combinations (represented in left panel), was assessed in yeast two hybrid experiments (right panel). Experimental conditions were as in (D-E).

FIG. 2. DDA1-GFP fusion localizes in nuclei and plastids.

(A) Quantitative RT-PCR analysis of DDA1-GFP expression levels in three independent oeDDA1-GFP lines compared to endogenous DDA1 in wild-type plants.

(B) DDA1-GFP associates to FLAG-COP10 in vivo. Immunoprecipitation of DDA1-GFP fusions was performed using total protein extracts prepared from 8-d-old oeDDA1-GFP, oeFLAG-COP10 and oeDDA1-GFP/oeFLAG-COP10 seedlings. Total extracts (Input) and immunoprecipitates (IP) were subjected to immunoblot analysis with anti-GFP and anti-FLAG. Panels labeled with an asterisk in (B and C) correspond to non-specific bands used as loading controls.

(C) DDA1-GFP associates to CUL4 in vivo. Immunoprecipitation assays were performed as in (B) using protein extracts from 8-d-old wild-type (Col) and oeDDA1-GFP seedlings. Anti-GFP and anti-CUL4 antibodies (Chen et al., 2006) were used to detect DDA1-GFP and CUL4, respectively.

(D-H) Confocal fluorescence images of roots from 5-d-old oeDDA1-GFP seedlings. (E) corresponds to a detail of the picture shown in (D). Nuclei were labeled with 4′,6-diamidino-2-phenylindole (DAPI) stain. Merge image (H) shows colocalization of DDA1-GFP fluorescence and DAPI stain.

(I-N) Confocal images of N. benthamiana epidermal cells expressing DDA1-GFP and a plastid (I-K) or endoplasmic reticulum (L-N) mCherry fluorescent marker (pt-rk CD3-99 and ER-rk CD3-959, respectively (Nelson et al., 2007)). DDA1-GFP localizes in both in nuclei and plastids in Arabidopsis roots and tobacco leaves. Stromules can be visualized as protuberances in plastids.

FIG. 3. DDA1 is essential for female gametophyte development.

(A) A diagram of DDA1 genomic region shows the position of the G to A mutation identified in dda1-1 plants, which affects the donor splice-site of the second intron and is predicted to impair proper splicing of DDA1 premRNA, yielding a truncated protein.

(B-C) Mature siliques of wild-type and dda1-1 hererozygous plants (showing ˜15% unfertilized ovules). Arrows indicate collapsed ovules. Scale bars represent 1 mm.

(D) Inflorescence images of 4-week-old wild-type and homozygous dda1-1 plants. dda1-1 lines show undeveloped siliques that do not set seeds. Scale bars represent 1 mm.

(E) Non-pollinated pistils from wild-type and homozygous dda1-1 plants are undistinguishable. Scale bars represent 1 mm.

(F) Homozygous dda1-1 mutants show aberrant ovule development. Nomarski images of cleared ovules from wild-type and homozygous dda1-1 non-pollinated flowers. Scale bars represent 200 μm.

FIG. 4. DDA1 interacts with ABA receptor PYL8.

(A) Y187 yeast cells transformed with pGBKT7-DDA1 were used to screen a cDNA library prepared from Arabidopsis seedlings in the pGADT7 vector and transformed into AH109 cells. Positive clones included truncated versions of ABA receptors PYL4 and PYL9. Yeast clones were grown in selective media containing different concentrations of 3AT (ranging 0.5 to 5 μM). Empty pGADT7 vector was used as negative control. (B-C) DDA1 interaction with full length PYL8 (B), PYL4, PYL5, PYL6 or PYL9 (C) was assessed using yeast two hybrid experiments as in (A). Physical association between PYL8 and other components of the CDD complex (DDB1a, DDB1b, DET1 and COP10) was also tested (B).

(D-E) Analysis of DDA1 and PYL8 interaction by BiFC. Confocal images of N. benthamiana epidermal cells expressing different construct combinations as indicated were obtained. Reconstitution of YFP fluorescence indicates that the corresponding DDA1 and PYL8 constructs directly interact. YFP fluorescence, DAPI staining of nuclei, and merged images, including plastid autofluorescence in the far-red channel, are shown. Scale bars represent 10 μm.

FIG. 5. DDA1-GFP over-expression reduces 3HA-PYL8 accumulation in both seedlings and seeds.

(A) Proteasome inhibitor MG132 stabilizes 3HA-PYL8. 9-d-old oe3HA-PYL8 (T₀) seedlings (T₀) were treated or not during 2 h with 50 μM MG132.

(B, C) Affinity purification of polyubiquitinated 3HA-PYL8. oe3HA-PYL8 protein extracts were incubated with Ub-binding p62 resin or with empty agarose resin (negative control). Anti-Ub was used to detect total ubiquitinated proteins. Anti-HA allowed detection of 3HA-PYL8 and its ubiquitinated forms. Wild-type (Col) protein extracts were used as immunoblot controls. An asterisk indicates the position of a non-specific protein detected by anti-HA.

(D, E) Time course of relative abundance of 3HA-PYL8 in 8-d-old seedlings treated with 10 μM cicloheximide (CHX) in the presence or absence of 50 μM ABA. Protein level analysis in (E) was carried out using ImageJ software.

(F) Immunoblots showing increased accumulation of 3HA-PYL8 in the presence of proteasome inhibitor MG132.

Immunoblots in (A and F) were performed using anti-HA to detect 3HA-PYL8. Panels labeled with an asterisk show Ponceau staining of Rubisco as a loading control.

(G) Immunoblot analysis of 3HA-PYL8 levels in seeds corresponding to oe3HA-PYL8/oeDDA1-GFP and control (oe3HA-PYL8) plants. Both lines were in the pyl8-1 background. Prior to protein extraction, imbibed seeds were maintained for 24 h in MS media with or without 3 μM ABA. Anti-HA and anti-RPT5 antibodies were used to detect 3HA-PYL8 and for loading control purposes, respectively.

(H) Protein level analysis of samples described in (F) was carried out using ImageJ software.

(I) Semiquantitative RT-PCR analysis to assess the expression levels of the oe3HA-PYL8 and DDA1-GFP transgenes in samples described in (G). ACTIN8 (ACT8) was used as a housekeeping reference gene.

FIG. 6. DDA1 over-expressing plants show reduced sensitivity to ABA.

(A) The percentage of seeds that germinated (radicle emergence) in the presence of 0.5 μM ABA at 72 h after sowing was compared for wild type (Col), oeDDA1-GFP and oeHAB1 (ABA-insensitive control) lines.

(B) Percentage of seeds that germinated and developed green cotyledons and the first pair of true leaves at 5 d. Same genotypes as in A were compared.

(C) Quantification of ABA-mediated root growth inhibition. Same genotypes as in A were compared together with pyl8-1 mutants.

(D, E) ABA-mediated shoot growth inhibition of seedlings that were either germinated on 0.5 μM ABA or germinated on MS medium and transferred to 10 μM ABA. Photographs from panel D or E were taken 10 or 20 d after sowing or after transferring seedlings to plates lacking or containing 10 uM ABA, respectively.

(F) Reduced sensitivity to ABA-mediated inhibition of root growth from oeDDA1-GFP plants compared to Col wild type. Bars correspond to 1 cm.

(G, H) Percentage of seeds that germinated in the presence of 150 mM Nacl or 400 mM Mannitol at 5 d after sowing. ABA-insensitive oral mutant plants were used as a control.

(I) Quantification of ABA-mediated shoot growth inhibition as displayed in (E). ABA-hypersensitive hab1-1 ab/1-2 double mutants were used as a control (Saez et al., 2006).

(J) Percentage of seeds that germinated and developed green cotyledons at 5 d in the presence of 1 μM ABA and/or 10 μM β-Estradiol (Estr). Genotypes corresponded to wild type (Col) plants, cra1 mutants (Fernandez-Arbaizar et al., 2012) and plants expressing DDA1 under the control of a β-Estradiol inducible promoter (iDDA1).

(K) Photographs of plants analyzed in (J) were taken 10 d after sowing. *p<0.01 (Student's t test) with respect to the wild type in the same experimental condition. MS media (MS) was used as a control in all analyses.

FIG. 7. Mutants in CDD complex components show enhanced sensitivity to ABA.

(A) Percentage of seeds that germinated (radicle emergence) in the presence of 0.5 μM ABA at 4 d after sowing.

(B) Percentage of seeds that germinated and developed green cotyledons and the first pair of true leaves at 7 d.

(C) Photographs from representative seedlings taken 10 d after sowing.

(D) Quantification of ABA-mediated root growth inhibition of (1) Col wild type compared with (2) hab1-1 ab/1-2, (3) det1-1, (4) cop10-4 and (5) ddbla mutants. Seeds were germinated on MS medium and transferred to 10 μM ABA for 10 U.

(E) Photographs of representative seedlings analyzed in D were taken 10 d after transferring seedlings to plates lacking or containing 10 μM ABA. * p<0.01 (Student's t test) with respect to the wild type in the same experimental condition.

(F) Immunoblot analysis of 3HA-PYL8 levels in seeds corresponding to wild type (oe3HA-PYL8) and cop10-4 (oe3HA-PYL8/cop10-4) plants. Both lines were in the pyl8-1 background. Prior to protein extraction, imbibed seeds were maintained for 24 h in MS media with or without 3 μM ABA. Anti-HA and anti-RPT5 antibodies were used to detect 3HA-PYL8 and for loading control purposes, respectively. Lower panels correspond to semiquantitative RT-PCR analyses to assess the expression levels of the oe3HA-PYL8 transgene. ACTIN8 (ACT8) was used as a housekeeping reference gene.

(G) Protein level analysis of samples described in (F) was carried out using ImageJ software.

FIG. 8: Alignment of AtDDA1 and orthologs in other plants. The average sequence identity is 66%.

FIG. 9: Phylogenetic tree.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA). RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA. The terms “peptide”, “polypeptide” and “protein” are used interchangeably herein and refer to amino acids in a polymeric form of any length, linked together by peptide bonds.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either

(a) the nucleic acid sequences encoding proteins useful in the methods of the invention, or

(b) genetic control sequence(s) which is operably linked with the nucleic acid sequence according to the invention, for example a promoter, or

(c) a) and b) are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815 both incorporated by reference.

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, breeding methods, stable transformation methods, transient transformation methods, and virus-mediated methods. Methods are known in the art for the targeted insertion of a polynucleotide at a specific location in the plant genome.

A transgenic plant for the purposes of the invention is thus understood as meaning, as above, that the nucleic acids used in the method of the invention are not at their natural locus in the genome of said plant, it being possible for the nucleic acids to be expressed homologously. or heterologously. However, as mentioned, transgenic also means that, while the nucleic acids according to the different embodiments of the invention are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. According to the invention, the transgene is stably integrated into the plant and the plant is preferably homozygous for the transgene. Thus, any off spring or harvestable material derived from said plant is also preferably homozygous for the transgene.

The aspects of the invention involve recombination DNA technology and in a preferred embodiment exclude embodiments that are solely based on generating plants by traditional breeding methods.

The inventors have characterized Arabidopsis DDA1 (AtDDA1) and have demonstrated that overexpression of DDA1 in transgenic plants reduces the detrimental effects associated with ABA induced stress response when a plant is exposed to stress conditions.

The inventors have identified the proteins with which DDA1 interacts and demonstrated the function of DDA1 on a molecular level which forms the basis for the phenotype observed in the transgenic plants. The inventors have shown that DDA1 associates with the CDD complex and Cullin 4 Ring Ubiquitin Ligase (CUL4) and is able to interact with specific protein targets. DDA1 was found to physically bind ABA receptor PYL8 in vivo and facilitates its proteasomal degradation. In this way. DDA1, together with the other CDD components (CONSTITUTIVE PHOTOMORPHOGENIC10 (COP10) and DEETIOLATED 1 (DET1)), acts as a negative regulator of ABA signaling. ABA treatment attenuates the effect of DDA1 on PYL8 degradation, suggesting that ABA not only activates PYL8 but also prevents its degradation, leading to increased ABA signaling. DDA1 function is also required for proper ovule development, indicating it may recognize additional targets involved in the control of plant reproduction. Thus, DDA1 mediates recognition of specific targets of CRL4 as part of a substrate adaptor module that comprises the CDD complex.

Thus, in a first aspect, the invention relates to a transgenic plant with an altered response to ABA wherein said plant expresses a nucleic acid construct comprising a DDA1 nucleic acid sequence or a functional variant thereof. The DDA1 nucleic acid sequence is preferably an isolated plant DDA1 nucleic acid sequence. As explained elsewhere, this can be genomic DNA, cDNA or coding sequence. In another embodiment, the DDA1 nucleic acid sequence is an animal, for example a mammalian, DDA1 nucleic acid sequence.

The term “functional variant of a nucleic acid sequence” as used herein, for example with reference to SEQ ID No: 1, 2 or 3 or homologs thereof, refers to a variant gene sequence or part of the gene sequence which retains the biological function of the full non-variant DDA1 sequence, for example confers increased growth or yield under stress conditions when expressed in a transgenic plant. A functional variant also comprises a variant of the gene of interest encoding a polypeptide which has sequence alterations that do not affect function of the resulting protein, for example in non-conserved residues. Also encompassed is a variant that is substantially identical, i.e. has only some sequence variations, for example in non-conserved residues, to the wild type sequences as shown herein and is biologically active.

Thus, it is understood, as those skilled in the art will appreciate, that the aspects of the invention, including the methods and uses, encompass not only a DDA1, for example a nucleic acid sequence comprising or consisting or SEQ ID NO: 1, 2 or 3 a polypeptide comprising or consisting or SEQ ID NO: 4, or homologs/orthologs thereof, but also functional variants of DDA1, for example of SEQ ID NO: 1, 2, 3 or 4 that do not affect the biological activity and function of the resulting protein. Alterations in a nucleic acid sequence which result in the production of a different amino acid at a given site that do however not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Each of the proposed modifications is well within the routine skill in the art, as is determination of retention of biological activity of the encoded products.

Generally, variants of a particular DDA1 nucleotide sequence of the invention will have at least about 50%-99%, for example 85%, 86%, 87%, 88%, 89%, 90%, 92%, 94%, 95%, 96%, 97%, 98% or 99% or more sequence identity or similarity to that particular non-variant DDA1 nucleotide sequence, for example to SEQ ID NO: 1, 2, 3 or to the protein sequence SEQ ID NO:4 or homologs thereof, as determined by sequence alignment programs described elsewhere herein and known in the art. Methods of alignment of sequences for comparison are well known in the art. Thus, the determination of percent sequence identity between any two sequences can be accomplished using a mathematical algorithm, including but not limited to CLUSTAL, ALIGN program GAP, BESTFIT, BLAST, FASTA, and TFASTA.

A biologically active variant of a reference DDA1 protein may differ from that protein by as few as 1-15 amino acid residues, as few as 1-10, such as 6-10, as few as 5, as few as 4, 3, 2, or even 1 amino acid residue. In certain embodiments. DDA1 proteins may be altered in various ways including amino acid substitutions, deletions, truncations, and insertions. Methods for such manipulations are generally known in the art. For example, amino acid sequence variants and fragments of the DDA1 protein can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. The deletions, insertions, and substitutions of the protein sequences encompassed herein are not expected to produce radical changes in the characteristics of the protein. When it is difficult, however, to predict the exact effect of a substitution, deletion, or insertion in advance of making such modifications, one skilled in the art will appreciate that the effect will be evaluated by routine screening assays.

For example, sequence identity/similarity values provided herein can refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”.

Also, the various aspects of the invention the aspects of the invention, including the methods and uses, encompass not only a DDA1 nucleic acid, but also a fragment thereof. By “fragment” is intended a portion of the nucleotide sequence or a portion of the amino acid sequence and hence of the protein encoded thereby. Fragments of a nucleotide sequence may encode protein fragments that retain the biological activity of the native protein and hence act to modulate responses to ABA.

In one embodiment, the transgenic plant expresses a nucleic acid comprising, consisting essentially or consisting of AtDDA1 (CDS, cDNA or genomic DNA as defined in SEQ ID NO: 1, 2 or 3) or a functional variant thereof encoding a AtDDA1 polypeptide comprising, consisting essentially or consisting of SEQ ID NO: 4 or a functional variant thereof. However, the invention also extends functional homologs of AtDDA1. A functional homolog of AtDDA1 as shown in SEQ ID NO: 4 is a DDA1 peptide which is biologically active in the same way as SEQ ID NO: 4, in other words, for example it confers increased yield/growth under stress conditions when expressed in a transgenic plant. The term functional homolog includes AtDDA1 orthologs in other organisms, preferably other plant species. In a preferred embodiment of the various aspects of the invention, the invention relates specifically to AtDDA1 or orthologs of AtDDA1 in other plants. AtDDA1 homologs/orthologs include homologs in Arabidopsis.

Homologs/orthologs of AtDDA1 are preferably selected from monocot or dicot plants, for example crop plants as further explained herein.

According to the various aspects of the invention, non-limiting preferred embodiments of homologs/orthologs of AtDDA1 as shown in SEQ ID NO: 1, 2, 3 and 4 include those shown in FIG. 8 and corresponding sequences for nucleic acids (CDS, cDNA or genomic DNA) and peptides according to SEQ ID NOs: 5-191 and also include a functional variants of these sequences. This list is non-limiting and other homologous DDA1 sequences of plants that are described herein, for example other DDA1 from preferred plants, such as crop plants, are also within the scope of the invention. DDA1 orthologs from cereals are one preferred embodiment. AtDDA1 orthologs in maize, rice, wheat, oilseed rape, sorghum, soybean, potato, tomato, grape, barley, pea, bean, field bean, lettuce, cotton, sugar cane, sugar beet, canola, broccoli or other vegetable brassicas or poplar are preferred embodiments within the scope of the aspects of the invention.

Thus, in one embodiment, the invention relates to a transgenic plant with an altered response to ABA wherein said plant expresses a nucleic acid construct expressing a peptide comprising, consisting essentially or consisting of a sequence selected from a sequence shown herein, specifically from SEQ ID Nos: 4, 8, 11, 14, 18, 22, 26, 30, 34, 38, 42, 45, 49, 52, 56, 60, 64, 68, 71, 75, 79, 83, 87, 90, 94, 98, 102, 106, 109, 112, 115, 119, 123, 126, 130, 133, 136, 139, 143, 147, 151, 155, 159, 163, 166, 169, 173, 177, 181, 184, 187, 191, 192 or a functional variant thereof. As described elsewhere, according to the invention, variants of a particular DDA1 nucleotide sequence of the invention, including of a homologs/orthologs of AtDDA1 as shown in SEQ ID NO: 1, 2, 3 and 4 have at least about 50%-99%, for example 85%, 86%, 87%, 88%, 89%, 90%, 92%, 94%, 95%, 96%, 97%, 98% or 99% or more sequence identity or similarity to that particular non-variant DDA1 nucleotide sequence. Corresponding nucleic acid sequences encoding these peptides and which can be used in expression constructs according to the aspects of the invention are shown herein.

In one embodiment of the transgenic plants, host cells and vectors of the invention, the homologs from glycine max, rice, sorghum and maize are disclaimed. In one embodiment, the sequences are not one of glycine max, rice, sorghum and maize as shown herein, for example any of SEQ ID Nos: 27-34, 140-147, 152-159 and 170-173.

The homolog of a AtDDA1 polypeptide has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 4. In one embodiment, the overall sequence identity is at least 66%. Preferably, overall sequence identity or similarity to AtDDA1 as shown in SEQ ID NO: 1, 2, 3 and 4 is 80%, 81%, 82%, 83%, 84%, 85%, 86%, 87%, 88%, 89%, 90%, 91%, 92%, 93%, 94%, 95%, 96%, 97%, 98%, or 99%, most preferably 90%, 91%, 92%, 93%, 94%, 95%, 96%, 97%, 98%, or 99%. In another embodiment, the homolog of a AtDDA1 nucleic acid sequence has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity or similarity to the nucleic acid represented by SEQ ID NO: 1, 2 or 3 or a variant thereof. In one embodiment, overall sequence identity is to the nucleic acid represented by SEQ ID NO: 1. In one embodiment, overall sequence identity is to the nucleic acid represented by SEQ ID NO: 2. In one embodiment, overall sequence identity is to the nucleic acid represented by SEQ ID NO: 3. Preferably, overall sequence identity is 80%, 81%, 82%, 83%, 84%, 85%, 86%, 87%, 88%, 89%, 90%, 91%, 92%, 93%, 94%, 95%, 96%, 97%, 98%, or 99%, most preferably 90%, 91%, 92%, 93%, 94%, 95%, 96%, 97%, 98%, or 99%. The overall sequence identity is determined using a global alignment algorithm known in the art, such as the Needleman Wunsch algorithm in the program GAP (GCG Wisconsin Package, Accelrys).

In one embodiment, the homolog of a AtDDA1 polypeptide has, in increasing order of preference, at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, preferably 85%-99% overall sequence identity to the amino acid represented by SEQ ID NO: 192 (DDA1 polypeptide consensus sequence). In one embodiment, a DDA1 homolog comprises one or more of the following domains: PHNFSQLRPSDPS (SEQ ID NO:193) or a domain with 95%, 96%, 97%, 98%, or 99% to this domain, RTLPPPDQVITTEAK (SEQ ID NO:194) or a domain with 95%, 96%, 97%, 98%, or 99% to this domain, NILLR (SEQ ID NO:195) or a domain with 99% to this domain and/or KLRPKRAA (SEQ ID NO:196) or a domain with 98% or 99% to this domain. In a preferred embodiment, the homolog comprises all of these domains or sequences with homologies to these domains as recited above.

Thus, in one embodiment of the various aspects of the invention, the DDA1 polypeptide comprises an amino acid having at least 50% sequence identity to DDA1 and which comprises an amino acid having at least 95% sequence identity to the amino acid represented by SEQ ID NO: 193, an amino acid having at least 95% sequence identity to the amino acid represented by SEQ ID NO: 194, an amino acid having at least 95% sequence identity to the amino acid represented by SEQ ID NO: 195 and/or an amino acid having at least 99% sequence identity to the amino acid represented by SEQ ID NO: 196. in one embodiment of the various aspects of the invention, the DDA1 polypeptide comprises an amino acid having at least 95% sequence identity to the amino acid represented by SEQ ID NO: 193, an amino acid having at least 95% sequence identity to the amino acid represented by SEQ ID NO: 194, an amino acid having at least 95% sequence identity to the amino acid represented by SEQ ID NO: 195 and/or an amino acid having at least 99% sequence identity to the amino acid represented by SEQ ID NO: 196.

Suitable homologs or orthologs can be identified by sequence comparisons and identifications of conserved domains. The function of the homologue or ortholog can be identified as described herein and a skilled person would thus be able to confirm the function when expressed in a plant.

Thus, the nucleotide sequences of the invention and described herein can be used to isolate corresponding sequences from other organisms, particularly other plants, more particularly cereals. In this manner, methods such as PCR, hybridization, and the like can be used to identify such sequences based on their sequence homology to the sequences described herein. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labeled with a detectable group, or any other detectable marker. Thus, for example, probes for hybridization can be made by labeling synthetic oligonucleotides based on the ABA-associated sequences of the invention. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing).

Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

Thus, the methods, vector and plants of the invention encompass isolated DDA1 homologs/orthologs that modulate the plant response to ABA and which hybridize under stringent conditions to the AtDDA1 or AtDDA1 homologs/orthologs described herein, or to fragments thereof.

For example, according to the various aspects of the invention, a nucleic acid construct comprising a nucleic acid encoding a DDA1 polypeptide may be expressed in said plant by recombinant methods. In another embodiment, an exogenous DDA1 nucleic acid from a first plant in a plant may be expressed in a second plant of another species as defined herein by recombinant methods, for example AtDDA1 may be expressed in a monocot plant, such as wheat. In another embodiment, a nucleic acid construct comprising an endogenous nucleic acid encoding a DDA1 polypeptide may be expressed a plant of the same species. For example, AtDD1 is expressed in Arabidopsis, wheat DDA1 is expressed in wheat, maize DDA1 in maize and barley DDA1 in barley.

In one embodiment according to the various aspects of the invention, the nucleic acid construct comprises a regulatory sequence or element. According to the various aspects of the invention, the term “regulatory element” is used interchangeably herein with “control sequence” and “promoter” and all terms are to be taken in a broad context to refer to regulatory nucleic acid sequences capable of effecting expression of the sequences to which they are ligated the term “regulatory element” also includes terminator sequences which may be included 3′ of the DDA1 nucleic acid sequence. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a -35 box sequence and/or -10 box transcriptional regulatory sequences.

The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern. For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes are known to the skilled person and include for example beta-glucuronidase or beta-galactosidase.

The term “operably linked” as used herein refers to a functional linkage between the promoter sequence and the gene of interest, such that the promoter sequence is able to initiate transcription of the gene of interest.

For example, the nucleic acid sequence may be expressed using a promoter that drives overexpression. Overexpression according to the invention means that the transgene is expressed at a level that is higher than expression of endogenous counterparts driven by their endogenous promoters. For example, overexpression may be carried out using a strong promoter, such as a constitutive promoter. A “constitutive promoter” refers to a promoter that is transcriptionally active during most, but not necessarily all, phases of growth and development and under most environmental conditions, in at least one cell, tissue or organ. Examples of constitutive promoters include the cauliflower mosaic virus promoter (CaMV35S or 19S), rice actin promoter, maize ubiquitin promoter, rubisco small subunit, maize or alfalfa H3 histone, OCS. SAD1 or 2. GOS2 or any promoter that gives enhanced expression. Alternatively, enhanced or increased expression can be achieved by using transcription or translation enhancers or activators and may incorporate enhancers into the gene to further increase expression. Furthermore, an inducible expression system may be used, where expression is driven by a promoter induced by environmental stress conditions (for example the pepper pathogen-induced membrane protein gene CaPIMPI or promoters that comprise the dehydration-responsive element (DRE), the promoter of the sunflower HD-Zip protein gene Hahb4, which is inducible by water stress, high salt concentrations and ABA or a chemically inducible promoter (such as steroid- or ethanol-inducible promoter system). The promoter may also be tissue-specific. The types of promoters listed above are described in the art. Other suitable promoters and inducible systems are also known to the skilled person.

In another embodiment, a root-specific promoter may be used. This is a promoter that is transcriptionally active predominantly in plant roots, substantially to the exclusion of any other parts of a plant, whilst still allowing for any leaky expression in these other plant parts. Examples of root-specific promoters include promoters of root expressible genes, for example the promoters of the following genes: RCc3, Arabidopsis PHT1, Medicago phosphate transporter, Arabidopsis Pyk10, tobacco auxin-inducible gene, beta-tubulin, LR)<1, ALF5, EXP7, LBD16, ARF1, tobacco RD2, SIREO, Pyk10, PsPR10.

In a one embodiment, the promoter is a constitutive or strong promoter. In a preferred embodiment, the regulatory sequence is an inducible promoter, a stress inducible promoter or a tissue specific promoter. The stress inducible promoter is selected from the following non limiting list: the HaHB1 promoter, RD29A (which drives drought inducible expression of DREB1A), the maize rabl7 drought-inducible promoter, P5CS1 (which drives drought inducible expression of the proline biosynthetic enzyme P5CS1), ABA- and drought-inducible promoters of Arabidopsis clade A PP2Cs (ABI1, ABI2, HAB1, PP2CA, HAI1, HAI2 and HAI3) or their corresponding crop orthologs.

In one embodiment, the promoter is CaMV35S.

Additional nucleic acid sequences which facilitate cloning of the target nucleic acid sequences into an expression vector may also be included in the nucleic acid construct according to the various aspects of the invention. This encompasses the alteration of certain codons to introduce specific restriction sites that facilitate cloning.

In another aspect, the invention relates to a non-transgenic plant with increased expression of DDA1 compared to a wild type plant wherein said endogenous DDA1 promoter nucleic acid or DDA1 nucleic acid carries a mutation introduced by mutagenesis which results in increased expression of the DDA1 gene or increased stability fn the DDA1 protein. The invention also relates to a method for increasing expression of DDA1. producing plants overexpressing DDA1. methods for mitigating the impacts of stress conditions on plant growth and yield and methods for producing plants with plant with improved yield/growth under stress conditions comprising the steps of mutagenising a plant population, identifying and selecting plants with an improved yield/growth under stress conditions and identifying a variant DDA1 promoter or gene sequence. In one embodiment such methods include exposing a plant population to a mutagen.

Mutagenesis procedures are well known in the art and include without limitation chemical mutagenesis and irradiation. In one embodiment, said chemical mutagen is selected from ethyl methanesulfonate (EMS), methylmethane sulfonate (MMS), N-ethyl-N-nitrosurea (ENU), triethylmelamine, N-methyl-N-nitrosourea (MNU), procarbazine, chlorambucil, cyclophosphamide, diethyl sulphate (DES), dimethyl sulfate, acrylamide monomer, melphalan, nitrogen mustard, vincristine, dimethylnitosamine, N-methyl-N′-nitro-nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde. In another embodiment, mutagenesis is physical mutagenesis, such as application of ultraviolet radiation, X-rays, gamma rays, fast or thermal neutrons or protons.

Isolated mutants of the wild type DDA1 gene nucleic acid sequence and DDA1 promoter nucleic acid sequence identified in this way are also included within the scope of the invention. Plants obtained by the method above are also included within the scope of the invention.

In a further aspect, the invention relates to a method for producing a mutant plant expressing a DDA1 variant and which is characterised by one of the phenotypes described herein wherein said method uses mutagenesis and Targeting Induced Local Lesions in Genomes (TILLING) to target the gene expressing a DDA1 polypeptide. According to this method, lines that carry a specific mutation are produced that has a known phenotypic effect. For example, mutagenesis is carried out using TILLING where traditional chemical mutagenesis is flowed by high-throughput screening for point mutations. This approach does thus not involve creating transgenic plants. The plants are screened for one of the phenotypes described herein, for example a plant that shows improved yield/growth under stress conditions. A DDA1 locus is then analysed to identify a specific a DDA1 mutation responsible for the phenotype observed. Plants can be bred to obtain stable lines with the desired phenotype and carrying a mutation in a DDA1 locus.

Another technique that can be used for targeted DNA editing is Clustered Regularly Interspaced Short Palindromic Repeats (CRISPR) (U.S. Pat. No. 8,697,359, Ran et al incorporated by reference). The CRISPR system can be used to introduce specific nucleotide modifications at the target sequence. Originally discovered in bacteria, where several different CRISPR cascades function as innate immune systems and natural defence mechanisms, the engineered CRISPR-Cas9 system can be programmed to target specific stretches of genetic code and to make cuts at precise locations. Over the past few years, those capabilities have been harnessed and used as genome editing tools, enabling researchers to permanently modify genes in mammalian and plant cells.

Thus, the invention relates to a method for generating a DDA1 mutant nucleic acid encoding a mutant DDA1 polypeptide wherein said method comprises modifying a plant endogenous genome using CRISPR. The invention relates to a method for generating a DDA1 promoter mutant nucleic acid wherein said method comprises modifying a plant endogenous genome using CRISPR. The method involves targeting of Cas9 to the specific genomic locus, in this case DDA1, via a 20nt guide sequence of the single-guide RNA. An online CRISPR Design Tool can identify suitable target sites (http://tools.genome-engineering.org. Ran et al. Genome engineering using the CRISPR-Cas9 system nature protocols, VOL.8 NO.11, 2281-2308, 2013). Target plants for the mutagenesis/genome editing methods according to the invention are any monocot or dicot plants. Preferred plants are recited elsewhere herein.

Plants obtained through such methods are also within the scope of the invention.

In another aspect, the invention relates to a vector comprising a DDA1 nucleic acid sequence or nucleic acid construct comprising a DDA1 nucleic acid sequence. The DDA1 nucleic acid is preferably a plant DDA1 nucleic acid. The DDA1 nucleic acid may comprise SEQ D NO: 1, 2 or 3 a functional variant or homolog of SEQ D NO: 1, 2 or 3. Homologs/orthologs of AtDDA1 are defined elsewhere herein. Preferably, the vector further comprises a regulatory sequence which directs expression of the nucleic acid. Expression vectors are well known in the art.

The invention also relates to an isolated host cell transformed with a nucleic acid or vector as described above. The host cell may be a bacterial cell, such as Agrobacterium tumefaciens, or an isolated plant cell. The invention also relates to a culture medium or kit comprising a culture medium and an isolated host cell as described above.

The nucleic acid or vector described above is used to generate transgenic plants using transformation methods known in the art. Thus, according to the various aspects of the invention, a nucleic acid comprising a DDA1 nucleic acid, for example SEQ D No. 1, a functional variant or homolog thereof is introduced into a plant and expressed as a transgene. The nucleic acid sequence is introduced into said plant through a process called transformation. The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker such as the ones described above. Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

Thus, the invention relates to a method for producing a transgenic plant with improved yield/growth under stress conditions said method comprising
a) introducing into said plant and expressing a nucleic acid construct comprising a DDA1 nucleic acid sequence, for example a nucleic acid sequence comprising SEQ ID NO: 1, 2, or 3 a functional variant or homolog of SEQ ID NO: 1, 2, or 3
b) obtaining a progeny plant derived from the plant or plant cell of step a).

The method may comprise the further steps of:
exposing the plant to stress conditions, such as drought;
assessing yield/growth;
selecting a plant or part thereof with increased stress resistance/ improved yield/growth;
optionally harvesting parts of the plant.

The invention also relates to plants obtained or obtainable with said method.

In another aspect, the invention relates to a method for reducing a plant response to ABA, said method comprising introducing into said plant and expressing a DDA1 nucleic acid or nucleic acid construct comprising a DDA1 nucleic acid, for example a nucleic acid comprising SEQ ID NO: 1, 2 or 3 or a functional variant or homolog of SEQ ID NO: 1, 2, or 3. The DDA1 nucleic acid is preferably a plant DDA1 nucleic acid sequence.

In another aspect, the invention relates to a method for modulating the interaction of the receptor PYL8 with ABA in a plant or in vitro said method comprising introducing into said plant or plant cell and expressing a DDA1 nucleic acid, for example a nucleic acid comprising SEQ ID NO: 1, 2, or 3 or a functional variant or homolog of SEQ ID NO: 1, 2, or 3. The DDA1 nucleic acid is preferably a plant DDA1 nucleic acid sequence. The interaction can be modulated by decreasing the presence of PYL8 as it will be degraded by DDA1.

The method may comprise the further steps of:
assessing the interaction of the receptor PYL8 with ABA;
selecting a plant or part thereof with modulated interaction;
optionally harvesting parts of the plant.

In another aspect, the invention relates to a method for increasing yield and/or growth of a plant under stress conditions said method comprising introducing into said plant and expressing a DDA1 nucleic acid, for example a nucleic acid comprising SEQ ID NO: 1, 2 or 3 or a functional variant or homolog of SEQ ID NO: 1, 2, or 3. The DDA1 nucleic acid is preferably a plant DDA1 nucleic acid sequence.

The method may comprise the further steps of:
exposing the plant to stress conditions, such as drought;
assessing yield/growth;
selecting a plant or part thereof with increased yield/growth;
optionally harvesting parts of the plant.

In another aspect, the invention relates to a method for mitigating the impacts of stress conditions on plant growth, development and/or yield said method comprising introducing into said plant and expressing a DDA1 nucleic acid, for example a nucleic acid comprising SEQ ID NO: 1, 2 or 3 or a functional variant or homolog of SEQ ID NO: 1, 2, or 3. The DDA1 nucleic acid is preferably a plant DDA1 nucleic acid sequence. The method may comprise the further steps of:
exposing the plant to stress conditions, such as drought;
selecting a plant or part thereof with increased stress resistance;
optionally harvesting parts of the plant.

Preferred homologs of SEQ ID NO: 1, 2, or 3 are listed elsewhere. In one embodiment, the homologous nucleic acid encodes a peptide selected from SEQ ID NO: 4, 8, 11, 14, 18, 22, 26, 30, 34, 38, 42, 45, 49, 52, 56, 60, 64, 68, 71, 75, 79, 83, 87, 90, 94, 98, 102, 106, 109, 112, 115, 119, 123, 126, 130, 133, 136, 139, 143, 147, 151, 155, 159, 163, 166, 169, 173, 177, 181, 184, 187 or 191 or a functional variant thereof.

According to the various aspects of the invention, the stress may be severe or preferably moderate stress. According to the various aspects of the invention, the stress is selected from biotic and abiotic stress. In one embodiment, the stress is drought or water deficiency. In another embodiment, the stress is salinity. In Arabidopsis research, stress is often assessed under severe conditions that are lethal to wild type plants. For example, drought tolerance is assessed predominantly under quite severe conditions in which plant survival is scored after a prolonged period of soil drying. However, in temperate climates, limited water availability rarely causes plant death, but restricts biomass and seed yield. Moderate water stress, that is suboptimal availability of water for growth can occur during intermittent intervals of days or weeks between irrigation events and may limit leaf growth, light interception, photosynthesis and hence yield potential. Leaf growth inhibition by water stress is particularly undesirable during early establishment. There is a need for methods for making plants with increased yield under moderate stress conditions. In other words, whilst plant research in making stress tolerant plants is often directed at identifying plants that show increased stress tolerance under severe conditions that will lead to death of a wild type plant, these plants do not perform well under moderate stress conditions and often show growth reduction which leads to unnecessary yield loss. Thus, in one embodiment of the methods of the invention, yield is improved under moderate stress conditions. The transgenic plants according to the various aspects of the invention show enhanced tolerance to these types of stresses compared to a control plant and are able to mitigate any loss in yield/growth. The tolerance can therefore be measured as an increase in yield as shown in the examples. The terms moderate or mild stress/stress conditions are used interchangeably and refer to non-severe stress. In other words, moderate stress, unlike severe stress, does not lead to plant death. Under moderate, that is non-lethal, stress conditions, wild type plants are able to survive, but show a decrease in growth and seed production and prolonged moderate stress can also result in developmental arrest. The decrease can be at least 5%-50% or more. Tolerance to severe stress is measured as a percentage of survival, whereas moderate stress does not affect survival, but growth rates. The precise conditions that define moderate stress vary from plant to plant and also between climate zones, but ultimately, these moderate conditions do not cause the plant to die. With regard to high salinity for example, most plants can tolerate and survive about 4 to 8 dS/m. Specifically, in rice, soil salinity beyond ECe ˜4 dS/m is considered moderate salinity while more than 8 dS/m becomes high. Similarly, pH 8.8-9.2 is considered as non-stress while 9.3-9.7 as moderate stress and equal or greater than 9.8 as higher stress.

The DDA1 polypeptides described herein may be used alone or in combination with additional polypeptides or agents to increase stress tolerance in plants. For example, in the practice of certain embodiments, a plant can be genetically manipulated to produce more than one polypeptide associated with increased stress, for example, drought tolerance.

Drought stress can be measured through leaf water potentials. Generally speaking, moderate drought stress is defined by a water potential of between −1 and −2 Mpa. Moderate temperatures vary from plant to plant and specially between species. Normal temperature growth conditions for Arabidopsis are defined at 22-24° C. For example, at 28° C. Arabidopsis plants grow and survive, but show severe penalties because of “high” temperature stress associated with prolonged exposure to this temperature. However, the same temperature of 28° C. is optimal for sunflower, a species for which 22° C. or 38° C. causes mild, but not lethal stress. In other words, for each species and genotype, an optimal temperature range can be defined as well as a temperature range that induces mild stress or severe stress which leads to lethality. Drought tolerance can be measured using methods known in the art, for example assessing survival of the transgenic plant compared to a control plant, or by determining turgor pressure, rosette radius, water loss in leaves, growth or yield. Regulation of stomatal aperture by ABA is a key adaptive response to cope with drought stress. Thus, drought resistance can also be measured by assessing stomatal conductance (Gst) and transpiration in whole plants under basal conditions.

According to the invention, a transgenic plant has enhanced drought tolerance if the survival rates are at least 2, 3, 4, 5, 6, 7, 8, 9 or 10-fold higher than those of the control plant after exposure to drought and/or after exposure to drought and re-watering. Also according to the invention, a transgenic plant has enhanced drought tolerance if the rosette radius is at least 10, 20, 30, 40, 50% larger than that of the control plant after exposure to drought and/or after exposure to drought and re-watering. The plant may be deprived of water for 10-30, for example 20 days and then re-watered. Also according to the invention, a transgenic plant has enhanced drought tolerance if stomatal conductance (Gst) and transpiration are lower than in the control plant, for example at least 10, 20, 30, 40, 50% lower.

Thus in one embodiment, the methods of the invention relate to increasing resistance to moderate (non-lethal) stress or severe stress. In the former embodiment, transgenic plants according to the invention show increased resistance to stress and therefore, the plant yield is not or less affected by the stress compared to wild type yields which are reduced upon exposure to stress. In other words, an improve in yield under moderate stress conditions can be observed.

The terms “increase”, “improve” or “enhance” are interchangeable. Yield for example is increased by at least a 3%, 4%, 5%, 6%, 7%, 8%, 9% or 10%, preferably at least 15% or 20%, more preferably 25%, 30%, 35%, 40% or 50% or more in comparison to a control plant. The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant. Thus, according to the invention, yield comprises one or more of and can be measured by assessing one or more of: increased seed yield per plant, increased seed filling rate, increased number of filled seeds, increased harvest index, increased number of seed capsules/pods, increased seed size, increased growth or increased branching, for example inflorescences with more branches. Preferably, yield comprises an increased number of seed capsules/pods and/or increased branching. Yield is increased relative to control plants.

In one embodiment, the methods relate to improving drought tolerance of plant vegetative tissue. In one embodiment, the methods relate to improving drought tolerance of plant non-vegetative tissue.

A control plant as used herein is a plant, which has not been modified according to the methods of the invention. Accordingly, the control plant has not been genetically modified to express a nucleic acid as described herein. In one embodiment, the control plant is a wild type plant. In another embodiment, the control plant is a plant that does not carry a transgenic according to the methods described herein, but expresses a different transgene. The control plant is typically of the some plant species, preferably the same ecotype as the plant to be assessed.

A control plant or plant cell may thus comprise, for example: (a) a wild-type (WT) plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest or (e) the subject plant or plant cell itself, under conditions in which the gene of interest is not expressed.

During seed development, ABA content increases and regulates many key processes including the imposition and maintenance of dormancy. ABA stimulates dormancy as well as adaptive responses to drought, cold and salt stress. As shown in the examples. DDA1 also controls PYL8 levels in seeds. Overexpressing DDA1-GFP seedlings were less sensitive to NaCl- or mannitol-mediated inhibition of seed germination than the wild type (FIG. 6G-6H), indicating that DDA1 over-expression effect is also evident under stress conditions that increase endogenous ABA levels. Therefore, in another aspect, the invention relates to a method for reducing plant seed dormancy said method comprising introducing into said plant and expressing a DDA1 nucleic acid, for example a nucleic acid comprising SEQ ID No: 1, 2 or 3 or a functional variant or homolog thereof. The DDA1 nucleic acid is preferably a plant DDA1 nucleic acid sequence. In another aspect, the invention relates to a method for modulating germination said method comprising introducing into said plant and expressing a DDA1 nucleic acid, for example a nucleic acid comprising SEQ ID No: 1, 2 or 3 or a functional variant or homolog thereof. Thus, the method can be used to advance or initiate germination. The DDA1 is preferably a plant DDA1 nucleic acid sequence. In one embodiment, seed dormancy is reduced and germination is altered under stress, for example moderate stress conditions.

The terms “reduce” or “decrease” used herein are interchangeable. Seed dormancy for example is increased by at least a 3%, 4%, 5%, 6%, 7%, 8%, 9% or 10%, preferably at least 15% or 20%, more preferably 25%, 30%, 35%, 40% or 50%

The methods described above preferably contain the step of obtaining a progeny plant derived from the plant or plant cell. The various methods of the invention may also include the additional step of evaluating growth and yield of the transgenic plant and comparing said phenotype to a control plant.

In another aspect, the invention relates to the use of a DDA1 nucleic acid sequence, for example a plant nucleic acid, for example a nucleic acid comprising or consisting of SEQ ID NO: 1, 2 or a functional variant or homolog, a vector comprising a DDA1 nucleic acid sequence, for example a nucleic acid comprising or consisting of SEQ ID NO: 1, 2 or 3 or a functional variant or homolog in reducing a plant response to ABA and/or increasing yield/growth under stress conditions. The DDA1 nucleic acid is preferably a plant DDA1 nucleic acid sequence.

The transgenic plant according to the various aspects of the invention, including the transgenic plants, methods and uses described herein may be a monocot or a dicot plant. The plant DDA1 nucleic acid according to the various aspects of the invention may be a monocot or a dicot plant DDA1 nucleic acid.

In one embodiment of the various aspects of the invention, the plant is a dicot plant. A dicot plant may be selected from the families including, but not limited to Asteraceae, Brassicaceae (eg Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower. Arabidopsis, broccoli, spinach, water melon, squash, cabbage, tomato, potato, yam, capsicum, tobacco, cotton, okra, apple, rose, strawberry, alfalfa, bean, soybean, field (fava) bean, pea, lentil, peanut, chickpea, apricots, pears, peach, grape vine or citrus species. In one embodiment, the plant is oilseed rape.

Also included are biofuel and bioenergy crops such as rape/canola, corn, sugar cane, palm trees, jatropha, soybeans, sorghum, sunflowers, cottonseed. Panicum virgatum (switchgrass), linseed, wheat, lupin and willow, poplar, poplar hybrids. Miscanthus or gymnosperms, such as loblolly pine. Also included are crops for silage (maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium. Nicotiana sp.) and plants and cut flowers for the home (African violets, Begonias, chrysanthemums, geraniums, Coleus spider plants, Dracaena, rubber plant).

In one embodiment of the various aspects of the invention, the plant is a dicot plant. A monocot plant may, for example, be selected from the families Arecaceae, Amaryllidaceae or Poaceae. For example, the plant may be a cereal crop, such as wheat, rice, barley, maize, oat, sorghum, rye, millet, buckwheat, turf grass, Italian rye grass, sugarcane or Festuca species, or a crop such as onion, leek, yam or banana.

In preferred embodiments of the various aspects of the invention the plant is a crop plant. By crop plant is meant any plant which is grown on a commercial scale for human or animal consumption or use.

In preferred embodiments of the various aspects of the invention the plant grain plant, an oil-seed plant, and a leguminous plant.

Most preferred plants according to the various aspects of the invention are maize, rice, wheat, oilseed rape, sorghum, soybean, potato, tomato, tobacco, grape, barley, pea, bean, field bean, lettuce, cotton, sugar cane, sugar beet, broccoli or other vegetable brassicas or poplar.

Polypeptide sequences for a non-limiting list of preferred AtDDA1 orthologs comprise or consist of SEQ ID NOs: 8, 11, 14, 18, 22, 26, 30, 34, 38, 42, 45, 49, 52, 56, 60, 64, 68, 71, 75, 79, 83, 87, 90, 94, 98, 102, 106, 109, 112, 115, 119, 123, 126, 130, 133, 136, 139, 143, 147, 151, 155, 159, 163, 166, 169, 173, 177, 181, 184, 187, 191, 192 or a functional variant thereof. Corresponding nucleic acids are set out herein. Alternatively, the AtDDA1 ortholog is a DDA1 isolated from any of the plants defined herein, preferably any crop plant, for example, but not limited to maize, wheat, oilseed rape, canola, sorghum, soybean, potato, tomato, tobacco, grape, barley, pea, bean, field bean, lettuce, cotton, sugar cane, sugar beet, broccoli or other vegetable brassicas or poplar.

In another aspect, the invention relates to a method for increasing expression of a DDA1 nucleic acid in a plant, preferably a plant DDA1 nucleic acid compared to a control plant by incorporating a heterologous nucleic acid which encodes a DDA1-related polypeptide. In one embodiment, expression is increased by a method comprising; crossing a first and a second plant to produce a population of progeny plants; determining the expression of the DDA1-related polypeptide in the progeny plants in the population, and identifying a progeny plant in the population in which expression of the DDA1-related polypeptide is increased relative to controls. In another embodiment, expression is increased by a method comprising; exposing a population of plants to a mutagen, determining the expression of the DDA1-related polypeptide in one or more plants in said population, and identifying a plant with increased expression of the DDA1--related polypeptide The methods can comprise sexually or asexually propagating or growing off-spring or descendants of the plant having increased DDA1-related polypeptide expression.

The term “plant” as used herein encompasses whole plants, ancestors and progeny of the plants and plant parts, including seeds, fruit, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

The various aspects of the invention described herein clearly extend to any plant cell or any plant produced, obtained or obtainable by any of the methods described herein, and to all plant parts and propagules thereof unless otherwise specified. The present invention extends further to encompass the progeny of a primary transformed or transfected cell, tissue, organ or whole plant that has been produced by any of the aforementioned methods, the only requirement being that progeny exhibit the some genotypic and/or phenotypic characteristic(s) as those produced by the parent in the methods according to the invention.

The invention also extends to harvestable parts of a plant of the invention as described above such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins. The invention also relates to food products and food supplements comprising the plant of the invention or parts thereof.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification, including reference to sequence database identifiers, are incorporated herein by reference in their entirety. Unless otherwise specified, when reference to sequence database identifiers is made, the version number is 1.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.The invention is further described in the following non-limiting examples.

### EXAMPLES

DDA1 is Present in Vascular Plants

To investigate whether DDA1 is conserved across plant families, we searched for DDA1-related sequences in plant genomic databases (see Methods). We successfully retrieved DDA1 homologs from 49 different plant species and subspecies (FIG. 8). On average, 66% aa sequence identity was found between DDA1 ortholog pairs. Phylogenetic analyses showed that DDA1 is conserved in vascular plants, including pteridophyte Sellaginella moellendorffii, and could not be found in algae or in the moss Physcomitrella patens (FIG. 9). In the case of Angiosperms, DDA1 was present in both monocots and dicots. In plant diploid species. DDA1 was usually found as a single copy gene, although in some cases (e.g. corn, soybean and cotton) we found two DDA1 gene copies.

DDA1 is a Component of the CDD Complex in Arabidopsis

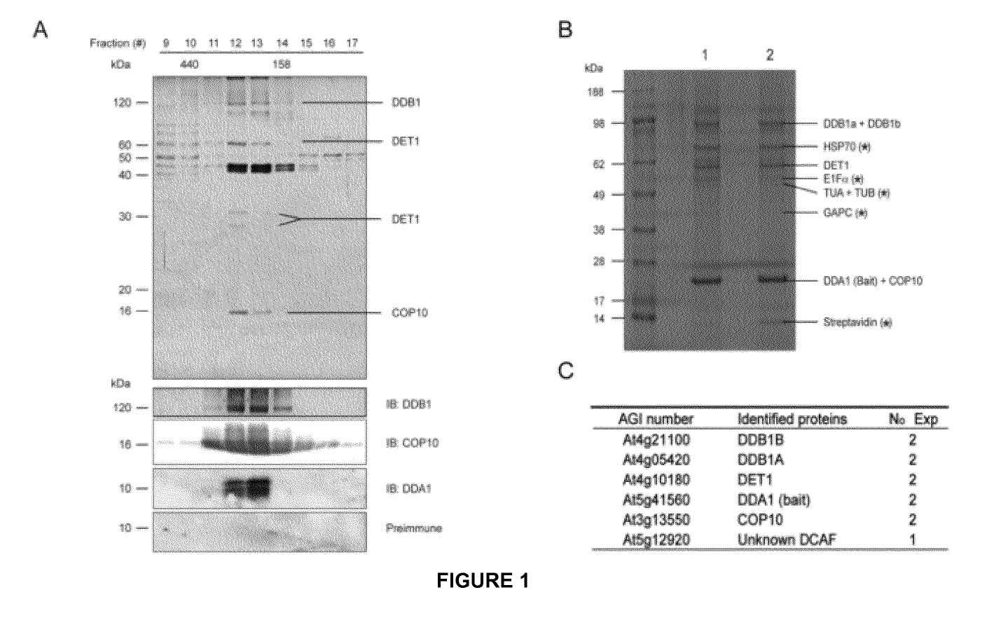

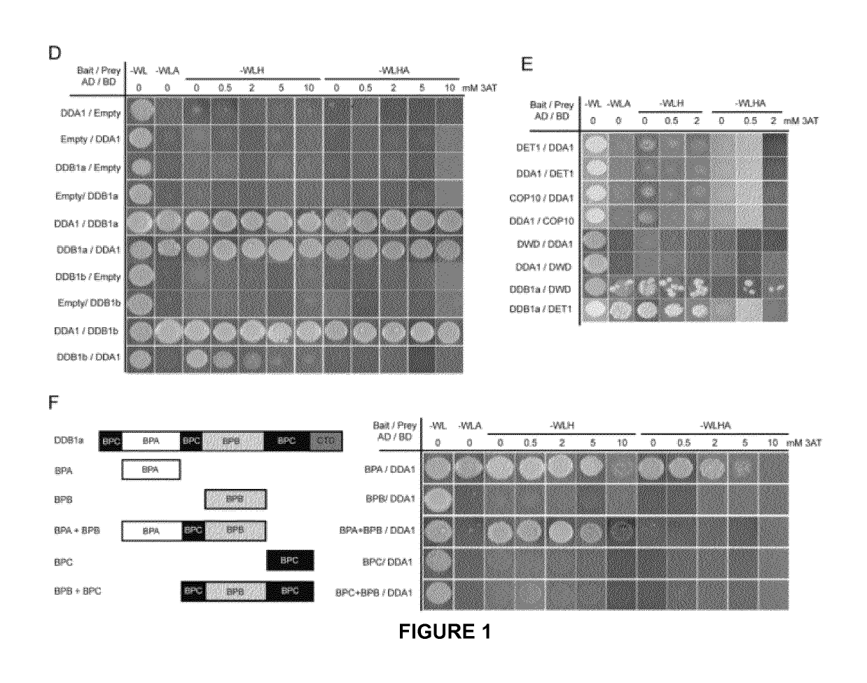

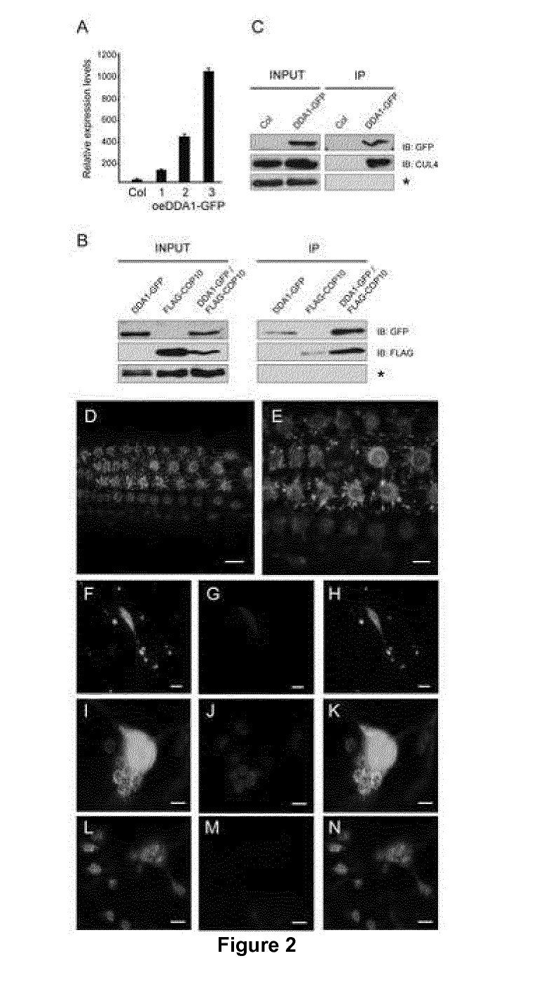

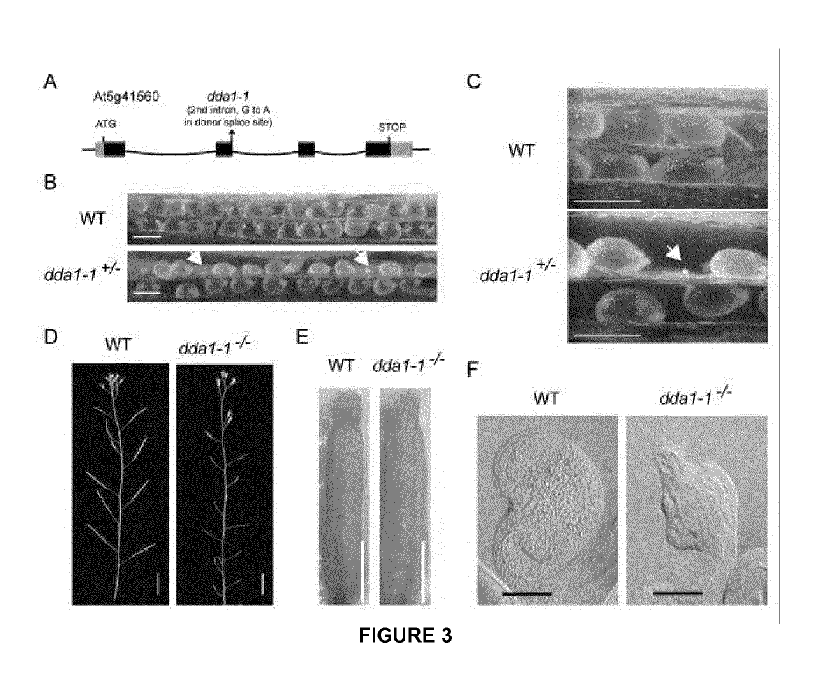

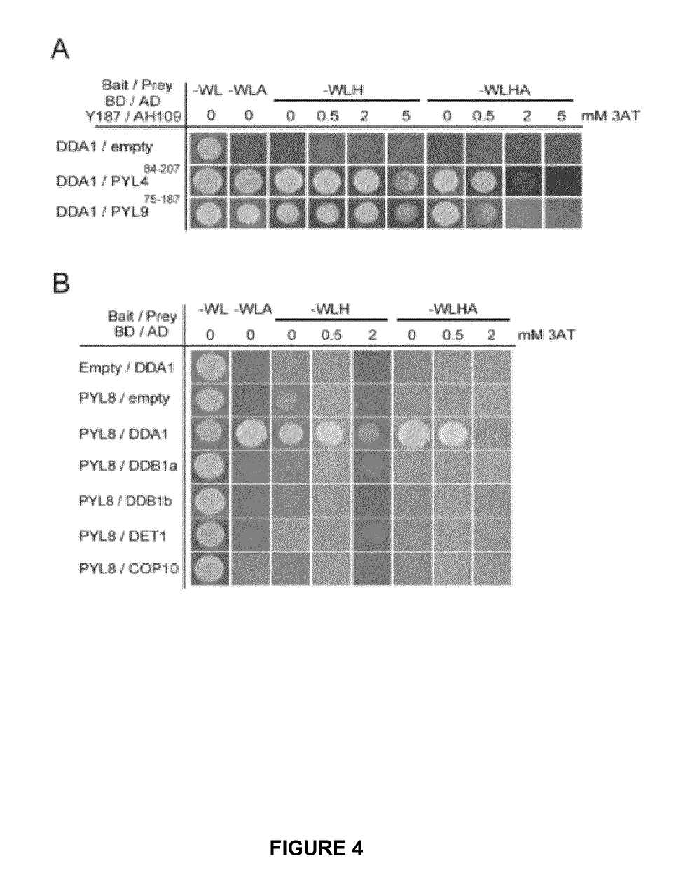

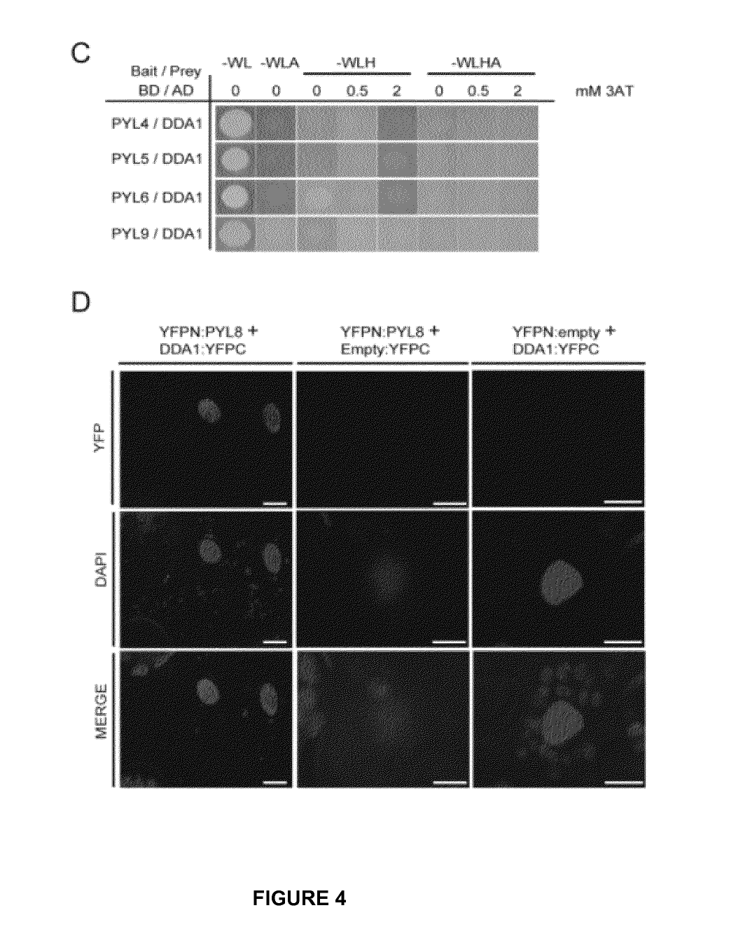

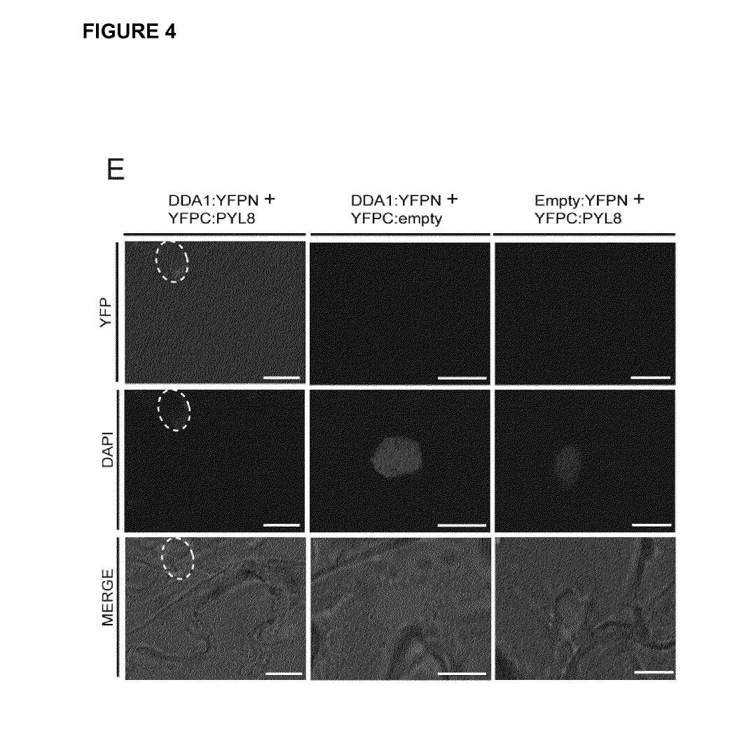

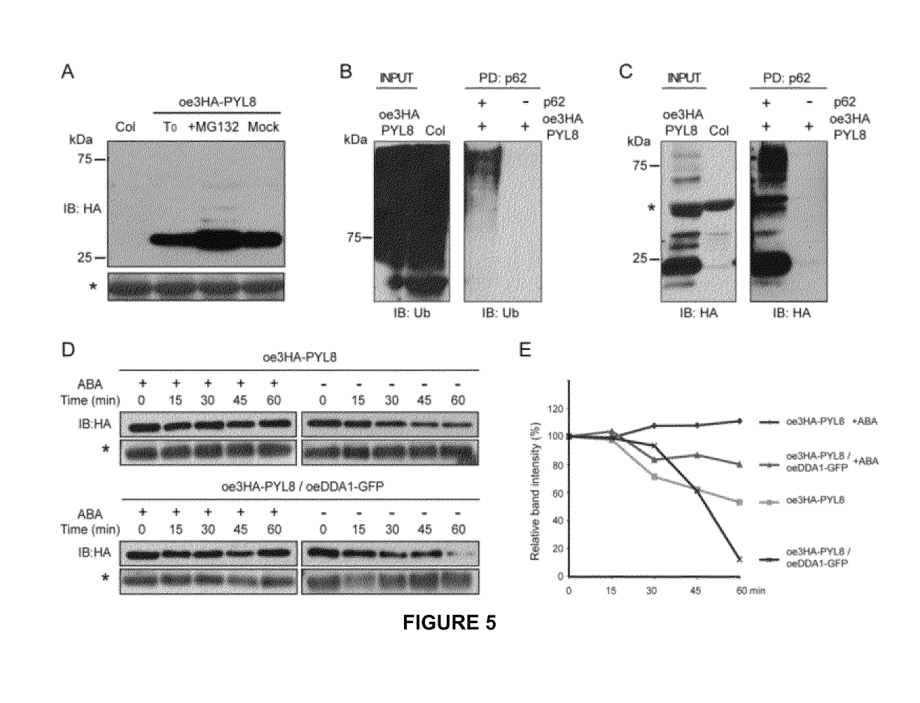

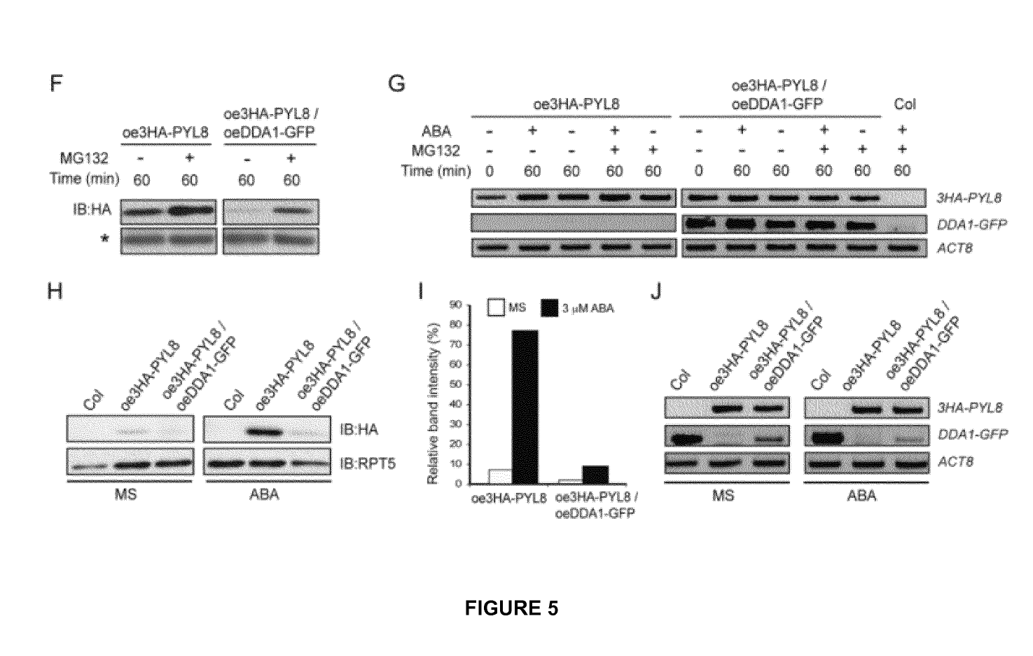

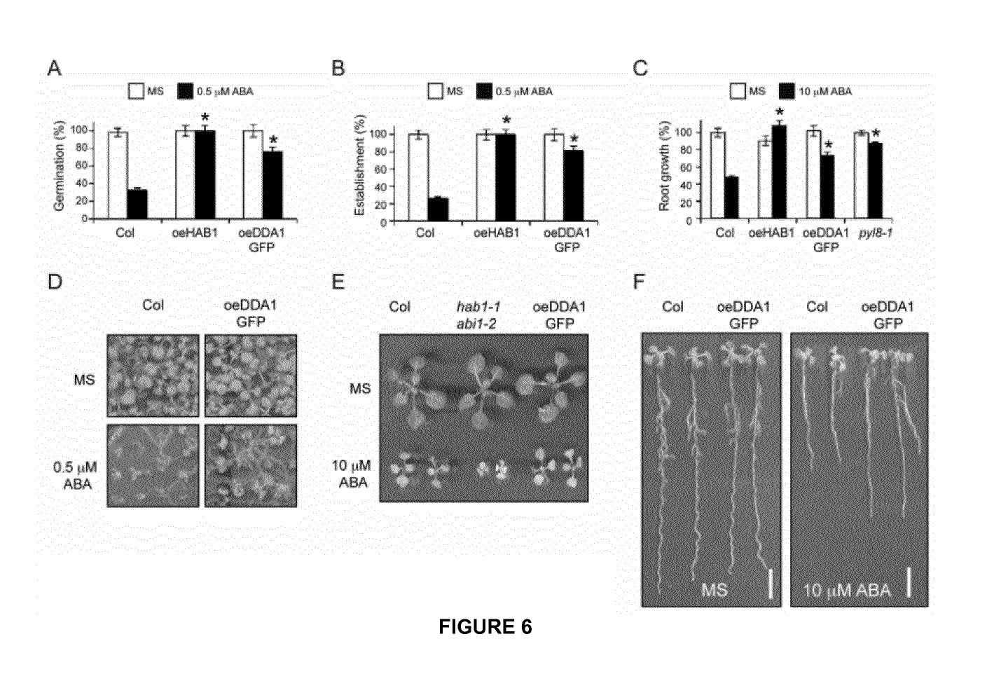

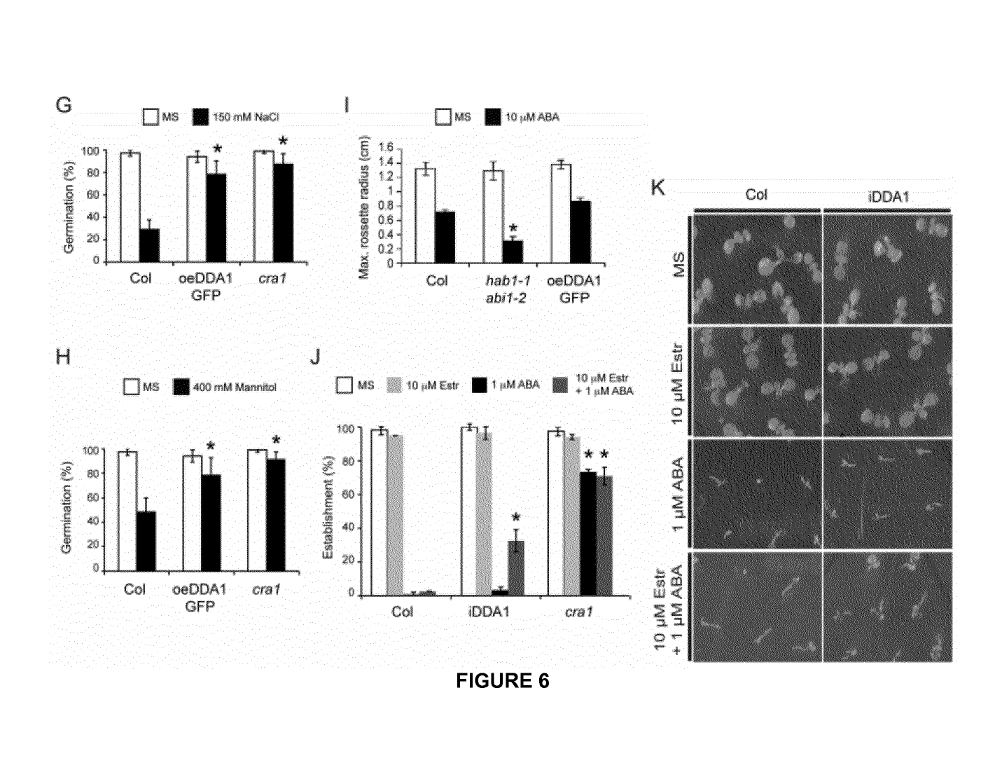

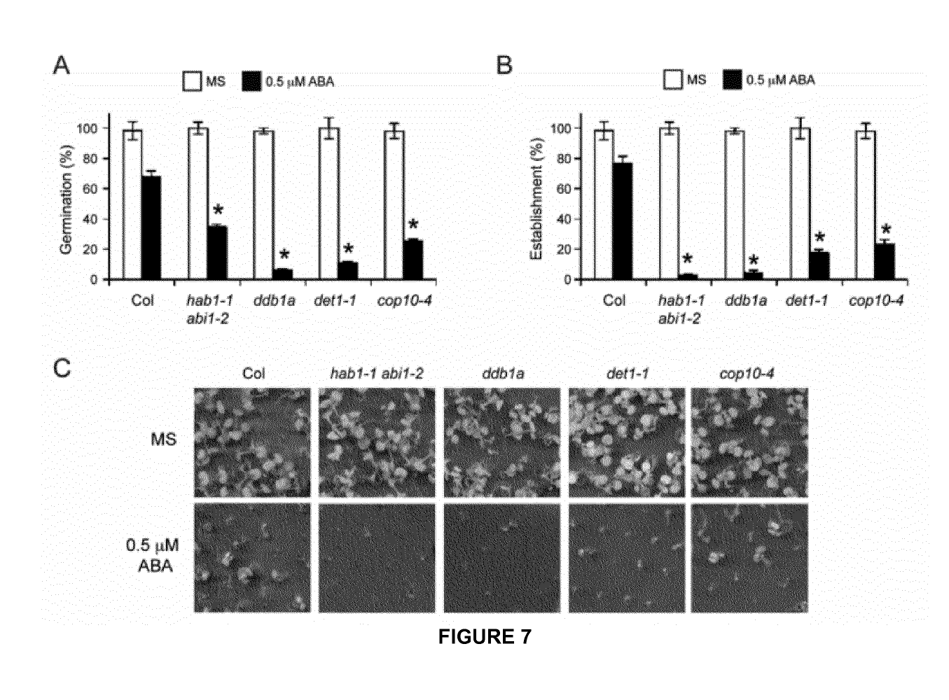

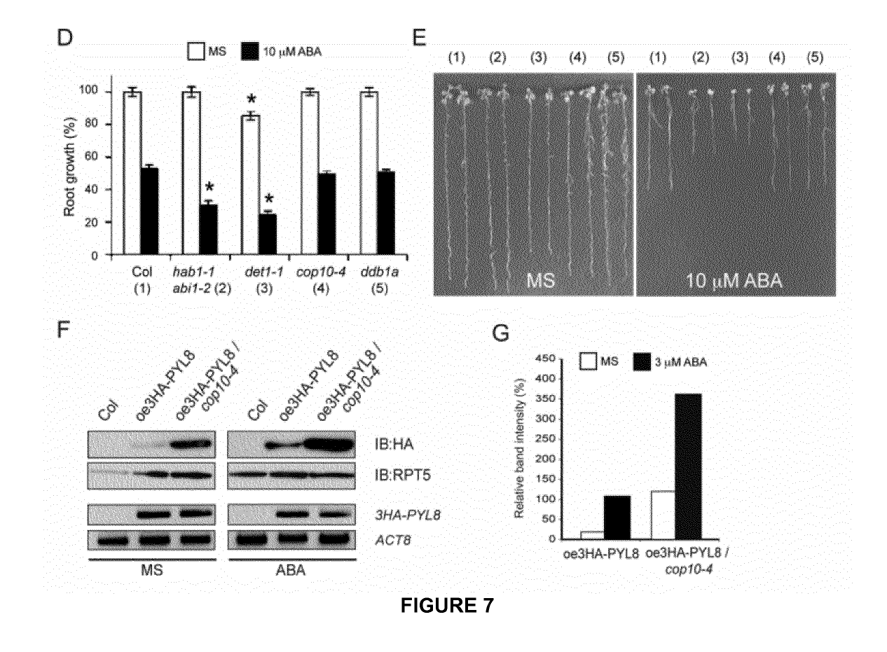

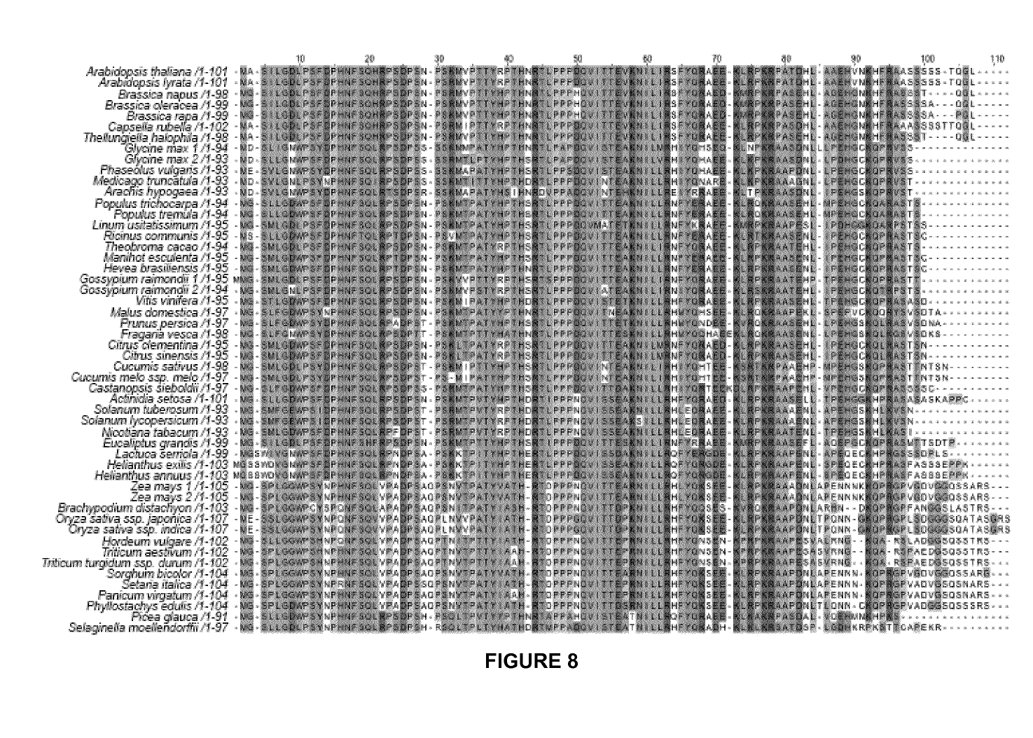

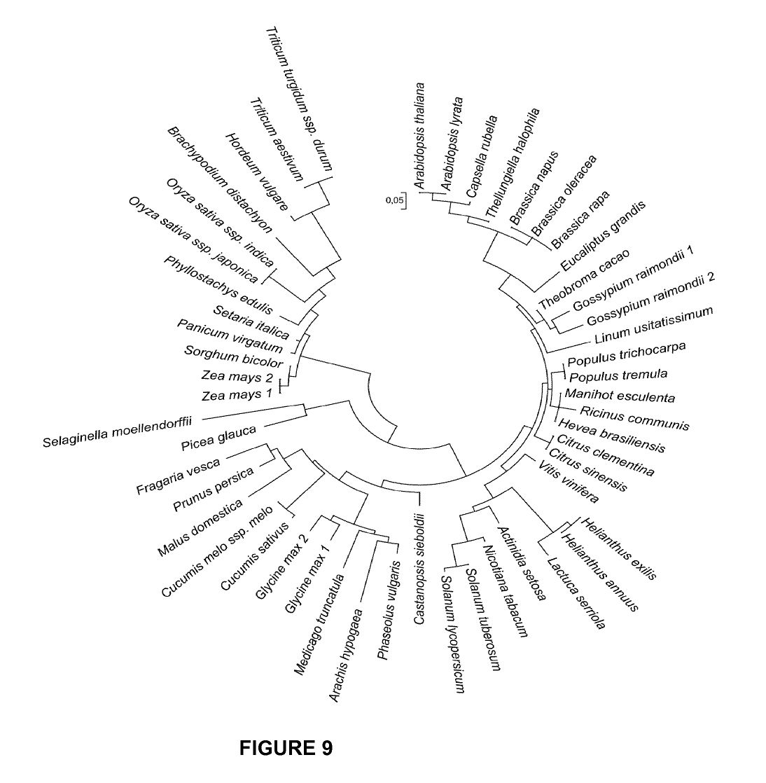

The CDD complex was originally isolated from floral meristems of cauliflower (a Brassica species related to Arabidopsis) using a biochemical purification procedure (Yanagawa et al., 2004). To determine whether DDA1 co-purifies with the CDD complex, we subjected the original gel filtration fractions, corresponding to the last step of CDD purification, to SDS-PAGE followed by silver staining or immunoblots using a specific antibody raised against recombinant His-tagged DDA1 (FIG. 1A). No additional bands than those previously reported were detected by silver staining of the SDS-PAGE gel. However. DDA1 could be immunodetected in fractions corresponding to the CDD as three protein bands of lower MW (10 KDa) than expected (16 KDa), indicating that, although apparently partly degraded, DDA1 is present in purified CDD samples. To further confirm that DDA1 binds to the CDD complex, we isolated DDA1-associated proteins using Tandem Affinity Purification (TAP) techniques. For this, C-terminal TAP-tagged DDA1 was expressed and purified from two Arabidopsis cell cultures. The identity of proteins that co-purified with DDA1-TAP was determined using mass spectrometry analysis. Together with DDA1. TAP-purified samples contained all CDD complex components (FIG. 1B-1C). In this regard. DDA1 was incorporated into CDD complexes that contained either DDB1a or DDB1b, as both proteins co-purified with DDA1-TAP. Next, we characterized DDA1 interaction with CDD complex components using yeast two-hybrid assays. In agreement with previous studies in mammalian systems (Jin et al., 2006; Olma et al., 2009; Pick et al., 2007), we found that DDA1 strongly binds to DDB1 proteins and that this interaction occurs through the β-propeller domain A (BPA) in DDB1 (FIG. 1D-1F). Association of DDA1 into CDD complexes was likely mediated by DDA1-DDB1 physical interaction, since we did not observe direct binding of DDA1 to neither DET1 nor COP10 (FIG. 1E). Upon DDA1-TAP purification, a DCAF protein (encoded by the At5g12920 locus; Lee et al., 2008) was also co-purified (FIG. 1B-1C). This DCAF protein interacted with DDB1a in yeast two hybrid assays but not with DDA1, indicating that DDA1-DCAF association is indirect and likely mediated by DDB1 proteins (FIG. 1E).

DDA1 Localizes in Nuclei and Plastids and Interacts In Vivo with CUL4

DDA1 has been shown to localize in nuclei of mammalian cells (Olma et al., 2009). In order to analyze DDA1 subcellular localization in planta, we first generated Arabidopsis transgenic plants expressing the cDNA of DDA1 fused to GFP under the control of the CaMV 35S promoter (oeDDA1-GFP). Using these lines, we examined DDA1-GFP expression levels relative to endogenous DDA1 of wild-type plants by quantitative real-time RT-PCR (q-RT-PCR; FIG. 2A). All three independent lines tested displayed high level of DDA1-GFP expression; ranging from 100- to 1000-fold the endogenous DDA1 transcript level in wild-type plants. Confocal microscopy analysis showed a similar pattern of DDA1-GFP fluorescence in root cells of all oeDDA1-GFP lines analyzed (FIG. 2D-2E). Thus, similar to previous studies in animals. DDA1-GFP was observed to localize in nuclei. Interestingly. DDA1-GFP accumulated in additional vesicular compartments unevenly distributed through the cytoplasm. To unveil the identity of these compartments we transiently co-expressed DDA1-GFP and different subcellular markers in Nicotiana benthamiana leaves using agroinfiltration techniques. We only found co-localization of DDA1-GFP and a fluorescent marker of plastids (FIG. 2F-2N). In agreement with our microscopy data. DDA1 is predicted to localize in both nuclei and chloroplasts according to protein subcellular localization prediction tools (SUBA3; http://suba.plantenergy.uwa.edu.au/). To test whether DDA1-GFP is able to associate with the CDD complex, we crossed oeDDA1-GFP plants (line 3; FIG. 2A) with a previously described 3xFLAG epitope tagged-COP10 expressing line (FLAG-COP10) and conducted immunoprecipitation assays. As shown in FIG. 2B, FLAG-COP10 coimmunoprecipitated with DDA1-GFP. Since DDA1 and COP10 do not directly interact, according to our yeast two hybrid assays (FIG. 1E), we concluded that DDA1-GFP and FLAG-COP10 were incorporated in the same CDD complexes. Moreover, we detected CUL4 protein in DDA1-GFP immunoprecipitates using specific anti-CUL4 antibodies (FIG. 2C). Taken together, these results indicate that DDA1, likely as part of the CDD, interacts with CUL4-containing (i.e. CRL4) complexes in plant nuclei, where both complexes are located (Chen et al., 2006; Molinier et al., 2008; Schroeder et al., 2002; Suzuki et al., 2002).

Null Mutation of DDA1 Causes Ovule Infertility

In order to functionally characterize DDA1 in plants, we searched for loss-of-function mutants in different Arabidopsis T-DNA insertion collections. All available lines contained the T-DNA integrated into non-coding regions in the DDA1 gene and displayed transcript levels similar to those of wild-type plants (data not shown), which precluded their use in further studies. Similar results were found when DDA1 gene silencing approaches were followed as all Arabidopsis transgenic lines obtained using two different RNA interference systems (Nilson et al., 2004; Karimi et al., 2002), displayed normal DDA1 mRNA levels (data not shown). We then screened a permanent collection of chemically induced mutants (TILLer; http://www.cnb.csic.es/˜tiller/; Martin et al., 2009) from which we isolated a heterozygous line that contained a point mutation (G to A) at the donor splice-site of the second intron (FIG. 3A). This mutation, hereafter termed as dda1-1, is predicted to impair proper splicing of DDA1 premRNA and to yield a truncated translation product lacking the C-terminal half of the protein. In order to remove extraneous mutations, dda1-1 plants were backcrossed with the wild-type seven times. Segregation analyses using the progeny of backcrossed plants showed that the frequency of homozygous dda1-1 plants recovered was much lower (3.84%) than expected (25%), suggesting that loss of DDA1 function causes partial lethality of embryos or reduced gamete transmission efficiency. The latter seems to be the case since the siliques of heterozygous dda1-1 mutants contained a larger number of unfertilized ovules (14.9%), compared to wild-type ones (4.3%), rather than an increased number of aborted seeds (FIG. 3B and 3C;). We aimed to test whether the transmission efficiency of the dda1-1 mutation through any or both gametophytes (pollen and ovules) was altered. For this, reciprocal crosses between heterozygous dda1-1 mutants and wild-type plants were carried out. Analysis of the F1 progeny showed that the transmission efficiency of the dda1-1 allele through the pollen is reduced significantly, but not through the ovule. However, we observed ovule development defects when homozygous dda1-1 plants were analyzed. Thus, although the homozygous mutants showed normal vegetative development and flowering, they were fully sterile (FIG. 3D). Any attempt to fertilize homozygous dda1-1 flowers using wild-type pollen failed, whereas fertilization could be attained when using mutant pollen and wild-type pistils (data not shown), indicating that dda1-1 mutation reduces ovule fertility. Accordingly, dissection of non-pollinated pistils fromhomozygous dda1-1 flowers showed they only contain arrested ovules (FIG. 3E-3F). Taken together, these results indicate that DDA1 plays a role in the control of both gametophytes function, being essential for ovule development.

DDA1 Physically Interacts with PYR/PYL ABA Receptors

The molecular basis of DDA1 activity is unknown. To get insights into its mechanism of action, we searched for proteins that interact with DDA1. With this aim, we conducted a yeast two-hybrid screen using DDA1 as bait. Thus, the full-length coding sequence of DDA1 fused to the binding domain of GAL4 was used to screen a cDNA library prepared from Arabidopsis seedlings. From over 15 million clones screened, 200 were identified as potential DDA1 interactors. Among them, 20 were subsequently confirmed by retransformation into yeast. Interestingly, among the DDA1 interactors we found two clones corresponding to members of the PYR/PYL/RCAR family of ABA receptors; PYL4 and PYL9 (FIG. 4A). The clones isolated in our screen did not correspond to full-length versions of these proteins but rather to truncated ones (PYL4, aa 84-207; PYL9, aa 75-187). We aimed to determine whether DDA1 interacts with full length PYL4 and PYL9 and with other Arabidopsis PYR/PYL/RCAR family members using yeast two hybrid assays. Although we did not observe DDA1 binding to full length PYL4 and PYL9 (FIG. 4C), suggesting that additional factors might be required for their interaction, we found that DDA1 strongly binds to PYL8, which we selected for further studies (FIG. 4B).

To confirm that DDA1 and PYL8 interact in planta, we performed bimolecular fluorescence complementation (BiFC) assays. N. benthamiana leaves were coinfiltrated with Agrobacterium tumefaciens cells to express DDA1 and PYL8 fusions with the Nor C-portions of the yellow fluorescent protein (YFP). The infiltrated leaves were analyzed under the confocal fluorescence microscope 3 d after infiltration.

Physical interaction between DDA1 and PYL8 was revealed by reconstitution of YFP fluorescence in cells coinfiltrated with constructs corresponding to DDA1:YFPC and YFPN:PYL8, whereas expression of DDA1 or PYL8 constructs alone did not restore the YFP fluorescence (FIG. 4D). Interaction between DDA1 and PYL8 seemed to occur exclusively in nuclei, since fluorescent signal resulting from their interaction colocalizes with 4′,6-diamidino-2-phenylindole (DAPI) staining (FIG. 4D).

PYL8 ABA Receptor is Ubiquitinated and Degraded by the Proteasome

The CDD complex has been shown to facilitate ubiquitination and subsequent degradation of specific protein targets by the Ub-proteasome system (UPS) (Castells et al., 2010; Chen et al., 2006; Osterlund et al., 2000). To determine whether PYL8 is a substrate of the UPS, we treated Arabidopsis seedlings expressing a 3×HA-tagged PYL8 fusion (oe3HA-PYL8) with proteasome inhibitor MG132. Immunoblots using anti-HA antibodies showed increased 3HA-PYL8 protein accumulation in MG132-treated samples than in mock controls (FIG. 5A). Moreover, upon proteasome inhibition several bands of high MW were detected, likely corresponding to ubiquitinated 3HA-PYL8 forms. To confirm PYL8 ubiquitination, Ub-conjugated proteins were purified from oe3HA-PYL8 plants using commercially available p62 resin (which displays affinity for Ub and binds it non-covalently. Immunoblots using anti-HA antibodies showed precipitation of 3HA-PYL8, as multiple high MW bands, when samples were incubated with p62 resin but not when the empty resin was used, indicating that 3HA-PYL8 is modified by poly-Ub chains in planta (FIG. 5B-5C).

DDA1 Overexpression Promotes PYL8 Protein Degradation

Because PYL8 is targeted for degradation by the proteasome, and DDA1 and PYL8 physically interact, we investigated whether DDA1 mediates PYL8 destabilization. For this, we compared the rate of degradation of 3HA-PYL8 after treatment of plants with cycloheximide (CHX) with that in plants that over-express both DDA1-GFP and 3HAPYL8 (obtained by crossing between oe3HA-PYL8 and oeDDA1-GFP line 3; FIG. 2A). DDA1-GFP over-expression increased 3HA-PYL8 degradation over the time compared to oe3HA-PYL8 controls (FIG. 5D-5E). In these experiments, treatment of plants from both genotypes with MG132 attenuated 3HA-PYL8 destabilization, further confirming proteasomal control of PYL8 stability (FIG. 5F). Interestingly, ABA treatments blocked 3HA-PYL8 degradation although this effect was reduced when DDA1-GFP was over-expressed. None of these effects on 3HA-PYL8 protein levels was caused by changes in the expression of the corresponding transgene, as indicated by semiquantitative RT-PCR analysis (FIG. 5G).

It has been previously shown that PYR/PYL/RCAR ABA receptors accumulate in seeds where they mediate ABA inhibition of seed germination (Gonzalez-Guzman et al., 2012). Thus, we tested whether DDA1 also controls PYL8 levels in seeds. Immunoblots of protein extracts from imbibed seeds showed that DDA1-GFP over-expression decreases 3HA-PYL8 accumulation in both ABA-treated and non-treated seeds (FIG. 5H-5J). Again, ABA led to increased accumulation of 3HA-PYL8. Taken together these results indicate that DDA1 and ABA play opposite roles in the control of PYL8 accumulation, whereas DDA1 facilitates PYL8 degradation, ABA prevents its destabilization.

Overexpression of DDA1 Reduces Plant Sensitivity to ABA

Our data indicate that DDA1 facilitates degradation of PYL8, and likely that of other PYR/PYL/RCAR receptors with which it interacts, pointing to a negative regulatory role for DDA1 in ABA signalling. To test this hypothesis, we characterized several ABA responses in oeDDA1-GFP plants (line 3; FIG. 2A), including ABA-mediated inhibition of seed germination, seedling establishment and root, and shoot growth. As a control, we used wild-type and oeHAB1 (over-expressing the PP2C phosphatase HAB1; used as ABA-insensitive control) plants in these experiments. oeDDA1-GFP plants showed a reduced response to ABA compared to wild-type plants in all cases, except for ABA-mediated inhibition of shoot growth (FIG. 6A-6I). In addition, oeDDA1-GFP seedlings were less sensitive to NaCl- or mannitol-mediated inhibition of seed germination than the wild-type (FIG. 6G-6H), indicating that DDA1 over-expression effect is also evident under stress conditions that increase endogenous ABA levels (Leung and Giraudat, 1998; Seo and Koshiba, 2002). To confirm that reduced sensitivity to ABA is due to DDA1 over-expression and not to an artifact caused by its fusion to GFP, we obtained Arabidopsis plants expressing the cDNA of DDA1 under the control of a β-estradiol-inducible promoter (iDDA1; FIG. 6J-6K). Seed germination rates of iDDA1 plants grown in MS media supplemented or not with ABA or with β-estradiol were completely indistinguishable of those of wild-type plants. However, seedling establishment rate increased in the iDDA1 line compared to the wild-type when both ABA and β-estradiol were added to the media.

Reduced CDD Function Causes ABA Hypersensitivity

Analysis of the effect of reduced DDA1 function in ABA signalling was hindered by the fact that homozygous dda1-1 null mutants were infertile and under-represented in an F2 segregating population (˜4% instead of 25%;). Additionally, RNAi approaches did not succeed to silence DDA1, as afore-mentioned. As an alternative, we sought to characterize mutants of other CDD components since DDA1 forms part of the CDD complex. Analysis of ABA responses showed that mutations that yield reduced function of DDB1. DET1, or COP10 (note that their total loss of function is lethal; Bernhardt et al., 2010; Schroeder et al., 2002; Suzuki et al., 2002) caused an opposite ABA phenotype to that of DDA1 over-expressing plants. Thus, ddb1a, cop10-4 and det1-1 mutants showed increased response to ABA-mediated inhibition of germination and seedling establishment than wild-type plants. In the case of det1-1 mutants. ABA hypersensitivity also extended to root growth responses (FIG. 7A-7E). Next, we determined whether ABA hypersensitivity correlates with increased accumulation of PYL8 in plants showing reduced CDD function. For this analysis, we used cop10-4 mutants as a representative of CDD deficient mutants. Immunoblots of protein extracts obtained from imbibed seeds showed that cop10-4 mutation increases 3HA-PYL8 accumulation in both ABA-treated and non-treated seeds (FIG. 7F-7G). Altogether, these results suggest that cooperation between CDD components exists to control ABA receptor stability and therefore, to regulate ABA responses.

Discussion

Noteworthy, ABA and DDA1 seem to play opposite roles in the control of PYL8 stability; where ABA and DDA1 prevent and promote PYL8 degradation, respectively. Since ABA signaling is obviously strongly dependent on the activity of PYR/PYL/RCAR receptors, an ABAdependent protection mechanism for receptor stability would serve to reinforce and sustain ABA signaling. In this context. DDA1-mediated degradation of PYL8 could contribute to desensitize the pathway when stress conditions disappear and ABA levels diminish. The molecular aspects underlying ABA-mediated protection of PYL8 are totally unknown. One possibility is that ABA binding-driven changes in receptor conformation disrupt DDA1-PYL8 interaction or PYL8 ubiquitination and/or degradation rates. Thus, it is known that PYL8 interacts in an ABA-dependent manner at least with five clade A PP2Cs in vivo (Antoni et al., 2013; Saavedra et al., 2010). The ternary complexes PP2C-ABA-PYL8 show high stability (Kd around 20-40 nM) and the interaction of PYL8 with ABA and the PP2C generates substantial changes in receptor conformation (Melcher et al., 2009; Santiago et al., 2009). Therefore, it is likely that such complexes protect PYL8 from DDA1-mediated degradation or effectively compete with DDA1-PYL8 interaction. Further biochemical and molecular studies should help us to unveil the precise details of such a protective mechanism.

Despite functional redundancy between PYR/PYL/RCAR ABA receptors, PYL8 has a prominent role in mediating ABA signaling at the roots (Antoni et al., 2013). Consistent with DDA1 control of PYL8 function, oeDDA1-GFP plants phenocopied the reduced sensitivity to ABA-mediated inhibition of root growth shown by py18-1 mutants. Notably. DDA1 overexpression also altered responses that are regulated by highly redundant PYR/PYL/RCAR family members, including seed germination and seedling establishment (Gonzalez-Guzman et al., 2012), suggesting an ampler role for DDA1 in controlling ABA receptor stability. In agreement with this. PYL4 and PYL9 were identified in a yeast two hybrid-based screen of DDA1 interactors. However, contrary to PYL8 results, full length versions of PYL4 and PYL9 did not bind DDA1 in yeast, which may suggest that additional factors are required for these interactions to occur in vivo. Indeed, we cannot exclude the possibility that DDA1 activity as part of CDD complexes is aided by other subunits, including other DCAF proteins. In fact, our TAP purification CDD complexes have been proposed to play a dual role in regulating CRL4 activity by enhancing the E3 activity of CRL4, likely through its COP10 subunit, and facilitating CRL4 target recognition (Yanagawa et al., 2004; Pick et al., 2007; Olma et al., 2009).

In the latter case, it has been suggested that CDD complexes may act as adaptor modules for additional substrate receptors (Lou and Deng, 2012). Our results on the biochemical and functional characterization of Arabidopsis DDA1 strongly support this model. Thus, we found that DDA1 associates with the CDD complex and CUL4 in vivo and is involved in direct protein target recognition for ubiquitination and subsequent degradation by the proteasome. Although DDA1 association with plant CDD complexes was presumed (Chen et al., 2010), no direct evidence had been provided yet. Here, we demonstrate that DDA1 is a component of CDD using two approaches. First, we were able to detect DDA1 in biochemically purified CDD fractions. CDD purification yielded partially degraded COP10 and DET1 products, as seems to be the case for DDA1 too, which might have precluded its identification in the study by (Yanagawa et al., 2004). Second, we found all CDD components in TAP-purified DDA1 samples. Similar to its human counterpart, DDA1 association with CDD, and therefore CRL4, is mediated by its interaction with the BPA domain in DDB1 proteins (Jin et al., 2006; Pick et al., 2007).

DDA1 biochemical activity has been a matter of discussion since its identification in mammalian systems (Pick et al., 2007; Olma et al., 2009; Chen et al., 2010). One hypothesis was that DDA1 might play a structural role as part of CDD/DDD-E2 complexes. However. DDA1 is apparently not required to maintain the integrity of these complexes, since the CDD complex could be reconstituted in vitro in the absence of DDA1 (Chen et al., 2006). Another possibility was that DDA1 might be necessary to activate certain CRL4s, by stabilizing DDB1 association with a specific subset of DCAFs. Indeed, immunoprecipitation assays showed that both endogenous and tagged hDDA1 associate with DDB1. CUL4 and several DCAF proteins, including Constitutively photomorphogenic 1 (COP1), AMBRA and Cockayne syndrome A (CSA) in human cells (Jin et al., 2006; Olma et al., 2009; Behrends et al., 2010). However, experimental evidence showing DDA1-mediated stabilization of DDB1-DCAF complexes has not been provided. In this study, we propose a different function for DDA1 as a novel type of substrate receptor for CRL4 ubiquitin ligases. In this regard, we identified the first known target of DDA1 activity, the ABA receptor PYL8.

Despite functional redundancy between PYR/PYL/RCAR ABA receptors, PYL8 has a prominent role in mediating ABA signaling at the roots (Antoni et al., 2013). Consistent with DDA1 control of PYL8 function, oeDDA1-GFP plants demonstrated reduced sensitivity to ABA-mediated inhibition of root growth, as is also the case for pyl8-1 mutants. Notably. DDA1 overexpression also altered responses that are regulated by highly redundant PYR/PYL/RCAR family members, including seed germination and seedling establishment (Gonzalez-Guzman et al., 2012), suggesting a broader role for DDA1 in controlling ABA receptor stability. In agreement with this, we found that DDA1 also interacts in vivo with PYL4 and PYL9, which may represent additional targets for DDA1. However, we did not observe interaction of DDA1 with PYL5 and PYL6 in yeast two hybrid assays, suggesting that a certain degree of specificity in DDA1 activity may exist. DDA1 function towards ABA receptors is very likely performed in the context of the CDD, as indicated by the increased sensitivity to ABA of mutants of other members of the complex. Accordingly, cop10-4 plants accumulated higher levels of PYL8 protein than wild-type plants, as it is expected for plants with reduced DDA1 function. However, no other CDD component was able to interact with PYL8 under our experimental conditions, highlighting the specificity and preponderance of DDA1 in ABA receptor recognition. These results are consistent with a model in which the whole CDD complex acts as a substrate adaptor module for CRL4 where DDA1 mediates recognition of specific targets. It is noteworthy that ABA and DDA1 play opposite roles in the control of PYL8 stability where ABA and DDA1 prevent and promote PYL8 degradation, respectively. Since ABA signaling is obviously strongly dependent on the activity of PYR/PYL/RCAR receptors, an ABA-dependent protection mechanism for receptor stability would serve to reinforce and sustain ABA signaling, particularly during the early stages of signaling. However, at later stages, plant desensitization to ABA likely occurs in order to prevent the adverse effects of continuous ABA responses (i.e. growth reduction or stomatal closure). Accordingly, it has been shown that ABA reduces PYL8 gene expression after 3 h of treatment (Saavedra et al., 2010). Interestingly, ABA treatment of oeHA-PYL8 seeds for 24 h also reduced HA-PYL8 transcript levels suggesting the implication of posttranscriptional control of PYL8 mRNA by ABA. Our results on DDA1 further emphasize on the complexity and sophistication of the regulatory network that modulates ABA signaling. Thus. DDA1-mediated degradation of ABA receptors should also contribute to desensitize the pathway when stress conditions disappear and ABA levels diminish. This regulatory mechanism might be also instrumental to impair ABA signaling during germination since it has been shown that ABA concentration in seeds is reduced upon imbibition The molecular aspects underlying ABA-mediated protection of PYL8 remain unknown. This mechanism apparently does not imply disruption of the PYL8/DDA1 interaction or a reduction of DDA1 levels, but rather a decrease in PYL8 polyubiquitination rates. One possibility is that changes in receptor conformation driven by ABA-binding limit PYL8 ubiquitination. Thus, it is known that PYL8 interacts in an ABA-dependent manner at least with five clade A PP2Cs in vivo (Saavedra et al., 2010; Antoni et al., 2013). The ternary complexes PP2C-ABA-PYL8 show high stability (Kd around 20-40 nM), and the interaction of PYL8 with ABA and the PP2C generates substantial changes in receptor conformation (Melcher et al., 2009; Santiago et al., 2009). Therefore, it is possible that formation of such complexes may hide specific lysine residues on PYL8 and thereby interfere with its polyubiquitination. Further biochemical and molecular studies should help us to unveil the precise details of such a protective mechanism. Definition of the structural details of DDA1 binding to specific PYR/PYL/RCAR proteins in the presence of CDD and CRL4 complexes, and/or PP2Cs and ABA, will also help to elucidate how DDA1 substrate specificity is attained (note that DDA1 lacks WDxR motifs usually required for substrate interaction) and to better understand the modulation of ABA signaling based on the control of ABA receptor stability.

Methods

Plant Materials and Growth Conditions

Arabidopsis plants used in this study, including mutants and transgenic plants, were of the Columbia-0 (Col-0) ecotype. Plants were grown in MS media (Murashige and Skoog, 1962) with 1% sucrose at 21° C. under a 16-h-light/8-h-dark cycle using cool white fluorescent light conditions (100 mmol m-2 s-1). Specific treatments were performed as stated in each experiment (see below and figure legends). Mutants cop10-4, det1-1, cra1, hab1-1 abi1-2, pyl8-1 and transgenic lines oe3HA-PYL8. FLAGCOP10, and oeHAB1 have been previously described (Antoni et al., 2013; Fernandez-Arbaizar et al., 2012; Peeper et al., 1994; Suzuki et al., 2002; Yanagawa et al., 2004). The T-DNA insertion line corresponding to ddb1a was obtained from TAIR (http://www.Arabidopsis.org; SALK_038757). To generate transgenic plants expressing DDA1, the DDA1 cDNA was amplified using Expand High Fidelity Polymerase (Roche) and Gateway-compatible primers:

DDA1-BF
(SEQ ID NO: 197)
5′-GGGGACCACTTTGTACAAGAAAGCTGGGTAGAATAGTGAGCA
ACTTTAAGTCGA-3′
and
DDA1-BR
(SEQ ID NO: 198)
5′-GGGGACCACTTTGTACAAGAAAGCTGGGTATAAGCCCTGAGT
AGATGAAGAAGAAGACG-3′.

PCR products were cloned into the pDONR207 plasmid using Gateway BP reaction kits (Invitrogen) and verified by Sanger sequencing. Then. DDA1 cDNA was transferred, using Gateway LR reaction kits (Invitrogen), to pGWB5 (Nakagawa et al., 2007) and pMDC7 (Curtis and Grossniklaus, 2003) destination vectors. The resulting plasmids were used to generate oeDDA1-GFP and iDDA1 lines, respectively. In all cases, plant transformation was performed by transferring the corresponding constructs to Agrobacterium tumefaciens C58C1 (pGV2260) competent cells (Deblaere et al., 1985). Transformation of Arabidopsis plants was performed by the floral dip method (Clough and Bent, 1998). T1 transgenic seeds were selected based on corresponding selection markers and T3 homozygous progenies were used for further studies. Lines oeDDA1-GFP/oeFLAG-COP10, oe3HA-PYL8/pyl8-1/oeDDA1-GFP and oe3HA-PYL8/pyl8-1/cop10-4 were generated by crossing the corresponding homozygous parental lines. F2 segregating progenies of these crosses were selected in the corresponding antibiotics to isolate homozygous plants for each construct. The ddal-1 mutant was isolated by screening of an Arabidopsis TILLING (Targeting Induced Local Lesions IN Genomes) mutant collection (TILLer; Martin et al., 2009; http://www.cnb.csic.es/˜tiller/). The dda1-1 mutant, originally identified in a Landsberg erecta background, was introgressed into the Col-0 ecotype after seven sequential crosses. Plants harbouring the dda1-1 mutation (either homo- or heterozygous mutants) could be identified by their distinctive restriction pattern compared to wild-type plants after genomic PCR using specific primers 5′-CTGGGTTTTGCTGCTTACTTGG-3′ (SEQ ID NO:199) and 5′-TCCTACGAAATCCTGTGTTATG-3′ (SEQ ID NO:200), and subsequent digestion with Hphl (Roche). For BiFC experiments, N. benthamiana plants were grown in soil in the green house at 22° C. under 16-h-light/8-h-dark photoperiod prior to agroinfiltration of leaves with the corresponding constructs.

Quantitative and Semiquantitative RT-PCR

Quantitative RT-PCR experiments were performed using RNA extracted from Col-0 wild-type and oeDDA1-GFP plants. Three biological replicates, consisting of tissue pooled from 15-20 plants from different plates, were taken. RNA extraction and cleanup was done with RNeasy mini kit (Qiagen) and DNase digestion to remove genomic DNA contamination. cDNA was synthesized from 1 μg of total RNA using a high-capacity cDNA reverse transcription kit (Applied Biosystems). Ten μL from one-tenth diluted cDNA was used to amplify DDA1 and the housekeeping gene ACTIN8 using FastStart Universal Probe Master (Roche). Primers used were:

DDA1-RTF
(SEQ ID NO: 201)
5′-CCCTCCGATCCTTCTAATCC-3′,
DDA1-RTR
(SEQ ID NO: 202)
5′-GCTGCGTATAAGAATGTTTTTCAC-3′,
ACT8-F
(SEQ ID NO: 203)
5′-GGTACTGGAATGGTTAAGGC-3′
and
ACT8-R
(SEQ ID NO: 204)
5′-GTCCAACACAATACCGGTTG-3′.

Quantitative PCRs were performed in 96-well optical plates in a 7300 Real Time PCR system (Applied Biosystems). The PCR conditions were as follows: 2 min at 50° C., 10 min at 95° C. and 40 cycles of 15 s at 95° C. and 30 s at 60° C.

Semiquantitative PCR experiments from seedlings were performed using RNA prepared as afore-mentioned. RNA extraction from seeds was carried out as previously described (Onate-Sanchez and Vicente-Carbajosa, 2008). cDNA from all tissues was synthetized as described above. Five pL of one-fifth diluted cDNA was used to amplify DDA1-GFP, 3HA-PYL8 and the housekeeping gene ACTIN8 using the following primer:

HA-F
(SEQ ID NO: 205)
5′-CTATGACGTCCCGGACTATGCA-3′,
PYL8-R
(SEQ ID NO: 206)
5′-GGTGAAGAGAGATGATTGAAG-3′,
DDA1-2F
(SEQ ID NO: 207)
5′-TCGTCCCTCCGATCCTTCTAATCC-3′,
GFP-R
(SEQ ID NO: 208)
5′-CTTGCCGTAGGTGGCATCGC-3′,
ACT8semi-F
(SEQ ID NO: 209)
5′-GGTACTGGAATGGTTAAGGC-3′,
ACT8semi-R
(SEQ ID NO: 210)
5′-GTCCAACACAATACCGGTTG-3′.

The PCR conditions were as follows: 1 min at 94° C., 35 cycles of 15 s at 94° C., 1 min at 58-62° C., and 1 min 30 s at 72° C., and finally, 5 min at 72° C. 25

TAP Assays

Cloning of a GS-tagged DDA1 fusion under the control of the constitutive cauliflower tobacco mosaic virus 35S promoter and transformation of Arabidopsis cell suspension cultures were performed as previously described (Van Leene et al., 2007). TAP of protein complexes was done using GS tag (Burckstummer et al., 2006) followed by protein precipitation and separation, according to Van Leene et al. (2008). For the protocols of proteolysis and peptide isolation, acquisition of mass spectra by a 4800 MALDI TOF/TOF Proteomics Analyzer (AB SCIEX), and MS based protein homology identification based on the TAIR genomic database, we refer to Van Leene et al. (2010). Experimental background proteins were subtracted based on approximately 40 TAP experiments on wild-type cultures and cultures expressing TAP-tagged mock proteins GUS, RFP and GFP (Van Leene et al., 2010).

Microscopy Analysis

For ovule observations, pistils from not-pollinated Arabidopsis flowers were opened longitudinally and observed using a Leica M165FC stereomicroscope. Photographs were taken with a Leica color camera DFC295. Then, pistils were cleared in chloral hydrate (2 mg mL-1) and ovules were observed under a Leica DMR microscope with differential interference contrast (DIC) optics (http://www.leica.com). Photographs were taken with an Olympus DP70 camera. To analyze DDA1-GFP subcellular localization, images of 5-d-old oeDDA1-GFP Arabidopsis roots and of Nicotiana leaves agroinfiltrated with DDA1-GFP and different organelle markers (Nelson et al., 2007), were visualized by a confocal microscope at 495-610 nm (Leica). To visualize nuclei, roots and Nicotiana leaves were submerged in a DAPI solution (1 μg mL-1 DAPI in 100 mM phosphate buffer, 0.5% Triton X-100).

Yeast Two Hybrid Experiments

The full length DDA1 cDNA was cloned into the pGBKT7 (Gal4 DNA binding domain, BD; Clontech). This construct was used to screen a whole seedling cDNA library (Bustos et al., 2010) prepared in the pGADT7 vector (Gal4 activation domain, AD. Clontech) to detect DDA1-interacting proteins. To confirm protein interactions, plasmids were co-transformed into Saccharomyces cerevisiae AH109 cells, following standard heat-shock protocols (Chini et al., 2007). Successfully transformed colonies were identified on yeast synthetic drop-out lacking Leu and Trp; these colonies were resuspended in water and transferred to selective media lacking Ade, His, Leu and Trp. Plates without His were supplemented with different concentrations of 3-amino 1,2,4-triazole (3AT; ranging 0.5-10 mM). Yeast cells were incubated at 30° C. during 6 days. Empty vectors were co-transformed as negative controls. To test the DDA1 interaction with specific DDB1a domains, DDB1a truncated versions were generated and cloned into the pGADT7 vector as follows: BPA (aa 16-350), BPB (aa 387-704), BPA+BPB (aa 16-704), BPC (aa 704-1002) and BPB+BPC (aa 350-1002). Full length DDB1a was used as a positive control.

BiFC Experiments

Different combinations of A. tumefacines clones expressing fusion proteins

YFPN:PYL8/DDA1:YFPC were co-infiltrated into the abaxial surface of 3-week-old N. benthamiana plants as described (Voinnet et al., 2003). The p19 protein was used to suppress gene silencing. The empty vectors were used as negative controls. Fluorescence was visualized in epidermal cells of leaves after 3 d of infiltration using a Leica sp5 confocal microscope. Nuclei were visualized after submerging the leaves in a DAPI solution (1 μg mL-1 DAPI in 100 mM phosphate buffer, 0.5% Triton X-100).

Genetic Analysis

To examine gametophytic transmission of the dda1-1 mutant allele, reciprocal test crosses were performed between wild-type (Col-0) and heterozygous dda1-1 mutant plants. Seeds harvested from crosses were germinated and grown on soil, and genomic DNAs from the F1 progeny were analyzed by PCR using the primer combination to detect the dda1-1 mutation. Transmission efficiency (TE) of the mutant allele via each type of gamete (TE male and TE female) was calculated as described previously (Howden et al., 1998).

Protein Extraction, Co-Immunoprecipitation Assays and Immunoblots

For protein extraction from seedlings, proteins were extracted in buffer containing 50 mM Tris-HCl pH 7.4, 150 mM NaCl, 10mM MgCl2, 1 mM PMSF, 0.1%NP-40 and 1× complete protease inhibitor (Roche). After centrifugation at 16,000 g at 4° C., the supernatants were collected. This step was repeated twice. For protein extraction from seeds, seeds were frozen in liquid N2 and then homogenized in buffer containing 7 M urea, 2 M thiourea, 4% 3-[(3-cholamidopropyl)dimethylammonio]-1-propanesulfonate (CHAPS; w/v), 18 mM Tris-HCl pH 7.5, 0,2% Triton X100, 1× complete protease inhibitor (Roche). After 10 min incubation at 4° C. with rotation. DTT was added to protein extracts (14 mM final concentration), prior to 20 min incubation at 4° C. Extracts were clarified by centrifugation as afore-mentioned. Protein concentration in final supernatants was determined using a Bio-Rad Protein Assay kit. For in vivo co-immunoprecipitation assays, normalized seedling protein extracts were incubated with 5 μl anti-GFP antibody (Living colors Full length A.V. Polyclonal Antibody, Clontech) for 1 h at 4° C. with rotation. 10 μl of protein A-coupled beads (prewashed twice with 0.1 M Glycine pH 2.7) were added to the samples and incubated for an additional hour at 4° C. with rotation. After washing three times with 500 μL extraction buffer, samples were denatured, separated on SDS-PAGE gels and transferred onto polyvinylidene difluoride (PVDF) membranes (Millipore). Membranes were probed with different antibodies: anti-GFP-HRP (for DDA1-GFP detection, Milteny Biotec), monoclonal Anti-FLAG M2 (for FLAG-COP10; Sigma), anti-CUL4 (Chen et al., 2006). For immnunodetection of 3HA-PYL8, anti-HA-HRP (Roche) was used. To confirm equal protein loading, membranes were stained with Ponceau reagent or immunoblotted using anti-RPT5 (Kwok et al., 1999).

CDD complex was purified as previously described (Yanagawa et al., 2004). For the analysis of purified CDD complex fractions, proteins in each fraction were separated onto 15% SDS-PAGE gels. Silver staining and immunoblots using anti-DDA1 antibodies were performed to visualize specific protein bands. For anti-DDA1 production see below.

Purification of Recombinant Proteins and Antibody Production

Recombinant His-DDA1 protein was expressed in the Escherichia coli BL21 (DE3) strain carrying a pET28-HisT7DDA1 construct. Bacteria were cultured in LB at 37° C. to an optical density at 600 nm of 0.6, at which time protein expression was induced with 0.2 mM isopropyl-D-thio-galactopyranoside for 3 h. Cell lysis was performed using a French Press and lysates were clarified by centrifugation at 16,000 g for 30 min at 4° C. His-DDA1 protein was purified from lysates with Ni-NTA-agarose beads under denaturing conditions (Qiagen) and eluted with a pH gradient as described by the manufacturer. Protein concentration in final eluates was determined using Bio-Rad Protein Assay kit. To raise anti-DDA1 antibodies purified His-DDA1 protein was introduced into two rabbits (1 mg/each). Rabbit preimmune serum was kept to check for anti-DDA1 specificity.

Affinity Purification of Ubiquitinated Proteins.

Isolation of ubiquitinated proteins was performed as previously described (Manzano et al., 2008) with small modifications. Briefly, proteins were extracted from oe3HA-PYL8 plants using buffer BI (50 mM Tris-HCl pH=7.5; 20 mM NaCl; 0.1% NP-40 and 5 mM ATP) plus plant protease inhibitors cocktail (Sigma), 1 mM of PMSF and 50 μM MG132. Protein extracts were incubated with 40 μL pre-washed p62-agarose (Wilkinson et al., 2001) or the agarose alone at 4° C. during 4 h. Afterwards, the beads were washed 2 times with 1 mL BI buffer once more with 1 mL buffer BII (BI plus 200 mM NaCl) and proteins were eluted by boiling into 50 μL SDS loading buffer. The eluted proteins were separated by SDS-PAGE and analyzed by immunoblotting using anti-Ub (Boston Biochem) to detect the presence of ubiquitinated proteins or anti-HAHRP (Roche) for 3HA-PYL8 detection.

In Vivo Protein Degradation Assays

Seedlings were grown in MS solid media for 8 d and then transferred to liquid MS media containing 50 pM cicloheximide (CHX; Sigma) in the presence or absence of 50 μM ABA (Sigma). The effect of proteasome inhibition was tested by adding 50 μM MG132 (Sigma) to the liquid MS. Whole plant samples were harvested at specific time points as indicated. Protein extraction and immunoblots were performed as afore30 mentioned. ImageJ v1.37 software (http://rsb.info.nih.gov/ij) was used to analyze protein band intensity.

Seed Germination and Seedling Establishment Assays.

After surface sterilization of the seeds, stratification was conducted in the dark at 4° C. for 3 U. Next, approximately 100 seeds of each genotype were sowed on MS plates lacking or supplemented with 0.5 μM ABA, 150 mM NaCl or 400 mM Mannitol. In the analyses of iDDA1 lines, β-estradial was added to media at 10 βM final concentration as stated. To score seed germination, radical emergence was analyzed at 72 h and 96 h after sowing. Seedling establishment was scored at 5 and 7 d as the percentage of seeds that developed green expanded cotyledons and the first pair of true leaves.

Root and Shoot Growth Assays.

Seedlings were grown on vertically oriented MS plates for 4 to 5 U. Afterwards, 20 plants were transferred to new MS plates lacking or supplemented with 10 μM concentration of ABA. The plates were scanned on a flatbed scanner after 10 d to produce image files suitable for quantitative analysis of root growth using ImageJ v1.37 software. As an indicator of shoot growth, the maximum rosette radius was measured after 20 d.

Accession Numbers

Sequence data from this article can be found in the Arabidopsis Genome Initiative database under the following accession numbers: DDA1 (At5g41560), DDB1A (At4g05420), DDB1 B (At4g21100), COP10 (At3g13550), DET1 (At4g10180), CUL4(At5g46210), PYL8 (At5g53160), PYL4 (At2g38310), PYL9 (At1g01360). Accession numbers are incorporated by reference.

### REFERENCES

Antoni, R., Gonzalez-Guzman, M., Rodriguez, L. Peirats-Llobet, M., Pizzio, G. A., Fernandez, M. A., De Winne, N., De Jaeger, G., Dietrich, D., Bennett, M. J., et al. (2013). PYRABACTIN RESISTANCE1-LIKE8 plays an important role for the regulation of abscisic acid signaling in root. Plant physiology 161, 931-941.

Burckstummer, T., Bennett, K. L., Preradovic, A., Schutze, G., Hantschel, O., Superti-Furga, G., and Bauch, A. (2006). An efficient tandem affinity purification procedure for interaction proteomics in mammalian cells. Nature methods 3, 1013-1019.

Bustos, R., Castrillo, G., Linhares, F., Puga, M. I., Rubio, V., Perez-Perez, J., Solano, R., Leyva, A., and Paz-Ares, J. (2010). A central regulatory system largely controls transcriptional activation and repression responses to phosphate starvation in Arabidopsis. PLoS genetics 6.

Castells, E., Molinier, J., Drevensek, S., Genschik, P., Barneche, F., and Bowler, C. (2010). det1-1-induced UV-C hyposensitivity through UVR3 and PHR1 photolyase gene over-expression. The Plant journal : for cell and molecular biology.

Clough, S. J., and Bent, A. F. (1998). Floral dip: a simplified method for Agrobacterium-mediated transformation of Arabidopsis thaliana. The Plant journal: for cell and molecular biology 16, 735-743.

Curtis, M. D., and Grossniklaus, U. (2003). A gateway cloning vector set for high-throughput functional analysis of genes in planta. Plant physiology 133, 462-469.

Chen, H., Shen, Y., Tang, X., Yu, L. Wang, J., Guo, L. Zhang, Y., Zhang, H., Feng, S., Strickland, E., et al. (2006). Arabidopsis CULLIN4 Forms an E3 Ubiquitin Ligase with RBX1 and the CDD Complex in Mediating Light Control of Development. The Plant cell 18, 1991-2004.

Chini, A., Fonseca, S., Fernandez, G., Adie, B., Chico, J.M., Lorenzo, O., Garcia-Casado, G., Lopez-Vidriero, I., Lozano, F. M., Ponce, M. R., et al. (2007). The JAZ family of repressors is the missing link in jasmonate signalling. Nature 448, 666-671.

Chinnusamy, V., Gong, Z., and Zhu, J. K. (2008). Abscisic acid-mediated epigenetic processes in plant development and stress responses. Journal of integrative plant biology 50, 1187-1195.

Deblaere, R., Bytebier, B., De Greve, H., Deboeck, F., Schell, J., Van Montagu, M., and Leemans, J. (1985). Efficient octopine Ti plasmid-derived vectors for Agrobacterium-mediated gene transfer to plants. Nucleic acids research 13, 4777-4788.

Fernandez-Arbaizar, A., Regalado, J. J., and Lorenzo, O. (2012). Isolation and characterization of novel mutant loci suppressing the ABA hypersensitivity of the Arabidopsis coronatine insensitive 1-16 (coi1-16) mutant during germination and seedling growth. Plant & cell physiology 53, 53-63.

Hauser, F., Waadt, R., and Schroeder, J. I. (2011). Evolution of abscisic acid synthesis and signaling mechanisms. Current biology: CB 21, R346-355.

Hirayama, T., and Shinozaki, K. (2010). Research on plant abiotic stress responses in the post-genome era: past, present and future. The Plant journal : for cell and molecular biology 61, 1041-1052.

Howden, R., Park, S. K., Moore, J. M., Orme, J., Grossniklaus, U., and Twell, D. (1998). Selection of T-DNA-tagged male and female gametophytic mutants by segregation distortion in Arabidopsis. Genetics 149, 621-631.

Kwok, S. F., Staub, J. M., and Deng, X. W. (1999). Characterization of two subunits of Arabidopsis 19S proteasome regulatory complex and its possible interaction with the COP9 complex. Journal of molecular biology 285, 85-95.

Lau, O. S., and Deng, X. W. (2012). The photomorphogenic repressors COP1 and DET1: 20 years later. Trends in plant science 17, 584-593.

Manzano, C., Abraham, Z., Lopez-Torrejon, G., and Del Pozo, J. C. (2008). Identification of ubiquitinated proteins in Arabidopsis. Plant molecular biology 68, 145-158.

Martin, B., Ramiro, M., Martinez-Zapater, J. M., and Alonso-Blanco, C. (2009). A high-density collection of EMS-induced mutations for TILLING in Landsberg erecta genetic background of Arabidopsis. BMC plant biology 9, 147.

Murashige, T., and Skoog, F. (1962). A revised medium for rapid growth and bio assays with tobacco tissue cultures. Physiologia Plantarum 15, 473-497.

Nakagawa, T., Kurose, T., Hino, T., Tanaka, K., Kawamukai, M., Niwa, Y., Toyooka, K., Matsuoka, K., Jinbo, T., and Kimura, T. (2007). Development of series of gateway binary vectors, pGWBs, for realizing efficient construction of fusion genes for plant transformation. Journal of bioscience and bioengineering 104, 34-41.

Nelson, B. K., Cai, X., and Nebenfuhr, A. (2007). A multicolored set of in vivo organelle markers for co-localization studies in Arabidopsis and other plants. The Plant journal: for cell and molecular biology 51, 1126-1136.

Olma, M. H., Roy, M., Le Bihan, T., Sumara, I., Maerki, S., Larsen, B., Quadroni, M., Peter, M., Tyers, M., and Pintard, L. (2009). An interaction network of the mammalian COP9 signalosome identifies Dda1 as a core subunit of multiple Cul4-based E3 ligases. Journal of cell science 122, 1035-1044.

Onate-Sanchez, L. and Vicente-Carbajosa, J. (2008). DNA-free RNA isolation protocols for Arabidopsis thaliana, including seeds and siliques. BMC research notes 1, 93.

Peeper, D. S., van der Eb, A.J., and Zantema, A. (1994). The G1/S cell-cycle checkpoint in eukaryotic cells. Biochimica et biophysica acta 1198, 215-230.

Pick, E., Lau, O. S., Tsuge, T., Menon, S., Tong, Y., Dohmae, N., Plafker, S. M., Deng, X. W., and Wei, N. (2007). Mammalian DET1 regulates Cul4A activity and forms stable complexes with E2 ubiquitin-conjugating enzymes. Molecular and cellular biology 27, 4708-4719.

Rubio, S., Rodrigues, A., Saez, A., Dizon, M. B., Galle, A., Kim, T.H., Santiago, J., Flexas, J., Schroeder, J. I., and Rodriguez, P. L. (2009). Triple loss of function of protein phosphatases type 2C leads to partial constitutive response to endogenous abscisic acid. Plant physiology 150, 1345-1355.

Saez, A., Apostolova, N., Gonzalez-Guzman, M., Gonzalez-Garcia, M. P., Nicolas, C., Lorenzo, O., and Rodriguez, P. L. (2004). Gain-of-function and loss-of-function phenotypes of the protein phosphatase 2C HAB1 reveal its role as a negative regulator of abscisic acid signalling. The Plant journal : for cell and molecular biology 37, 354-369.

Saez, A., Robert, N., Maktabi, M. H., Schroeder, J. I., Serrano, R., and Rodriguez, P L (2006). Enhancement of abscisic acid sensitivity and reduction of water consumption in Arabidopsis by combined inactivation of the protein phosphatases type 2C ABM and HAB1. Plant physiology 141, 1389-1399.

Suzuki, G., Yanagawa, Y., Kwok, S. F., Matsui, M., and Deng, X. W. (2002). Arabidopsis COP10 is a ubiquitin-conjugating enzyme variant that acts together with COP1 and the COP9 signalosome in repressing photomorphogenesis. Genes & development 16, 554-559.

Van Leene, J., Hollunder, J., Eeckhout, D., Persiau, G., Van De Slijke, E., Stals, H., Van Isterdael, G., Verkest, A., Neirynck, S., Buffel, Y., et al. (2010). Targeted interactomics reveals a complex core cell cycle machinery in Arabidopsis thaliana. Molecular systems biology 6, 397.

Van Leene, J., Stals, H., Eeckhout, D., Persiau, G., Van De Slijke, E., Van Isterdael, G., De Clercq, A., Bonnet, E., Laukens, K., Remmerie, N., et al. (2007). A tandem affinity purification-based technology platform to study the cell cycle interactome in Arabidopsis thaliana. Molecular & cellular proteomics: MCP 6, 1226-1238.

Van Leene, J., Witters, E., Inze, D., and De Jaeger, G. (2008). Boosting tandem affinity purification of plant protein complexes. Trends in plant science 13, 517-520.

Voinnet, O., Rivas, S., Mestre, P., and Baulcombe, D. (2003). An enhanced transient expression system in plants based on suppression of gene silencing by the p19 protein of tomato bushy stunt virus. The Plant journal : for cell and molecular biology 33, 949-956.

Wilkinson, C. R., Seeger, M., Hartmann-Petersen, R., Stone, M., Wallace, M., Semple, C., and Gordon, C. (2001). Proteins containing the UBA domain are able to bind to multi-ubiquitin chains. Nature cell biology 3, 939-943.

Yanagawa, Y., Sullivan, J. A., Komatsu, S., Gusmaroli, G., Suzuki, G., Yin, J., Ishibashi, T., Saijo, Y., Rubio, V., Kimura, S., et al. (2004). Arabidopsis COP10 forms a complex with DDB1 and DET1 in vivo and enhances the activity of ubiquitin conjugating enzymes. Genes & development 18, 2172-2181.

Sequence listing

AtDDA1 nucleic acid sequence, CDS
SEQ ID NO: 1
ATGGCGTCGATTCTGGGTGATTTGCCTTCCTTTGATCCTCACAATTTCAGTCAACATCGTCCCTCCGATC
CTTCTAATCCCTCTAAGATGGTTCCTACCACCTATCGTCCTACTCACAACCGTACACTTCCACCACCAGA
TCAAGTGATAACTACAGAAGTGAAAAACATTCTTATACGCAGCTTCTATCAACGAGCTGAAGAAAAGTTA
AGACCAAAGAGACCGGCTACAGATCATCTGGCAGCGGAGCACGTGAACAAGCATTTCCGTGCTGCGTCTT
CTTCTTCATCTACTCAGGGCTTATAA
AtDDA1 nucleic acid sequence, cDNA
SEQ ID NO: 2
GTCTGAAGAGAAGGAAAGATCATCAATCACGATTCCAATGGCGTCGATTCTGGGTGATTTGCCTTCCTTT
GATCCTCACAATTTCAGTCAACATCGTCCCTCCGATCCTTCTAATCCCTCTAAGATGGTTCCTACCACCT
ATCGTCCTACTCACAACCGTACACTTCCACCACCAGATCAAGTGATAACTACAGAAGTGAAAAACATTCT
TATACGCAGCTTCTATCAACGAGCTGAAGAAAAGTTAAGACCAAAGAGACCGGCTACAGATCATCTGGCA
GCGGAGCACGTGAACAAGCATTTCCGTGCTGCGTCTTCTTCTTCATCTACTCAGGGTTTATAAAAAACTT
AAGTTCAAGCCTATAACAATGGTCATTTGTATGAGTACCTCTTATTAGTGTTTTCAATGTAGAAAAAAAA
AGATAAGAGCAGTTCATGAGAGAAATGTAGTGAAAATGTGTGTGTACATAACATTAATGTTTCTTTTATT
TCTTGACTTAAAGTTGCTCACTATTCA
AtDDA1 nucleic acid sequence, genomic DNA
SEQ ID NO: 3
GTCTGAAGAGAAGGAAAGATCATCAATCACGATTCCAATGGCGTCGATTCTGGGTGATTTGCCTTCCTTT
GATCCTCACAATTTCAGTCAACATCGTCCCTCCGATCCTTCTAATCCCTCTGTTAGTTTCTTCCCCCAAA
TTCAATTTTTCAATTTTACGGATCTGAGTTAGGCTTTACTTGGTCGTATTGGAAAAAAAATGTGTCCTTT
GTTGATTCAAAGAGATGTAATTCGAATGTGTATCTGGGTTTTGCTGCTTACTTGGTCAGTTCCAAAAAGT
TCCATCTTTTCAATTATCATCGAGTTTGCTGTTGGATCTTGTGAAAAACCAATACAAATTAGCCATTTTT
GTCAGATTGATTGATCTTAGAATCATAATTCTGATTCCATTTGGCCATAATTTAGCTGCTAAGTGACGAA
GACAAGTTTCAACTAGCTTGTATCAGTTAAAGATTAGAGTTTTGATCTGTTATCGAAGGTTTGAGTTTTT
TGTTCATGTTTTCTTGCAGAAGATGGTTCCTACCACCTATCGTCCTACTCACAACCGTACACTTCCACCA
CCAGATCAAGGTGAAACAAAAAATCGTGCTTTTTTGAAAAACCTTGCGTGTTTTTTCGGCTAGAGATTTT
AGAATTTCGTTACATTTTATATATAGTGTCAAAGATTTGTCTTATGAAGTTGTGATCTTGAACTTGCTTT
GATTGAGTGATTTAGTTACTGTCTTTACTATGTATCACTTCTTAGAATCTCTAGGCAAATTGGTGTTAAT
CAGATTCAACAGTCTCGAGTTTTCACAGATCATGTCTTATGTTTTTACTAATTTGTATCTTGTTCGTTAT
GGTTGTAGTGATAACTACAGAAGTGAAAAACATTCTTATACGCAGCTTCTATCAACGAGCTGAAGAAAAG
GTGAAACAGCTCTCAACTCTCATTCCTCATAGTTTGTGATTCTTTGATTCAAATCTCCGTTGTTTCCCTC
GTATATATAGTTCATAACACAGGATTTCGTAGGAAAATAGACAAAAGAAAACGATATAGAACAATCTTGA
ACTTCTTCAGATAACAAAACTGTTGATTTTGGTTGTGTTATCCGAAATCTTAATTTGTTTTGTCAAATTT
GTCAATTGCAGTTAAGACCAAAGAGACCGGCTACAGATCATCTGGCAGCGGAGCACGTGAACAAGCATTT
CCGTGCTGCGTCTTCTTCTTCATCTACTCAGGGTTTATAAAAAACTTAAGTTCAAGCCTATAACAATGGT
CATTTGTATGAGTACCTCTTATTAGTGTTTTCAATGTAGAAAAAAAAAGATAAGAGCAGTTCATGAGAGA
AATGTAGTGAAAATGTGTGTGTACATAACATTAATGTTTCTTTTATTTCTTGACTTAAAGTTGCTCACTA
TTCACT
AtDDA1 (Arabidopsis thaliana) peptide sequence
SEQ ID NO: 4
MASILGDLPSFDPHNFSQHRPSDPSNPSKMVPTTYRPTHNRTLPPPDQVITTEVKNILIRSFYQRAEEKL
RPKRPATDHLAAEHVNKHERAASSSSSTQGL
Arabidopsis lyrata, CDS
SEQ ID NO: 5
ATGGCGTCGATTCTGGGGGATTTACCTTCCTTCGATCCTCACAATTTCAGTCAACATCGTCCCTCCGATC
CTTCTAATCCCTCTAGGATGGTTCCTACCACCTATCGTCCTACTCACAATCGTACACTTCCACCACCAGA
TCAAGTGATAACTACAGAAGTGAAAAACATACTTATACGCAGCTTCTATCAACGAGCTGAAGAAAAGTTA
AGACCAAAGAGACCGGCTACAGATCATCTGGCAGCCGAGCACGTGAACAAGCATTTCCGCGCCGCGTCTT
CTTCATCTTCTACTCAGGGCTTATAA
Arabidopsis lyrata, cDNA
SEQ ID NO: 6
ACGATTCCAATGGCGTCGATTCTGGGGGATTTACCTTCCTTCGATCCTCACAATTTCAGTCAACATCGTC
CCTCCGATCCTTCTAATCCCTCTAGGATGGTTCCTACCACCTATCGTCCTACTCACAATCGTACACTTCC
ACCACCAGATCAAGTGATAACTACAGAAGTGAAAAACATACTTATACGCAGCTTCTATCAACGAGCTGAA
GAAAAGTTAAGACCAAAGAGACCGGCTACAGATCATCTGGCAGCCGAGCACGTGAACAAGCATTTCCGCG
CCGCGTCTTCTTCATCTTCTACTCAGGGCTTATAAAAAACTTACCCTGGTGAGCCTATGATAATGGTCAT
TGTGTATGAGTTCTTATTAGCATTTTCAATGTAGAGAAAGAAAGAAAGAAAGATAAGAGCAGTTCATG
Arabidopsis lyrata, gDNA
SEQ ID NO: 7
ACGATTCCAATGGCGTCGATTCTGGGGGATTTACCTTCCTTCGATCCTCACAATTTCAGTCAACATCGTC
CCTCCGATCCTTCTAATCCCTCTGTTAGTTTCTTCCCCAATTCTCATCTTCGATTTTTTCTTTCTTCCAA
TTCTTCAACTCTCGGATGAAAATTTCAGATGTCTTCTGATTCTGTTTTGTCTGAATTCGTATGAGAACTC
TTTTTATACAGATCTGAGTTCGGTTTACTTTGGTCGTATTGAAAAAAATGTTGATCCAATTTTGATAATT
CAAATTGTTCAATGAGAAAGTAATTCGAATGTGTATCTAGGTTTGCTTGTTTACTTGATCTGATCCAAAA
GTTCCATGTTTTCATCGATTTTGCTGTTGGATCTTGTGAGAAACCTAGACAAAAAGTAGCCATTTTTGAC
AGATTTATTGATCTTAGAATCATAATTCTGTTCCCAATTGGTTCCCATTGGCCTTAATTTAGCTGCTAAG
TGACTAAGGAGTATGCAAGTTTTCATTTTTCTTGCAAATTCCAATTAGCTTGTCTCAGTTAAAGATTATA
TTTTTGATCAGTTATCGAAGATTTGAGTTTTGTTCATGTTTTCTTGCAGAGGATGGTTCCTACCACCTAT
CGTCCTACTCACAATCGTACACTTCCACCACCAGATCAAGGTGAAACAAAATTGCAGTTTTTTATTTATT
TTAAATCTTGCGTGTTCTTCGGCTAGAGATTTTAGAACTTTGTTACATTTTGTAGTCTAAAATTGGCCTT
TTAAAGTTGTGATCTTGGCTAGAGACTGATCTTGAACTTGCTTTGATTGAGCAATCTAGTTACTGTCTTT
ACCATGGATCACTTCTTAGAATCTCTAGGCAAATTGTTGTTAATCGGATTCAACAGTCTCCAATTTTCAC
GGGTCACGTCTATGTTTTTGTTTGTTTTGGTTGTAGTGATAACTACAGAAGTGAAAAACATACTTATACG
CAGCTTCTATCAACGAGCTGAAGAAAAGGTAAAACAACTCTCATTCCTCATAGTTTGTGATTCTTTGATT
CAAATCACCGTTGTTTCCCTCGTATATATAGTTCGTAGGAAAATAGACAAAAGAAAACGATATAGAACAA
ATCTTGAACTTCTTCAGATAACAAATCTGTTGATTTTTGGTTGTGAATTATCCAAAATCTTTATGTTTTT
TTTTGTCAAATTTTTCATTTGCAGTTAAGACCAAAGAGACCGGCTACAGATCATCTGGCAGCCGAGCACG
TGAACAAGCATTTCCGCGCCGCGTCTTCTTCATCTTCTACTCAGGGCTTATAAAAAACTTACCCTGGTGA
GCCTATGATAATGGTCATTGTGTATGAGTTCTTATTAGCATTTTCAATGTAGAGAAAGAAAGAAAGAAAG
ATAAGAGCAGTTCATG
Arabidopsis lyrata
SEQ ID NO: 8
HASILGDLPSFDPHNFSQHRPSDPSNPSRMVPTTYRPTHNRTLPPPDQVITTEVKNILIRSFYQRAEEKL
RPKRPATDHLAAEHVNKHFRAASSSSSTQGL
Brassica napus, CDS
SEQ ID NO: 9
ATGGGGTCGATTCTGGGAGATTTGCCGTCCTTCGATCCTCATAATTTCAGTCAACATCGTCCCTCTGACC
CTTCTAATCCCTCTAGGATGGTTCCAACAACCTATCATCCAACCCACAACCGTACTCTTCCACCTCCACA
TCAAGTGATAACTACGGAAGTAAAGAACATACTCATACGCAGCTTCTATCAGCGAGCTGAAGATAAGATG
AGACCAAAGAGACCGGCTTCAGAACATCTGGCCGGTGAGCACGGTAACAAGCATTTCCGTGCCTCTTCAT
CTACTCAGGGTTTATAA
Brassica napus, cDNA
SEQ ID NO: 10
GGTTTCTGAGCCGGTCCCTGAGGTTAAACACGAGGAAGCGGAGAAGAAACCTAGTCTCCTTGAGAAGCTT
CACCGAAGCGACAGCTCTTCTAGCTCCTCAAGCGAAGAAGAAGGTGAAGATGGTGAGAAGAGGAAGAAGA
AGAAAAAGGATAAGAAGAAGATTGCTACTGAAGGAGAGGTGCAAACAGAAGAGGCGAAGAAAGGGTTTAT
GGACAAGCTCAAGGAGAAGCTTCCAGGACACGGAAAGAAACCCGAAGATGACTCAGCCGTTGCGGCTGCA
CCGGTTGTTGCTCCTCCTGTGGAGGAAGCGCATCCGGCTGAGAAGAAGGGGATCTTGGAGAAGATTAAAG
AGAAGCTTCCAGGATACCACTCAAAGACCGTTGAGGAGGAGAAGAAAGATGATCACTGAAAACATGAATA
CTAATGATGATGAGAGACATCTCGTGTTGTTTGTGATGGATGATTATCATCTTTTTCTTTTGTGCTGTTG
AAGTTTGTTGGCTTCTTTATAGTTTATTTTGCAGTTTCCCTATTTTTCTCTTTGTTGTGTGTTTAGTGTA
TGGTTTCAAGGTATTTTGAAGTTATGAATTCCTTGA
Brassica napus
SEQ ID NO: 11
MGSILGDLPSFDPHHFSQHRPSDPSNPSRMVPTTYHPTHNRTLPPPHQVITTEVKNILIRSFYQRAEDKM
RPKRPASEHLAGEHGNKHFRASSSTQGL
Brassica oleracea, CDS
SEQ ID NO: 12
ATGGGGTCGATTCTGGGAGATTTACCGTCCTTCGATCCTCACAATTTCAGTCAACATCGTCCCTCCGACC
CTTCTAATCCCTCTAGGATGGTTCCAACAACCTATCATCCAACTCACAACCGTACTCTTCCACCTCCACA
TCAAGTGATAACTACGGAAGTAAAGAACATACTCATACGCAGCTTCTACCAACGAGCTGAAGATAAGATG
AGACCAAAGAGACCGGCTTCAGAACATCTGGCGGGTGAGCACGGGAACAAGCATTTCCGTGCTTCCTCAT
CATCTGCTCAGGGTTTATAA
Brassica oleracea, cDNA
SEQ ID NO: 13
ATGGGGTCGATTCTGGGAGATTTACCGTCCTTCGATCCTCACAATTTCAGTCAACATCGTCTATGGGGTC
GATTCTGGGAGATTTACCGTCCTTCGATCCTCACAATTTCAGTCAACATCGTUCCCTCCGACCCTTCTAA
TCCCTCTAGGATGGTTCCAACAACCTATCATCCAACTCACAACCTCCCTCCGACCCTTCTAATCCCTCTA
GGATGGTTCCAACAACCTATCATCCAACTCACAACUCGTACTCTTCCACCTCCACATCAAGTGATAACTA
CGGAAGTAAAGAACATACTCATACGCCTCGTACTCTTCCACCTCCACATCAAGTGATAACTACGGAAGTA
AAGAACATACTCATACGCUAGCTTCTACCAACGAGCTGAAGATAAG
Brassica oleracea
SEQ ID NO: 14
MGSILGDLPSFDPHNFSQHRPSDPSNPSRMVPTTYHPTHNRTLPPPHQVITTEVKNILIRSFYQRAEDKM
RPKRPASEHLAGEHGNKHFRASSSSAQGL
Brassica rapa, CDS
SEQ ID NO: 15
ATGGGGTCGATTCTGGGAGATTTGCCGTCCTTCGATCCTCACAATTTCAGTCAACATCGTCCCTCCGACC
CTTCTAATCCCTCTAAGATGGTTCCAACTACCTATCATCCAACTCACAACCGTACTCTTCCACCTCCACA
TCAAGTGATAACTACGGAAGTAAAGAACATACTCATACGCAGCTTCTATCAGCGAGCTGAAGATAAGATG
AGACCAAAGAGGCCGGCTTCAGAACATCTGGCGGGTGAGCACGGTAACAAGCATTTTCGTGCTTCCTCAT
CATCTGCTCCGGGTTTATAA
Brassica rapa, cDNA
SEQ ID NO: 16
ATGGGGTCGATTCTGGGAGATTTGCCGTCCTTCGATCCTCACAATTTCAGTCAACATCGTCCCTCCGACC
CTTCTAATCCCTCTAAGATGGTTCCAACTACCTATCATCCAACTCACAACCGTACTCTTCCACCTCCACA
TCAAGTGATAACTACGGAAGTAAAGAACATACTCATACGCAGCTTCTATCAGCGAGCTGAAGATAAGATG
AGACCAAAGAGGCCGGCTTCAGAACATCTGGCGGGTGAGCACGGTAACAAGCATTTTCGTGCTTCCTCAT
CATCTGCTCCGGGTTTATAA
Brassica rapa, gDNA
SEQ ID NO: 17
CCACACATGTTCTTTTGGTACGTGGATACCTTCTCATGCAACAAATGATATGTTATTTTTGGGTGGTTTG
AATGATTTGTTTACCGATCTATAGTCATTTTTTTCTGTGTTTTATTCTTACCATCACAAGATCACTTTCT
GATACATTATTAATTTTAAAACTATTTTGTCCTCAAACGCTTAATCCTGAAATACTACTTCATCTCAAAC
ACTCAACTTTAAAAATTTATATGAATATATAACCGAAAGATAAGTATTTTAGTGTCAAAATCATTTTTAA
GCTTTTTCCATGCACTAATATATATAAAAAAAACCGAAATGTTATAATTTTGTGTTATAGTATAGTTATT
CGAACAACAAAAAATTTATAACTACCAATAAAATTTATAGATATATACCTTTTTAACTCTTAAAAATAAT
AAGGTTTATAACAAGTCATAAACTAAACCTTAAAAATCTAAATTGATAAGATATTCATATAAAATGTTTT
TTTCAGGCTGGTTCTAGAGTGAGCCCAGCGGAAAGCCCATATAAGTCTTTCTATTATCTCTACTTCCCTT
GCGATTCCGAGAAGGAAGATCGCAGAGCTCAAATTCCAAAATGGGGTCGATTCTGGGAGATTTGCCGTCC
TTCGATCCTCACAATTTCAGTCAACATCGTCCCTCCGACCCTTCTAATCCCTCTGTAAGTCCTTCCCTGA
TTTTTAACTGTAATCATTTTGAATTTGGCTAAAATCGGATCTGAGAATAATATGAATTGAGACTTGGTTT
CTAAATCTGATTCAAATATTGATATTTTCGAATTTTGTTTCAATGAGAGATTGGAAGAATGTTACTGGGG
GGCTTGTTTTGGGTTACATATTCATCTATTTTTTCTATTGGACCTTTGTGAGGAATCAAGAAAGTAATCG
TTTTTTTCTTCTAACCAGATTGATTGATCTTACATGTTGTTCCATTCTCATTTTGATTGCATGTTGTTGT
TAAATTAACAATCTTTTTTTTTGTATGATCTTACAGAAGATGGTTCCAACTACCTATCATCCAACTCACA
ACCGTACTCTTCCACCTCCACATCAAGGTATACCCAAAACAAAGTCTAACTACTTTACATAGTTTAACTT
TTGTCTTTATTGAGTGATCTTATTATAGACAACTTTGAAGGACTATCTTTACCAGGAATCACTTTTTTTT
TGTTCGTAGAGAGTATTGAGTCTTCATGGATTATAGATAGTTCATTCCTAGTTTGCTACGGTTTATATTC
TCTTCTTCTTTTTGTGTCAGTGATAACTACGGAAGTAAAGAACATACTCATACGCAGCTTCTATCAGCGA
GCTGAAGATAAGGTACTAAATTTCTAAAATTCAACATGTGCGGTATAGAACAAGTCCTCAAACTCTTTTC
TTTTTTTTTTTTGTTTTTTGCGAAAAGCAGATGAGACCAAAGAGGCCGGCTTCAGAACATCTGGCGGGTG
AGCACGGTAACAAGCATTTTCGTGCTTCCTCATCATCTGCTCCGGGTTTATAAAAAGCTTCTCCTGCTTC
CAAAGCCTGGCTATAATGGTCGTCACTTGTGCTACTCTTCTTATTAGTGTTTTTTTTACAAAGAATGCTT
TGAATGTAGAGAGAAAAGATGAGAGCAGCTCACTATGTGATTGCAGGGAAAATGTTGTATGAGTTATATG
TACATAACATTTATGTTTTTATTTTTAATTTTAAAATATTCGTCCAAATCACATTGTTAGCTTTTGTCTC
TTCTAGAATGTAAACTGAATTCTTTGTTTGCTTCCAACGAATTACAAGAGGAATCTGAGAGTAGTGGCAC
TCACTAGCCATAGTGATATCCCCGACTTTTTGTTCGCATGTTTCTCCTCACCTTGTAACGAGCACAAGTA
CTTGTTATACACTGCAGACCATTTTCCATGATTTATTTTTTGTTGATGATGTGGGTTTCTACAGGAGCTG
TTTCCTTGAAGTGOATCCTAAGCTAGATCCCTTAGTTGAAGAGGATGAGCGACACTATATCTGAATTCAT
TTGCAAGGGAAATGATGACCAGGTGATTGAATTGTGGTGTACAGACCATGGCAGAGAAAGAAGAAGAAGA
AGACGGCCTGGTGAAATCTAATG
Brassica rapa
SEQ ID NO: 18
MGSILGDLPSFDPHNFSQHRPSDPSNPSKMVPTTYHPTHNRTLPPPHQVITTEVKNILIRSFYQRAEDKM
RPKRPASEHLAGEHGNKHFRASSSSAPGL
Capsella rubella, CDS
SEQ ID NO: 19
ATGGCGTCGATTCTTGGAGATTTGCCTTCCTTCGATCCTCACAATTTCAGTCAACATCGTCCCTCTGATC
CTTCTAATCCCTCTAGGATGATTCCTACAACTTATCGTCCTACACACAACCGTACACTTCCACCACCAGA
TCAAGTGATAACTACGGAAGTGAAAAACATACTTATACGCAGCTTCTATCAACGAGCTGAAGAGAAGTTG
AGACCAAAGAGACCGGCTTCAGACCATCTGGCAGCCGAGCATGGGAACAAGCATTTCCGCGCTGCTGCGT
CTTCTTCGTCAACTACTCAGGGATTATAA
Capsella rubella, cDNA
SEQ ID NO: 20
TCTTGAACTCTGAAGAGAAGGAAACATTATCACGCACACCACGATTCCAATGGCGTCGATTCTTGGAGAT
TTGCCTTCCTTCGATCCTCACAATTTCAGTCAACATCGTCCCTCTGATCCTTCTAATCCCTCTAGGATGA
TTCCTACAACTTATCOTCCTACACACAACCGTACACTTCCACCACCAGATCAAGTGATAACTACGGAAGT
GAAAAACATACTTATACGCAGCTTCTATCAACGAGCTGAAGAGLAGTTGAGACCAAAGAGACCGGCTTCA
GACCATCTGGCAGCCGAGCATGGGAACAAGCATTTCCGCGCTGCTGCGTCTTCTTCGTCAACTACTCAGG
GATTATAAAAAACTTTCCCTGCTTCAAAGCCTTGTAAGTTGTAACAATGGTCCTTTGAATGTGTTCTTAT
TAGTGTTTTCAATGTAGAGAAAAAAGATAAGAGCAATTCACGAGTGGAATGTACATAACATTGATGTTTC
TTTTATATTTTAC
Capsella rubella, gDNA
SEQ ID NO: 21
TCTTGAACTCTGAAGAGAAGGAAACATTATCACGCACACCACGATTCCAATGGCGTCGATTCTTGGAGAT
TTGCCTTCCTTCGATCCTCACAATTTCAGTCAACATCGTCCCTCTGATCCTTCTAATCCCTCTGTTAGTT
CTTTCTTCCCCCTAATTCTCACTCTTTCATCTTCGACTTCTTTTTTTTTTAGTTCCTTTTCTTCGCTTCT
TAGATGAAAGTGCTTAGCTGTTCTATGATCTGTGTTTTGTCTGAATTCGTCTGAGAACTCTTCAAACTAT
TCAATTCTACGGATCTGAGTTCGTTTTACTTGGTCGTATTGTGAAAAAAATATGTATCTTTTGTTGATCA
AAATTTGATATTCAAATTGTTTAAAGAGTATACATTCGAATGTGTGTATTTGGTTTTGCTGGTTACTTGA
TCTGATCCAAAAGTTCTTCTTTTTCATTAATCTTGTGAGAAACCAAGAAAAGAAAAAAAGCCATTTTTGG
TCTTGCTGAATGATCTTAGAATCATAATTTTGATTCAATTGGCCTTAGTTAGCTCCTAAGTTATATGACA
AGATCTTGTTATCTCATATCAACTTACTAGTATGAAACTTTTCATTTTTATTTTATTTTGATTGAGCGAA
AGATTAGATTTTTGATCTTTAAATTAAAGATTTGAGCTTTGTTCATATTTTCTTGCAGAGGATGATTCCT
ACAACTTATCGTCCTACACACAACCGTACACTTCCACCACCAGATCAAGGTGAAACAAAGAATCGCGGTT
TTTTTAAATTTTGCATGTTCTTAGGCTAGAAGATTTTCGAATTTTGTTTACATTTTATAGTGTCTAAAAT
TGGCCTTTTGAAGTTGTGATCTTGGCTAGAGACTGATATTTGAACTTGCTTTGATAGAGCGATCTAGCTA
CTCTGTTTACCATGAATCACTTCTTAGATCTCTAAGGCAACTGGGAGTTAATCAGATTACAATAGTCTCG
AGTTCCCACACATCTACGCCTTTACTAATGTGTACCTTGTTTGTTTTGGTTGTAGTGATAACTACGGAAG
TGAAAAACATACTTATACGCAGCTTCTATCAACGAGCTGAAGAGAAGGTAAACCAACTCTCATTCGTCTT
TAAGTTCTTGATTCTTTTATCCAAATCATTTCCCTCATATAGTCTTATAACACAGGATTTCTTAGGAAAA
CAGACAAGAACGATATCGATCAAATCTTAAACTTCTTCAGATAACCAAACTGTTGGTTTTGATTGTTTTA
TCCTAAATCTCAGTTCCTTTTTTTTTTGTAATATTTATTATTTGCAGTTGAGACCAAAGAGACCGGCTTC
AGACCATCTGGCAGCCGAGCATGGGAACAAGCATTTCCGCGCTGCTGCGTCTTCTTCGTCAACTACTCAG
GGATTATAAAAAACTTTCCCTGCTTCAAAGCCTTGTAAGTTGTAACAATGGTCCTTTGAATGTGTTCTTA
TTAGTGTTTTCAATGTAGAGAAAAAAGATAAGAGCLATTCACGAGTGGAATGTACATAACATTGATGTTT
CTTTTATATTTTAC
Capsella rubella
SEQ ID NO: 22
MASILGDLPSFDPHNFSQHRPSDPSNPSRMIPTTYRPTHNRTLPPPDQVITTEVKNILIRSFYQRAEEKL
RPKRPASDHLAAEHGNKHFRAAASSSSTTQGL
Thellungiella halophile, CDS
SEQ ID NO: 23
ATGGCGTCGATTCTGGGTGATTTGCCTTCCTTTGATCCTCACAATTTCAGTCAACATCGTCCCTCCGATC
CTTCTAATCCCTCTAGGATGGTTCCAACTACTTATCATCCTACTCACAACCGTACACTACCACCACCAGA
TCAAGTGATAACTACCGAAGTCAAAAACATACTTATACGCAGCTTCTATCAACGAGCTGAAGAAAAGTTG
AGACCAAAGAGACCGGCTTCAGAGCATCTGGCGGGTGAGCACGGGAACAAGCATTTCCGTGCTTCATCAT
CTACTCAGGGATTATAA
Thellungiella halophile, cDNA
SEQ ID NO: 24
GAAGAAGAGAAGGAAACATCCGCAAATTCGAAATGGCGTCGATTCTGGGTGATTTGCCTTCCTTTGATCC
TCACAATTTCAGTCAACATCGTCCCTCCGATCCTTCTAATCCCTCTAGGATGGTTCCAACTACTTATCAT
CCTACTCACAACCGTACACTACCACCACCAGATCAAGTGATAACTACCGAAGTCAAAAACATACTTATAC
GCAGCTTCTATCAACGAGCTGAAGAAAAGTTGAGACCAAAGAGACCGGCTTCAGAGCATCTGGCGGGTGA
GCACGGGAACAAGCATTTCCGTGCTTCATCATCTACTCAGGGATTATAAAAGCTTTCAACCCTATAATAG
TCATGTTGTATGAGTCTTTTGTTAGTGTTCTTATTAGTGGGTTGTTTACAAAGTGAATGCCTTTAATGGT
AGAAAAAAAGATAAGAGCAGCTCTTGTAACAGGATTGTAGAGAAAATGTGTATCCTGAAACAGAGTGTTC
CTCATAGACTTTGGTGGAAACATGCTGTTTGTATTTTCCTGTACAGTTACATTCAAATATTCCTTTTGA
Thellungiella halophile, gDNA
SEQ ID NO: 25
GAAGAAGAGAAGGAAACATCCGCAAATTCGAAATGGCGTCGATTCTGGGTGATTTGCCTTCCTTTGATCC
TCACAATTTCAGTCAACATCGTCCCTCCGATCCTTCTAATCCCTCTGTATGTTCTTCCCTCTATTTTCAG
TCAAACACTCTCATTTTTTCGGTGAATTTGGGTAGAATCGGATCTGAGTTCGATTTAATTAGTTTTCCTG
AAAATTGTGGATTGAGTCTCAGTTTCCTTAATCTGGAATCTGATCCAATTTTGATCATGTTGGATCCTGT
GAGAAACCATCAAACCAAGAAAGTAGCAATTTTTTTTTCTCACCAGATTTGATTTCTGAATCAGAATTCT
GTTTCCACTGGCCTTAATTAGCTGTTTAATTACTTAATTCTGTTATCTCATATCAGATGAAAAGTTTTCA
TTTTTATTGCATATAACAGTTGAATTTAAAAGAGCTTAAAGATTAGGAATTTTTTTTAATATCTGTTATC
GAAGATTTTGAGTTTTTGTTCAATGTTTTTTTTTTTTGAAACAGAGGATGGTTCCAACTACTTATCATCC
TACTCACAACCGTACACTACCACCACCAGATCAAGGTAAAATAAAAAAGTCACCTTTTTTTTGGTCTTGC
CTTCTTCAGTAACTACGTTATATATAGTCTACATGTGGCCTTGAACTTGTGATGTTGGCCAGAAACTGAT
CCTGAACTTGTATTAAGTGAGTTATCTGATTATGAAGAACTATCTTTCACATGATTCACTTCTTAGAATA
GACTCGGAGATTTGAACTCGTGACAAATGGGTGCTAATCAAATACAATATAGTCTTGAGTTTTATGGATA
GCTCATTGCTGATTTTCTAGATTTCATCTCTTTATTCCGTTGATTTTTTGTGGCAGTGATAACTACCGAA
GTCAAAAACATACTTATACGCAGCTTCTATCAACGAGCTGAAGAAAAGGTGATAGAGATTCAATCCTCAA
AGTTTCTTAATTTTTTTTATTTATTTGAATCGTTCATCCTCCTCGTAGTTCATAACAAAGCTAGTTTCTT
AGGAAGAAATTTGTTTTACCGTGTGAGATATAGAACAAGTGTTAAACTCTGTTTTTTGTGGGTCAAATTT
TGTCATTTGCAGTTGAGACCAAAGAGACCGGCTTCAGAGCATCTGGCGGGTGAGCACGGGAACAAGCATT
TCCGTGCTTCATCATCTACTCAGGGATTATAAAAGCTTTCAACCCTATAATAGTCATGTTGTATGAGTCT
TTTGTTAGTGTTCTTATTAGTGGGTTGTTTACAAAGTGAATGCCTTTAATGGTAGAAAAAAAGATAAGAG
CAGCTCTTGTAACAGGATTGTAGAGAAAATGTGTATCCTGAAACAGAGTGTTCCTCATAGACTTTGGTGG
AAACATGCTGTTTGTATTTTCCTGTACAGTTACATTCAAATATTCCTTTTGA
Thellungiella halophila
SEQ ID NO: 26
MASILGDLPSFDPHNFSQHRPSDPSNPSRMVPTTYHPTHNRTLPPPDQVITTEVKNILIRSFYQRAEEKL
RPKRPASEHLAGEHGNKHFRASSSTQGL
Glycine max 1, CDS
SEQ ID NO: 27
ATGGATTCTCTGATTGGTAATTGGCCATCCTACGATCCTCACAACTTCAGTCAGCTTCGACCTTCCGATC
CTTCTAGTTCTTCTAAAATGATGCCGGCCACTTACCATCCTACTCACAACAGGACTCTTCCAGCACCTGA
TCAAGTGATAAGTACTGAAGCCAAAAACATCCTCATGAGACATATTTATCAGCATTCTGAGCAGAAGTTG
AATCCAAAAAGAGCTGCATCTGATAACCTTCTTTTACCAGAGCATGGATGCAAGCAACCTAGGGTTTCCA
GCTGA
Glycine max 1, cDNA
SEQ ID NO: 28
GGATTTTTCAGCTTTTCATTTTGCCCCACCTTCTCCTTTCATCTCACAATCATAACTTGAGTTGAGCACG
TTCCCGAGACATCTCAAATTTCCTCTGCTGAGAATTTCACAAGTTTATGAGCCACAAGTGCAAATGGGAA
TGAAATGAAGAATGGGGTTTAGAGTTAGTCAAGACAAGTGGTOTGGTGTCCTATGTTATGACTGCACACA
TGTGAAGTGAAGTAGAGACTATTCAGTCCACAGCAGCTGTTTCTAGTGTGTGTGTCATTGCATTCCTCAT
CCTTTTCCTCTTTTTICACGCCTTAATTTCTTTCTCTCTTTCTCCCTCTTCCTCTCTGGAATTTGGAGCA
TCAGCCAGCACTCTATGGATTCTCTGATTGGTAATTGGCCATCCTACGATCCTCACAACTTCAGTCAGCT
TCGACCTTCCGATCCTTCTAGTTCTTCTAAAATGATGCCGGCCACTTACCATCCTACTCACAACAGGACT
CTTCCAGCACCTGATCAAGTTCCAACAGTGATAAGTACTGAAGCCAAAAACATCCTCATGAGACATATTT
ATCAGCATTCTGAGCAGAAGTTGAATCCAAAAAGAGCTGCATCTGATAACCTTCTTTTACCAGAGCATGG
ATGCAAGCAACCTAGGGTTTCCAGCTGACACTTGCCCGTTTCCATTGACCTTTGTGATCGTGAGCAAGTT
TCCAAAAGATCAGAACTTACAAGAGTGAACTTACAAGAGTTTGGTGCTTGTTTGGAACTCTGAACTATTT
TGGCAGCTATATAGACTTGGGAGCGTGTGAAAATAAACCCTGTTAATGCCCATCAAAGTTTACCATGAAT
GAAATAATGATATGCTTTTGTTGTTTATTTTTGTGTCATGCTTGTTGTACTCTACCCACAACTGATTGTC
CACAATAGTTGGGAAGAGATAAGTGCTTGATTGAGGATTTTCAAAATCTATCTATCTTTTGGACTCAGGA
GCATATTCTGGGGCCATAATGTTCTCCATCTAACTACAATTATTTATATGGCGCTTTT
Glycine max 1, gDNA
SEQ ID NO: 29
GGATTTTTCAGCTTTTCATTTTGCCCCACCTTCTCCTTTCATCTCACAATCATAACTTGAGTTGAGCACG
TTCCCGAGACATCTCAAATTTCCTCTGCTGAGAATTTCACAAGGTAATGTCTCACACTCACACCGATTTT
TTGCACCCAACTTTTTGTGCTGTGTTTAGATTCTGATTATTTCTCACGCTTCCACATATGCCCCCATTGC
TGTTTTCCAATTGGTTTCTCCTACCAGTACCAGGTGGTTTATTCAACCAATAGTGGATCTTCAATTTATT
GGTTTCAGTGTTCTTGTATGTGTGTGTGTTTTCATTTTGATGAATTCATGGTAAGCCTAATAATATGTAT
TCTTTATTAATTGATAATTAAAGTTCAGATTTTTGGTGAATTTTCATACATTGGGAAGATAGTTTTAGGT
AATTTTCTCATTGTTTTTAAAAGGGTGTTCACTTGTCCCTTTTCCATGAATTGCAGTTTATGAGCCACAA
GTGCAAATGGGAATGAAATGAAGAATGGGGTTTAGAGTTAGTCAAGACAAGTGGTGTGGTGTCCTATGTT
ATGACTGCACACATGTGAAGTGAAGTAGAGACTATTCAGTCCACAGCAGCTGTTTCTAGTGTGTGTGTCA
TTGCATTCCTCATCCTTTTCCTCTTTTTTCACGCCTTAATTTCTTTCTCTCTTTCTCCCTCTTCCTCTCT
GGAATTTGGAGCATCAGCCAGCACTCTATGGATTCTCTGATTGGTAATTGGCCATCCTACGATCCTCACA
ACTTCAGTCAGCTTCGACCTTCCGATCCTTCTAGTTCTTCTGTAAGTTGCTGTTGTTGTTGTCTATGAAA
TTGATAATCCTGGTAATAATTACTCATTCCAAGTATGCAAATGTATGCTTTGTTAGAGCATGTTTGGTTT
TCTGTTGCAAAAGTATCGTTTGAGGCAAAATTAATTTTGTTAACGAACCAAGACCCTTGCTTTTGCGTTG
AATTTGCATTGGAATGTGTGTCAACATTTCCAACTGAAAACCAAACATGCACTCAATTGTTGAAAAATCT
GATTGTGTATGATTCTGAGTTTTTGGTTTACTACTATCTATTTGCATTGATCCCGGTTGAAAGATTAGTG
AAGGAAGGGATATTAGCCTCTTTTTCTTAATTTCCCAACCCCCTTGAGGACAAAATTTGGTTGTTGTCCA
TAAATATAAGAACTTCTTCTACTTGCAACTTTGAATCCCACTATTCAAATTTGAGAATATTTGTTAAATT
TTTAAGATGGTACATGGTAGAGTCAGACATTAGAGATGTGTAGATTAGAAAACCTGTATTTGCAATCTTT
AGAATTTTATGTTTGCTTCTTTTTTGTCAAAAGGTAGAGTATTTGTTGACATTCTAACTGTTATTACTAC
TTGGATGGTCATAATTCACAATAGAGTAACAGCATTGTGCAATTTCTTGTGTTGAAGTCTTCACCATTGA
TGTTATATGCAGCATTGTCTGTGGGAACATGTCCATTTAATCTCAATAACTATACTGTTTTTCTTTCTTC
GCTATTCTGCAAAGTTTCTGTAAAGGATTTTAAAGTTTGAAACTTTTCCAGCATGGGATGATTTTCTTTT
TCCTTTTTTGTTTCTTGTCATTTTTTACTCTGAAAGTAGTACTGTATAACTTGGCTTTCTGTTCAATATT
GTAACAACAATTATCTTCAGTGACAAAAGTGTTTTACTTTTATTTCTAATTAGTTGAAAAAACTAGAGTT
ATATGTAATTTGTCCTTTACAGCATATCAGTTTCCATGATAGCCTGACACAAATTTAGTTGTTTTCACAG
TACCTTCTGATGAATGTAGTCGTTCTTGTCTTTACAATAGATTTTCTAATTTACTCTATTATTGCTAATC
TATAAAACTACAAGCAAATACATATTGCCCATGAAATTTGAAAATGAAGGAGACCATTGGATTCATTTGA
TACCTTTGCTGAAGCGTACAAGTATGTTTACACCATACATACAAATTCTGGTTTTATGTTTTTTAACAAT
AGATAACGTGTTTCTTCTCTAGTTGATTACATTTGATAAGAAGGAAGCTCAAGTATGAGTATGACAGATT
TGATCTTGGAGATAAACATACTGAATGGGTCACTGTTATTCCTTACCTTCTTGCTAACCAAGTCATGAAT
AAACCAAGCATATTTAAGGCCAACTTCTTTTTATGAAACTTGCTACTCATTTTGTTCTAAAATGTATTAA
GGATCACCTAATGATATATTTTCATTCACACTTTGAAGATGAAATTTTTATTTTAACACAGAAAATGATG
CCGGCCACTTACCATCCTACTCACAACAGGACTCTTCCAGCACCTGATCAAGGTAGTCAGGATAACTTTC
TTCTACTAATGCTTTGCTCTGTATTTATTTGATTGATAATCTGTTGAAAAACTGTATTATTCCTTTGGAT
TATTCCCTTCATCATGTTTTTGGAGGAGTATCCAAACTGGATGCATTAAATCTATTAATTTTTTGTTTGC
CACACACAAGTTTATAGTAGAGCTTCTCCAATGTATATGAACTCAAATTGGGTTCTTAACTCTCACATAG
TGGGTGCCTGTTGCCACATAGGATTTAAGAACACTCACTATTTTCACTCCGAAGTTAAAACTCAACTTGA
GTTCTTAACTTATTTTTTAGTCTCATCTTATCTCTCTGTTTACACATGGACCCCATTTTGACTTGGAAAG
TTATTATTTTAATGGTTAAGCAACCTTTTTGTAGAAGGGTTCTTCTATAATAACTCTAGTTCATTTTCTC
CAATGTTATTTTTCAAGTTGCTTAACTTTTGTTTTTTTAATTTCGGTAGACTCAATGGTGTTTTCTAATT
CAAAGTTTGCTAACCTGTTGAAGTTCCAACAGTGATAAGTACTGAAGCCAAAAACATCCTCATGAGACAT
ATTTATCAGCATTCTGAGCAGAAGGTTAGTGACTTGATGGTTAAAGAGCATGTGTTTTGGTGCAGTTAGG
TATGCATATGCTTGATGCTCATAACCTTTTGTGTTTTTCAGTTGAATCCAAAAAGAGCTGCATCTGATAA
CCTTCTTTTACCAGAGCATGGATGCAAGCAACCTAGGGTTTCCAGCTGACACTTGCCCGTTTCCATTGAC
CTTTGTGATCGTGAGCAAGTTTCCAAAAGATCAGAACTTACAAGAGTGAACTTACAAGAGTTTGGTGCTT
GTTTGGAACTCTGAACTATTTTGGCAGCTATATAGACTTGGGAGCGTGTGAAkATAAACCCTGTTAATGC
CCATCALAGTTTACCATGAATGAAATAATGATATGCTTTTGTTGTTTATTTTTGTGTCATGCTTGTTGTA
CTCTACCCACAACTGATTGTCCACAATAGTTGGGAAGAGATAAGTGCTTGATTGAGGATTTTCAAAATCT
ATCTATCTTTTGGACTCAGGAGCATATTCTGGGGCCATAATGTTCTCCATCTAACTACLATTATTTATAT
GGCGCTTTT
Glycine max 1
SEQ ID NO: 30
MDSLIGNWPSYDPHNFSQLRPSDPSSSSKMMPATYHPTHNRTLPAPDQVISTEAKNILMRHIYQHSEQKL
NPKRAASDNLLLPEHGCKQPRVSS
Glycine max 2, CDS
SEQ ID NO: 31
ATGGATTCTCTGCTTGGTAATTGGCCATCCTTTGATCCTCACAACTTCAGTCAGCTTCGACCTTCCGATC
CTTCTAGTTCTTCTAGAATGACGCTACCCACTTACCATCCTACGCACAGCAGGACCCTTCCAGCACCTGA
TCAAGTGATAAGTACAGAAGCCAAAAATATCCTCGTGAGACACATTTATCAACATGCTGAGGAGAAGTTG
AAACCAAAAAGAGCTGCATCTGATAACCTTTTGCCTGATCATGGATGCAAGCAACCTAGGGTTTCTAGTT
GA
Glycine max 2, cDNA
SEQ ID NO: 32
GTTGTAAGGGTGGCTGGAATTTTCAGCTTTTCATTTTGCCCCCACCTTTTCCTTTCATTTCACTATCATT
ACTTGAGTTGAGCACGTTCCCGAGACATCTCAAATTTCCTCTGCTGAGAATTTCACGAGTTTATGAGCCA
CAAATGCAAGTGGGAACGAAGAATGGGGTTTGGAGTTAGTTTGGACAAGTGGTGTGGTGTGGTGTCCTAT
GTAATGAGTGCACATATGTGAAGTGAAAATTCCTCATCCTTTTCCTCTTTTTTCACGCCTTAATTTCTCT
CTCTCTGGAATTTGGAGCAACAGCCAGCACTCTATGGATTCTCTGCTTGGTAATTGGCCATCCTTTGATC
CTCACAACTTCAGTCAGCTTCGACCTTCCGATCCTTCTAGTTCTTCTAGAATGACGCTACCCACTTACCA
TCCTACGCACAGCAGGACCCTTCCAGCACCTGATCAAGGTAACACGACGAACTTTCTTATGATTCCTCCA
GCAAATTAGCCATGCTTTGTCCCGTATTTACTTGACTGATCATTTTTTGGAAAAATGTATTTTTCCTTTG
GATTATTACCTTCATCATGTTTTTGGAGGAAAATCCAAACTACATGCATTAAACCTGTTACTTAAACTGA
TTTTCTGTCTAGCTCAAGTCTTGTTAGGGTAGACTGACTGGTGTTTTGTAATTGAAAGTTTTGCTTGAAA
ATTGTTCAAACAGTGATAAGTACAGAAGCCAAAAATATCCTCGTGAGACACATTTATCAACATGCTGAGG
AGAAGGTTAGTGACTCTTGATGGTTAAACACACATGTGTTTTGGTGCAGTTAGGTTTGCACATGCTTGAT
GCCCATAACCTTTTGTATTTTTTCAGTTGAAACCAAAkAGAGCTGCATCTGATAACCTTTTGCCTGATCA
TGGATGCAAGCAACCTAGGGTTTCTAGTTGACAGTGGCCATCAACCAACTGTTGTATGTGATTGTGAGTA
AGTTTCCAAAAATATATCCTTAGAAGAGTTTGGTGCTTGTTTAGAATTTGAACGATTTTGGCCAGTTATA
TAGACTTGGGAGTGTGTAAACATAAACCTAATAATCAGTCAAAATTTAAACTGAATGAAACAATGACGAT
TTCATTGTGTATGTTTATGCCATATCATTAACAACTGATCGTGCAAGTATCTTTTGTACTCGC
Glycine max 2, gDNA
SEQ ID NO: 33
GTTGTAAGGGTGGCTGGAATTTTCAGCTTTTCATTTTGCCCCCACCTTTTCCTTTCATTTCACTATCATT
ACTTGAGTTGAGCACGTTCCCGAGACATCTCAAATTTCCTCTGCTGAGAATTTCACGAGGTAATGTCTCG
CACTCACCATTTTTTTGCAGCCAACTTTTTTACTGTGTTTGGATTCTGATTATTTCTCACGCTTCCACAT
ATGCCCCTATTGCTGTTTTCCAATTTGGTTTCTCCTACCAGTATCAGGTGGTTTATTCAACCAACAGTGG
ATCTTCAATTTGTTGGTTTCAGTGTTCTTGTATGTGTGTTTTCATTTTGATGAGTTCATGGTGAGCCTAA
TAATCTGTATTCTGTATTAATTTGATAATTAAAGTTCCAATTTTTAGTGAATTTTCATACATTGGAAAGA
TAGTTTTAGGGAATTTTCTCATTGTTTTTCAAAAGGGTATTATTCACTTGTCCCTTTTCCATGAATTGCA
GTTTATGAGCCACAAATGCAAGTGGGAACGAAGAATGGGGTTTGGAGTTAGTTTGGACAAGTGGTGTGGT
GTGGTGTCCTATGTAATGAGTGCACATATGTGAAGTGAAAATTCCTCATCCTTTTCCTCTTTTTTCACGC
CTTAATTTCTCTCTCTCTGGAATTTGGAGCAACAGCCAGCACTCTATGGATTCTCTGCTTGGTAATTGGC
CATCCTTTGATCCTCACAACTTCAGTCAGCTTCGACCTTCCGATCCTTCTAGTTCTTCTGTAAGTTGTTG
TTGTATATGAAATTGATAATCCTAGTAATAATTACTCATTCCAAGTATGCAAATGTATGCTTTCTCAATT
GTTGAAAGTCTGATTGTGCGATGCTGAGTTTTTGGTTTAATGCTTTCTATTTGCATCGATCCCCCGTTGA
AATATCAGTGAAGGAAGGGATATTTGCCTTATTTCCCAACCCCCTTGAGGACAAAATTTGGTTGTTAACA
ACCTTCTTCTTGTTGACACACTTGCATATTTGAATTCCACCATTCAAATCTGAGAATATTTCTTCATTTT
TTAAGATGGTAAGTGGTAGAGTTAGACTAGAGAAGTGTAGAATAGAAAACCTGTATTTGCAATCTTCAGA
ATTTTAAGTTTGCTTCTTGTTGTCAAAAGGTAGAGTATTTGTTGACATTCTAAATGTTATTGCTACTACT
TAGAATGATCATAATTCATAATAGAGTAACAGCATTGTGCTATTTGTTGTGCCCGAGATTGAATTCTTCA
CCATTGGTGTTATATGTAGCATGTATGTGGGAACATATCCATTTAATCTCAATAACTTTACTGTTATTCT
TTTTTTATTTTTCCTCTTTATTTTGTGCGGTTCTGCAAAAGTTTCCGTAAAGGATTTTAAAGTTTGAAAC
TTTTCCAGCATGGGGTGGTTTTCTTTTTCCTTTCTGTCTCTTTTAATTTGTGACTCTGAAAATTGTACTG
CAAAACTTGACTTTGCTGTTCAATATTGTAGCAACAATTAATTCTTCAGTGACATAATTGTTCTACTTTT
ATTTCTAATCAGTCTCCGTGTTACCTTGACACAAATTTAGTCGTTCTTGTCTTTTCCATAGATTTGCTAA
TTGCTAATCTACAAACTTACAAGCACATACATATTACCCATGAAATTTAGCGGTTGAAAATTGAAAAAAT
GAAGGAGATCATTGGATTCATTTGGTACCTTTGCTTACACCGTGCATACAAATTCTTGTTTTATGTTTAA
CAATATGTGATATGTGATATGCTTCTCTAATCCATTGCATTTGATAAGAAGGAAGCTCAAGCATGAGTCA
GCTTTGATCTTGGAGGTTAACACACTGAATGAGTCACATTTATTCCTTACCTTTTTCTAATCAAGTCTGG
GATAAACCAACATATTTAAGGCCAACTTCTTTTTATGCAACTTCTATTCTTGTTCTAAAGTGCATCATGG
GTCACCTAATGATATATAATTTTATAAGCGTTATGAAGATGAAATTTCTCTAATTTCATACAGAGAATGA
CGCTACCCACTTACCATCCTACGCACAGCAGGACCCTTCCAGCACCTGATCAAGGTAACACGACGAACTT
TCTTATGATTCCTCCAGCAAATTAGCCATGCTTTGTCCCGTATTTACTTGACTGATCATTTTTTGGAAAA
ATGTATTTTTCCTTTGGATTATTACCTTCATCATGTTTTTGGAGGAAAATCCAAACTACATGCATTAAAC
CTGTTACTTAAACTGATTTTCTGTCTAGCTCAAGTCTTGTTAGGGTAGACTGACTGGTGTTTTGTAATTG
AAAGTTTTGCTTGAAAATTGTTCAAACAGTGATAAGTACAGAAGCCAAAAATATCCTCGTGAGACACATT
TATCAACATGCTGAGGAGAAGGTTAGTGACTCTTGATGGTTAAACACACATGTGTTTTGGTGCAGTTAGG
TTTGCACATGCTTGATGCCCATAACCTTTTGTATTTTTTCAGTTGAAACCAAAAAGAGCTGCATCTGATA
ACCTTTTGCCTGATCATGGATGCAAGCAACCTAGGGTTTCTAGTTGACAGTGGCCATCAACCAACTGTTG
TATGTGATTGTGAGTAAGTITCCAAAAATATATCCTTAGAAGAGTTTGGTGCTTGTTTAGAATTTGAACG
ATTTTGGCCAGTTATATAGACTTGGGAGTGTGTAAACATAAACCTAATAATCAGTCAAAATTTAAACTGA
ATGAAACAATGACGATTTCATTGTGTATGTTTATGCCATATCATTAACAACTGATCGTGCAAGTATCTTT
TGTACTCGC
Glycine max 2
SEQ ID NO: 34
MDSLLGNWPSFDPHHFSQLRPSDPSSSSRMTLPTYHPTHSRTLPAPDQVISTEAKNILVRHIYQHAEEKL
KPKRAASDNLLPDHGCKQPRVSS
Phaseolus vulgaris, CDS
SEQ ID NO: 35
ATGGAGTCTGTACTGGGTAATTGGCCGTCCTATGACCCTCACAACTTCAGTCAGCTTCGACCTTCCGATC
CTTCAAGTTCTTCTAAAATGGCACCGGCCACTTACCATCCTACTCACAGCAGGACCCTTCCACCATCTGA
TCAAGTGATAAGTACTGAAGCCAAAAATATCCTCCTGAGACATATCTATCAGCATGCTGAGGAGAAGTTG
AAACCAAAAAGAGCAGCACCTGATAACCTTTTACCAGAGCATGGATGCAAGCAACCTAGAGTTTCCAGCT
GA
Phaseolus vulgaris, cDNA
SEQ ID NO: 36
CGGAGGTGATGAGTAGCTCCAAATGATGATCAGTTGGTAATGGTGGCTGCAATTTTCAGCTTTTCCTTTT
CCTTTTCTTTCACTTCTCAACCAAACCATAACATAACTTAACTTAACTTTATCACATTCTTCATAGATCT
GAAATCCCTTCTCAGAATTTCACAGGTTTACCAGCATCCTGTGCAAGTGGGAATGAAGAATTGGGTTTAG
AGTTAGGACAAGGGGTGTGGTGTGGTATCCTATGCAATTGGTGCACACATGTGATGTGAAGTTCAGTCCA
CAACAGCTGTTTTTGGATTGGGTTTTGTGTTGTGTGTCATTGTCTTCCTCATCCATTTCCTCTCTTTTTT
CACGCCTTAATCTCTCTCTCTGAAATTTGGAGCAGCAACCGCCACTCTATGGAGTCTGTACTGGGTAATT
GGCCGTCCTATGACCCTCACAACTTCAGTCAGCTTCGACCTTCCGATCCTTCAAGTTCTTCTAAAATGGC
ACCGGCCACTTACCATCCTACTCACAGCAGGACCCTTCCACCATCTGATCAAGTGATAAGTACTGAAGCC
AAAAATATCCTCCTGAGACATATCTATCAGCATGCTGAGGAGAAGTTGAAACCAAAAAGAGCAGCACCTG
ATAACCTTTTACCAGAGCATGGATGCAAGCAACCTAGAGTTTCCAGCTGACACATGTCATTGACCATATG
TTGCATGTGATTGTGAACTACTTTCCTATAGATATACCCTTATTTTTCAAGAGAGTTTGGTCCTAGTTTC
AAATTGTGAACTATTTGCCAATTATACACTGGGGAGTTTTGTAAATACAAAGCCTGTTATTGCCCATCAA
AATTTACAGTGAACGATATTTTTGTGCCATGCCTTATTGTGCTAGACAGGTAACAACTGATTGTCCACAT
TAGTTAGGAAGAGATTCGTGCTTTAGTTAAAGATTTTCAAAATGCATCTGAGTCTTTTGGACTCAGGAGT
ATGCTTGTGCCATA
Phaseolus vulgaris, gDNA
SEQ ID NO: 37
CGGAGGTGATGAGTAGCTCCAAATGATGATCAGTTGGTAATGGTGGCTGCAATTTTCAGCTTTTCCTTTT
CCTTTTCTTTCACTTCTCAACCAAACCATAACATAACTTAACTTAACTTTATCACATTCTTCATAGATCT
GAAATCCCTTCTCAGAATTTCACAGGGTAATTTATTGTCTCACACTCACCAATTTTTCTACTGTCTTCCG
ACTCAGATTATTTCTAACGCTTACACTTCTCCTGTTAGTGTTTTTCCAATATCTGCTTCATTCAACAAAT
ACTGGATCTTCAATTTTTTTGTTTTCAGTGTTCTTGTATGTTTGTGTTTTCATTTTGACGACTTCATCAG
TGAGCCTGTGTATTGATTCATAATCTGATATAGTTCAGAGTTCTGGTGAATTTTATTTCTCTTGCATTGG
GAAGATGATGTTAGGGATTTTTCTCCTTTTTTCTTTAATTGGAATTCACTTACCCCTTTTTCTTAATTTG
CAGTTTACCAGCATCCTGTGCAAGTGGGAATGAAGAATTGGGTTTAGAGTTAGGACAAGGGGTGTGGTGT
GGTATCCTATGCAATTGGTGCACACATGTGATGTGAAGTTCAGTCCACAACAGCTGTTTTTGGATTGGGT
TTTGTGTTGTGTGTCATTGTCTTCCTCATCCATTTCCTCTCTTTTTTCACGCCTTAATCTCTCTCTCTGA
AATTTGGAGCAGCAACCGCCACTCTATGGAGTCTGTACTGGGTAATTGGCCGTCCTATGACCCTCACAAC
TTCAGTCAGCTTCGACCTTCCGATCCTTCAAGTTCTTCTGTAAGTTGTTGTAGTATAAGAAATTGATAAT
CCTGGTAATAACTCATTTTGATGCAAATGTATGCTTTGTCACTTGTTGAGAAATCTGATTGTTTGTGATT
CTGAATTTTTGGTTTACTACTGTCTAATCTACAATTATTTGGGTTGGAAATTTAGGGAAGAGACAATTGT
CTTGTTTTTTATTCTTATCCCTCTTCTCCCTTCAGGACAAAATTTGGTTGTTAACCTTCAAGATAAGACT
TTCTTCTAGTTAACACACTTCCATATTTGAATCCCATCTTCTAATTTTGAAACTGTTCGTCAATGTTTTA
AGTTGGTACATGGTAGAGAAGTTATAGTGTAGAAGAACATGTATTTGCAATCGTTACATTTTTAGTTTGT
TTCTTTTTATCGCAAGGGACAGAGTATTTCTTGACATTCTGATTGTTATTACTAATTCATAATAGGCTTA
TGGCATTGTGCAATTTGTTGTTCACCTGACGTTGAAGTCTTCACCATTGATGTTATATGCCGCTTTGTCT
TTGGGGACATATCCATTTAGTCTCAATAACTTCACTGTTTATCCTTTTTCCCCCTCTACATCTTTTTTGC
ATTCTGCAATATATTCATAAATTTGAAAGTTTTAAACTTCTCAAGCATGGGATGATTTGCTGTTTCCTGT
TGTTTCTTGTTCTTTTTCACTCTAAACATAATGCTGTTAAATTTGGTTTTCTTGTTCAATATTGTAGCAA
CAATAAATTCTTCTATTTAAAATAACTATTTCACTTTTTTAAAAATAGTTAGAAAGGCTAGTTTTATACG
TAATTTGTCCTTTATATTATAAGAAATATCTTTCTAGCTTTCAGGCATATATTAGTTTCCATTAGCCTGA
CACAAATTTAGTTGCTTCAAAATACCTTTTGCTGAATGTAGTTTTTCTTTTCCTTTCCAAAGATTCTTAG
ATTCGCTAATCCATAAAAGTATGATTCCACTATACGTACATGCATACATAATTCTTGTTTTATGTTAAAC
AAAGGTAAGATGTTTCTCTATTCAATTATATTTGATAAGAAAGAAGCTCAAATAAGAGAATGACTGGTGT
GATATCGAAGATAAACACACGGAATGAGCTACTGTTATTCATTTCCTTTTTCCTAACCAAGCCATGGATA
AACAAAGCAGTTTTAGGGCCAACTTCTTTGTGCATACATGGATTGAAGTTTGCAACTTAAGTTTGAATGA
ACTTTATGTTACGAACTAAGTTGGTAAAACAACTTTGTCTTTTCTTCCATTTCAAACTAAATTTCAATTC
AGACTTGAGGCAAAACTCAGTTTACAATCCTACAACCTGAAAATCAAGCAAGAGATTTTCTCAGATTTAG
TTTTTGAATGCATGTTAGGGGTCTCTCAACTGAAAACTAAACGTGCCTTTTTTGTACCTCTGGCACTCTT
ATTTTTTCTGAAGTGTACTATGGATCACTTAATCATATATTTTCATTTGCTCTCTGAAGATGAAACTTCT
ATTTTCATGCAGAAAATGGCACCGGCCACTTACCATCCTACTCACAGCAGGACCCTTCCACCATCTGATC
AAGGTAGTCAGTATAACTTTTTCTACTTCCGTGGCAATGCTTTGCTCTGTTTTTATCTGATCGATAATCC
GTTTGAAAAATGTATTTTCCTTTAATCCTGATCTTGGAGGAGGAGCCAAACTAGATGCATTAAACCTATT
ACTTTAACTTATTTTTCTCTGTTTGCCACACACAAAAAGTTTTGTGTATATAGAAAGGTAACAAACATGA
AAAACATTGGTTGCAGGTTTTTAGTTTTTCTTTTTTTGACAGATGTACATCCCTCATTTTCTGTCACTCT
TGTTTTTAATTTGGGTAGAGTTATTACTGGGTGTTTTGTAACTGAGGGTTTGCTAACTTGCTGAAATTTG
TTCTGACAGTGATAAGTACTGAAGCCAAAAATATCCTCCTGAGACATATCTATCAGCATGCTGAGGAGAA
GGTTAGTGACTCTTGAAAGTTAAATCCAATATGTTTTGGTACAGTTAGGTCAGCACATGGTTGTACACAT
TCCTACCCTCCACTCATCAGCCCACAGGGATCTAATAGATGTCAATGATCTTTCATCTTTTCAGTTGAAA
CCAAAAAGAGCAGCACCTGATAACCTTTTACCAGAGCATGGATGCAAGCAACCTAGAGTTTCCAGCTGAC
ACATGTCATTGACCATATGTTGCATGTGATTGTGAACTACTTTCCTATAGATATACCCTTATTTTTCAAG
AGAGTTTGGTCCTAGTTTCAAATTGTGAACTATTTGCCAATTATACACTGGGGAGTTTTGTAAATACAAA
GCCTGTTATTGCCCATCAAAATTTACAGTGAACGATATTTTTGTGCCATGCCTTATTGTGCTAGACAGGT
AACAACTGATTGTCCACATTAGTTAGGAAGAGATTCGTGCTTTAGTTAAAGATTTTCAAAATGCATCTGA
GTCTTTTGGACTCAGGAGTATGCTTGTGCCATA
Phaseolus vulgaris
SEQ ID NO: 38
MESVLGNWPSYDPHNFSQLRPSDPSSSSKMAPATYHPTHSRTLPPSDQVISTEAKNILLRHIYQHAEEKL
KPKRAAPDNLLPEHGCKQPRVSS
Medicago truncatula, CDS
SEQ ID NO: 39
ATGGATTCTGTCCTTGGTAATTTGCCATCTTATAACCCTCACAATTTCAGTCAGATTCGACCTTCAGATC
CTTCTAGTTCTTCTAAAATGACAATAACTACTTACCATCCTACTCACGACAGGACCCTTCCACCACCTGA
TCAAGTGATAAACACTGAAGCAAAAAATATTCTCCTAAGACATATTTATCAGAACGCTCGGGAAAAGTTG
AAACCAAAAAGAGCTGCAGCTGGTAACCTTTTACCAGAACATGGATGCAAGCAACCTAGGGTTTCCACCT
GA
Medicago truncatula, cDNA
SEQ ID NO: 40
TTAACGAGTTCATGTGTAGGATACAGTTGGTTTTGGTGACCAGGTTAAACGGGTCGGATTCGTGAAAGTG
GATCATTGATTGGAGGGTAAACCTTACTTGGTCATTTCAGTCCTTAGTGGTCTAGTGTTTTTTTCTCTTC
TTAACTCGCGGTTGTAACAGCAGTATGAATACTCACCCCTGGTAAAAATGATCGTAACTACATAGCTGGC
CGAAAGAGCAAAAGTTTTCATCTTTTTTCTCTCATGTTGAGGAGGACAACGTTCCAGAGAGATCTCAATA
ACTAATTCATAATTACTCCACTAGGGTAATATTGCCTAACGCTTATTCATGTTCATGATTTTTCAATTTT
TTTTTCTCACTCTTTTGATTTGTTTTGTGCCTTTGAAATTTTGATCAAATAGGTAGCAATTCATGGATTC
TGTCCTTGGTAATTTGCCATCTTATAACCCTCACAATTTCAGTCAGATTCGACCTTCAGATCCTTCTAGT
TCTTCTAAAATGACAATAACTACTTACCATCCTACTCACGACAGGACCCTTCCACCACCTGATCAAGTGA
TAAACACTGAAGCAAAAAATATTCTCCTAAGACATATTTATCAGAACGCTCGGGAAAAGTTGAAACCAAA
AAGAGCTGCAGCTGGTAACCTTTTACCAGAACATGGATGCAAGCAACCTAGGGTTTCCACCTGACAGTGT
TCATTGACCAACTAGTGCATGCAGTTCTCAGCTACTTTCTCGAATGATATATACTCTTATTTTATTACCA
AGATTTTGGTGCTTGTTTGAAATTGTAAACTATTATTAGTCCGCTACATACTTGGAGTGTGTAAATTTGA
CAACTCCCCCACCAATCAATCAATATATACAACAACTGAAATTATCATGCTTTTATTGTGTATATTTTT
Medicago truncatula, gDNA
SEQ ID NO: 41
TTAACGAGTTCATGTGTAGGATACAGTTGGTTTTGGTGACCAGGTTAAACGGGTCGGATTCGTGAAAGTG
GATCATTGATTGGAGGGTAAACCTTACTTGGTCATTTCAGTCCTTAGTGGTCTAGTGTTTTTTTCTCTTC
TTAACTCGCGGTTGTAACAGCAGTATGAATACTCACCCCTGGTAAAAATGATCGTAACTACATAGCTGGC
CGAAAGAGCAAAAGTTTTCATCTTTTTTCTCTCATGTTGAGGAGGACAACGTTCCAGAGAGATCTCAATA
ACTAATTCATAATTACTCCACTAGGGTAATATTGCCTAACGCTTATTCATGTTCATGATTTTTCAATITT
TTTTTCTCACTCTTTTGATTTGTTTTGTGCCTTTGAAATTTTGATCAAATAGGTAGCAATTCATGGATTC
TGTCCTTGGTAATTTGCCATCTTATAACCCTCACAATTTCAGTCAGATTCGACCTTCAGATCCTTCTAGT
TCTTCTGTAAGTTATCATTATTTTCCTTTAATTTTATCAAAATATAGTTATTTGATAATCTTGGTAGAGC
TTATTCAACCATGCAAATTTATATTGTATTATTCTGATTGTGTATGATCCTGTGTTTTTATATTGCTAAT
GCTACCCTATTTATGAGTCCCACCTTAAATGAGATAAGGTCTGAACATGGGTCTATGCAATCCTCACCTT
ATAAGCCGGTCTTGTAGGTTTATTTAATTTAATTTGGACCAATTCAAAATTATAATATGGTATCACAGCC
TATGCAAAATCCGTCGGGTTACCTGCTATCAGATCACCACTTTCAAACCACTCGGGGCTTCAAGTTGTCA
ACCAGCAAGGCCGGGTTATAATCAGTGTTAAGAATTAGCATTAAGCTATAGTACATGAGCTTGGAGCTGA
TGTACCTTTACCGAGGGTGTGTTTGGTTCTAGGGTGACAAAAATTGATTTTGACTAAATTGATTTTACAA
AATTGACTTTGGTTGGAAGTGAATTGAAGGTAAAACGAGTTATGTTTGGATACATTCATTAAAAAAATTA
ATTTTTATCAGTTTATGTTTTGGATCAGAATTGCTTTTTTGTGGCTTTATTTGTCAAAAAAATTGGCAAT
TTATTTTATCTTACCACGGTAACTAGAAATATTAGCTTTTAGGTAGATTGATTTTGGGGCTGGATTCGAT
TTTAAAGCTATAAGTTAAACATAACAATTTATTTGTCAAATCTAGTCAAATTTGATTCTGGGAGGTACAA
ACATGGAACCAAAGACAGGTTAAAATGTACTATATGCTTAACCAAGCAATACATGCATAGAGAGACTTGT
CATACAGCTTATCTTATCCTAGTAATACTTTTCTCCCACTCCCTTGTGCTTTCCTTACTCTCTTTACATA
ATTGGCAGAGTATTTCTTCAATTTTTAAGTTTATACTTTGCAGAGCTTGAGATGAAAAACTTATATTTGC
AAACTTTAGGAATTTAAGTTTGTTTGTTTTATTCCATGGAGGAGTATCTCGTGACATGTTGATTGGTTTT
CCAAGTTACTTGGATGACCATAACTTATAATAGAATAATGATGTGGTGGTGCAATTTGGGTCAGCCCTAT
TGTTGTATGCAGCATTGTCTTTTAGAAGATATCCTTTTCATTGGAATAACTTAAACTGTTTGTCAAAATA
AGCGTAAAGGAATTGAATGTTTAAAACTTTTCAAACATGGCATGATATTCTTTTTTTCCCTTAGTTACTA
CTCATTTCATTCTAAAACTGATACTGTGAATTTAACTTTCTTTTCCAAATTGTAGCTTAGATAAATTCTT
CAGTAACAGAAGAGTTTTACTGTTTTTTCTAATTAGTTGGAAAACCTAGAATTATACGTATTTGTATTTT
CTATCAATAACAAAATATCTCCCCGGCTTTCAGCCATATCAGTTTCCAATGATACCCTGTCATATAATCA
GTTGTTTTCATATTTTACAATCTGATGATTATTGTTGTTATTGTTTTTTCTATAGATTTGTTTAACATAT
TACTAGCAAATGACATTATTATCGATATAATGAACCGTTAGCTGTTGCAAATCGAGGTCTTTGGATTCAT
TATATACCTCTGCTAAAGGATATAAAGTATGTTCACAGAATGTATAAGTGATCGTTGCTTTATGTTAACA
AAATTATTGATGGGGAAGATGCTCCTCTAGTCAACTGGATTTGTCTAGGAAGCTTAAATATAAAAGGTTC
GATCTTGAAGTGCAAAATACTGAATGGGTCACATTTTTTCCTTGCCTTCTTTCTCACCAAGTGAGGAGCA
TACCAATTTCGGGAGCGTGGTGTGTCATGTCGTGTGTGTGTTGTTTCTGATTCGTTTGTAAAGTGAAATT
CACTATTTTAAAATAAGTGTTTTCGGGTTCAACCGTTTTTTCTTATTCAACTTGTTACTCTTTCTGTTCC
AAAACATATTATGGATCGCCTAATCATATATTTTCATTTGCTTTGTTGAAACAAAATTTCTATTTTCATG
CAGAAAATGACAATAACTACTTACCATCCTACTCACGACAGGACCCTTCCACCACCTGATCAAGGTAGAC
AAGAGCTTTCTTCTACTTCTGTTGTAATGTTCTGCTATTAGTTGATTGATAATCTATTTGAAAAATTGTA
TCTTTCCTCTGGATTATTCTTTTCCCCTATATCACTATTTGGAGGAAAAGAATTGAAAACAGAAAATGTT
TCCACALACCAAATGTACCATTACAATTTTAACTGTGGTTGCAGTTTTTCTTCTTTACGAAGCTGTATGC
TGCAAATTTTCTCTCAGCGTTATTCTTTTGATTGATTTGGGATACTGTGATTGAGGGTTTTCTAACTTGT
GGAAAATTCTTTTGACAGTGATAAACACTGAAGCAAAAAATATTCTCCTAAGACATATTTATCAGAACGC
TCGGGAAAAGGTTAGTTTTGAAAGTTTGTTTTAGCACAGGTAAGGTAGGTATGCACATGAATGTGCAATA
CTCATACATCACCATATAGTGGCCCAACTGAACTGCTGCATATGTCCATTTTTCATTGCAGTTGAAACCA
AAAAGAGCTGCAGCTGGTAACCTTTTACCAGAACATGGATGCAAGCAACCTAGGGTTTCCACCTGACAGT
GTTCATTGACCAACTAGTGCATGCAGTTCTCAGCTACTTTCTCGAATGATATATACTCTTATTTTATTAC
CAAGATTTTGGTGCTTGTTTGAAATTGTAAACTATTATTAGTCCGCTACATACTTGGAGTGTGTAAATTT
GACAACTCCCCCACCAATCAATCAATATATACAACAACTGAAATTATCATGCTTTTATTGTGTATATTTT
T
Medicago truncatula
SEQ ID NO: 42
MDSVLGNLPSYNPHNFSQIRPSDPSSSSKMTITTYHPTHDRTLPPPDQVINTEAKNILLRHIYQNAREKL
KPKRAAAGNLLPEHGCKQPRVST
Arachis hypogaea, CDS
SEQ ID NO: 43
ATGGATTCTGTCCTTGGTAATTGGCCATCTTATGATCCTCACAACTTTAGCCAGCTTCGAACTTCAGATC
CTTCTAGATCTTCTAAAATGGCACCGGCCACTTACCATTCTATTCACAACAGGGACGTACCACCAGCCGA
TCAAGTGATAAATACCGAACACAAAAATATCCTTCTAAGAGAAATCTACCGGCGTGCAGAGGAGAAGTTG
ACACCCAAAAGAGCTGCATCCGATAACCTCATACCGGAGCATGGATCCAAACAACCAAGGGTTTCAACGT
GA
Arachis hypogaea, cDNA
SEQ ID NO: 44
CAAAAGTTTGTTGCAGCCTCTGCCTCATGGATTCTGTCCTTGGTAATTGGCCATCTTATGATCCTCACAA
CTTTAGCCAGCTTCGAACTTCAGATCCTTCTAGATCTTCTAAAATGGCACCGGCCACTTACCATTCTATT
CACAACAGGGACGTACCACCAGCCGATCAAGTGATAAATACCGAACACAAAAATATCCTTCTAAGAGAAA
TCTACCGGCGTGCAGAGGAGAAGTTGACACCCAAAAGAGCTGCATCCGATAACCTCATACCGGAGCATGG
ATCCAAACAACCAAGGGTTTCAACGTGAAAATTTTTCTTTGACCAACAAATGATGAATATGGTTTGTGAA
CAACTCTTTCAGAAGTCAGATATGCCCTTATGTAACAAAGAAGACTTTGGCATGTTTGGTATTGTAAACT
ATCTTTTCAAGTATAGAGTTGGTTAGCCCCAGCATAATTATTCAGTGAATGAAGTGAGATAGTGATTATG
AATTTCATTGTAGATTTTGTGCT
Arachis hypogaea
SEQ ID NO: 45
MDSVLGHWPSYDPHHFSQLRTSDPSRSSKMAPATYHSIHNRDVPPADQVINTEHKHILLREIYRRAEEKL
TPKRAASDHLIPEHGSKQPRVST
Populus trichocarpa, CDS
SEQ ID NO: 46
ATGGGGTCTTTGCTTGGTGACTGGCCTTCATTTGACCCTCATAACTTTAGCCAACTTCGACCTTCTGATC
CTTCTAATCCTTCTAAAATGACTCCTGCCACCTATCATCCTACTCATAGCCGGACTCTTCCCCCACCTGA
TCAAGTGATAACTACTGAAGCAAAAAATATTCTGCTGCGAAATTTCTATGAGCGAGCTGAAGAGAAGTTG
AGACAAAAGAGAGCTGCCTCTGAACATCTAATGCCAGAGCATGGATGCAAGCAGGCTAGGGCTTCTACCT
CATAA
Populus trichocarpa, cDNA
SEQ ID NO: 47
ATGGGGTCTTTGCTTGGTGACTGGCCTTCATTTGACCCTCATAACTTTAGCCAACTTCGACCTTCTGATC
CTTCTAATCCTTCTAAAATGACTCCTGCCACCTATCATCCTACTCATAGCCGGACTCTTCCCCCACCTGA
TCAAGTGATAACTACTGAAGCAAAAAATATTCTGCTGCGAAATTTCTATGAGCGAGCTGAAGAGAAGTTG
AGACAAAAGAGAGCTGCCTCTGAACATCTAATGCCAGAGCATGGATGCAAGCAGGCTAGGGCTTCTACCT
CATAA
Populus trichocarpa, gDNA
SEQ ID NO: 48
TTCAAATGCATTAATTCCACTTGTGAGTTTAATATGGATTGGAATCAATATCAAAGTCTATCCATATGGT
CAAAAAAAATTAGCCAGGATAAAAAGAAAAGCATAACTAATGATCTCACTCGOTGGTCATATGATGACCC
GGTGAATCTGTTGTTTGTACACAACTCTACTTGCATTGCTCAACAGTTTAGAACAATAAGCAAGCCCTGC
TCAGTCAACCAATTGGCACACTGGGCAGTGATGTGGTTTGTATTTACAACGAGAGTCGACATGCCAGACT
ATTAAGTAACATACGAAAGCTTATTATGAGGTAGAAGCCCTAGCCTGCTTGCATCCATGCTCTGGCATTA
GATGTTCAGAGGCAGCTCTCTTTTGTCTCAACTGCAACAAGAACGGGAATGACAAGCTAAAATAAGTACG
CGTCCAATGGGTGCACAAATAGAAGAGAAAGATCAAATATGTGAAAACATTTTAATTTACCAACCTTCTC
TTCAGCTCGCTCATAGAAATTTCGCAGCAGAATATTTTTTGCTTCAGTAGTTATCACTGCCAAAGGAGTT
TAAGCCAGATTAACCGCAGATTATGAAATCACCTTCAGATAAAATGAAGAACAAAAAACTGAATTACAAT
AACAAAATGCAGGAAGTTCAGCTGATCAAACTTGCATAAGCATGTCATAATCAATTGCACATCATCCCAA
GCTTTTCAGAATGCCCAGGCAATATCCATGATGATACAAAAGAAGGCCCATGGAAATTCTTCGCCATCCC
AAGTGGTTAATCATGGTCAAAGACCAACTCATGGAGCAAGAGGCATGCAGATTAGAGATAGTGAAACTGC
TCCATCCAGCACTGAAACATGTAAAATTCAACATCGATTGCAGAAAACCCCCCCGACTTTAGGCCAGATG
CTTTGTCACTCACATCCAAAGTAAACTAAAGCTCCTTGTTGTTTTATTAGGATGTTATAATGAGATTGCA
CCAGTTTTATCCAGAATGTCGTATTGATTTCTCCCAACTTCATGCACACCATGGACTTTGAAAGGGCGGC
CCCAAGCCTTTACAAGAATCTATGCCGGTTTTGCTCATGCATGATTGTTGGATTCAATCTCCCCACCTCC
CCATCTTTTATATTCTACATTTTTGCCTTTCAAGAATCTTTCTTAATGAAGTCACAAAATATTAATATCT
TGATGAACAGGAATCGGAAGATAGATCAAAGAGCAAAACGCTAGCATCTAAGCTCATCAAACATTTACAT
TCATAACAAGAGAGACATTATGATAACATGCACCTTTGAATAACATGTCCAGATAAAGATGTCAAATTTG
GCAGCCATATTTAAAGGTCATCCCATGTCTTAGAAAACAAAATATGCCAGGTATTCCCTATCTTCACCCA
AACAGAAAATTTGGGGAGAAGAAACTGCAAGCAGATAGAAAGTTCTGTTCTTTACCTTGATCAGGTGGGG
GAAGAGTCCGGCTATGAGTAGGATGATAGGTGGCAGGAGTCATTTT
Populus trichocarpa
SEQ ID NO: 49
MGSLLGDWPSFDPHNFSQLRPSDPSNPSKMTPATYHPTHSRTLPPPDQVITTEAKNILLRNFYERAEEKL
RQKRAASEHLMPEHGCKQARASTS
Populus tremula, CDS
SEQ ID NO: 50
ATGGGGTCTTTGCTTGGTGACTGGCCTTCATTTGACCCTCATAACTTTAGCCAACTTCGACCTTCTGATC
CTTCTAATCCTTCTAAAATGACTCCTGCCACCTACCATCCTACTCATAGCCGGACTCTTCCCCCACCTGA
TCAAGTGATAACTACTGAAGCAAAAAATATTCTGCTGCGAAATTTCTATGAGCGAGCTGAAGAGAAGTTG
AGACAAAAGAGAGCTGCCTCTGAACATCTAATGCCAGAGCATGGATGCAAGCAGGCTAGGGCTTCTACCT
CATAA
Populus tremula, cDNA
SEQ ID NO: 51
GATTGTATGGACTTATAAAGACTAAGAAATTTATCATGCCAACCTGCGGAGGTTGGTTCTAGAATCAGAC
CATTGTTGTCTCTCATAATCTCTCTATCTCGCATTCTAATGGGGTCTTTGCTTGGTGACTGGCCTTCATT
TGACCCTCATAACTTTAGCCAACTTCGACCTTCTGATCCTTCTAATCCTTCTAAAATGACTCCTGCCACC
TACCATCCTACTCATAGCCGGACTCTTCCCCCACCTGATCAAGTGATAACTACTGAAGCAAAAAATATTC
TGCTGCGAAATTTCTATGAGCGAGCTGAAGAGAAGTTGAGACAAAAGAGAGCTGCCTCTGAACATCTAAT
GCCAGAGCATGGATGCAAGCAGGCTAGGGCTTCTACCTCATAATAAGCTTTCGTATGTTACTTAATAGTC
TGGCATGTCGACTCTCATTGTAAATACAAACCACATCACTGCCCAGTGTGCCAATTGGTTGACTGAGCAG
GGCTTGCTTA
Populus tremula
SEQ ID NO: 52
MGSLLGDWPSFDPHNFSQLRPSDPSNPSKMTPATTHPTHSRTLPPPDQVITTEAKNILLRNFYERAEEKL
RQKRAASEHLMPEHGCKQARASTS
Linum usitatissimum, CDS
SEQ ID NO: 53
ATGGGGTCTATGCTTGGTGACTTGCCTTCATTTGACCCCCACAACTTCAGCCAACTTAGACCCTCCGATC
CTTCCAATCCGTCCAAAATGACTCCTGCAACCTATCATCCAACACACAGTCGTACTCTTCCACCACCTGA
TCAGGTTATGGCTACTGAAACGAAGAATATCCTTTTAAGAAACTTCTACAAGCGCGCTGAAGAGAAGATG
AGACCGAAGCGAGCTGCACCAGAGAGCCTTATACCGGATCATGGTGGCAAGCAGGCGAGGCCTTCTACCT
CAAGCTAA
Linum usitatissimum, cDNA
SEQ ID NO: 54
ATGGGGTCTATGCTTGGTGACTTGCCTTCATTTGACCCCCACAACTTCAGCCAACTTAGACCCTCCGATC
CTTCCAATCCGTCCAAAATGACTCCTGCAACCTATCATCCAACACACAGTCGTACTCTTCCACCACCTGA
TCAGGTTATGGCTACTGAAACGAAGAATATCCTTTTAAGAAACTTCTACAAGCGCGCTGAAGAGAAGATG
AGACCGAAGCGAGCTGCACCAGAGAGCCTTATACCGGATCATGGTGGCAAGCAGGCGAGGCCTTCTACCT
CAAGCTAA
Linum usitatissimum, gDNA
SEQ ID NO: 55
ATGGGGTCTATGCTTGGTGACTTGCCTTCATTTGACCCCCACAACTTCAGCCAACTTAGACCCTCCGATC
CTTCCAATCCGTCCGTAAGCTCACACTTTTTCCCCCATCTTTCATTTACCGCCCCAACCTTTTCTTTTTC
CTTGAATTGTGTTGAATCTTGAGAGTTGATTCGGTTCGCATCTGAAGTTCTGATTTTTTCTTTGATTTAG
TCTCAATTTTTCCTATCCGAGTGCTACATATGACCCTAATATGGGTTGAGAACTGATGAATTTTATGTTT
TTTTAATCCAATGAGTTTATCGGTTGTTTCCCCCTTTCTTCTTGAGGTTTGGTATCCGTCTTTTGTCGAA
TGCTAGGGCTAATATTCGAATTAGATTCCATCTATTAGACTTTTCAGTGAAATTTGCTTGATTCTTCAAT
TGAATACTAGGAACGAGTCAAATTTTTGCGATGCAGAATGTAATGCTATTCTCAATTTGCCTAATGAGTT
TAATCTGCCTATTATTATCGCTGTTTATATTCCTGGATCTTTTTGTTCGAATTAGGGCTCTCGTTGGCAA
GTTATTACTGTAACTGCTTCTCCAGAAGTTCCATAGAATTAATTGCATCCATAATTTGATAGGACGAGTT
GATTTAAGAAAAATTAAGTTTTTGACAGTCAACAAAGGATTGATTGTGATACTGAAATGCAGAATGTAGG
TAGGCTAATTAGGACTTAGGAATGATAAAAGTTCGCTTCTTCATGTAAATGAAGAGATGTGCATCAAGGA
TAAAGGCCATCTTAATACTCGTAGGTTGAGTCTTTTTTAAGAATAATGCAGTTGCTGATGCGGCAAGAAA
AAGTTAAGCTAGTTGAAGAAGAAAGGAGTCAGAAATGTATAGATATTTCCTTCCCTTTGAGTATTTCTCT
TCTCTGTAACTGCTTATTGCTTCAAAGTTTTGGCCCTTGTTCTAATAATCACAACCTTTCTATAGGAACA
TCTAATGGCTAGTGCTTGATAATTCCATGTATATACCTCTTACTGACATGCTTGTATTTTGGTTACCATT
TCTGTTGGTTTAAGTCGTCAAATGGCATAGATTCAATAGGGCACTCATAAACAAGTAACTACAATGCATA
CTAGAGTAGGTGTCTGAATGCATTGCAAGCTTCCAACATTTGGTTGTTCAGGAACTCTGGAATTGTTCCA
CTAGCATGTTCAATGCAAGATATTCATGTTCAGTGTTAAACTGTGTAAGTGAGGAGAGTTTAATTCATTG
TACGCATGCCTTGGGGGTGTCAGTACGAGGATTTAAGCTCAATTTATCAAGTCTGTCATTAAACTAGTAC
TTCAGCAGGGACTTGACCTGTCAATAATCTGTCATATGAATGCTATCATGTTCGTCAGTTTTAAGGAAGA
GAAACTCTTTAAATGTGCTTATCATGAAGTTATTGGATTCGGTTTTATTTATTCTGACATTTTCCTACGA
TCATTTGCAGAAAATGACTCCTGCAACCTATCATCCAACACACAGTCGTACTCTTCCACCACCTGATCAG
GGTAATGACTAGCTTTCCTTTATAATTTGTGTTTCCTGCCTTAAAAGTTTCCACCTTTTGTAGAAAGAAC
GTGGTGTTTCAACACTTGACTACTCAAGGGATAGTAATTGTTAATTACTTTTTTAACTGTGTTTATCGGG
CATGGGGACATTTGAAGTTCTTATAGTTTAAAACATCTTATTGCAACTCAAAATCCTTCAACTTCCATAA
GTAATAGCTAACCTAGAGAACTCAAGGTCTCTAACCCAGTTGAAGCGGTTCGATTTAATCTGCATACATT
CTTTTCCATCGCTATCGGTTTCTGTTTCACATTAGAAGTGAGTCTCTCCGATTAAGTCCTCGAATCTCTG
GCCTAGCTAGTTGGCCGCGCTTAGGAAAAGTCTGCTACTTTTAAATCTGTGTCGTAAAGCAAGCTTGATT
AGCTGAGCCCATTCAAGGTCTCTAGTTCATGTTCCTTGTTCTGGCTCTTTTGCAGTTATGGCTACTGAAA
CGAAGAATATCCTTTTAAGAAACTTCTACAAGCGCGCTGAAGAGAAGGTTTGAGTCACTCATTGGCATCG
CATTGTTAGCCTTGGTTTCATATGTACATTATTATTTGCAACGATGTGTATGTCACTGAGCTTATTTGTG
TTTTTCCAGATGAGACCGAAGCGAGCTGCACCAGAGAGCCTTATACCGGATCATGGTGGCAAGCAGGCGA
GGCCTTCTACCTCAAGCTAA
Linum usitatissimum
SEQ ID NO: 56
MGSMLGDLPSFDPHNFSQLRPSDPSNPSKMTPATYHPTHSRTLPPPDQVMATETKNILLRNFYKRAEEKM
RPKRAAPESLIPDHGGKQARPSTSS
Ricinus communis, CDS
SEQ ID NO: 57
ATGAGCTCTCTGCTGGGTGACTGGCCGTCTTTTGACCCTCACAACTTTACCCAACTTAGACCGACTGATC
CTTCTAATCCTTCTGTAATGACTCCTGCTACTTATCATCCAACTCATAGCCGGACTCTTCCACCACCCGA
TCAAGTGATAACTACTGAAGCCAAAAATATCCTTCTGAGAAACTTCTATGAGCGAGCTGAAGAGAAGTTG
AGAACAAAAAGAGCTGCCTCTGAAAATCTAATACCGGAGCATGGATGCAAGCAGCCTAGGGCTTCTACCT
CATGCTAA
Ricinus communis, cDNA
SEQ ID NO: 58
ATGACTCCTGCTACTTATCATCCAACTCATAGCCGGACTCTTCCACCACCCGATCAAGTGATAACTACTG
AAGCCAAAAATATCCTTCTGAGAAACTTCTATGAGCGAGCTGAAGAGAAGTTGAGAACAAAAAGAGCTGC
CTCTGAAAATCTAATACCGGAGCATGGATGCAAGCAGCCTAGGGCTTCTACCTCATGCTAA
Ricinus communis, gDNA
SEQ ID NO: 59
TTAGCATGAGGTAGAAGCCCTAGGCTGCTTGCATCCATGCTCCGGTATTAGATTTTCAGAGGCAGCTCTT
TTTGTTCTCAACTGCATAGAGAAAAAATACCAATATGAAGCTAGAAGTATGTGTAGCAATCAGATAAAGC
AAATGCTATATCTGAATTATGTGACATTGTGTATCATTAACCAACCTTCTCTTCAGCTCGCTCATAGAAG
TTTCTCAGAAGGATATTTTTGGCTTCAGTAGTTATCACTGCCAATTTTCAAACAAATGAATCACAAAATT
TTCTTTTGATCATTCAATACCATAAACTGTATTATAATATCAGAALAACAGAAGCTAGGAAAGTTCAGCT
GATTAAACTTGCTTAAAATTTAAAATCTAGACACCAACCCTGTAGTACAAGCTTTTCAAAATGTCCCTAA
AATATCAACAGTGAAAAACACTAATTAAGACCCATAAACATTTTATGCAATTTCTGGCTGAAATCGGGGA
TGGCAAGCAACTTATGGAGGCAAAAGGCAAGCAGTATCAGATGTATGTGAATCCAACATTGATCTCAAAG
TCCATCCCTCTAGCATCAAATATATGTGAATCCAACATTGATCTCAAAGTCCATCCCCCTAGCACTAAAT
ATATGTGAATCCAAGGGCTCTATCCAGCAATGAATATGTGTGAACCCAACATTGATTTCAAAATTATCCA
CCCCCTAGCATAAAACTGAATGCATGTCACTCATATTTAAATGTTAGACAATAGCCCTTACCAATTCATG
GATATAGAATAATCACAAAGTGACATTGGGTCATCGGAATAATTTCCTGATCCCAAGTCCCACTCTCACA
TCAATTTAATTTAGGAAAAGCATTCCATATGTAGTTAGTTAACACTTAACAGCATTTTTTCTTTACTCTT
TCAAACTTGAATAGCTTGGATCATTTTTCAGCAGTACCTAGGTCTTGAGGTCTTGAGGAGTTAGACTAGA
CTCTAGAGGAGCTCAACATCATGTCCTCACGCATGGTTGTCAGACTCAATCTCTCTACTCTGATCATGCA
AATATATTCTTGTTTATCCTAGATTTTTGCATTTTAGTACACTTTCTTAGTCCAAGGAGGTTTTTCTTTC
CCATTCTTTATTCTGTTGTACTAGAGCATTTTGTTTTGAACAACTTAACTCTTATCCAAGAAAATAATAG
GAAGAGCAGAAGCATCTCCGAATAGCATGAGTAAGCTTAAGAAAGAAGTTAACAGGTAGATGAAATTGCA
AAGGCTGGCATTTAGCAGAGACAAGCATACGTCACACACAACACCAGAAACAAAACACATTTACACACTC
ATGTATGATTAAATCACAGGATAACAACTAGTTCCAGCATTCAAAGAATCACACACCAACTAATTCTTAC
CTTGATCGGGTGGTGGAAGAGTCCGGCTATGAGTTGOATGATAAGTAGCAGGAGTCAT
Ricinus communis
SEQ ID NO: 60
MSSLLGDWPSFDPHNFTQLRPTDPSHPSVMTPATYHPTHSRTLPPPDQVITTEAKNILLRNFYERAEEKL
RTKRAASEMLIPEHGCKQPRASTSC
Theobroma cacao, CDS
SEQ ID NO: 61
ATGGGGTCTATGCTCGGTGACCTGCCGTCGTTTGACCCCCATAACTTCAGCCAACTTCGTCCCTCCGATC
CTTCTAATCCTTCTAAAATGACACCTGCCACCTACCGCCCTACTCATAGCCGGACTCTTCCACCACCTGA
CCAAGTTATAACTACTGAGGCCAAAAATATACTTATAAGAAATTTCTATCAGCGTGCTGAGGAGAAGTTG
AGACCAAAGAGAGCAGCCACTGAACATCTAATACCAGAGCATGGATGCAAGCAACCTAGGGCTTCTACCT
CATAG
Theobroma cacao, cDNA
SEQ ID NO: 62
ATCATCCAGCACTAGTACGAAAAAGGCTGAGTCTAGAATCGGGGCAGGCATTGTTGTGGTTTCTCTCCCA
TTTTCTCAATTGTCCCAATCTCTCTCCGGAGATTTTCTGGGTGCAGAAACCAGCATATTCTTTTTCCCCA
ATGGGGTCTATGCTCGGTGACCTGCCGTCGTTTGACCCCCATAACTTCAGCCAACTTCGTCCCTCCGATC
CTTCTAATCCTTCTAAAATGACACCTGCCACCTACCGCCCTACTCATAGCCGGACTCTTCCACCACCTGA
CCAAGGTATTGAACTGATATTTTTCTCCTTGTTTTTACTTGTGAAACAATATTCCCGAGGAAATATAAGA
TATTATTGGCCTTATAAACTGTCTGCAATGGTACCTTCTAGGATGTTGAATGTTGACTTCTGTTTGAGAG
CAGCAAGTGCTGGAAATTATGTGGAGATGTCTGAATTGGAACTGGATATGATGTCATTTTTCTGTAAAAA
TGGTATTGCCTTGACAAATGGGCTTCAAATAATTGCAAAACCCACCCCCACTACGATCTCCAACAAGTCC
ATTTTGTTGCCTAATCCTCGTATCATAACCGCCAGGCATCATAATAACATCCTACAATCAGCATCATCAT
CATCATCTTCTTCAGCTTTTACTCTTACAGCTTCAATTTCACCGGGTGCTACTTCGGTTGCAGTCGATGG
ACCCACCACCTCCACGAAACCTTCCAAGTCTTTGCCGTTTAGAGTGGGCCATGGCTTCGACCTTCATCGT
TTGGAGCCTGGCTACCCTTTGATCATTGGTGGGATTGATATTCCTCATGATAGAGGCTGCGAGGCTCATT
CGGATGGAGATGTGCTGCTTCATTGTGTTGTGGATGCAATACTGGGAGCTTTAGGGCTTCCTGATATAGG
GCAGATATTTCCTGACTCTGATCCCAAGTGGAAAGGAGCTCCATCTTCTGTCTTTATCAAAGAAGCTGTG
AGACTCATGCATGAAGTAGGCTATGAGATTGGAAACTTAGATGCCACCTTAATTCTTCAAAGACCAAAAT
TAAGTCCACACAAGGAGGCTATCAAAGCCAACTTGTCTGAGCTGCTGGGAGCCGACCCATCTGTTGTCAA
TCTTAAAGCAAAGACTCATGAGAAGGTCGACAGTCTTGGTGAAAATCGAAGTATTGCAGCCCATACTGTG
GTCCTACTGATGAGGAAGTAAATATAGGTCTCGGATATCAGTCTCGAGTATGGAAATTGTATGGCATACC
ATGAGCATTAGTTGTAAAACTGCCATAAATTATGGCATTGCTAAGTATGAAAGCTTGATGTGTTTGGTTG
GACCACAATGTTAGAGTTGTGTTTTCAACATTTTACCAAAACGACTTGAACAACAACGATGTGAGTTAAC
GAGTGAACCTACATCTACAACACGGTACCGTGTGAGTCAAATCTGTCGGACCTTTATTGCGGAATTAATT
CGGGAAACAAATTTTTTTTTTGAAA
Theobroma cacao, gDNA
SEQ ID NO: 63
ATCATCCAGCACTAGTACGAAAAAGGCTGAGTCTAGAATCGGGGCAGGCATTGTTGTGGTTTCTCTCCCA
TTTTCTCAATTGTCCCAATCTCTCTCCGGAGATTTTCTGGGTGCAGAAACCAGCATATTCTTTTTCCCCA
ATGGGGTCTATGCTCGGTGACCTGCCGTCGTTTGACCCCCATAACTTCAGCCAACTTCGTCCCTCCGATC
CTTCTAATCCTTCTGTAAGTATCCCAGAATCCTTTTTAAACCCAACCCCATTAACAAAAATACATGAAAA
TATGAATTCTTTTCTGGTATGATCTGAATAAGTTGTTCGTTTCAACTGTTCTGATACTGCAATGAAACCC
ACATGCGGTTTTAGATGAGTAGTGAAGAACTGTTAGATTTTTATGATTAGGTTTCAAGGTTTACCCAGAT
ATGATGTTGGTGGATTCTTTTCTCAAAGCGCTTTTCTGAATTTGGACCTTAAAAAATGCTGTAGTCCATA
TAATCAGTTAGTCGTCAGAAGCTTTTTGGATAAAGTTCGTTTGTGGTTACAAATGAATATCTTGCTTTTG
TATTTATAGGGGTTAAATGATTCTCGGAATTTCTACTCCGGTGTTATTACACGTTTAGTGCTTTTGTTGT
TTGCATTGCAAGATACATTTAACACTAGTATGTTTATTAATTTGAATGAATGGAATGTAATATGGTGATG
TTACCATGTGAAGCATCTAAGTTATGCAAATAGGACTTGTTATTATCTGTTTGCTAAAATGACAAAGATC
TTTATACAGCACAAGCATTAGCGTGGAAATGCTTTCTTGTATGGGAATGGCAGTTTCCTTAAAATTGTAG
GGTAACTATTCATGAGCTTGTGATTTTGACCACTGCATGCTACTGTCAGCTTCTAAATCTACCAAGATTT
TAAGTTCATGCTCTAGAAGCTTTTCAGATCTTCCTGTGTACTTGGTAGTTTAAGTTCTTAAGACTGGTTC
TTAACAATGATCAAGAAGTTCTATATTTCAGATCTTCCTGTGTACTTGGTAGTTGAAAGTTTGTTTCTAT
GTGGGTATTGAGAGAATTGGTTTGAGAATTTGCTGTTTTCTTGATAAAAAATTAATCCTGTCCATGACAT
GGACATGCTATTGACAGAACGCATGAAATTGAGCTTTTGCATTCTAACTTGGGCACCGTTTTACTGGTGA
TTCGTATTGAGATAATGATTGTGTTGATGACATTTGGGCAGTCGCTTGAAAATATTGAAATGTCAGGAGA
AAAGAGAGAAGGTGAAGGGAAAGCCCAAAAGGAACGAGAGATATTAGATCACCCTTTTTCTTTCCTTTAT
CTTCTTCTTTTACTTGGTCTACCTCCTGATCATTTTCTTGAAATTCTCACTAAATTCTAGTTTTGTTTAG
ATCTTGAATTTGTATAGGGTAATATAATTGCCGAAAAGAGTTCAGTAAGGCAGGGTTTCACCTGGTAAAG
AAGCTGCATGGTGAATTTTGAATTGCTGTCCTCTAGCACATGGTGCACTACAGGATATCAATTTCCTTTC
ATAGCATGCATACATGTAGGCTTATATGAATTTATGTATCTATTTTTGTTTCAATAGTTTATAGTGGTTC
CCTGCCTAACTGTTATAAGGTTTCCGATTAAGTAAGAGTTGTTGAAATGTCCAAAGGGTATAGAACATTT
TTCACCAGACTTTCTATCCTTTTTAGCTATTTTAGCATGTGAGATGCTATGCTAATGGATGGAGTTCATA
TGGAGCTCCTTTATGTTTTCAAATGAAGAATTTATGACAAATTAGTCTTTTTGCTTTCAGATTGATAGGT
TGGTCTTTTAACAAAATTACACTTGTCTTTTTGCTTTCAGATTGATAGGTTGGTCTTTTAACAAATTACA
CTTACTTGTATGGTTTGTTACTTTGCTTGAATATTTTAGGAGCATAAAATGCTTCTCCTTACTTTTCAGT
CATGTTAAGAATTGATTGCACTTTAACTTTTGTACTTAATCTTCTCTTTTTGGCTAGAAAATGACACCTG
CCACCTACCGCCCTACTCATAGCCGGACTCTTCCACCACCTGACCAAGGTATTGAACTGATATTTTTCTC
CTTGTTTTTACTTGTGAAACAATATTCCCGAGGAAATATAAGATATTATTGGCCTTATAAACTGTCTGCA
ATGGTAACATGATAACCTTTGGTTGGCTGATTCTTTCAGACTTTGTCTACATGGTGATAATATATTTTAA
AAATTCATGTCATATACTGTGATAATTATTTGGCTAGGTACCTTCTAGGATGTTGAATGTTGACTTCTGT
TTGAGAGCAGCAAGTGCTGGAAATTATGTGGAGATGTCTGAATTGGAACTGGATATGATGTCATTTTTCT
GTAAAAATGGTATTGCCTTGACAAATGGGCTTCAAATAATTGGTATTGGACTGAGAACTTGATTTTTACA
TGGCACTCTATGCTTGCTACCACTGCTACTTGCTTACATTTTTTTTGTCATCTGTCTCTGAAATGGTGGA
ATTGCTTTACCTTGTTTCTTTAGTTCTTTGTTTCCTTCTGCAGTAATCTAAATGTCGTAGAGTACTCATC
TGGAATTGTTCATCTTCTCTCAGTTTCATTTTGATATCGACGAAGAATGGAGATTTCTAATTGGAAACCA
GTGATTGATTGCGTACTTGATTGAAATTGCTGTTTGAAGTTTATCAATTGGAATGAAATTGTTAAGTTGT
ATGCGTGTTCTAAATTGCCTGTGCTACAAACACAAAGGCACTACTAAATGAGATTGCATTCTGGTGCAGT
GCCACAGCTCATTGGCTTAAATTCTTGATTTCTTATCCTTGTTTTTTTAATTGACCTGTGAACATTTTCT
TTTTGTGTAACTGACCTCTCAAACACTTTCAACAGTTATAACTACTGAGGCCAAAAATATACTTATAAGA
AATTTCTATCAGCGTGCTGAGGAGAAGGTTAGTAAATTGTTTATGATTTTTCATGTTATAACACTGCATT
CAAATAATTTCAGTCACTGTTACAAATTCAAGGACACATGCATGCACCTTGAGAGTGGGTGTCTGGGTTC
ATTTAGTTTCTTTTTGCTTTTCTCATTGCAGTTGAGACCAAAGAGAGCAGCCACTGAACATCTAATACCA
GAGCATGGATGCAAGCAACCTAGGGCTTCTACCTCATAGTCGTGATGAGATTTTCTTGGGTTCCTTTGAT
GCTCATGTAAATGTATATTCTCATATAAATGTGTTGTAGAAACTGTGCGGCAGTTGTTGTAAAACGGGAC
ACCACACATGTACATTTTGTGTGTAGAAATCAAACTTATAGCTGGGTTATCCCAATAACAAGGAGTGTTC
TGTTTAGTTTTACATGTTCCGTTTGGTATTTCATTCCATGTGGCGTGGCTAAATTGCTGAAAAGCTAAAT
CCATGTATGCTCATTTTATATGATGTGCACAGTTGCATCTCTAAGTAATTACAGGTTTGAATATATTGCT
TGGGCTCAAGGAGCTGCATATTTTAATATAATATCCATGCTCCTGTTGCTTATGAAGTTAGTTTATGCCT
GTTTCAATTACATAAATTGAAGGTTTTCCCTGTGGGGTTACAGAAGATAAGAGTAGTTTGGGAATCAACT
GAGATTCAAAACAACAGGTAGAGGTACGGTAGTGTGTTGCATCACTTCTTAACAGAATATTCCATGAGGT
TTAAGACTTAAAGATCTGATGACCATTCATGTTTCACGGAGTTAAGGGTCGTTTTCTGTCTAGGGTCTCC
CAAATTTGGCAACATTAGGCCTCTCAAAGTCTTGAGTTTGGCACCAATTAGCCCACACGCAATATCCTGA
TGAAAAAACGTTTCCGAACAGTTACTTGAGAATGTGGGACAAAATCATATATTGCAGTGGCTGTGTTTGC
ACTTGAATGAATTTCGGTAAACACATTGGAAATGGGCCATTCACCGGAGTTGCACCCAGCGGTTGACAAA
TGGAAAGTAGACAACGAATGATATTATTTGGTTCCTGCAATTAAATACCTACCTTTAAAATAAAAATTAA
GGAAAAAAAAAGGAAACAAATTACTGCCACAAATACCAAAAAAGAGAAAAAAAAAAAAAAAGGAAAAGGA
AAAACGAAGGGAACCCACGAAACAGAGTCAACAAAGAAACTGAAACTACTCCACAAAGAAACAGAGCCTT
TTCTCTGCCAAAGCAAAGATTTCAACAGCTATGGCCACTCACTTTTACAGTTGTTCTCCAATTCCAGCAA
AACCCACCCCCACTACGATCTCCAACAAGTCCATTTTGTTGCCTAATCCTCGTATCATAACCGCCAGGCA
TCATAATAACATCCTACAATCAGCATCATCATCATCATCTTCTTCAGCTTTTACTCTTACAGCTTCAATT
TCACCGGGTGCTACTTCGGTTGCAGTCGATGGACCCACCACCTCCACGAAACCTTCCAAGTCTTTGCCGT
TTAGAGTGGGCCATGGCTTCGACCTTCATCGTTTGGAGCCTGGCTACCCTTTGATCATTGGTGGGATTGA
TATTCCTCATGATAGAGGCTGCGAGGCTCATTCGGATGGTATGTTCACATTCCAATCGTCCAATTTTGCT
TTATTTTTGTTTTTCCCAACTTTAATATCCATTTTTTATATCTTTTAGTTTCTTGTATTTGGATAGTTTT
AATTTATCAAAGTTCTTTTTTTTAAACCCCGTATATGTTTTCTCGGAATCTGTATCTCTTGGTTGTATTA
TCTGTTTAAATTATCCACAATTTGCTTTTGATGCTAACTATATGGGGATTAACGTGTTGCAGGAGATGTG
CTGCTTCATTGTGTTGTGGATGCAATACTGGGAGCTTTAGGGCTTCCTGATATAGGGCAGATATTTCCTG
ACTCTGATCCCAAGTGGAAAGGAGCTCCATCTTCTGTCTTTATCAAAGAAGCTGTGAGTAATTTTGGGTA
ATTTTGAGAAATTCGTTTTTTTTTTTTGGGTATTTTATTTTCAGTGTATGCTTTGACTGGTTCACTGTTG
GATACCTTAAAAACAGAGTTCATAACACACAGACACACACAAAAGAAAAAAAAGCTTTATTTGCCATACT
ATGAAACTTCTACTTAAAATCTGGCCTCAACTTGGACGGATTGTGTTAGGTTGGGAGAAGGGTCCTATAT
TAGAACACTAGTACTCTATGAACTCTTGTTGAAGAGAAAGTAAACGAAAAGGCAACACAGAGCTGAATTA
TGCAATTATTTATTTTGGGTGCATAGTTAAGGAAGGAAAATATTAATCATTCTGAAAGTCATAGGCTCCA
ATAATGTACTTTGACACAACATTTAAGTATAAAAATCTTATGAAATATCCTTTTTAGCCTTGTTGATAAG
AAAAATAAAATAAAAAGGAATTCCTTGAGTGGATTGGACAGACTACACAGTCAGTTTTTGTAGTCAACCC
ATATATGATATAGCACCACTTATTGATCTCTTGAGGGGCATGGCTGGTTAATTTCTATCTGCTTAGAAAC
AAAAACTGAGTCTGCTAGTTCATAGCATTTGGTAATTTGTATTAGATGCATTAGCAGAACAAATCAATAG
AGGGCTGCCTGTATTGAGGAGTGGAGAAGTGAGTATGATACTGCTGCATCAGAGCAAAACTCTGATTGTC
TGACACTGATAGTGACTTTCCAACCACAATGCAATGAACCTTGTACTTTATGTGATACAATGAATCTAAC
AGTGCAATGAATCTTTCTTCAGAACAGCTTAACTTGTGTATGGATGGAAATTGTATTAAGTTCTTTATTG
TCGACTGTACTTACTGATAAGGGCATGTGCATGTATATAACTGAAGAAAATTCTGTATAACCTTGTATTC
TGCCTAGCCTGCAAAGTAATACTTTGTGCAGATGCACCTTGTAAACATGATGTTTGACTTAAAAGTTTTA
GGTAAAATAAGTTAATAAAAGTTGATGCTTTTAAGAGCAATGTATCGGGTATGATATTGCTTCACTTTTG
AATTCTGTAAAATCTCTGATGCTTTTTGGACAGTGACTTCACCAACCACAATTCAATGAACCTTTCTTCA
GAACAGCCTAATTTGTTTGTGATTGGAAATAGAATCAACTTTTTATTATTGACTGAACTATTGCATATTC
ATGTATGTGCATGAAAACCCTGTATAACCTTAAATTTGTATTTATCCATCTTCCAAACTGATGCTATATG
CTGATGCCAGTCTAGGCTGTAAAGTAAAAAGCTATATTACATCTTATGCCATATGCTTCATCTAGAAGTT
CTCATGTAGTTGTCCCTTTGTATAGTATAGTGGAGGATCCTTGCTGAACCTGCATTTGCATGTATAGTGA
ATTCAGTCAGAGAAGAGACTAAAGCCCTGTTGGGAGCACAGGCTAGTGTGGTGGGATTCCCCCCCAAAAT
GGCACTGGACCCATATGGCTCTCACTCCTAAATAGCCTCTAAATCAATAATGTGTTCTCCTAATGCCTCT
GCCCTTCCACCTCTTTTAATGCTACTTTCTTCACAAGGATTGATTGTTTGGCATTCTAAAAGTTTTAACT
TAATGTGAGGTTGCCAACCTTAAATTGGTAGGATAGATCTATCTTGAGTTGACCTAGAACTGGCATAAGG
AAGAATGATTAACCGCAAACTACAAAGCTGGGGAACTGTAATGATCTGAGTCCAAGCACCACTGGCCCAA
CAACTTTTTGAGCCTAAACCTCGTAGTTCTTAAAAGTTTAAGCTGAAGTTACTCTTAGTGATTGACTTAG
ACTCTTATATAGGTTTTAACAATCACATTTGCATCTAATGTGGGATTGGGTATCACTATCTCACTCACTC
AAATCCTTGATGTCCTCATCAAGGCCACACATCACAATCACAATCACAATCAAAGCCCACTCTACAGGAT
CAAACATCTCACCAGCACAATGTGAAAGTCCACACCAAACAAACACATGTTCTAATACCAATTATAATGT
TCTGGGTCTAAGCACCACCAGCTTAACAACTTCTCAGACTTAAACCATGTTCTATTACCATTTATTCTTT
TCATAGGTTATTTCCATGGTGTGTTAAATAAAGTAGCTAATACATGTAAGTTGGTACTAGTAATTGACTT
AGACCCTTCTATAAGGCTTAATAATCACATTCACATATGATGTGATAGGAGCAAATGACAACATGATCAC
TTTGAAATGTGGATGTGGATGTGGATGACCCACAAACTGATTTGATTAAGGAAACTGATATTTTTACTTT
CTACTTTATACTGAATTCAAATCATAGAAATTAGTGATAAATAACTAAATATTTATATCAGTTGAAGAAC
TTCCTGACTAAGTTTGTGCAGACCAATATGCTAAGCATGATCAAACAGAAATAATAGAGTATTATGTAAA
AAATGAAATGAGTATAAATTGTTTGTGTTGGAATCCTTTTTATCCTCATGCATTATCTACCCTGCTCGTG
TTAGTCATGCTAGGCTTTCATTGTTTATATCTTCCATTCTAGGAATATAGGATTATGTCTCCAATTTTAG
CTGTTCATACAATTTAAATTAATCATACTCATACCCTGCAGCATGGTTTTTTTCAGGTGAGACTCATGCA
TGAAGTAGGCTATGAGATTGGAAACTTAGATGCCACCTTAATTCTTCAAAGACCAAAATTAAGTCCACAC
AAGGAGGCTATCAAAGCCAACTTGTCTGAGCTGCTGGGAGCCGACCCATCTGTTGTCAATCTTAAAGCAA
AGACTCATGAGAAGGTCGACAGTCTTGGTGAAAATCGAAGTATTGCAGCCCATACTGTGGTCCTACTGAT
GAGGAAGTAAATATAGGTCTCGGATATCAGTCTCGAGTATGGAAATTGTATGGCATACCATGAGCATTAG
TTGTAAAACTGCCATAAATTATGGCATTGCTAAGTATGAAAGCTTGATGTGTTTGGTTGGACCACAATGT
TAGAGTTGTGTTTTCAACATTTTACCAAAACGACTTGAACAACAACGATGGTAAGTTTTGACGAGGCTAC
GGTTTCCCGATTGGTCATTAGTCTATGACGTTTGTCAAAGGCTCAAAACATGAAGTAGAATACAGACCAA
AGTCAAATTAAGCGTTTATATCTATGTTCTAAACAGTTTCCCAACTTCAATGTTGAATTCCTAGTTTCTG
CTGCATTAAAGTGACTATGGCTGCGTTCACGTGTTCAACTTTGGTGAATTCACCGGTTTCCTGATTGAAT
TTGAATTGTTCTCAAGTTCCACATTAAACCATCATATGCGTGACTACCATGAATTTCACATGACTATTGA
TTGACGTTTGGTGTGTTTTCTCTTTTCAAATATTGTTTGCTGCATGCGCCCAACACGAAAACTACGAAGG
AAATATGAATTGTATTTTTGGAAACTTTTTTGTTGTTTTGTTGGACAAGGAATGAGAATGTTCCCCTCAC
CCTCAGGTCAAAGTAGAAGCCGATTTTAAAACTCTCTGACCATTGCCAATTAATCCCATTTTTCTGATGA
TTTTCCCGATATGATTGTCGCCGTATGTTCATGTTTATGGAGTGTCATCCACTAAGAATAACCCAGAAAA
AGTCCCTCAAGACCATTTCAGAGGCGGAATTCAGACTCTTTGTAACTGGTTTTTCAAATAGCCAAAGTGT
TATCGTACCAGATTAAGAATTTTCAACGCTACAGGGAGCAGGAATCCAATCTGTTGACTTCCTGAAAAGA
TTAATTACAAGCTTTCAACTCAAACTGAAAGAGAAAGTTAGGCAAGTGGGTCGATTTGGTCGCCTATGAA
GGCTTCAAACTGCACATCTCTGATCAATCTCTTACCCTGAGAATCTCAGGAGGAATGACCCCCCACTCGC
CGGCCTCCCCTGAGACTGACAAAGCTTGTAACCAAGTAGCAACTTGTAGAAGCCACCAAGGATTTTTTTG
TCTTCTTCTAGTTCCAAAACTTGTCATATGTTCCCTGGAGAAAAGGCTGAATGTCTCCAAACTCAATATT
TTGTGTAACTTTCTGCCGATGTCACACGATTAATTGTTCTGTACTTTATCATGTTGAATACTACTGGGTA
TTCAATAGCGGCAGTTTGGAGAACAAAGTAAATTTTTTTAAAAAAGTTAAAGAGGAATTCCTTAACAAGG
AAGGGAGTATTAAATGCATGAAGAAGGTAACGTTGCAGTCAAAGGTTGATAGAGGAAGTAAGCTAATTAA
CCCAGCTGACAAAGTAATTAATTAGCTGGTAATACAGAAAATCTTGATCAAATCTTCCCTTGAAAGATGA
AGGCATCAACGGAGATATTAAAAGGATTAGCTTAGCTTTGTGTAATCCAACTGTGCTCAAAGGGACAACA
AGAAATAAAAAAATGGGGATAGGCACAATAAAGAATTGAATGCTTATGTCAGATCAGACCCGGCCAATTA
TGCCACCATCGCTCGTTGCCCTTTCTCGGCACTCTTTTGAGTAAACACTATTAATATAGCATTTATAAAA
TGGGGCCATAGAGAGTTGAATTATTCTATAGAAAGGGCCATAGCAGATAAGCCAGATTGGGACCTTCTAC
TGAATTGATCCAGGCACACAGCAAAATTTGACACATATTAAAAGGTTTCTCTTCGCTGTTTCAAGTCCTC
CCAATCTGAAGTCGGTCTTCAATTTTTTACAATTAATTTATTGTCGGTGATTACGGATTTTCCATTTTAA
ATACTTAGTAAAGCAATGGAATGCCTCCTGATGTCGCTATGCTTTCTGCAAATACTCTAATCCTGGGTTA
CAACCAATCCTCTAGAAGTGTCCACTCCACTAATCTTGATTCCACTTTTCAAAGTCATTGAACTAATCAG
ATGACTTCACCTTAATCATTGTTTAAGCAGACACTAAGCAAGAGGGTCCAAAGGGTTGGGAGAACAGCAC
CAATGACTTGCAATAGTTGTCTGCACCAGCCCTGCCTCTGATCACCTGAGATGGCTCGTGCTCCATGTTT
AACATATGAGAATGGAGCTCAGTAAAATGCCCACATGGGCGAGGCAAGCCGAAAGCTGAATCTCTTCATT
GACAAAGATGATTACCCTTGACCTTACTATACAAAGTACTAGTATGATAATCACATGGGACGGTTCTGTA
GTTCACTTTGTGACTTGCAGTGAGTTAACGAGTGAACCTACATCTACAACACGGTACCGTGTGAGTCAAA
TCTGTCGGACCTTTATTGCGGAATTAATTCGGGAAACAAATTTTTTTTTTGAAA
Theobroma cacao
SEQ ID NO: 64
MGSMLGDLPSFDPHNFSQLRPSDPSNPSKMTPATYRPTHSRTLPPPDQVITTEAKNILIRNFYQRAEEKL
RPKRAATEHLIPEHGCKQPRASTS
Manihot esculenta, CDS
SEQ ID NO: 65
ATGGGGTCTATGCTCGGTGACTGGCCTTCCTTTGACCCTCATAACTTTAGCCAACTTAGACCTACTGATC
CTTCCAATCCCTCGAAAATGACACCTGCTACTTATCATCCTACTCACAGCCGGACTCTTCCGCCCCCTGA
TCAAGTGATAACTACTGAAGCCAAAAATATTCTTCTGAGGAACTTCTATGAGCGGGCGGAAGAGAAGTTG
AGACCAAAGAGAGCTGCCTCTGAAAATCTAATACCAGAGCATGGTTGCAAGCAGCCTAGGGCCTCTACTT
CATGCTAA
Manihot esculenta, cDNA
SEQ ID NO: 66
ATGGGGTCTATGCTCGGTGACTGGCCTTCCTTTGACCCTCATAACTTTAGCCAACTTAGACCTACTGATC
CTTCCAATCCCTCGAAAATGACACCTGCTACTTATCATCCTACTCACAGCCGGACTCTTCCGCCCCCTGA
TCAAGTGATAACTACTGAAGCCAAAAATATTCTTCTGAGGAACTTCTATGAGCGGGCGGAAGAGAAG
Manihot esculenta, gDNA
SEQ ID NO: 67
ATGGGGTCTATGCTCGGTGACTGGCCTTCCTTTGACCCTCATAACTTTAGCCAACTTAGACCTACTGATC
CTTCCAATCCCTCGGTACGTATGCTCCCTATCTATCCTTTTAACTCCTCAAATCATTTTAAGTTGTATAT
ATATGTTTTTTTTGTTTAATGATCCGTCTAGTTTATCTTAATTCTTTGCTGAGTTTTGTGCCTCCTAGTG
GTTCAGATAAGGTTTTGGCTTGAGTTTAACAGCTATCAAATAAAATTTAGATTTTGGCGATTCTTTCTAA
TCCCATTTTTAGATTGCTTTCTGGGTTTACTTAGCTGAGCTATGTTAATGGGGCCTGATTAGGAAAAATT
GGTCCTATTATTATAGACACGTTAAATTTCTGGATTTATGTAGTTTTTTTTTTTTCATTATTAGATTGGG
TTTTCCTGCGGGAGATGCAAGTTGAAAAGATTTTTGGCTGTTCAAAATGTAGTATTCCTATTCAACTTTT
TTGTTAATTTTGGTTAGATTGGCATTTGCAGCACACGGATTATGAGACATGTAGATTGTGAATTCATGAG
ATGGGACAATGTCTTTTGTGGTAAAGCTAATAATGTTACGGCTTTGAAAATCAGATCTTAGATTGTGGGA
AATTACCTCGTTGTTAGGTCCAAGCAGCTGTAATGAGGACTTATCTAAATTCAACTGCAATTTGAAATTG
TGAGTGTATCTAATGGCAATTTGAACATTAATGATATGAATATCAAATGTTGCATGTGAGCATAAGCTGC
ATTTTATTCAAAATAATGTACCAGATCCAGCGAGATTGAGATGCTTTTATTTGTGAATGTGTTGGTTTCA
AAAAGCTGGTGTCGCTAAGACTAAGCTACCAACTCCTTAAAGCAAGAAACATCTTCTGCATTCTGTATGC
TCACCTGCATGAGTAGACTTTTACATTCCAATTATTCATTAGCTAAATACATTAGTTGTTTGACTGTGAG
TTAATTTTTTTTTCTGATGTTATTATTTATTGTCAAATGAATTATGCATGCCTCATTTTTTCTTAGATTC
AACCTCGCATTGATTATGGCCCATAGCCAACTAGATTTGCTTGATAAATTTGGCACCATTATATCTTTAA
AGGTAAGTATGTAAATGAAGTGGAATAGAAAGGTCTTCGCCCTACTATTTCTTTGCTTCCCTGATCCTTC
ACCTTCTTACTGCGTCTGCTGTTCAGTTGTATACATTCCCAAAACAATTTCTTTGCTTTATTCTCTCAAG
TGAAATAGAAACACTTCTGGCCAATATATGAAGCATTAGCTCTTTTACCATGCAAACCGATGGTCCATCA
ACTATGAAGTTCGAAAATTTGACATGTCCCATAGTTAATGAATTTTGTAGATTATTTGTGTAGATAATCG
GGTAAATCCTTTTGGAATGTGAATTCTCATACATATTGTTGATTTTGGGAAACAAGCTAGGGCTCATTTT
GCAGCTTCTCCAGCTGAGGCAAAATATTTGGTTTGATACTGTAATAGCAACTATATTAAGTTTTGAAATA
ACCAGAAAAAAAAAAAAAAGAAAAGGCAACAAATAGAGGAGTCAAGCCTGCGTATATAAGAGGAACAGGA
CGAATATCACAGTACTCTATTTACCTGTTGATATACCTACGCTAAGATGAAATTCTTTTTTTAAACCATC
TAAGCATATAGGCACTTGTATAAACGTTTGCTTGTTTGTTATTAACTTTGTGATATTGTTCTCTGTGCGC
TTTGATGGTTACACTTGTGATATTGTTCTCCATGTGCTTTGATGGTTACACTTCTGGATTGGTGCCTGCC
ATTAGAATTGGTCTTATTAATTCGGAAATCTCTAAAGCAAGTGATTGTGTCCTATATGATATCCTCTAGT
GCTTACAAGTTCTGGGATGAGGCTGCTGTGCTATGGCAGTTTGACTGTTAAAATTTCCATTTCAAGATAT
AAAGAAAATATGGTTTGTGAAACATGTGTGCTGGTTAGTGATGAACCTTAAATATACGACTCAATACATT
GTCCCATCTTTCCAGATTGATAATGTTTCATTGTTTGTCAACTTTAGGTCTACTTGAAATGCTTCTCATC
ACATTTTCAATGATTCTTATTCTGAGAATGGAATGAACTTCTATTCTTATTTAGAGTTATAATTATCTTT
TATGCTTTTCTGTAGAAAATGACACCTGCTACTTATCATCCTACTCACAGCCGGACTCTTCCGCCCCCTG
ATCAAGGTAATAAACAAGACTTTCCAATTTTCATCTTCAGGAACTTGTTTTCCATAAATATAAGGAAAAT
TTGTTTGCCATGTAATGCCATTTGAATATTTTTGCAAGATATTTTTTTTTTCGGGTAGTGATATTGCATG
AGTTTGTATTTCTGTTGTATCTCTTTCACTGTATGAAAATACGTTATAATATATTAAATGCCAACGATTG
CAACGCCATGTATGATCTATCTATTAAATTCTTTGCATCAAGTCATATTTACAAATTTATGCAGATGTCG
CAGTCTCTTTTGTTTGAAAACATTGTGAAGCTACTGTAGCATTAAGCTTGTTCTTGGTATCTAGAAATAC
TTCTCTTGATTTTCTTAGAAATGAGTGGAATAGAAACACTTCCTTGAGCTAAGAATGCGTGCTAAAATGG
AAAATCTAGCAAATATAAGCATAAAATCACACAGGGTGAGTAGGAAGATTGAACTAGATTCCCATGCGCG
AGGGAAAAAATGTTGTGATGAGATTACTATATTGAAAGAATTATTTTTCTTAGTTGGTTAGCATTTTAAG
TGACTTGAGCCTACTTGAAAAATCAAGAAACCAACATTGATTAATCTTTTTAGTTCAATGATAAAGGTTG
AGTTGAGCTCAAGCTCGACTTGGATTTAAACAGCCAAACTTGAACATTGTGATGCTTGACCAAAGACCCA
GGAGTTGTGACCAGTTCACAAGCTACGTGATATGTGTGAAGCAACCTTGGATGTTATATAACCACTTAAA
TGAACAGATGTTTTCATTTAATGCTCGGGAGATATTTTAAGGTAAATGTGGGATTCAGATCCATTACTGG
ACGGACCAGTTTGGCTCCCTGTAATCTGCATGCCTTTTCCCTCTATGAGTTGCTTATCAATGTTAATTGC
AATCTGGACACTATAAAGACCTTTGTTGGCTTTTGTCACTGTGCATATTGTAAGGTCATACTCAAAAGCT
TGAGCTACAGAGGTTGTGTTTAGATTTCAAAAACTTGAGCAAGTTTGGTGGTTGCACTTTTGATTTTGTA
ATGCAGTTTTTCTCGTATATGGTGACGATTTCCTCATTTGTCTGAAATCATCTTGCCAGTGATAACTACT
GAAGCCAAAAATATTCTTCTGAGGAACTTCTATGAGCGGGCGGAAGAGAAG
Manihot esculenta
SEQ ID NO: 68
MGSMLGDWPSFDPHNFSQLRPTDPSNPSKMTPATYHPTHSRTLPPPDQVITTEAKNILLRNFYERAEEKL
RPKRAASENLIPEHGCKQPRASTSC
Hevea brasiliensis, CDS
SEQ ID NO: 69
ATGGGGTCTATGCTCGGTGACTGGCCTTCCTTTGACCCTCACAACTTTAGCCAACTTAGACCCACTGATC
CTTCCAATCCATCGAALATGACTCCTGCTACTTATCATCCTACTCACAACCGTACTCTTCCACCACCTGA
TCAAGTGATAACTACTGAAGCCAAAAATATTCTTCTGAGAAACTTCTATGAGCGAGCTGAAGAGAAGTTA
AGACCAAAGAGAGCTGCCTCCGAAAATCTAATACCAGAGCATGOTTGCAAGCAGCCTAGGGCTTCTACTT
CATGCTA
Hevea brasiliensis, cDNA
SEQ ID NO: 70
CTATCGCTCTTCCTCTTCTGATATCTTTCTCTGTCTAGATTTGGCCACCGAAACCCCGCATTCCATGGGG
TCTATGCTCGGTGACTGGCCTTCCTTTGACCCTCACAACTTTAGCCAACTTAGACCCACTGATCCTTCCA
ATCCATCGAAAATGACTCCTGCTACTTATCATCCTACTCACAACCGTACTCTTCCACCACCTGATCAAGT
GATAACTACTGAAGCCAAAAATATTCTTCTGAGAAACTTCTATGAGCGAGCTGAAGAGAAGTTAAGACCA
AAGAGAGCTGCCTCCGAAAATCTAATACCAGAGCATGGTTGCAAGCAGCCTAGGGCTTCTACTTCATGCT
AAGCTTTGTTTACTGTTGGAAGTACAACATGCCGGTTGTCAATGTAAATACAAGTCAAGTCATGTCATGT
CATGCCAAATGTGTCAATTTGTGTGAAAGGGATTTGCTTGCGCAATGCTCTGAACTTTAGAGCCATACAT
GTAGATATGTGTGTAGAAGTGAGATTTACAGCTGAGTAATCAAATATAA
Hevea brasiliensis
SEQ ID NO: 71
MGSMLGDWPSFDPHNFSQLRPTDPSNPSKMTPATYHPTHNRTLPPPDQVITTEAKNILLRNFYEREEKL
RPKRAASENLIPEHGCKQPRASTSC
Gossypium raimondii 1, CDS
SEQ ID NO: 72
ATGATGGGGTCTATGCTCGGTGACCTGCCGTCATTTGACCCCCACAACTTCAGCCAACTTCGTCCCTCCG
ATCCTTCTAATCCTTCTAAAGTGGTACCTACCACCTACCGCCCCACACATAGCCGGACTTCTCCACCTCC
TGATCAAGTTATAACTACCGAAGCCAAGAATATACTTATTAGAAATTTTTACCAGCGTGCAGAGGAGAAG
TTGAGACCGAAGAGAGCTGCTACTGAACACCCAACACCGGAACATGGATGCAAGCAACCTAGGGCATCCA
CCACATGA
Gossypium raimondii 1, cDNA
SEQ ID NO: 73
GTTTATTATTAATTAAATAAATTATAGAAGAATTTTGAAGTCCCCAGCATTAGCGGGGATCCATGCTAGT
TATATAAGCATCAATTTACCCATTAATGATCCAGCTTCAGCACAAAGAAGGCTGATTCTAGAACCGAGTC
AGCCATTGTCCTTTTTTTCTCTCTCTTGGCCGGGTTCTCTTTGTAATCTCCGGTGATTTTTTGGGTGCAA
GCCACAAAACCAGCAATTTTTTCTTCTTTTCCGATGATGGGGTCTATGCTCGGTGACCTGCCGTCATTTG
ACCCCCACAACTTCAGCCAACTTCGTCCCTCCGATCCTTCTAATCCTTCTAAAGTGGTACCTACCACCTA
CCGCCCCACACATAGCCGGACTTCTCCACCTCCTGATCAAGTTATAACTACCGAAGCCAAGAATATACTT
ATTAGAAATTTTTACCAGCGTGCAGAGGAGAAGTTGAGACCGAAGAGAGCTGCTACTGAACACCCAACAC
CGGAACATGGATGCAAGCAACCTAGGGCATCCACCACATGGTCATAATGAGATTTTCTTTTGGTTTTTCA
ATGCTCATCTAAATGTATCTTCTCATACCAAATGTGTGTTGTAGAATCTGTGAGGAAAGTTGCTTTGCAC
ATTGTTGTTAATCAGGATGCCATGCTTGTGCATTGTATGTGTACAAATTAAACTCATAGGTTAATCAATA
ACAAGAAGTGTTGTTATGTTTCTGCATTCTCTTCTGGGTATTATTGATTCTTGTCGGC
Gossypium raimondii 1, gDNA
SEQ ID NO: 74
GTTTATTATTAATTAAATAAATTATAGAAGAATTTTGAAGTCCCCAGCATTAGCGGGGATCCATGCTAGT
TATATAAGCATCAATTTACCCATTAATGATCCAGCTTCAGCACAAAGAAGGCTGATTCTAGAACCGAGTC
AGCCATTGTCCTTTTTTTCTCTCTCTTGGCCGGGTTCTCTTTGTAATCTCCGGTGATTTTTTGGGTGCAA
GCCACAAAACCAGCAATTTTTTCTTCTTTTCCGATGATGGGGTCTATGCTCGGTGACCTGCCGTCATTTG
ACCCCCACAACTTCAGCCAACTTCGTCCCTCCGATCCTTCTAATCCTTCTGTAAGTAACCTCATAATCCC
TTTTAACCTAACCCCATTTTCCATAACATGTGAATTTTGTTTCTGGGTTTGAATTGCAATGAATTAATCC
CCACATGCAGTTTAGATAAATAGTGAAAAACCTTTTAAATTTTTATGTTTATGTTTCTCGGTTTACCCTG
ATATGATGTTATTGTTATCTGTTTTTTTTTTTTGTGAAAAGTATTTATTATTCATGAATTTCGGACCTTA
ATTGTCAGAATCTTTTTGGATAAAGTTGTTTGTGGTTACAAATGACTAGGGGTTTAACTTTAAATGTTTC
ATAGAATTCCAACTCTGTTATTCCTACACTTTGTTTTTTTTAATGGTTTGCATTGCAAGATATATTATTT
ATTATTTAGATGTTGATTAATCTAATTGATTGTTGTGTAATGCTTCTATATGAAGCATGTAAATAATGCT
AATACTTGTTATTATTTGTTTGCTAAAGTTATAGTTATATGTTCACACAAGGATCATGCTTCTTCTTTTT
TTTTTGCATGGGATGATGATGGATTTTCCTTTAAAACTTCAAGTTCATGCTTTCGAAGCTTTCTTATCTT
GCTGTGTATGTGGTAGTTTTTGTTCTTAGAGCTGGTTCTTAACAATGGTAACAAAGACAAGTTCTATGAT
TAAGACACAAATGAGAATTTGTTTCTATGTGGGTATACATATGCATAGACAATTAGATATTTAGACATAT
TGGTATTTATGTATTGGTTTTTTTGTTCCAGTGGTTTACAGTGGTTCCTTGCCTATCTGTTTAACCATTT
TTGAGTAAGCTTAGACTGGTTGAAAGGTCGAAATGGTTTAAAAAAAAAAAAAAAACAATTTTCACCTTCC
CTTCTATCTTTTTGAGCTATTTCAGCATGTGAGATGCTATAATGTTCATGGAGTTCATATTGAGCTCCTC
TATGTTTTAATCTGAACAGTTATATTATGATAAATGAGTCTTTTTGCTCTCATTGGTTAGCCTTTAACTA
AAATTACAGACTTCCCTTGTATGGATTTTATTTTGCTTGAACATTTTCGGGGCATAATTTGCTTGAATTT
TTTTTTGCTAGAAAGTGGTACCTACCACCTACCGCCCCACACATAGCCGGACTTCTCCACCTCCTGATCA
AGGTATCGAACAAATATTCTCTTTCTCCTTTTTTTCACTACGAAAACAATATTCTATTCTGAGTTAAAGT
AAGATACTATTGGCGCTTTAAACTGTTTGTGGTGATAATATGGTAACTTTGGTTGGTTAAATTACTTTCA
GGCTTTATCCACAAAGCATGCCATTGCCATATTACATGATAATTATTAGTCTGGGTTTCCTTCTAGGATG
TTGAATGTTGACTTATGTTTGAGACCATCCAGTGCTGGAAAATTACATTGATATGTCTGAATTGGAACTT
GATATGAAATAATTTTTCTGAAAACCTTGTGTTGTGTTCTCCCTTTGCATCCTTTTTTTCTTTCTCTCAG
ATTTCTTGATTGGTAGTGATTTGGTTCATCTGTCTAACTTTGGTTGACACTGAGCAGCAAAGTGTTTTGG
ATATTTCAGGTTCAAATCACAACAAATTGGTATTGAAGTAAATTATTAGAAATCAATTGGTGTTGTCTTT
CGTAATAGCTTTTGGTAATGCTAGCATTAGCAAGAGCTTTGGTGTGAGAATGTCATTGAAGATGGATGAA
AAATTCAGTGTTAGAAAAGTAGTCTTATTACGGTTCGTTTCTTTTTTCAAGTTTTTTTTATTGGTAAAAC
TCAGTTTATCAGTCTAATTTCTAAGTGGATTACTTTTAGCAGCTTCTGCTTCAATGTGTTTTGGATATTT
TTGGTCTGAATCCCAGAAAACTGGGGTCGAGCAAATTCTTGGAAATTGATTGGCTTTCAATCTAGCACTA
GGGAAGAGTTTGATGCGGGATGATGTCGAATATGGATGTCAACTAATTTGATGGTAGAAAAGATTTTAGC
CTTTGTCATGTAAAAGTAGAGTTGTTCTTATCTCCTTCTCAATAGCCATTGTTGTGTAAGATCAACTCTT
ACACGCCAAACGTTACAATATTTTTTACTATCAAATTTGTTAACATCCATCTTTGATGTCATCCTTTTAC
CCAAACACTTGCTAATCTTAAGATTATCGAAAGCCATGACGAAATGATTGTTATTGCACCCGAACTCTTG
CTAGTCTTAAGATTATGGATAAGCTCACACCACAAAATGCCTCAACCGGATTCCCAAGTGTTTCTCATTA
GCTAATGATAGTACACAACTAAAACCTGACCCAAGAGACAATTTACGAAACTTAAAATCTTATCTTTTCA
TTCTTTTCATTTTCACTCTCTCATCCCTTTGCCAAAACCAAGCATCAGTGCACCCCTGCCATCTTTAATT
GTCAACACCCAACCTCATGTCCCAATTGCCAACTTGTCTTACACTTTGCAAACTTCTTTAGTTGAAAATT
TCGGCTAGTTTCAATAAATATAAAGGTACTAGTGATTGAAATTGCATTATGATGCAAAATGGCACAGCTC
ATTCTCTAAATCCTCGAGTTTTTAAGTTGAATACGATTTTTAGTTCACCTTTGAACAGCTTTTTTATTGG
AGACTGACTTCTGAAGCACTTTTTTACAGTTATAACTACCGAAGCCAAGAATATACTTATTAGAAATTTT
TACCAGCGTGCAGAGGAGAAGGTCAGTAATTCATTTATGATTTTCCATAGTCATAGTTTGAAACTCATAG
ACACATGCATAAACTTTGAGAGTGAGCGTGTTGATACTTCATTTTGATTCTTACTGCCGTTATCATTGCA
GTTGAGACCGAAGAGAGCTGCTACTGAACACCCAACACCGGAACATGGATGCAAGCAACCTAGGGCATCC
ACCACATGGTCATAATGAGATTTTCTTTTGGTTTTTCAATGCTCATCTAAATGTATCTTCTCATACCAAA
TGTGTGTTGTAGAATCTGTGAGGAAAGTTGCTTTGCACATTGTTGTTAATCAGGATGCCATGCTTGTGCA
TTGTATGTGTACAAATTAAACTCATAGGTTAATCAATAACAAGAAGTGTTGTTATGTTTCTGCATTCTCT
TCTGGGTATTATTGATTCTTGTCGGC
Gossypium raimondii 1
SEQ ID NO: 75
MMGSMLGDLPSFDPHNESQLRPSDPSNPSKVVPTTYRPTHSRTSPPPDQVITTEAKNILIRNFYQRAEEK
LRPKRAATEHPTPEHGCKQPRASTT
Gossypium raimondii 2, CDS
SEQ ID NO: 76
ATGGGGTCTATGCTCGGTAACCTCCCGTCCTTTGACCCCCACAACTTCAGCCAACTTCGTCCCTCCGATC
CTTCTAATCCTTCTAAAATGGTTCCTTCCACCTACCGTCCCACTCATAGCCGGACTCTTCCACCACCTGA
TCAAGTTATAGCTACTGAGGCCAkkAATATACTTATTAGAAATATCTACCAGCGTGCTGAGGAGAAATTG
AGATCGAAACGTGCTGCCACAGAACATCTAATACCAGAGCATGGATGCAAGCAAACAAGGCCTTCCACCT
CTTAG
Gossypium raimondii 2, cDNA
SEQ ID NO: 77
TAAGCCACATAGCTCTTTAAATACACATCAAATTACGGATTACTCATGAAGCAATAGCCCAACACAGGGC
TGATTCTAGAACCAGGTCAGGCATTATGGTGGTTTCTCTCTCATCTTGTCAATCATATGCATCCCAATCT
CTCTGACATTTTAGGTGCAGGCCAGAAACCATCGTTTCATTCTTCCCCAATGGGGTCTATGCTCGGTAAC
CTCCCGTCCTTTGACCCCCACAACTTCAGCCAACTTCGTCCCTCCGATCCTTCTAATCCTTCTAAAATGG
TTCCTTCCACCTACCGTCCCACTCATAGCCGGACTCTTCCACCACCTGATCAAGTTATAGCTACTGAGGC
CAAAAATATACTTATTAGAAATATCTACCAGCGTGCTGAGGAGAAGGTTAGTAGTATTGAGATCGAAACG
TGCTGCCACAGAACATCTAATACCAGAGCATGGATGCAAGCAAACAAGGCCTTCCACCTCTTAGTTGTAA
CTCTTCGTTTTTTTCTTTGAGGCTGGTGTAAATGTATCTTCTCATATCAAATGTGTTGTAAACTGTGAAA
AAAAGTTGCTTACCCACTGTTGTAGACTGGGACACCATACATACGTGTATATTTTGTGTATAAATCAAAC
TTATAAATGGTTATCATTATAATTTTTATGGCGACCACTGTTATTTGTAGTTCA
Gossypium raimondii 2, gDNA
SEQ ID NO: 78
TAAGCCACATAGCTCTTTAAATACACATCAAATTACGGATTACTCATGAAGCAATAGCCCAACACAGGGC
TGATTCTAGAACCAGGTCAGGCATTATGGTGGTTTCTCTCTCATCTTGTCAATCATATGCATCCCAATCT
CTCTGACATTTTAGGTGCAGGCCAGAAACCATCGTTTCATTCTTCCCCAATGGGGTCTATGCTCGGTAAC
CTCCCGTCCTTTGACCCCCACAACTTCAGCCAACTTCGTCCCTCCGATCCTTCTAATCCTTCTGTAAGTA
TCCTTGTCATCCTTTTTCAACCCAACTCCAGTTTCCAAAATACATCCCATATGTATTTTATTTCTGGGTT
TTACCTGAATAACTTGTTTATTTTAGCTGTTCTGATATTGCATCAAAACCCCATATGCAGTTTACATGAG
TAGTAAACTTTTGTTACATTTTATGATTTGGTGGGTTTACCCAGATATGAACTACGGATTTCTTTTTATG
AGTAAATTTCCTAAATTCTTACCTTAATGCTATTGCCCATATTATCGGTTACTTCTCAGAACCTTTATGG
ATAAAGTTGTTTGTGGTTTCAAATAAATATATTTGCTTCCCGATCTATATTGGCTTTAAATGATTCATGT
AATTCCAACTCTGGTGTTATTACACATTAGTCATTTTATTGTTTGCATTGCAAGATGCATTTATTATTAG
GATGTTGATTAAACTGATGGAATCAAGTTTAATATGGTAAACTTTCTATGCAAAGAATCTAAGTAATGCT
AATAGTAAAACTTTTTTTTTTTTGCTAAAGTAACAAAGATCTTTGCCAACAACCTCTTGGCCTAATGGCA
AGAGTATTAGGTTGTGAGGCATGAGAGCTTGGGTTCCATCCCAAGCAACCCCATCCCCAACCCAATTATA
AAAAAAAAAGAAAAAAGAAAAAGAAGTGACAAAGATCTTTATACAACACCAGTATAAACTTGGAAATGCT
TTCTTGCACTAGATAATAATAGGAGTTTCCTTAAGATGGAAGTCTCTATTCTTTGAGAGCTTGAGATTTT
CTCCAGTGTTTCAGTTTAAAACTACTAAGACTTTAAGTTCATGCTTTAGAAGCTTTCCAGATATTGATGT
GTAGTTGGTAGTTTTAGTTCTTACAAGTTAAGACCGGATCTTAACAGTTATCATGTAGTTCTTTGTTTTA
TTTAAGATACTAGCTGAGAATTTGTGTCTATGTGGCCATATGCATAGGTATACTTGTAGACAATAATTTA
TTTATATGTATCTATATTTGTTTCCATAGTTTGCAGTGATTCCTTGCTTCTGTTATAATGTTTCCGGTTA
AGATCATAACAGTTGAAAGGTCCAAATGGTGTTGTTATGACTGTGAATTTGGAATTCAGTCACGGATGAT
GGATGAGAAAATTCCTCAAGCTCATCACAGGGAGCCTAACCTTAGAACTTGTTACAGAGAAAACCCAGAT
CTCTTTTCAGGGTTTTGATAAAAATTCGACTTCAGTTTTATTATTCCATTCTTCTCAGTTTTATTACATA
ATTAGGATTTTTTTTTTGGAAAGGAAAGAAATCAATTGAAATAAAATCTCAATCGAGATACCTGAGGATA
AACTGAAATAACATATTAAAATCATAATTGAGAAACCTGAGCCTTAATTAGAGAACCCAAATTGATGGCC
TATCCTAACATAGGTTTGGTCCACAGAGCATCCACAATAGGCGTACAACAATTTTCTCCTGCTTCTTATC
CTTTTAAGCTATTTTAGTATGTGAGATATAAGCTATTCTACGGAGTTCATCTTGAACTCCTCTATGTTTT
TAAATGAACAACTTATGACAAAGTAGTCTTTTTGCTGTCATGGATTGGTCTTTAACAAAATTACACTGGC
TTCACTTGTATGGTTTGTTACTTTGTTCAAACATTTTAGATGCATAACACACTTCTTATTTTTAAGTCAT
GTTAAAGAGTTGATTGCACTAATATTTTATATTCTAACATTTTCTTTTTGGCTAGAAAATGGTTCCTTCC
ACCTACCGTCCCACTCATAGCCGGACTCTTCCACCACCTGATCAAGGTATCGAACGGATATTCTTTCTCC
ATGTTTTTACTTTTGAAACAATAGATCATTTTGAGTTAAAGAGAGATATTCTTGGCACTTTAAACTTTTT
GTGATGAAAATATGGTAACCTTTAGTTGTGAATACTTTCAGGCTCTACCCATATGGTGATAAATTGATAA
TATATTTTCATAATGTGTGTCGTATAGCTCTGGCAACTTTTTGGCTAGGTTACTTTCAGGGATGTTGAAT
GTTAAGTTGATGTTTGAGAGCAGAAGTGCTGGAAAAGTATGCTGAGATGTCTGAATTGAACTTATTAATA
CATGATTTAGTTTTGTTGGAAAAATGGTATTTTCTTCACAAATGGGCTTCAAATAAATAGCTATGGATTG
AGAAATTGATCTTTATGTAGCACTTAATGGATGCCATTGCTACTATTTGTCACACTTTTTTTTTTTTATA
ATCTTCCTCTGAAATGGTGGAATTGATTTATCTTGTTTGTTTAGTTCTTTGTTTCCTTCAGTAATCTATA
CGCCATAGATTGATTATTTGAAATTCTTCATCTTCTCTCACTTTTGCTTTGATTGATATCAACAAAGAAT
AGAATTTTTTTTATCAGAAAAATCATTAGTTGCTTTACATGATTGAAATTGTTTTTGAAGTTTTAACAAC
CAGAATGAACGTGTTTGAGGTTCTTATCCGTACACGTGTTATGCTACTGATATAAAGGCACCAGTGGTTG
GAATTTCATTCTGTTGCAAAATGCCACAGCTCATTGACTTAAATTCTTGACTTTCTTTATATTTTTTTCC
CATATATGATTGACCTGTCGAATACTTTTTAACAGTTATAGCTACTGAGGCCAAAAATATACTTATTAGA
AATATCTACCAGCGTGCTGAGGAGAAGGTTAGTAGTAGTAGGTTTTCCTTCTTTTGATGTTATAATACTG
CATATACAAATAATTTAATCATCGTAAATAGAAATACACAGGCACATGCATGCACAGATAATTCATCTAG
TTTCTTACTGCTTTCCCATTGTAGTTGAGATCGAAACGTGCTGCCACAGAACATCTAATACCAGAGCATG
GATGCAAGCAAACAAGGCCTTCCACCTCTTAGTTGTAACTCTTCGTTTTTTTCTTTGAGGCTGGTGTAAA
TGTATCTTCTCATATCAAATGTGTTGTAAACTGTGAAAAAAAGTTGCTTACCCACTGTTGTAGACTGGGA
CACCATACATACGTGTATATTTTGTGTATAAATCAAACTTATAAATGGTTATCATTATAATTTTTATGGC
GACCACTGTTATTTGTAGTTCA
Gossypium raimondii 2
SEQ ID NO: 79
MGSMLGNLPSFDPHNFSQLRPSDPSNPSKMVPSTYRPTHSRTLPPPDQVIATEAKNILIRNIYQRAEEKL
RSKRAATEHLIPEHGCKQTRPSTS
Vitis vinifera, CDS
SEQ ID NO: 80
ATGGGGTCTACATTGGGCGACTGGCCTTCGTTCGACCCTCACAATTTCAGCCAGCTTCGGCCCTCCGATC
CTTCAAATCCATCAAAGATGATCCCTGCCACGTATCATCCTACTCACGATCGGACCCTTCCACCACCTGA
TCAAGTGATATCCACTGAAACCAAAAACATCCTTCTTAGACATTTCTACCAGCGCGCTGAAGAGAAGTTG
AGACCALAGAGAGCTGCCTCAGAACACCTGACACCAGAGCATGGATGCAAGCAACCCAGAGCTTCTGCCT
CAGACTGA
Vitis vinifera, cDNA
SEQ ID NO: 81
CGGGACTGGAAAGAATGGCGCCAAAACGACGTCGTTTGTTGTATTTGCAACCGTTCGCGATAACTCCTGC
GTAGAATCCAGACGACTGCGAACATCAGGTGCCTCTGTCATCCGGCTCTCTCTCATGGGGTCTACATTGG
GCGACTGGCCTTCGTTCGACCCTCACAATTTCAGCCAGCTTCGGCCCTCCGATCCTTCAAATCCATCAAA
GATGATCCCTGCCACGTATCATCCTACTCACGATCGGACCCTTCCACCACCTGATCAAGTGATATCCACT
GAAACCAAAAACATCCTTCTTAGACATTTCTACCAGCGCGCTGAAGAGAAGTTGAGACCAAAGAGAGCTG
CCTCAGAACACCTGACACCAGAGCATGGATGCAAGCAACCCAGAGCTTCTGCCTCAGACTGAGCTTTTCT
CCATTGGGAAGTCAAATATCGTCTTCAGCTTGTATATAACTATATATGTATTCCCATACTCAAATGTGTA
AACTGAAAGAAGACTTGCTTTATCATTATCGCAAAAAATGCTTAGCCACAGGCTAGTAGATGTTGGGTGT
AAAAATCAGATTAAGATATAGCTGGATTATTCCCATCCCAGACAGTGAAATTATGAAATTGTCTTTCTTC
TCATA
Vitis vinifera, gDNA
SEQ ID NO: 82
CGGGACTGGAAAGAATGGCGCCAAAACGACGTCGTTTGTTGTATTTGCAACCGTTCGCGATAACTCCTGC
GTAGAATCCAGACGACTGCGAACATCAGGTGCCTCTGTCATCCGGCTCTCTCTCATGGGGTCTACATTGG
GCGACTGGCCTTCGTTCGACCCTCACAATTTCAGCCAGCTTCGGCCCTCCGATCCTTCAAATCCATCAGT
ATGCTTTGGCTTTATCTAAATTTTATTCATTTATTTATTTATTTTGGGTTTCTCTTCACTCGATTTGATT
GTGTGCACAGATGCATTTCATCATTCTTCTTCCATAGTTGCATCTTAGGTTTTCTGGGTGCCCCTGGCTG
AGTTCATTAGAATTTCAGGGGCTTGTTGAATTCAAAATATGTATAACCTTTCGTTTCTGAATTTGGACCC
TAATCTGTTGTATAGCACTGATCAATCAAGCACTGTGTGTGGAAAATGTTCTGGTTTGAGAGTTCTTAAG
TCAAGTAGTAGTATTAGACTATTATCCTTTCTGATTATGGCTGAAGCTATTGACTCTCTTGGTACGTGGA
AATAAGATTTGGGAATGGGAAATCTATCCATTTCCCGTTTGTTACCGTGATTGGATTTCTTATTAGAAAT
TAGAAATGGGGATAGGAACATGGAAACCAAAACCCACCCTTTTAGAATTTTGATTCCTCTCTTCAACATG
GCAATCTGATTCACTTCGATTCCGATTTATACTTCCATTCCTATTCCCAAGTCTCATTTTTGGTCTCACC
TGATGGCAACAAAACTGATCTTTCTAGATTTTGTTTGCTCATGTTATTGTTTAATCTTTCAGCTTATTGA
TCAACATTTTTCTCCTTTCAAGAATCCATTTTAGGCCCTTATTCCTAACTGTTTAATGATGAGTCCAACA
TATCTTTTCCTCTACTTTTCCATGTACTAAATGCTTATGCTTGTAGAAAATGAACACTCGTTAGCATGAA
TTATAATTTAACCAAGGCATATGTTCATTTATTAATTTAGACTTGACAATGCACTGGAAATCTCTGAATT
GGTTTGAAGCTGTTAAGGGGTTCCAATAGCTTATAAAACTATTGAAGATTGAAAAAAGGTCTATTGATGT
ATGCTAATGTTCAAATAGATTTGTTCAGGACTAGATTATGTTATAATTTTTAGTGAATTTGTCTGAATAT
CTGCTCTTTATTTGTTTACCCTTGTTGTTTATTCAGTCTAGCCCATTTTCTGACATGGTGTAAGGGTAAT
TGTTTCTGAGACACATGCCAACCCAGTTTAAGCTCTGTTTCCCTGCTAATGGGAGAGTTGGACTACAACA
TAGCAGGTTGGGTCAGGTTAAAGATTTAACCGAGGGTCAATCTTATGCATAAAGGCTCAGTCATTGGCTA
AGCTCAACCTGTGCTCCAGCAGTAGTTGCTTAACCTGGGTTGAACCCAAGTAATTTTTTATCTTGAGTGA
TGTGGGTCAGGGTTAGGTGAGGTTTGGTTGGGTTAAATTAGGCTTTTGGTTGCCAAAGAAAGGTCAAATG
CAGGTTGTGCTTTTTCAAAATAACTAAAGGAGAAGAGGAGTGTGTATTAACAAAATTTAGACGGCAGCAT
TAGCAATGTGGTGATGGTGATCAGAGATGGCATGATGCAAAATTGGTGATGGGGAAGATTGGTGGGTCTG
TGGTTTCCCACATGATACTTTGAGAGAAAATGATATTGATTTGAGAGTTTATCAGCAAGGGTTATTTGCT
TTATTTTCTTAAGTCACAAACTCTATTGAAAACCCCAAATGAAACAACGAATGCACAAGAGGTCAGATTC
AGGTTACTCAACACCTCAGACCCAATCCACCACCAAAGTAAAATTGTTTTGGAGATTTTCCTGCCCATGC
AGCTCCCATGGATCAGGTTGGGTGGGCTCAAATCTGCCTAAGTTGGAGCTCTATATGAGGGGACTATTTC
TTTGAAGACAAAATGAAAAGTAAGCATTCATTGAATGTAAGCAAGCAATCATTATTGTGAAACGTACTTT
ATAGCGCATTCATTGTGAATTATAGTTGTGGCTACTTGCTTGTGGTTTTCACTCTCTTATTCTCTTGGAA
AACAAGGACCAGGGGAATGAGGGGTGAGTTTCCTTGCAAGTACTAAGGGGTGAAATGCACTAATTATTTG
TAGTTATTTGGATATATGTTATTAGGCTTAGATTTGATTACAATTTAGCTAGTGATTATTTTGGAATATT
TTCTTTTTGTTTCTGTTTAAGATACATACTGAATTTAACCCGTGTTTATTTTTGTGGAAATTAATTCCAC
CTTTTCTACAATCTGTCAAAGATATTTCCCCCAATTGCAATAAAGTGCATTGTCTTTATTTTTCTTAAGA
TGCATTTATGTTTTGTATCAGAAGATGATCCCTGCCACGTATCATCCTACTCACGATCGGACCCTTCCAC
CACCTGATCAAGGTAATGGACTCCTTAGTCTTTCATTTTTGGATTTTTTTTCTTTTTTTTCATTTGTTTG
TTTGATTTATTTATTTACTTTTTGGGAAGGTGTGGTTGGTATATCCACATATTGATAATCATTGTAATAA
ATCCATATAGAAACTGGTTGATGCTACTGGATCTCGTCTAATTATTTGGTGATGTTATTGTGAATATTTT
GTTTTTAACATGTCTTTTGACTGGTATGCCTTTTTTGCTTCTTGAGAATATTTAACTAGAGGGACAGATG
TTTGCCCCAATTCAACACCATTATGTTGCAAAATGAAACAATTTTAACAGTGTTAATCCATGAAGTTATA
TGGGCATCCACTTTTCTTTATGGGGAAACCCTAGATAAATAGGCTTCAGAATCACATTTAGATTGGAAAC
ATACATTTATTTGTAGTGCCTGTAGTTAAATAAAATTGGGAGGTTTCTCCCCACTTACACTGAATTTGAA
TCCAACATCCTTCTTACAGAAACCTATAGTCTCCTAGTGATATGATATCCTTGATGTGTACTCCAGAAAG
CACAAGTTCAAAAAAAAAAGGGGGGGAAAGACTTGTATGTGAACTTCAGATCTTAAACTTGATGCCAGAG
GATTGAGGTAATAGGGGAATTCAAATCCTAAATGCATGACCATCTCATGATGTGCATGATGTGGGTGCTT
TACTCATTTCTTGCTTCATAAGTAGTCATAACATGACAGGTACACTTGTGGTAACCTTGGTCACTGAGGC
TCAAGCCTTAAGGTTAACCATCAAGGGCCTCAAAGCAATTCTTTGTGATGCTGGCCTGGAGGCATAGGGG
TGTCCACCACATTGATTGTCCTTTATAGTTGTTGTTCAATGTTCTTTGATAAGTTTCAGGTGAGTCTCTT
TAAACTATTACTCTTTTTATCTTCAGGGTTGTCATTGCACTTCCATCAAACAGTATTTCAAAGTACAGAT
GGTCTTTCAGTGAAAATTTCAACTATGATTTAAAAAAAAGGTCCTAATGCCCTGTAGTTGTGCAACTGAC
ATCTTATTACTTTAAGAAGATTCTCAAATAAAGGTTCTAAATTTGCTGCACTTTGGGGTTTGAAATCTGA
TTTCAATAACAGTGAAAGAAAGGCGTAATTGCAGCATTTTTGTATTTGAAACCTATTCAATGAAAGGTGA
TCATGTTGGGGTGCAATAATGCCCACTCTTAGGCCTGGGATAAATCTCCCAAATGATGGTGATGTTGAAT
ACCATAAAAGGCTTGACCTATCTCATTGGATACCAATTGGTTTTCAAATGAGATGGTCAGAGCCTGATTC
AAGAATTTGTATAAGAGCCAATGTCATAGGTTTGGCTCATGGGAGCCTCTTTTTGGGTTACACAAATGAG
GCTAAATACCATAAAAGGTTTGTCCTACATTCACTGGACATTAATTGGTTTTCAAATGAGATGATTGGAG
TCCGGTCCAAGAAACTAAGTAACTCTACTTCCTGTAATTTGGATGTTTGCTTCATAAAGTTTGATTCTAC
CTAGCCACTTTAGTTTCATCTTGCATCTCACCCATCTAAATCCTCATGGCAGTGATATCCACTGAAACCA
AAAACATCCTTCTTAGACATTTCTACCAGCGCGCTGAAGAGAAGGTTAGAATTCAGTTCCTTATGTTGAA
TCAATAAGCACACATAGCAGAACCTAGTTTTTTTAGCAACTCATTTCCTTCCTACCTGCAGTTGAGACCA
AAGAGAGCTGCCTCAGAACACCTGACACCAGAGCATGGATGCAAGCAACCCAGAGCTTCTGCCTCAGACT
GAGCTTTTCTCCATTGGGAAGTCAAATATCGTCTTCAGCTTGTATATAACTATATATGTATTCCCATACT
CAAATGTGTAAACTGAAAGAAGACTTGCTTTATCATTATCGCAAAAAATGCTTAGCCACAGGCTAGTAGA
TGTTGGGTGTAAAAATCAGATTAAGATATAGCTGGATTATTCCCATCCCAGACAGTGAAATTATGAAATT
GTCTTTCTTCTCATA
Vitis vinifera
SEQ ID NO: 83
MGSTLGDWPSFDPHNFSQLRPSDPSNPSKMIPATYHPTHDRTLPPPDQVISTETKNILLRHEYQRAEEKL
RPKRAASEHLTPEHGCKQPRASASD
Malus domestics, CDS
SEQ ID NO: 84
ATGGGGTCTTTGTTCGGTGACTGGCCGTCGTACAACCCTCACAACTTCAGCCAGCTCCGACCATCCGATC
CTTCAAACCCTTCTAAAATGACACCTGCAACCTACTATCCTACTCACAACCGGACTCTTCCGCCACCTGA
TCAAGTGATAACTAATGAAGCCAAGAATATCCTTTTGAGGCACATGTATCAGCATTCTGAAGAGAAGTTG
AGACAAAAGCGGGCAGCGCCAGAAAAACTCTCACCGGAGCCTGTATGCAAGCAACAGAGGTATTCTGTCT
CAGATACTGCCTAA
Malus domestics, cDNA
SEQ ID NO: 85
ATGGGGTCTTTGTTCGGTGACTGGCCGTCGTACAACCCTCACAACTTCAGCCAGCTCCGACCATCCGATC
CTTCAAACCCTTCTCAATGTCATTGTAAATTTGTAATGCTGAAGAGTGCTGGCTGCTTTGCTGTTGGACC
TGCTTTTGGTCACGGCCCCAGATTAGGATGGAATGTTCATTGCTCAAGTAATATTTATAGCCTTTCATGG
GTCCCTAGGAAAATGACACCTGCAACCTACTATCCTACTCACAACCGGACTCTTCCGCCACCTGATCAAG
TGATAACTAATGAAGCCAAGAATATCCTTTTGAGGCACATGTATCAGCATTCTGAAGAGAAGTTGAGACA
AAAGCGGGCAGCGCCAGAAAAACTCTCACCGGAGCCTGTATGCAAGCAACAGAGGTATTCTGTCTCAGAT
ACTGCCTAA
Malus domestics, gDNA
SEQ ID NO: 86
ATGGGGTCTTTGTTCGGTGACTGGCCGTCGTACAACCCTCACAACTTCAGCCAGCTCCGACCATCCGATC
CTTCAAACCCTTCTGTGAGTTTTCACTTTCTGAAATTCTGAATCAAACCCCTTTTTCCACCTTCTTATTC
AAGCTGAATAGTCGTGCAAAGATTTTCGTTTTTGTTGAAGTTCTTGATTTTTCTGAATTGGGTGGTTCTT
AATTCAGTTAAAGGTGAGGAATTGTGGTTTTTCTGTCTGCATCAAGTTTATTTCTGGGACCTGTTTGAAT
TTCATAGGAAATTTGGAGTAATTTTTGTAATTAGTTATAACTAGGAGATTTTTGTGCAGATTTTACTTCT
AAGTTTATGGTAATCGTAATTGATGTTGCAGCAATGTCATTGTAAATTTGTAATGGTAAAGTGTGTGTAG
CTTAATATAAATATAGAATTTCAGGTCATAAATTTACCACTTTTGTTGAATTTCACAATCCTGAAATCGG
CTTAATGATATTTGGTTAAGATGGTGCGACTGTTTGGAATTGGATCATCGTCTTGAATTATGCTATATGT
TTGATTTTGTGATCAATGAACAATAAGCTGTTGTTGCTTGTGATTTGTAATATTTGGATCTAAATTACGT
TCGATATTTCTGTTAATCACTTCACTGATCTCTAAAAGTTTCGTGCTTTGTTATTGCATTGATCTTATAT
AAGTGTAATTATCAAGATTGGTGCTTTGTTCCAGTTACGATATACTGTACTGTATTGCTCAATTCTCATA
GAGTTCGTAAGAATTTTGAAATCAGAAGCATAATTCATAGCTATGCAATATCTCATAATTTTTAACATTC
GGATTGAAGTTACATGGTTAATTTACCATTTTCATGAAGCTGATTCACTGGCTTTCTTAGTTAAGGTAGA
AATACTAGCATTTAAAGAGCGTCCTAAATTAGTTTTGCCTTCCTGAATCCGAGCTATAGAGAATATCCGC
ATAACCTGTCAGATATAAGGTGTTTTTCTTGTCTTCTGATTGGCGGGTTCTTGTCAAAACCAACCCAAAA
CCAAAACCAATCGGTAATGGGTGGAGAGACCTGATTCAAGTTTAAACTGTGAGATGTGTGGTTACTATAT
TCTCATCATTGCCATTCACGTATAGTGGAACTAAGACCAACACGTGTGGTTAGCAATGTTAGGGCACATG
AAGTGTGTGTGAACAAAGACACGGAATGAATTGTCCACTGCCATGTTTAATTTTCAACATAAACTTCTGG
TGCTTGAGCTTTTTCCGGAAATGGTTGATATGTGCTATAGCTGAATTTTGGATTTGTTTCAAACCAACTG
AGCTGGCATTGTGGGCCACTAGGACGAAATCACTGAAAACGTTACAATGTTACAAACATAGAAATATAGT
AAGTCGGAGAAAATTACACGTGCTCTTTACATCTCATACTCTAATTATAGAGAGAAGCACGATTTTTGGT
AAAAGGTTGCTTCGTGATGATTCTGAGTCATACGTTCATACATTCTGGTGGGTATCAGCCATTGTTTGGC
TTGAGTTAGTGTTGATGTAGTGAAACGAGCCTATCTAATGAAAATTATCTCTCTGTTCCAACCACCAGAC
TAGTATATATTTGAGCTTATATTCTGCTAATAGAATTATAAACAATTTAATTACAGCTGAAGAGTGCTGG
CTGCTTTGCTGTTGGACCTGCTTTTGGTCACGGCCCCAGATTAGGATGGAATGTTCATTGCTCAAGTAAT
ATTTATAGCCTTTCATGGGTCCCTAGGGTACTCGTAGATGCATCTTCAAAAACTGAAGGGATATAATCAA
TCTAATTATTGCTTTCTTGTTCTTTTAGCAGCCAAAAGAAAAAAGGAAAACGAGAAGATGTTATGAGTTA
TGCTATGATGTAAAGTGATCAAAGTCAAATGCATTCTTTTCTTCTTTTTAAGTAATGCTTCCATTTTTGT
CTGTAGAAAATGACACCTGCAACCTACTATCCTACTCACAACCGGACTCTTCCGCCACCTGATCAAGGTA
TTAATGAATTTGTATTTCCTTAGTGTTTCATTCTGGATTGTTATTCTTTTCTATTATTTCCCTATTTTTC
CCATTTCTGTTGATTAATTTTCCTTTCCGTGTTGTATTATTGTGACTCTTTGAGAAAGGTCCATTCTAGT
TAGTTAAGCTTTTTAAAATAAATCCTGAAAACTGCAAGTTGATCTGTTCTGGACGTGGTTTTGACTCTAG
ATATTCGTTAGGCAATGTGAAATCGTGTAGTTTGACAACGTGAAATTGACCCTATCAAAACCCCTTTGCA
GTGATAACTAATGAAGCCAAGAATATCCTTTTGAGGCACATGTATCAGCATTCTGAAGAGAAGGTACTTG
ATCAGCATTACATCCGCACTTGACGCCCGGTTTCCCCCTTTTCACATGAATGCTTACATACACATGATTG
AGAGAGTGAGAGAGGCTTTCTTCTAGTTTTTCATGGACTTTTTCATTGCAGTTGAGACAAAAGCGGGCAG
CGCCAGAAAAACTCTCACCGGAGCCTGTATGCAAGCAACAGAGGTATTCTGTCTCAGATACTGCCTAA
Malus domestica
SEQ ID NO: 87
MGSLFGDWPSYNPHNFSQLRPSDPSNPSKMTPATYYPTHHRTLPPPDQVITNEAKNILLRHMYQHSEEKL
RQKRAAPEKLSPEPVCKQQRYSVSDTA
Prunus persica, CDS
SEQ ID NO: 88
CCGATCCTTCCACTCCTTCTAAAATGACACCTGCTACCTATCATCCAACTCACAGCCGGACCCTTCCCCC
ACCTGATCAAGTGATAACCACCGAAACCAAAAATATTCTTTTGAGGCACATGTATCAGAATGATGAAGAG
AAGTTGAGACAAAAGCGAGCTGCATCAGAACATCTTTTACCAGAGCATGGATCCAAGCAACTTAGGGCTT
CTGTCTCAGATAATGCATAA
Prunus persica, gDNA
SEQ ID NO: 89
TTTTGCATTATCTGAGACAGAAGCCCTAAGTTGCTTGGATCCATGCTCTGGTAAAAGATGTTCTGATGCA
GCTCGCTTTTGTCTCAACTGCAATAAACAGTCCATGAAAAAACTAGGAGAAAGCCTCTCTCTCTCTCTCT
CAATGTATGTCCGCAGTTGTGTGCAATGGGGAAAAGGGGGCATAAAGTACTCATGTAATACTTATACTCA
CCTTCTCTTCATCATTCTGATACATGTGCCTCAAAAGAATATTTTTGGTTTCGGTGGTTATCACTGCAAA
GGGTTCAGACAGGTTAATTTCATTTTGTGAAACAGCACGATTAGCAAATTGCTAATCAATCTCAAGGGTA
AAAACTACACCCATAACAGTCAACTTTCAGTTGTCAGGATTAATTTCAAAGCTTAACTGGAATGGCCTTT
CTTAAAGAATCATGAGTTGTTGATACAGGAACTAAGCCAATGGGTAAGGATCCAGAATGAAACAATGAGG
AGTTATAAAGTTTTCATTATTACCTTGATCAGGTGGGGGAAGGGTCCGGCTGTGAGTTGGATGATAGGTA
GCAGGTGTCATTTTCTACAGATAAATGGAAGCATAATTTTAAACAAAGAAAGAGAATGCGTTTAATTCTG
ATCTATTTGCATCATAAACCTAACTCATGACATGTTCTTTTTGTTTCCTTTTTAAAATTTGTGGTTGTTG
AAAGAACAAGCAAATAACAAATTTTGCATTAGTTAAATATGTTCAAATTCTGAAGATGCATCCGTGAGCG
CCCGAGCGACGTACAACAATCTATAATCTGGATTTGTGACCGGAAATAGGTCCAACAATGAAGCACCCAG
AACTCTACAGCTGTAAATGATAACTTTATAAAATTATTAGCAGAATATAAGCTAAACTGCAGAATAGTCT
GGTGGTTTTGGAACAGAGAAACATTTTCCATCAGATAGGCCTGTTTCACCACATCAACACTAATGAAGCT
AAATGATGGTTCATACACACCAGAATGTATGTTTCAGACACATCACTAAGCAACCTTCTACCAGGAATCC
TGATTCTCTCTACAAGTTGGCTAGACTTAAAAGTTTTGCTCTTCGGTTCATCTCATAGCACTGAGTCCTA
CTGTAACACAGAATCTTCACCTTAACTTTGAGAAATGCTATTATTCTTAAGTATTGGGAAGTAATGTATA
AATAGAATTTATAAGTAGAAAACAATATTTTTCGGACTTCCTCACTAAATTTCTATGTTGAAAACATCAA
GCAATATGTCCTAGTGGCCCACAATGTCAGCCCAGTTGGTTTGAAACAAATCCAAGATTTAGTTACCTAG
CATGCATCAACTATTTTTGGAAACACTTAGGGTTTTCTTAATGTATAAAGTCTAAGCCACTCTGCATATT
TTCATTGGTTTTGGATGGACTAACATGGCACCAAAGTGCACAGTTGGTCATGTGTCAGACCCGTTTTGGT
CGTACGTGCTTCAAGTCACCTAAGATAATTAATGCATGTGTTATACTAAACGCGAAAGAGAATGCAGAGG
ATATAGAAACTTGAAACTTAATATATACGAATCCCAGGCCTCTCCACCCAATTACTAATTGTGGGATGCT
TGAACTCAAGAGTTTCTAAACAACTTACAAACTAGAAGAAAAAGTAAACACCTTATATCTGGCAGGTATT
TATGGGGATTTTCTCTATAGCCCATATTTAGGAATGATAAACATAATTCGGACGCTGTTTAAATACTAGA
ACTTCTACCTTAGCTTAGAAACGGACTAAATGAGCTTCATGCATATGGTAAATTAATCATGCAGTATCCA
TGATCCCATATCATTCCAATATATGATATTTCAATCCAACTGCTATAAAATCTGAAGTTAAAAATAATGC
ATCTGATTTCAAATTTCTTGTGAACTCTACAAGACTAGAGCACTATATGTTAGCTGCAGAACAAAGCATC
AATCCTAATAAATATAAGACCAACGTAATAACAAAGCATGAAACTTTCGGAGATCCGGGAAGTGAAACAA
TGCGCGTAGTTTAAGTGAAAGACTACAAATCAGAGACATCAAAGCACAAACAACAGCAGCATATTGTTCC
CTGATCCAAATATCAAACGAATAGCATTTCTTATGAATTCATATGAGATATGGAAGAAAACAAATGAAAA
TATACAAGAAGATGTTCCAATTCAAACCACACTCATTTGCACACGATTTATTACAATGAAAGATTTATCA
TTTCAATGACATTGCTGCAACATCATTTACCATAAAATATAAATAAAATAAAAAGAAGCAAAATCTGCAC
TAAAATCTATCTTTTTGGATGTACAAACAAAAATCATTTCAAACTTTCCATAAAATTCTTTCAAACTTTC
CATAAAATTCAAACCGGGTCTCGAAATTTTTTGATGAAAATAACAGGAAGCCCTTACCTTGAGCTGAACA
GTATCATAAAAGGCAGAATCTTTTTTTCCCACAACAACTCAGTTCAGAACAATCAACAACTTCAGCAATT
TCACCATAACAGATGTACAAAACCAAACAAAAAGAAAGCAAAGAACTTCACAGGTAAAGAAAAATCAAAG
CTTTGCATGTTTATATACAGCTTAAAAGAAAAAGAATGGCAAATTGGGTTTGATTGAGATGCTGAAAACT
CACAGAAGGAGTGGAAGGATCGGCGGGCCGGAGCTGGCTGAAGTTGTGAGGGTCATATGACGGCCAGTCA
CCGAACAAAGAACCCAT
Prunus persica
SEQ ID NO: 90
MGSLFGDWPSYDPHNFSQLRPADPSTPSKMTPATYHPTHSRTLPPPDQVITTETKNILLRHMYQNDEEKL
RQKRAASEHLLPEHGSKQLRASVSDNA
Fragaria vesca, CDS
SEQ ID NO: 91
ATGGGTTCTTTGTTCGGCAACTGGCCCTCATATGACCCTCACAACTTCAGCCAGCTCCGACCCTCGGATC
CCACTACTCCTTCTAAAATGACTCCTACAACCTATCATGCTACCCACAACCGGACCCTTCCGCCACCCGA
TCAAGTGATAACTACTGAATCCAAGAACATTCTTCTGAGGCACATGTATCAGCAGCATGCTGAAGAGAAG
TTGAGACAAAAGCGAGCTGCATCAGAAAACCTTTTACCAGAGCATGGATCAAAGCAACTTAAGGGTTCTG
TCTCAGATAAGTCCTAA
Fragaria vesca, cDNA
SEQ ID NO: 92
GGTGGGACAAGAAAGAATTAGAACAGGATCGTAGGCTCTATATAAAATGGCACACATGGATTGATTCATA
GATACCAACTCTGTGCATAATTCAGGGTTTGTCTCTAGAAACCAACAGGCCATTCTCTCTGTTTCCGATT
TGGTTTGCTGCATTTCATTTCATGGGTTCTTTGTTCGGCAACTGGCCCTCATATGACCCTCACAACTTCA
GCCAGCTCCGACCCTCGGATCCCACTACTCCTTCTAAAATGACTCCTACAACCTATCATGCTACCCACAA
CCGGACCCTTCCGCCACCCGATCAAGTGATAACTACTGAATCCAAGAACATTCTTCTGAGGCACATGTAT
CAGCAGCATGCTGAAGAGAAGTTGAGACAAAAGCGAGCTGCATCAGAAAACCTTTTACCAGAGCATGGAT
CAAAGCAACTTAAGGGTTCTGTCTCAGATAAGTCCTAACAAGCAAAACTGCCTTTATCACTTCCAACTGC
TCATTTGTTCTCACATGGATACTGGAAGTTCAGCATTCCCATCAGTGTGAATATTAGTGTCACAGGCAAA
AGATGTGTAGACTGTACCCTGTCGTAGATAGAAGGGGTATTTGATTGCACTTAGTTGTAAAAGTTGCTTC
ACTAGACATGTAGACTTGCGTGTACGAATTAGATTACAGCTTTAAACAAATAAAATGAATAGTTACAAGG
TTTGCTTGTGTTCTGGTTCTATATGTCTTTACAAATGTTAGTTCCATGCTCATTTAAATCGAATGAAGAA
CATGCTTCCCCCAAAATTGCTTGTATCACGTGACTGCGGGTTTGGAAAATACATAAAACTGATAAAAGAC
AGCATATGTCAAC
Fragaria vesca, gDNA
SEQ ID NO: 93
GGTGGGACAAGAAAGAATTAGAACAGGATCGTAGGCTCTATATAAAATGGCACACATGGATTGATTCATA
GATACCAACTCTGTGCATAATTCAGGGTTTGTCTCTAGAAACCAACAGGCCATTCTCTCTGTTTCCGATT
TGGTTTGCTGCATTTCATTTCATGGGTTCTTTGTTCGGCAACTGGCCCTCATATGACCCTCACAACTTCA
GCCAGCTCCGACCCTCGGATCCCACTACTCCTTCTGTAAGTCTTCACTCTCCCAATAACCAATCTTTGAT
TTGATTTGATTTCTTGTCAAAGTTTTCTGCTTTAATCGTCTTGTTTAATAAGATGTAGTGTTTGTTGCCA
AGTTCTGTTTGTTTTGCTCTTTCTGAACCAAGTTGTGTGAAAAGAAGGTTGCTTTTTGTTGTAATCTTAT
TCAGTTCTAGATGAGGGCTTCTGGGTATGTGCATTAAGAAACTTTTGAGGCCCAGTTTGAACTGTATCAG
AATTATGGGTTCTGGTAGTAACTATAATCTTGGTTCTTGTCAAGAATAGTGTAAGTAAATACAGAATTCT
AGCATCCCAAGAACTTATCAGTTCTTGAATTGTCACTAGATTAGCTTAATCCATATATTACAGTCCCTTA
TGTTGCTCGAGTTAGTCAGAATTTATCAGATGGATTTTCTGTTTGAGCTTTTGATCATTGAACAATGTGT
TGATCTTAGTTATGGCTTACTGTGATTGTTAACAATCATGTCAATTAGATCATCATTCCTCCGTAAAGTT
TCATTCTTTTTTACTATATTGATACAATTAAAAATGTTTCCAGCCAAATGAAGCTTTGTTCTTCAGTTAA
CATATAGTGTTGTATTCAAATCTTAGAGTTCACAAGAAATTTGCAAACAGATGCATCAGTTACAACTAAG
TGCAGTTTGATATATTTTGGCATTTGGACCTCTTCTACATGGCAATGATGAGTGATTATAGCTTCTACTG
ATGAATATTCCATATACATGAAGCTCATTTAATACCTTGCTAAGTTACAGAGGGAGAACTAGTATTTGTA
AAGATGCGTATTAGGTTTTTCCTTCCTATATATGGCTGTTATAGAAAATATCTCCAGAATTGTCTGCCAG
ATAGAATGCGTTTAATAAGGTTTTGTTGTATTTAGTAAGATATTTTGCCTTTCCTTATACCTAGAAGACA
ACTTAGCAACATTTATGTTTAAGGTGAAGGTTATGTTTCTTGTATGACTGATTGCTAAGAAAAGAATGAA
TTGAAACTGCTAAGAGTTTATACCTACCTTGTAAAGAGAAACAGGATTCTCAGTGAATAGGTTGCTTAAC
AATACTCTGAGACACATGTTAGTATGGGTATCAACCATCATTCAGCTTGGCTAATTGGCTTAGTGTTGAT
GAGGTGAAAAAGTCATATCTGATTCCATTTACCAGAATACATATTATATCTCATATATACTAATAATTAC
AGCTGCAGCATATTGGCTACTTATTCTGATCACGAATCTAGATTAGAAGGGAAATTGAAGTATTAATTAT
AGCTTGCCATAGGTCTCTCATCAAAATTTGAAAATGATGTGAATCAATGCTTGCATGTCATGATTTATGG
CTATGATTGCAAAGTGATTGGAAGTAAATGCTTTTTTTCTTTCTTAAAATCATGTCTCCATCTGTCTGTA
GAAAATGACTCCTACAACCTATCATGCTACCCACAACCGGACCCTTCCGCCACCCGATCAAGGTAATGAA
CAAATATTTCTATCCCTTAGCTTCAATAATAGCTTCAGACCTAAAAAATAAGCTTTAATAATAGCCTTTT
GTTTGACGTCACATTTACTGTTGTGAGCATTTGGTTCCTGCATCATTATTCAGGATCTTTAAGAACGATC
CCATCAGGGGTGTTGTTTTACAATTGTAAACTTACCTGATTGGAACCCCTTTGCAGTGATAACTACTGAA
TCCAAGAACATTCTTCTGAGGCACATGTATCAGCAGCATGCTGAAGAGAAGGTACTAATCCCTTTATGCC
TCCTTTTTCCGACTGCTTACATTTGCTGAAGTGCACACAACTGAGATTAGTAAGAGAGAAGCTTTATTCT
AGTTTTCATGACTTCTTGCTGCAGTTGAGACAAAAGCGAGCTGCATCAGAAAACCTTTTACCAGAGCATG
GATCAAAGCAACTTAAGGGTTCTGTCTCAGATAAGTCCTAACAAGCAAAACTGCCTTTATCACTTCCAAC
TGCTCATTTGTTCTCACATGGATACTGGAAGTTCAGCATTCCCATCAGTGTGAATATTAGTGTCACAGGC
AAAAGATGTGTAGACTGTACCCTGTCGTAGATAGAAGGGGTATTTGATTGCACTTAGTTGTAAAAGTTGC
TTCACTAGACATGTAGACTTGCGTGTACGAATTAGATTACAGCTTTAAACAAATAAAATGAATAGTTACA
AGGTTTGCTTGTGTTCTGGTTCTATATGTCTTTACAAATGTTAGTTCCATGCTCATTTAAATCGAATGAA
GAACATGCTTCCCCCAAAATTGCTTGTATCACGTGACTGCGGGTTTGGAAAATACATAAAACTGATAAAA
GACAGCATATGTCAAC
Fragaria vesca
SEQ ID NO: 94
MGSLFGNWPSYDPHNFSQLRPSDPTTPSKMTPTTYHATHNRTLPPPDQVITTESKHILLRHMYQQHAEEK
LRQKRAASENLLPEHGSKQLKGSVSDKS
Citrus clementine, CDS
SEQ ID NO: 95
ATGGGCTCTATGCTCGGCGACTGGCCCTCTTTTGACCCTCACAACTTCAGCCAACTTCGTCCCTCCGATC
CCTCTAATCCGTCTAAACTTACACCTGCCACCTATCGTCCTACTCACAGCCGTACTCTTCCACCACCTGA
CCAAGTGATTACTACTGAAGCCAkAAATATTCTCATGAGAAATTTCTATCAGCGAGCTGAGGATAAGTTG
AGACCAAAAAGAGCTGCCTCAGAGCATCTAATTCCAGAGCATGGATGTAAGCkACTTAGGGCTTCTACGT
CAAACTGA
Citrus clementine, cDNA
SEQ ID NO: 96
GGCTAAGCTAAGTCTAGAATCGTGCGGGGCATTGTGCTCGTGGGCGCTCTCTCTCTCTCTCTTTCTCTGT
GTCTGTCTGTCTGTCTGTCTGTCTGTCTGTCTGTCTGGTGGTGGCTCTTGAAATTAGATTAGGGTGCATA
AACCGGCATTTGCAATGGGCTCTATGCTCGGCGACTGGCCCTCTTTTGACCCTCACAACTTCAGCCAACT
TCGTCCCTCCGATCCCTCTAATCCGTCTAAACTTACACCTGCCACCTATCGTCCTACTCACAGCCGTACT
CTTCCACCACCTGACCAAGTGATTACTACTGAAGCCAAAAATATTCTCATGAGAAATTTCTATCAGCGAG
CTGAGGATAAGTTGAGACCAAAAAGAGCTGCCTCAGAGCATCTAATTCCAGAGCATGGATGTAAGCAACT
TAGGGCTTCTACGTCAAACTGAGATGGACGCATGCAACTAGGCTTCCACCTTACATAAGTTTTCCTGCTT
TACCCAGGAACCCAACTGTTACTAAATTTCCATGGGTGTGTGTGTGTGTGTGTGTATCTCGTAATGGTGT
CATATATATTGTAATCTGTTGAGTTCAGATATGTACATTTTTTGTGTACTAATAATATTTGCTTGGGTGA
TCCCTTTTACAAGGTTCCGGGATGATCAGTTAATACTTTGCACTCCTTCCTGTGCTGGTATCATTTTATG
TGAATGACTGATGCAGGCCTTCACATCACATGCACATTTAATTGCATGAGGCTAGTGTGTTTATATATGG
GTTTGCTGCATTTGATTTT
Citrus clementine, gDNA
SEQ ID NO: 97
GGCTAAGCTAAGTCTAGAATCGTGCGGGGCATTGTGCTCGTGGGCGCTCTCTCTCTCTCTCTTTCTCTGT
GTCTGTCTGTCTGTCTGTCTGTCTGTCTGTCTGTCTGGTGGTGGCTCTTGAAATTAGATTAGGGTGCATA
AACCGGCATTTGCAATGGGCTCTATGCTCGGCGACTGGCCCTCTTTTGACCCTCACAACTTCAGCCAACT
TCGTCCCTCCGATCCCTCTAATCCGTCTGTACGTACTTCACTATACCCATTTTTTTTGTCCTTAATGATT
AATTTTCTTATCAATCAGAAATAAGCAAAATACTACAGAGCTGATCCTGATAAGATTTTCTGGAGCTTGT
GCAGTGAAATAAATTATTATCTTTTTCTAGAGTCGGTTCTGGGTATTTCTCATGTGAATTATTAGAGTCT
ATTAAAGATTTAAAAAAGAAAAAAAAAGAGCACATCATTTGGGTAGCTTATGCTTCCTGTTGTGCATTAA
AGAAAAAAGTGCCATTTTAAAAGTCTGGTAGTGTAGATATTGTTGTGGTGTTTGTTTTTCATTTTGATGC
ATCCTAGCTTGGGATGTCTGGTTCGATCAAATCTAGTGTAATCACAGAAATGTTGGTTGATGATGACTTT
AACTGCAGCATTTCTAGTAGGTTAATTGTGGTGTCTACTTTGTGACTATGGTGTTAAATGCTATTGATGT
ATGCTTAGTTTTTCTATGAAGTTGTGGAGGTGTAACATATCAGATTGATCTTTCACTTAGAAGCTTTGGC
GGCCGCTAACCAGAAGTTGTCAGAGTCTCCTACCTAAGAGGCATTGTGTATATTTCAACATGCTTACCCA
AGCAAACTTAGTGCATGTTTGGGATTGTCATGGATTTTCAAACAATCACTTATTTGAGAATCTACTTGCC
AATTGTGCATGTTACTAGTCGTTTAAGTTGTTGGAAACAAATTGCTGCATTGATACCTACGTTAGAATCT
TTTTAATTATGCTTTGCCTATATTATTTTAATTTAGTTAGGCAAGATTTTTGGTTGTAGCCCTTGTCAAG
AGAGCCGATATCTTGATTGAGAATTTGCTCAAGGATGATACTGACAGAAATGGTAGCTGGATAGCTTATT
GTGATAATTAATCATCAGAGATGTTGAATCATGGCAAGATTGAAGAGAGAGATTTGTAAATGATGACATT
AACTTCTTTAACCTACTTTTGGCTCTTGGAAGTGACATAATTGGTATTGAAATAATGGACACACTAATGA
AGTATGACTCTGAGGTCGTGAACATGTAGAGAACTTATATGTAGGAGCAAAATCAGGAAGAGGGATAGAG
TGAGTTAATGATCGAGTATTTTATTGGCGCACCCTCTTGTTCTGTCCTCTATTTTATGTTCTTCTGCCTC
TTGATTGCTTCTTGTTTTATCTTCAGTTTGATCCGAGTTTCAATGAAGAAATGGCCACGGTAACTAAATA
ACTTAAGACACTCTTGTTTGGAAAGTTGTTATTGCGTTGCATTAGATTATATGATACATTTGATTCAACA
TGAATTTTGATATCTTTACTCGCTAACATATATATTAATATATTTAATTGGCTTAAAGATTACTGTTGTT
AAATCAATGACCCATCAAATAAGGTGAACCAAGTTCTCTCATGTATAATTTCCTTTTTTTGTGTCTGTCT
AGAAACTTACACCTGCCACCTATCGTCCTACTCACAGCCGTACTCTTCCACCACCTGACCAAGGTATTGA
CACAATCTTTGTATCTCCTTATTGCATCAAAAGCTTCTTGCCAGAAGGGTTATTCCTGGCTTTTTGAATC
ATCTGCTGTATCATATACATAGTAATCTTTAAATTGATTTTGTGACAATTCCTCTCTTCACGTGGTGTAT
ATTTTCATGATACGATCTTTCAATCTGTTAAACTTGTTTTGCTAGGTTTGGTTTCATGGATGATGTTGAA
ATTTTTATGTTTGTGTATATCAATGCCAGCACTTCCATACATGCTGAATCCATTAACTATCTTTTTAGAA
AATATTGATGGTGTACAACTATATATGGATAAGCCAGTTTGGTTTTTAGAAAAGACTGAAAAATTAGGGT
AGAGACTCTCTATTATAACAGAGAGATCATGATTGCTTGGGTAGTAAAAGAATTCTTTATTTAAATCTTG
ACCAAGATGCGATTATTAATGGGATTGTAGTTGTGCGTTTGTCTGTTGTCAATGTGTACATTGTATGTAA
TTCTGGTAGAACTTACTTTTGTTATAGCTTCCCATGCTGTTTTTGTTTCGCCACATAATTACTATGGAGA
CAAATAGACAATGAACACTGTTTTTGGCAGTGATTACTACTGAAGCCAAAAATATTCTCATGAGAAATTT
CTATCAGCGAGCTGAGGATAAGGTTAGTATCATTTAAGATGTATCTTGTGCCAGTACATGTGTAGCAAGA
GAGTGAATTTACAAACACTTCTTTAACTTCTTCCTCTCTTTTGGCAGTTGAGACCAAAAAGAGCTGCCTC
AGAGCATCTAATTCCAGAGCATGGATGTAAGCAACTTAGGGCTTCTACGTCAAACTGAGATGGACGCATG
CAACTAGGCTTCCACCTTACATAAGTTTTCCTGCTTTACCCAGGAACCCAACTGTTACTAAATTTCCATG
GGTGTGTGTGTGTGTGTGTGTATCTCGTAATGGTGTCATATATATTGTAATCTGTTGAGTTCAGATATGT
ACATTTTTTGTGTACTAATAATATTTGCTTGGGTGATCCCTTTTACAAGGTTCCGGGATGATCAGTTAAT
ACTTTGCACTCCTTCCTGTGCTGGTATCATTTTATGTGAATGACTGATGCAGGCCTTCACATCACATGCA
CATTTAATTGCATGAGGCTAGTGTGTTTATATATGGGTTTGCTGCATTTGATTTT
Citrus clementina
SEQ ID NO: 98
MGSMLGDWPSFDPHNFSQLRPSDPSNPSKLTPATYRPTHSRTLPPPDQVITTEAKNILMRNFYQRAEDKL
RPKRAASEHLIPEHGCKQLRASTSN
Citrus sinensis, CDS
SEQ ID NO: 99
ATGGGCTCTATGCTCGGCGACTGGCCCTCTTTTGACCCTCACAACTTCAGCCAACTTCGTCCCTCCGATC
CCTCTAATCCGTCTAAACTTACACCTGCCACCTATCGTCCTACTCACAGCCGTACTCTTCCACCACCTGA
CCAAGTGATTACTACTGAAGCCAAAAATATTCTCATGAGAAATTTCTATCAGCGAGCTGAGGATAAGTTG
AGACCAAAAAGAGCTGCCTCAGAGCATCTAATACCAGAGCATGGATGTAAGCAACTTAGGGCTTCTACGT
CAAACTGA
Citrus clementine, cDNA
SEQ ID NO: 100
TGCGGGGCATTGTGCTCGTGGGCGCTCTCTCACTCTCTCTTTCTCTGTGTCTGTCTGTCTGTCTGTCTGT
CTGTCTGTCTGGTGGTGGCTCTTGAAATTAGATTAGGGTGCATAAACCGGCATTTGCAATGGGCTCTATG
CTCGGCGACTGGCCCTCTTTTGACCCTCACAACTTCAGCCAACTTCGTCCCTCCGATCCCTCTAATCCGT
CTAAACTTACACCTGCCACCTATCGCCCTACTCACAGCCGTACTCTTCCACCACCTGACCAAGTGATTAC
TACTGAAGCCAAAAATATTCTCATGAGAAATTTCTATCAGCGAGCTGAGGATAAGTTGAGACCAAAAAGA
GCTGCCTCAGAGCATCTAATACCAGAGCATGGATGTAAGCAACTTAGGGCTTCTACGTCAAACTGAGATG
GACACACGCAACTAGGCTTCCACCTTACATAAGTTTTCCTGCTTTACCCAGGAACCCAACTGTTACTAAA
TTTCCATGGGTGTGTGTGTGTGTGTATCTCGTAATGGTGTCATATATATTGTAATCTGTTGAGTTCAGAT
ATGTACATTTTTTGTGTACTAATAATATTTGCTTGGGTGATCCCTTTTAC
Citrus clementine, gDNA
SEQ ID NO: 101
TGCGGGGCATTGTGCTCGTGGGCGCTCTCTCACTCTCTCTTTCTCTGTGTCTGTCTGTCTGTCTGTCTGT
CTGTCTGTCTGGTGGTGGCTCTTGAAATTAGATTAGGGTGCATAAACCGGCATTTGCAATGGGCTCTATG
CTCGGCGACTGGCCCTCTTTTGACCCTCACAACTTCAGCCAACTTCGTCCCTCCGATCCCTCTAATCCGT
CTGTACGTACTTCACTATACCCATTTTTTTTCCTTAATGATTAATTTTCTCATCAATCAGAAATGAGCAA
AATACTACACAGCTGATCCTGATAAGATTTTCTGGAGCTTGTGCAGTGAAATAAATTATTATCTTTTTCT
AGAGTCGGTTCTGGGCATTTCTCATGTGAATTATTAGAGTCTATTTAAGATTTAAAAAAGAAAAAAAAAG
AGCACATCATTTGGGTAGCTTATGCTTCCTGTTGTGCATTAAAAAAAAAAAAGTGCCATTTTAAAAGTCT
GGTAGTGTAGATATTGTTGTGGTGTTTTTTTTTCATTTTGATGCATCCTAGCTTGGGATGTCTGGTTCGA
TCAAATCTAGTGTAATCACAGAAATGTTGGTTGATGATGACTTTAACTGCAGCATTTCTAGTAGGTTAAT
TGTGGTGTCTACTTTGTGACTATGGTGTTAAATGCTATTGATGTATGCTTAGTTTTTCTATGAAGTTGTG
GAGGTGTAACATATCAGATTGATCTTTCACTTAGAAGCTTTGGCGGCCGCTAACCAGAAGTTGTCAGAGT
CTCCTACCTAAGAGGCATTGTGTATATTTCAACATGCTTACCCAAGCAAACTTAGTGCATGTTTGGGATT
GTCATGGATTTTCAAACAATCACTTATTTAAGAATCTACTTGTCAATTGTGCATGTTACTCGTCGTTTAA
GTTGTTGGAAACAAATTGCTGCATTGATAGCTACGTTAGAATCTTTTTAATTATGCTTTGCCTGTATTAT
TTTAATTTAGTTAGGCAAGATTTTTGGTTGTAGCCCTTGTCAAGAGAGCCGATATCTTGATTGAGAATTT
GCTCAAGGATGATACTGACAGAAATGGTAGCTGGATAGCTTATTGTGATAATTAATCATCAGAGATGTTG
AATCATGGCAAGATTGAAGAGAGAGATTTGTAAATGATGACATTAACTTCTTTAACCTACTTTTGGCTCT
TGGAAGTGACATAATTGGTATTGAAATAATGGACACACTGATGAAGTATGACTCTGAGGTCGTGAACATG
TAGAGAACTAATATGTAGGAACAAAATCAGGAAGGGATAGAGTGAGTTAATGATCGAGTATTTGATTGGC
GCACCCTCTTGTTCTGTCCTCTATTTTATGTTCTTCTGCCTCTTGATTGCTTCTTGTTTTATCTTCAGTT
TGATCCGAGTTTCAATGAAGAAATGGCCACGGTAACTAAATAACTTAAGATACTCTTGTTTGGAAAGTTG
TTATTGCGTTGCATTAGATTATATGATACATTTGATTCAACATGAATTTTGATATCTTTACTCGCTAACA
TATATATTAATATATTTAATTGGCTTAAAGATTACTGTTGTTAAATCAATGACCCATCAAATAAGGTGAA
CCAAGTTCTCTCATGTATAATTTCCTTTTTTTGTGTCTGTCTAGAAACTTACACCTGCCACCTATCGCCC
TACTCACAGCCGTACTCTTCCACCACCTGACCAAGGTATTGACACAATCTTTGTATCTCCTTATTGCATC
AAAAGCTTCTTGCCAGAAGGGTTATTCCTGGCTTTTTGAATCATCTGCTGTATCATATACATAGTAATCT
TTAAATTGATTTTGTGACAATTCCTCTCTTCACGTGGTGTATATTTTCATGATACGATCTTTCAATCTGT
TAAACTTGTTTTGCTAGGTTTGGTCTCATGGATGATGTTGAAATTTTTATGTTTGTGTATATCAATGCCA
GCACTTTCATACATGCTGAATCCATTAACTATCTTTTTAGAAAATATTGATGGTGTACAACTATATATGG
ATAAGCCAGTTTGGTTTTTAGAAAAGACTGAAAAATTAGGGTAGAGACTCTCTATTATAACAGAGAGATC
ATGATTGCTTGGGTAGTAAAAGAATTATTTATTTAAATCTTGACCCAGATGCGATTATTAATGGGATTCT
AGATGTGCGTTTGTCTGTTGTCAATGTGTACATTGTATGTAATTCTGGTAGAACTTACTTTTGTTATAGC
TTCCCATGCTGTTTTTGTTTCGCCACATAATTACTATGGAGACAAATAGACAATGAACACTGTTTTTGGC
AGTGATTACTACTGAAGCCAAAAATATTCTCATGAGAAATTTCTATCAGCGAGCTGAGGATAAGGTTAGT
ATTATTTAAGATGTATCTTGTGCCGGTACATGTGTAGCAAGAGAGTGAATTTACAAACACTTCTTTAACT
TCTTCCTCTCTTTTGGCAGTTGAGACCAAAAAGAGCTGCCTCAGAGCATCTAATACCAGAGCATGGATGT
AAGCAACTTAGGGCTTCTACGTCAAACTGAGATGGACACACGCAACTAGGCTTCCACCTTACATAAGTTT
TCCTGCTTTACCCAGGAACCCAACTGTTACTAAATTTCCATGGGTGTGTGTGTGTGTGTATCTCGTAATG
GTGTCATATATATTGTAATCTGTTGAGTTCAGATATGTACATTTTTTGTGTACTAATAATATTTGCTTGG
GTGATCCCTTTTAC
Citrus sinensis
SEQ ID NO: 102
MGSMLGDWPSFDPHNFSQLRPSDPSNPSKLTPATYRPTHSRTLPPPDQVITTEAKNILMRNFYQRAEDKL
RPKRAASEHLIPEHGCKQLRASTSN
Cucumis sativus, CDS
SEQ ID NO: 103
ATGGGGTCTATGCTCGGTGACCTGCCGTCATATGACCCTCACAACTTCAGCCAACTCCGACCCTCTGATC
CTTCAACTCCTTCTAAGATGATTCCTACAACCTATCATCCAACCCACAGTAGGACCCTTCCCCCACCAGA
TCAAGTTATAAATACTGAGGCCAAAAATATACTTATACGACACATTTATCAGCATACAGAAGAAAAGTCA
AGAACAAAGAGACCTGCAGCCGAGCATCCCATGCCCGAGCACGGAAGCAAGCAACCAAGAGCATCTACTA
CCAACACTTCAAATTGA
Cucumis sativus, cDNA
SEQ ID NO: 104
AGTTGTAAATCCAATGGCGATGTGATTCCTAATGATTCCCTTCTAGAAAAACCACTTCTTCTTCCTTTTT
CTTCTTCATCTTCTTCTTCTCCTCTGTAGATTTCGAACAATCAACATATATTCAGCAGCATTTTCATGGG
GTCTATGCTCGGTGACCTGCCGTCATATGACCCTCACAACTTCAGCCAACTCCGACCCTCTGATCCTTCA
ACTCCTTCTAAGATGATTCCTGCAACCTATCATCCAACCCACAGTAGGACCCTTCCCCCACCAGATCAAG
TTATAAATACTGAGGCCAAAAATATACTTATACGACACATTTATCAGCATACAGAAGAAAAGTCAAGAAC
AAAGAGACCTGCAGCCGAGCATCCCATGCCCGAGCACGGAAGCAAGCAACCAAGAGCATCTACTACCAAC
ACTTCAAATTGAGCTTGGGAGGACATTTTCCTCCAACAAATTAAAGCTATTCATGTTGTGATAGATGACT
CCTGTATAATGAGAGTGAATTGCCTGCTCACTGAAGAAGAAACGGCTCGGCCAGACATTTACATGTTGTA
TATAGATTTTACTCCTTGTAAGATTACCCTAAACTCAACCACATCAAATTGTTGTCAAAATCATAAAACT
CAGTTGAAGAATTGTAACTATATGCGTGTGCTTCCAAACAATATTATTGGAGGCCTCCTTCCTATAAAGC
AAAAGATCCTCACCTTGTTCTTTTTCCTGGTTTGGAT
Cucumis sativus, gDNA
SEQ ID NO: 105
AGTTGTAAATCCAATGGCGATGTGATTCCTAATGATTCCCTTCTAGAAAAACCACTTCTTCTTCCTTTTT
CTTCTTCATCTTCTTCTTCTCCTCTGTAGATTTCGAACAATCAACATATATTCAGCAGCATTTTCATGGG
GTCTATGCTCGGTGACCTGCCGTCATATGACCCTCACAACTTCAGCCAACTCCGACCCTCTGATCCTTCA
ACTCCTTCTGTAAGTTCTCAATAATGGCCTAATCATCATACCCTTTCTTTCTCTTCTTCTAATTCATTTC
TCTATTTCTTTAAACCCTTCACTCCTTTTCTTGATCTTGGGTGTTTCCCTCCGTTTTGCATGATTCTTTG
TTTCGTTTCTATTAATGGGAAGTTGCTCGACTTGTGGTAGGGTGTGAATTGTGATGGGGTTCTGATTAAA
TTTAACCTCCTCTTGATCTTCTTTGCTCTTTCGTTGTTGGGGCTCAGCTAATTTTTGGTTGGGATTATTG
GCATTATTGATCTGTTTTTTGTTGTTGTCTTATTTGGAGATTCCCATTGCGTGATACTTGGAAAATCTTG
AATTTTGGCATGTGGGTTCTTTGTTTAGGCATGTTTGTAGATGTGGGTTACAATTAAGTTCCGCATTTGA
CGTGTTTGAATGTTCTATGAATTTTCCAAATGTTTCTCGTAGGTAGAAGTTGAACTTTGTTTACCCCCCC
ACTTCCCCACCCTGTTGGAACAAATGAATCAGATGGTGTTTTTGTTTTAATTTTCACTCTCTCTAGGTAT
TATAATCTTGTGAAGTCAATCCTCTGAAAGAAACGGTTCCGTTAACCCAGTCGCAAAGAATTCGCTATAT
CATGATACGAAGGGGACAGAAATCTGAAGGAACAATAGCACTGAGTTACCGATCTTCCGGAGAAACGATT
CTATTTAATTCTCATATGTAGCCCATCTTCAAGTTTCGGAATTCTACGATTAGGATCCCTAGTGCTTTTA
CCCTTTGAATGGAGCAGCCTGCAACCAATTTAAGCTATCTCTTCCTTTGAATATAATAAAACTCATTGGG
CCAAAATGAAAAGCCCTTGAATTGCAGGGTTATGAATTTCTTCTATACTGCGAATAGTTATGCTATGCTC
ATGCTTTCTCTGATTTCTTGAGTGGTTTACACTTTGCTTTCAAGGGTTTAAACTTAAACGTCCCAATTAA
ATACTTGATATAAAGTTTGAAGTTATTCAAACAGTACTCCTTTGAGCCACTTTAGTATTTTTTTGCCTTT
GTCCTTCTGCCCTGAATGGAAATATTTAGATAATGCAGAAGGATTAACAACTCTAAGAAACATTAGTGAG
AACTGAGAAGCTGTTGACTGAAGAGATTCTAAAAGCATCTCAACTAAAGCTACCAGATGTGGTATCCCTT
TTGGATATAAAGAAAGGAAGCAAATAGTTTATAGTTTCTACATTAAAGTAAGGTCAAATCAAATTCTCCT
TGTAATTGTGTGTACAAACTTGTGCATGCTTTGGATTTATCTCCCATCTTTGAATTGATCTCAAATCCGA
CACCTCATGGGTGGCCATCCCTTGTTAATTTGCGGTAGATTGAGTGAACATGTGAGTGGAAGAGCTGCTG
TCTGGATCAACTAAAATGCTTTCATTCAAAGCTTAATCTGCGCTATAATGCAACATATTGTTTACTGTAT
CTGCTAGCTGCAAGTAAAGTAGAACAAGAAGATGAATTAACTATTTTTCAATGTAAGGAATAAGTTGATG
TTGGAATTACAATGCTAAGTTGGTTTTATTTTTATGTAGAAGATGATTCCTGCAACCTATCATCCAACCC
ACAGTAGGACCCTTCCCCCACCAGATCAAGGTAAGCAAGAAAAGTTGTTTCTTCACTACTCTACCGATAA
CTTTGTATCTTGCTCGGATAACAGTATTCTTTAGTCAGTAGTTTTCCACTGTTGGCTTTAGTTTCCATCT
TTCTTCTGCTTTTTACTCAAAAAAGAACTCCTCACTGATTTTTACTGCAGTTATAAATACTGAGGCCAAA
AATATACTTATACGACACATTTATCAGCATACAGAAGAAAAGGTTAGTAAAAGAAATCTGTTATGCTTTG
ATCTGAAATACAATCCATACATGTAGTAAGCTACCTTGTGAGACCACTCACCCATTCCCTGTGGACTTCC
CCTGGTCTTTGTAATGGCAGTCAAGAACAAAGAGACCTGCAGCCGAGCATCCCATGCCCGAGCACGGAAG
CAAGCAACCAAGAGCATCTACTACCAACACTTCAAATTGAGCTTGGGAGGACATTTTCCTCCAACAAATT
AAAGCTATTCATGTTGTGATAGATGACTCCTGTATAATGAGAGTGAATTGCCTGCTCACTGAAGAAGAAA
CGGCTCGGCCAGACATTTACATGTTGTATATAGATTTTACTCCTTGTAAGATTACCCTAAACTCAACCAC
ATCAAATTGTTGTCAAAATCATAAAACTCAGTTGAAGAATTGTAACTATATGCGTGTGCTTCCAAACAAT
ATTATTGGAGGCCTCCTTCCTATAAAGCAAAAGATCCTCACCTTGTTCTTTTTCCTGGTTTGGAT
Cucumis sativus
SEQ ID NO: 106
MGSMLGDLPSYDPHNFSQLRPSDPSTPSKMIPTTYHPTHSRTLPPPDQVINTEAKNILIRHIYQHTEEKS
RTKRPAAEHPMPEHGSKQPRASTTNTSN
Cucumis melo ssp. melo CDS
SEQ ID NO: 107
ATGGGGTCTATGCTCGGTGACCTGCCGTCATATGACCCTCACAACTTCAGCCAACTCCGACCTTCTGATC
CTTCAACTCCTTCTATGATTCCTGCGACCTATCATCCAACCCACAGTAGGACCCTTCCCCCACCAGATCA
AGTTATAAATACTGAGGCCAAAAATATACTTATACGACACATTTATCAGCATACAGLAGAAAAGTCAAGA
ACAAAGAGACCTGCAGCCGAGCATCCCATGCCCGAGCACGGAAGCAAGCAACCAAGAGCATCTACTACCA
ACACTTCAAATTGA
Cucumis melo ssp. melo cDNA
SEQ ID NO: 108
AATGGCGATGTGATTCCTAATGATACCCTTCTAGAAAAACCACTTCTTCTTCCTTTTTTTCTTCTTCATC
TTCTTCTTTTCCTCTGTAGATTTCCAACAATCGTCTTATATTCAGCAGCATTTTCATGGGGTCTATGCTC
GGTGACCTGCCGTCATATGACCCTCACAACTTCAGCCAACTCCGACCTTCTGATCCTTCAACTCCTTCTA
TGATTCCTGCGACCTATCATCCAACCCACAGTAGGACCCTTCCCCCACCAGATCAAGTTATAAATACTGA
GGCCAAAAATATACTTATACGACACATTTATCAGCATACAGAAGAAAAGTCAAGAACAAAGAGACCTGCA
GCCGAGCATCCCATGCCCGAGCACGGAAGCAAGCAACCAAGAGCATCTACTACCAACACTTCAAATTGAG
CTTAGGAGGACATTTCCTTCCAACAAAGTTAAAGCTATTCATGTTGTGATAGATGAGTCCTGTATAATGA
GAGTGAATTGCTTGCTCACTGAAGAAGAAACGGCTCGGCCCGACATTTACATGTTGTATATAGATTTTAC
TTCTTGTAAGATTGCCCTAAACTCAACCACATCAAATTGTTGTGAAAATCATAAAACTCAGTTGAAGAAT
TGTAACTATATGCGTGTGCTTCCAAACAATATTATTGGAGGCCTCCTTCCTATAAAGCAAAAGATCCTCA
CCTTGTTCTTTTTC
Cucumis melo ssp. melo
SEQ ID NO: 109
MGSMLGDLPSYDPHNFSQLRPSDPSTPSMIPATYHPTHSRTLPPPDQVINTEAKNILIRHIYQHTEEKSR
TKRPAAEHPMPEHGSKQPRASTTNTSN
Castanopsis sieboldii, CDS
SEQ ID NO: 110
ATGGGTTCTCTCTTTGGTGACTGGCCGTCATTTGACCCTCACAACTTCAGCCAACTCCGACCCTCCGATC
CTTCTAGTCCTTCTAGAATGACACCTGCAACCTATCATCCTACTCACAGCCGCACGCTTCCACCACCTGA
TCAAGTGATCACTACTGACGCCAAAAACATTCTCTTAAGGCACATCTATCAACGTACTGAAGAGAAGGAT
CTGAGACCGAAGAGAGCTGCGCCAGAACATCTTGTACCTGAGCATGGATGCAAGCAACCTAGGGCATCTT
CCAGTTCCTGCTGA
Castanopsis sieboldii, CDS, cDNA
SEQ ID NO: 111
CATTGTGCTCTCTTTCTCTCTCCCCCTAGATTTTTTGTGCCGAAAGAAACCAGCATTTTATGGGTTCTCT
CTTTGGTGACTGGCCGTCATTTGACCCTCACAACTTCAGCCAACTCCGACCCTCCGATCCTTCTAGTCCT
TCTAGAATGACACCTGCAACCTATCATCCTACTCACAGCCGCACGCTTCCACCACCTGATCAAGTGATCA
CTACTGACGCCAAAAACATTCTCTTAAGGCACATCTATCAACGTACTGAAGAGAAGGATCTGAGACCGAA
GAGAGCTGCGCCAGAACATCTTGTACCTGAGCATGGATGCAAGCAACCTAGGGCATCTTCCAGTTCCTGC
TGAGCCCTTATCTTGTTATATGGAACCCCAAAATAGTTAATTCGTGTAAATGTTTTTGTCATGCCAAATA
TGCGTGAGTTTCTTGTGGGTTGAAAAGGGGTTTTATTTTGCTTGATCATTGCTGTAAGCAGCTTAACCAG
AAGTGTAGATTTTGTGTGTATAATTCATAAATACTATAGAGTTGGGTGATCCCTATTACAGTTTACATGG
ATGATGAAATGAAAGTAATAGATATTATT
Castanopsis sieboldii
SEQ ID NO: 112
MGSLFGDWPSFDPHNFSQLRPSDPSSPSRMTPATYHPTHSRTLPPPDQVITTDAKNILLRHIYQRTEEKD
LRPKRAAPEHLVPEHGCKQPRASSSSC
Actinidia setosa, CDS
SEQ ID NO: 113
ATGGGTTCTTTGCTCGGGGACTGGCCTTCCTTCGACCCTCACAACTTCAGCCAACTCCGACCCTCCGATC
CTTCAAATCCTTCAAALATGACGCCTGTCACTTATCATCCTACTCATGATCGGACCATTCCACCCCCTAA
TCAAGTGATTTCTTCCGAAGCCAAAAATATACTTCTGCGGCATTTCTATCAGCGTGCCGAGGACAAGCTG
AGACCAAAGAGAGCTGCGTCGGAACTTCTGACACCCGAACACGGAGGCAAGCATCCCAGGGCCTCGGCTT
CTGCTTCAAAAGCGCCTCCCTGCTGA
Actinidia setosa, cDNA
SEQ ID NO: 114
CCCCAAACCACTCCATTGTTCTCTTCCTTTATCTCGATTCTTCCATTGAAATCGCAGCTTCCAATCCATG
GGTTCTTTGCTCGGGGACTGGCCTTCCTTCGACCCTCACAACTTCAGCCAACTCCGACCCTCCGATCCTT
CAAATCCTTCAAAAATGACGCCTGTCACTTATCATCCTACTCATGATCGGACCATTCCACCCCCTAATCA
AGTGATTTCTTCCGAAGCCAAAAATATACTTCTGCGGCATTTCTATCAGCGTGCCGAGGACAAGCTGAGA
CCAAAGAGAGCTGCGTCGGAACTTCTGACACCCGAACACGGAGGCAAGCATCCCAGGGCCTCGGCTTCTG
CTTCAAAAGCGCCTCCCTGCTGAGCTTTCCTGCTATTGCTTGAAGAATATCTCAAGAGTCAAGTTCTATT
GAATGTCATTGTGAATATTCCCATCATCATATTACCAATTTGTGTTTTCCGCAATTATAAAGGGTATTTC
TGTGCTCATTGTACATTTTGCATGTATAAACTCCAGTTGTTCACCTTCCCCTTTTCAAGTGCTGATGTAG
AATCTAGTCTCATCGCATGCTTCTCCCCTTTGCCTGTGTTGGGCATTACATAGTCGT
Actinidia setosa
SEQ ID NO: 115
MGSLLGDWPSFDPHNFSQLRPSDPSNPSKMTPVTYHPTHDRTIPPPNQVISSEAKNILLRHFYQRAEDKL
RPKRAASELLTPEHGGKHPRASASASKAPPC
Solanum tuberosum, CDS
SEQ ID NO: 116
ATGGGGTCAATGTTTGGTGAATGGCCCTCAATTGACCCTCACAATTTCAGCCAGCTTCGCCCTTCTGATC
CCTCAACTCCTTCTAGAATGACACCCGTGACTTATCGCCCTACTCATGATAGGACTCTTCCTCCACCAAA
TCAAGTTATTAGTTCAGAAGCCALAAATATACTTCTGAGACACCTAGAGCAGCGTGCTGAAGAGAAGTTG
AGACCAAAGCGAGCTGCGGCTGAAAATCTGGCACCCGAGCATGGGTCGAAGCATCTTAAGGTATCCAACT
GA
Solanum tuberosum, cDNA
SEQ ID NO: 117
CACAATATATATATTTGTGCTCTCTCTTTAAAGAGTGGCATTGTTCTCTGGATTCTTCCCATTTTGGGTG
CTATGGGGTCAATGTTTGGTGAATGGCCCTCAATTGACCCTCACAATTTCAGCCAGCTTCGCCCTTCTGA
TCCCTCAACTCCTTCTAGAATGACACCCGTGACTTATCGCCCTACTCATGATAGGACTCTTCCTCCACCA
AATCAAGTTATTAGTTCAGAAGCCAAAAATATACTTCTGAGACACCTAGAGCAGCGTGCTGAAGAGAAGT
TGAGACCAAAGCGAGCTGCGGCTGAAAATCTGGCACCCGAGCATGGGTCGAAGCATCTTAAGGTATCCAA
CTGAGATGCTTTTCTTTTTGGTGCTACCCCCGGGCGGGAAGAAGATGAGGTAATGCGAAACAGGACGATA
CACAACTTGGTTTTAGAAGAGTAACTAACTTCTAAATAGGTTAAAATCTTCTGGTTTTCTGCATATTTCT
GTAAATATTGCTGTAATGATGCAGATGCATGTTGTTGTAAAACTATGAAGAGCTTGTTTATCACTAGTCA
TATAGCAAATGAGATGTACACTAGAGAAAATGTTGTTGGATGAGATTTCTCTGCATGAGTATTGATAAAT
GTTTCATGCTGAGGGTTTATCGGAAACAA
Solanum tuberosum, gDNA
SEQ ID NO: 118
CACAATATATATATTTGTGCTCTCTCTTTAAAGAGTGGCATTGTTCTCTGGATTCTTCCCATTTTGGGTG
CTATGGGGTCAATGTTTGGTGAATGGCCCTCAATTGACCCTCACAATTTCAGCCAGCTTCGCCCTTCTGA
TCCCTCAACTCCTTCTGTAAGAACCCTTTTCATTTTTTTTCAATTTTTTTTTATATAAAACTTAAATCTT
TGATTTTTTTTAACACCCTTTTCCCCATTCAATCTGTTTTTTGAATTCTACTGTGTTTTTAGCTGATTTA
TGTTCGACCCATTTTTGTCTGATAGCAAAAAATGCATTCTTGGGATAATTTTAGCTGATTTGTGTTCCTG
AATGGAGCAGAATGAAATCCAGCTAATTTGGGACTGAAATGTTGTTGATTATTTTGTTGACCCATTTTTG
CTATGTTTGTTTTATGCAAAAAAAAAAAAAAAAGTGTTGGAACAATTTTGTGGGTGTATTTGAAAAATTC
TTGATCTTTTCAAGTTAGAGTTTTTATTTGGAGAAACTTTGATTTGTGTTAAGAGATCAATTGTTTAGTA
TTTGAAATGAAATGGGTCCGAACGGATCAGAATGAATATGTATATATAGGGAGAGAATTCATATAGCTGA
AGTGTTGAACCTAGCTAATTTGGGATTGAGATGTATTACGTTTCCCTCTGTTTTAATTTGTTTGTCTTAC
TTTCCTTTTCAGTTTGTTTAAAAAAGAATGTCTCTTTACTTTTTTGTCAGCTCTTTAATTTCAACTTTCA
AGATTAGAAGGCATTTTGGTATATTCTACGTATGTTGAGTTTAAGTCTACTTTACTTTCGTAAGCTCCGT
GTCAAGTCAAAATTAGACAAACAAATTGAAAAAGAGGGAGTATTAGTTAATTGACTGTCGTTTTTTGCAT
GCTTATGTGGTGTTGTGTTACTAAAGCTAGCATAAAATACTCTGATTTAGGCTTAAGGCTGATATTTCAG
TTTGAGAATGTTTTCTGATGTAATGATGTTTGATTTGCAAATGGGCTATAGGGTTACCATAATGGTGCCT
AATCTATAAAAGAGTGATATTTAAGCTATATGTAATTTAATGCGGATTTTGTATAAGTGTATTCTAGTTT
TGGTGCATTAACTTCTATCAGATAGACGGCATATACATATATCAATGCGTGTGGAGATATCATATTACCT
CAAGCAATAATGTGGATCATTATTTATCGGAAAAATGAAACAACAAGAAGAATTGAGTACAATCTATGTA
GCTGTCTTCTTGATGGTTTGGTGGTGTCTAGGTTTAATTTGTCGCTTCTGTGAGACTTAATTGCATGCTA
TGCTCAGCTTTGTGTAAACAATCCCTCTTTGCTAGTGTAGACTCACACCACGTGGCATTTGCTGCCATAA
TACATTGTACACTAGCTTAATGATAATTGCGTATTAGATTGTTATCTATTATGACATTGGTTATAAGCAT
GCACTGCTTATGTTACTGTTATTTCAGTTACTCTTACTTCTGAACGCATATTGATAAATATGTAGGAGAG
GAATCAGAAAACAATAATATTCTAAGACTTCGTAGAAAAGATATATAATACTTGTTTCAATGTATTTTTA
CTATCTGTTGCTTCATTTTTCTTTGTATCTTGTCATCTTTTTCTTCCCTCCCCGGACACCACTTGTGGGA
TTCCATTGGGTATGTTGTTGTTGTTGTTGTAGGGTATAACATGTTAGTATGAAATGTCTTACTCTCAAGA
GAAGCGGAAAAAAGAAGCCTTATCGGCTCTTGTATTACCATAACAACATTTTCATTAACTGATACAACTT
TTTTCCTTTTTTGAGTTCCTTCTTACCTTTTCTCATTGTGCTTCTTGGCTCCTTGAATATGATCCCATTG
ATCCTTCGGCTTTCAAGCCTCAAGAAGCAATTGAAGACCTCATCTTCGTCCCCCGTTATGTCGACCAATC
GTTTACCTCGCTTTAATGTAGTTCTTTTGATCCACGCTGAAGCTACCACCTTTAATCCTTTATCTGTGGT
GGCTAATTTAATTGCTTGGAGAGAAGTGAGAAGGTTGATTTTCTTCTTGTGGTGACAGTCTTTTTCTCAG
TGGTGCCATGCTTTGCTTCTATTTACAGCATTCACTTTAATTTCTCTAGAACCGTGTATGTTACAACACT
AGGTTTATGATTTACCTTGTGAGCTTTTGATGTAGAAGAGCTGCAAATTATTTGAAGCATCTATTATCCA
CAATAAACTTTTTCTATATTATTGTTGTGTATACAGTCTGGTCTCCCTATTTCCTTGCCCTTTCCCCCCT
TTCTGGTAGTTAAAACTTAAAAACAAAAATAATGAAGGTAAATATAGTGGAGTGTTTGAAGATAAAGTTA
CTTGGATAACTCATACATTGAGGCTCATTATGTCTCCAAAGGAGTTGTCGTGAAGCTGAATTTGTGAAGT
TACTTACTACTTACTATAATGGTTTCCTATAACAATCGGCTTGAGGGACTCTCTTCACGGGAAGTCATGC
CTGGGCAAGGGTAGTACTAGGATGGGTGACCCCCAAGGAAGTCCTTGTGTTGCATCGTTGCTTTGAATGG
CATGATAAGTGAAGTGAACTAACATTTGTAATCTGCATATGCACTTCCAAATCTCTTTAAAAATTATTAT
AGCATTACATAAGTGTTCCATGATGAAGTTATTTGTTGGTAAACCTTTTGTACTTTGACTTTTAGTAGCT
GAAACAATCTCTCTGTTTCTTTTTTTTCCAGTTTCGAGGATACTAGTTTTTTTTTTTTTCCACTTATCTA
ATCCTGTGTTCTTTCTTTTTCTAGAGAATGACACCCGTGACTTATCGCCCTACTCATGATAGGACTCTTC
CTCCACCAAATCAAGGTAAATCAAGAACATGATTCTTTTTGTTTCTTACACATCTTTTGATATCATAATT
TACATTTATCTTTTAGTTGCTCTTACCCTCTACCTTTATCAGGTATTTGGATTATATACATTTGACTAGA
ATCCTTGAGCCAAGGGTTTGTCAGAAATAGACTCTCTACCTTCCAAGGTAGGGGTAGGGTCTCTATGCAC
ACTACCCTCCTCAGACCCCACTTATGGGATTACATTGGGCATGTTGTTGTTGTATATATTTGACTATACT
AACCTCCCACCCTTCCTCCCTAAGTGGATGGTGAATGGAATAACTTTAGCTCAGCTGCTGTTATTGGTTA
TTCTTATGAGTTGGTCCTTAGAATTAAGCCAGTCATCCTCTTGAGGATTTGAACTTCAGTTTATTCACTT
TAGAAGTTGTTTCAACCTCAAATGATTGTGTCGCAAAAGCATTGGGCCCGGATCAATAAGGTTGCCAACT
GGCAATGAGTTGTTTATGAGTAGCAGAATCAAGCACCCGTAATTACTAGTTTTGAATTTTTCGCTCCGGC
TTTTGTGAAGATAAACAGTCTAAAAAGTTCTCTGTGGATGCTAGTGTTGAGGCTTTCATCGAAACAAAGA
GTGAGACCTGTCTGGTGCTTTTTTCTATGTTAGTCAGGTGAAAGTAAATAGATTAGTGTATTAAGTAAGG
CACTGCTAAAGCCTCTATTCCCGTTTTTTGAGCGATAAAACTCTTAGCTTCCAGCAAGCTAGGTGCATAA
GCTTTTTTTACTACATAAAAGATTCCAAGAAATGCAGGCAGGGTAGAGCTCGGCAGAGGGTTCGAGAATA
GGGTAGGGTAGGGTCCTTAAGATTCAATAAGAAAGAGTGTGCCTTTAAAGCAAATAATGCAGCCCTTTTC
TTTCCTCATATGGCTATATTTAATTCAAAACTCCTGTAAATGTGTGTGTTTACAAGTGACAATTTGTCAT
GTTCATCTCCTGTTATGAAATTGTGGTTTAATTCTTTCGGATAATATTATAATTATCTTGCCCCACTACC
ACATCTCTATACTCGACTAAGAATGATTTCAATTGCACTAGGATAAACTACTTTTCAATAGCTTTAATTG
CATCATCTTATGTAAGATCCTATTACCGAAAAGGAAAAAAGGGAATTCTCTTTGGTTGTAACATTTGGTA
AATATAGTATCAATAGCCATCTAAAGTACATTCATGTTATTGGTATTAAACTTTTATGAGGATGCACATA
TTCGTGAGCCGAGGGTCTATCGAAAACAACTTCTCTACCTTTGAAGGTATGGGTAAGTTGCATACACACC
ACCCTCCCCAGACCTAATCATGAAAATCTAATGTAATGTGCACGTTTGTTTGTCGGTTTACTTTCTCATG
CTTCATCTCAATCTTTTCTTTTTTTCTTTATTGAATCTCGATTAATCAGATAAGGGAAGTATACTAGATA
TGAATTGCGTGAGCTTAAAACTTTTGCACTTAAGGTATGCTTATATCTAACTCAACATATACTAATGACC
TGCATTTGAATTTCCAGTTATTAGTTCAGAAGCCAAAAATATACTTCTGAGACACCTAGAGCAGCGTGCT
GAAGAGAAGGTCAGTATTCATTTTGATCGACATTTATTCCTTCTTTTGATGGATATTTGGCAATTTGTAA
TTGTGTTATATTGTGAGGATTCATCTTAAAGTGACTAGTTTTACAATGGCTATCAACCTATTGCAATGTT
TCTCCTTTATCCAGTCTTGTGACCGACAATATGAGCAAACTCACACATAATCAATGAGGATTCGTATAAC
CAACCCTAGCTTCCTTGGGATTGAGGTGTTGTTGTTGTGAAATGTGTTCTATCCCTTTACGGTTAAAATG
CTTTTTTGTGTTGTAGTTGAGACCAAAGCGAGCTGCGGCTGAAAATCTGGCACCCGAGCATGGGTCGAAG
CATCTTAAGGTATCCAACTGAGATGCTTTTCTTTTTGGTGCTACCCCCGGGCGGGAAGAAGATGAGGTAA
TGCGAAACAGGACGATACACAACTTGGTTTTAGAAGAGTAACTAACTTCTAAATAGGTTAAAATCTTCTG
GTTTTCTGCATATTTCTGTAAATATTGCTGTAATGATGCAGATGCATGTTGTTGTAAAACTATGAAGAGC
TTGTTTATCACTAGTCATATAGCAAATGAGATGTACACTAGAGAAAATGTTGTTGGATGAGATTTCTCTG
CATGAGTATTGATAAATGTTTCATGCTGAGGGTTTATCGGAAACAA
Solanum tuberosum
SEQ ID NO: 119
MGSMFGEWPSIDPHNFSQLRPSDPSTPSRMTPVTYRPTHDRTLPPPNQVISSEAKNILLRHLEQRAEEKL
RPKRAAAENLAPEHGSKHLKVSN
Solanum lycopersicum, CDS
SEQ ID NO: 120
ATGGGGTCAATGTTTGGTGAATGGCCTTCAATTGACCCTCATAATTTCAGCCAGCTTCGCCCTTCTGATC
CCTCAACTCCTTCTAGAATGACACCGGTGACTTATCGCCCTACTCATGACAGGACTCTTCCTCCACCAAA
TCAAGTAATTAGTTCAGAAGCCAAAAGTATACTTCTGAGACACCTAGAGCAGCGTGCCGAAGAGAAGTTG
AGACCAAAGCGAGCTGCGGCTGAAAATCTGGCACCCGAGCATGGATCGAAGCATCTTAAGGTATCCAACT
GA
Solanum lycopersicum, cDNA
SEQ ID NO: 121
AACCCACAATATATATATTTGTGTTTTCTCTTTAGAGAGTGGCATTGTTCTCTGGATTCTTCCCATTTTG
GGTGTTATGGGGTCAATGTTTGGTGAATGGCCTTCAATTGACCCTCATAATTTCAGCCAGCTTCGCCCTT
CTGATCCCTCAACTCCTTCTAGAATGACACCGGTGACTTATCGCCCTACTCATGACAGGACTCTTCCTCC
ACCAAATCAAGTAATTAGTTCAGAAGCCAAAAGTATACTTCTGAGACACCTAGAGCAGCGTGCCGAAGAG
AAGTTGAGACCAAAGCGAGCTGCGGCTGAAAATCTGGCACCCGAGCATGGATCGAAGCATCTTAAGGTAT
CCAACTGAGATGCTTTTCTTTTCGGTGCTACCCCTGGGCGGGAAGAAGATGAGGTAATGCGGTTGCAAAC
AGGACGATACACAACTTGGTTTTAGAAGATTAACTAACTCTTCTAAATAGGTTAAAATCTTCTGGTTTTT
CTGCATATTTCTGTAAATATGACTGTAATGATGCAGATACATGTTGTTGTAAAACTATGAAGAGCTTGTT
TGTCAAGTAGTCATATAGCAAATGAGATGTACACTAGATAAAATGTTGTTAGATGAGTATTGATAAATGT
TTCCCTCCGAGGATTTATCGGAAACAACCTCTGTGATCCGTCCTCTGTGACCCTAGAAAATGATATTTTA
TCATAATGATCAAACTTTTTAACATTGA
Solanum lycopersicum, gDNA
SEQ ID NO: 122
AACCCACAATATATATATTTGTGTTTTCTCTTTAGAGAGTGGCATTGTTCTCTGGATTCTTCCCATTTTG
GGTGTTATGGGGTCAATGTTTGGTGAATGGCCTTCAATTGACCCTCATAATTTCAGCCAGCTTCGCCCTT
CTGATCCCTCAACTCCTTCTGTAAGAACCCTTTTCATTTTTTTTCTATTTTTTTTTTCATATAAAACTTC
AATCTTTGATTTTTTCTGAACACCCTTTTCCCTATTCAATCTGTTTTTTTGAATTTTAATGTGTTTTTTA
GCTGATTTATGTTCGACCCATTTTTGTCTGATAGCAAAAAGATGCATTCTTGGACAGTTTTAGCTGATTT
CATTTGTGTTCCTGAATGGAGCAGAATGAAATCCAGCTGGTACTATGTGTTAATTCTCCGTTTTAAGAAC
GTCTCTTTTTTCTTTTGTCAGCTCTTTAATTTCAACTTTCTACTGACATGTCTAAGGCCACAAGATTAAA
AAAGAGCAATTTTGGTACTATGTCTGTTAATTTAAGACCACAAGTTTCAAAAGTTTACTTTACTTTCTTA
AACTCCGTAGAGGCAAACAAATTGAAACGGAGAGAGTAGTTTATTGATTGTCATTTTTTGCGTGTTTATG
TGGTGTTATGTTACTTAAGCTCTCTGATTTAAGCTTAAGCCTTATGTTTCAGTTTGAGAATGTTTTCTGA
TGTAATGATGTTTGCTTTGTAAATGAGATATAGGGTTACGGTAATGGTGCATAATCTATAGAAGAATGAT
ATTTATGCTTTTAAGTAAATTCAACGTGGATTTGGATTAAGTATATTCTAGTTTTGGTGCATTAACTTCT
ATGGGATAGATGGCATATACATATATCAATGCCTGTAGAGATCTAATGTTATCTCAAGCAACAATGTGGA
TCATTATTAATCAGAAAAATGAAACAACGAGAAGAATTGAATAGAATCTATGTAGTTGTCTTATTGATGG
TTTGGCATAGTCTAAATTTGTCTCTTGTGTGAGACTTAATTGCACGCTATGCTCAGCTTTGTATAAACGA
TCTTCTTTGATGGTGTAGAGTGTAGACTCACACCACTTGACATTTACTGCCATATTACATTGCACTCTAG
TTTAATGATAACCACATACAGATTGTTATCTATTAAGGCATTGATTGTAAGTATGCACTGCTTATGTTAC
TGTTATATTTCAGTTATTCTTCTGAATGCAGATTGATAAATATGTAGGGGAGGAACAGAAAACCATGTTA
TCCTAAAACTTTAAAGAAAAGATACATAATACTTGTTTCAATGTGTTTTTACTATCTGTTGCTTCTTTTT
GCTTTGTATCTTGATATTTTACTGTCATTTTTTTCTCCCTCCCCAGATACCACTTGTGAGATTCCATTGG
GATGTTGTTGTTGTTGTGGGGTATATCATGTTAGTATGAAATGTCGTACTCTTAAGAGAGGCGGAAAAAA
GAAGCCTTATCAGAACTTCTATTACCATGACAATTTTCATTAACTGATGCAGCTTTTTTCCTTTTTTGAG
TTCCTTCTTACCTTTTCTTCTTGTGCTGCTTGGCTCCTTGAATATGATCTGCCCATTGATCCTTCAGCTT
TCAAGCCTCAAAAAGCACTTGACGACCTCATCTTCATCCCCCGTTAGTTAACCAATTGTTTACCATGCTT
TAATGCATTTCTTTTGATCCACGTCGAAGCTGCCACCTTTTTCGTGGTGGCTAATTTAATTGCTTGAACA
GAAGTGAGAAGGTCAATTTTCTTCTTGTGGTGACGGTCTTTTTCTCAGTAGTGCCATGCTTTGCTTCTTT
TTACACCATTCACTTTAATTCCTCTAGAATCGTGTATGTTACAGCACTAGGTTTATGATTTACCTTGTGA
GCTTTAGATGTAGAAGAGCTGCAAATTATTTGAAGCATCTATTATCCACAATGAACTTTTTCTGATTATT
GTTGTGTATGCAGTCTGGTCTCCCTTTTCGTTGCCCTTTTTTCCCCTTTCTGGTAGTTATCACAAAAATA
AGGAAGGTAAATATAGTTGAGTATTTGAAGATAAAGTTACTTAGATAACTCATACATTGAAGATCATTAT
GTCTCCAAAGGAGTTGTCATGAAGCTGAATTTGTGAAGTTCCTTACTACTTACTATAATGGTTTCCTATA
ATAATCGGCTTCTGGGACTCTCTTCTCTGGAAGTCATGCCTGGGCAAGAGTAGCACCATAATGATTGACC
CCCAGGAAGTCCTCGTGTTGCATAATTGCTTTGAATGGCATGATAAGTAAAGTGAATTAACATTTGAAAC
CTGCATATGCACCTCAAAATCTCTTTAAGAATTATTAGAGTTACATAAGTGTTACATGATGAAGTTATTT
GTTGAACTTTTGTACTTTCGCTTTCAGTAGCTGATTTTTGTCCAGTTTTGAGGATACTAGTTATTTTATT
TATCCACTTATCCAATTCTGTGTTCTTTCTATTTATAGAGAATGACACCGGTGACTTATCGCCCTACTCA
TGACAGGACTCTTCCTCCACCAAATCAAGGTAAATCAAGAAAATGATTTTTTTTTTTGTTCCTTAGACGT
CTTTGATATCATAATTTACATTTATCTATTGGTTGCTCTTACCCTCTACCTTTATCAGGTTTTCGGATTA
TATAGATTTGACTAGAATCCTTGAGCCGAGGTTTTGTCAGAAATACCTCTCTACCTTCCAAGGTAGGGGT
AAGGTCTCTATACACACTACCCTCCTCAAACCTCACTTGTTTAATATGGATAAAAATAATAATACCAATA
ATAGAAAACAATACTCCTATATTTTTTGGTTAATAAATAAAAATGGTTGAAATAATAAAGATAGTGATTG
GACATTATTTGGACAAGATTTACCTAATCTACTTTTCAAAATTAAAATCTTTATTCAAAGAGACACAAGA
CTAATTATTCATTAAGGCAAGGGATTTAATATTGGATCTCTAAAGTCTTAAATAACTAAATCAAGGTGAT
ATTGATCCATTTTAATCATGATTAATTACAACTGCTCAACGCTTAAACATCCAACAATGTGTCAGAGTGA
TACGTATAAAGTATTGTTGGATGTTATTGTCCAACAATACTTTATATTTGGGCATGTTATTGTTGTATAT
ATTTGACTAGAATCCTACCCTTCCTCCCTAAGTGAATGGTGAATGGAATAACTTTAGCTCAGCTGCTGTT
ATTGGTTATTCTTATGAGTTGGTCTTTAGAATTAAGCCAGTCATCTCTTGAGGATTTGAACTTCAGTTTA
TTCACTTTAAAAGTTGTTTCAACCTCATATCATTTTGTGTCGCAAAAGCATTGGGCATGGATCAATAAGA
ATGTCAATTGGCAATGAGTTGTATATGGGTAGCAGAATCAAGCACCCCTAATTACTAGTTTTGGATTTCA
AGCACCCCTAATTACTAGTTTTGGACCTTCTCCCTCTGCTTCTGGTTTTGGTGAAGGTAAAATAGTCTAA
GAAGTTCTCCGAGTATGTCTGTGTTGAGGCTTTCATCGAAACAAAGAGTGAGGCCTGCCTGGTGTTTTTT
CTATGTTAGTCAGGTGAAAGGATTGGTTCACTATAAATAGTTTAGTGTATTAAGTAAGGCACTACTAAAG
CCCCCATTCCCCTTTTTGGGCGATAAAACTCTTAGCTTCCAGCAAGCTAGGTGCATAAGCTTTTTTTATT
ACATAAAAGATTCCAAACAATGCAGGCAGGGTAGAGCTCGGCAGAGGGTTCAAGAATTGGGTAGGGTAGG
GCCCTTAAGATTCAATAAGAAAGAATATGCCTTTCAAGAAAATAATGCAGCTATTACCTTTCCTCATATG
GCTATATTTAATTTACAACTCCTGTAAATGTGTGTGTTTACAAGTTACAGTTCTCATGTTCATCTCCATT
CCCTTTTAGAAATCTTGGTTTAGTTCTATCGGATGATATTATAATTATCTTGCCGCGTTACCACATTTCT
ATACTCAACTAAGATTGATTTCACATGCACTAGGATAAACTACTTTTCAATAGCTTTAAGATGCATCATC
TTTTTTTATTTAGACATGGCTAGTAACTATATGTAAGATCCTGTTACCGAAAAGGAAAAAAAGGGATTTC
TCTTAGGTTAAACAATTGGTAAATATAGTATCATAGCCATCTAAAGTACATTCATGTTATTGGTTAAAAA
ACTTTTATGAGGATATGCACATATTCGTGAGCCAAGAGTCTATTGGAAACAGCCACTCTACCTTCACAAG
GTATGGTAAGGTTGCGTATACACCACCCTCCCCAGACCTAATCATCAAAATCTAATGGAATGCGCAAGTT
TGTTTGTCAGTTTACTATCTCATGCTTCATCGTCAATCTTTTCTTTTAATTGAATCTCAATTAATCAGAT
AAGAGGAGAAATATATTGGATATGAATTGCATGTGCTTAAAGCTTTTGCACTTAAGCTATGCTATTTCTG
ATTCAACATATACTAATGACCTGCATCTGAATTTCCAGTAATTAGTTCAGAAGCCAAAAGTATACTTCTG
AGACACCTAGAGCAGCGTGCCGAAGAGAAGGTCAGTGTTCGTTTTGATCGACATTTATTCCTTCTTTCGA
TGAATATTTGGCAATTTGTAATTGTGTTATATTGTGAGGATTCATCTTAAAGTGACTAGTTTTACAATGG
CTATCAACCTATTGCAATTCTTCTCCTTTATCCAGTCTTGTGACCGACAATATGAGCAAACTCACACATA
ATCAATTGAGGATTGAGGCGTTGTTGCTATCCCTTTACGGTTAAAATGCTTTTCTGTGTTGCAGTTGAGA
CCAAAGCGAGCTGCGGCTGAAAATCTGGCACCCGAGCATGGATCGAAGCATCTTAAGGTATCCAACTGAG
ATGCTTTTCTTTTCGGTGCTACCCCTGGGCGGGAAGAAGATGAGGTAATGCGGTTGCAAACAGGACGATA
CACAACTTGGTTTTAGAAGATTAACTAACTCTTCTAAATAGGTTAAAATCTTCTGGTTTTTCTGCATATT
TCTGTAAATATGACTGTAATGATGCAGATACATGTTGTTGTAAAACTATGAAGAGCTTGTTTGTCAAGTA
GTCATATAGCAAATGAGATGTACACTAGATAAAATGTTGTTAGATGAGTATTGATAAATGTTTCCCTCCG
AGGATTTATCGGAAACAACCTCTGTGATCCGTCCTCTGTGACCCTAGAAAATGATATTTTATCATAATGA
TCAAACTTTTTAACATTGA
Solanum lycopersicum
SEQ ID NO: 123
MGSMFGEWPSIDPHNFSQLRPSDPSTPSRMTPVTYRPTHDRTLPPPNQVISSEAKSILLRHLEQRAEEKL
RPKRAAAENLAPEHGSKHLKVSN
Nicotiana tabacum, CDS
SEQ ID NO: 124
ATGGGGTCAATGCTAGGTGATTGGCCTTCTTTTGACCCTCACAATTTCAGCCAGCTTCGCCCTTTCGATC
CCTCCACCCCTTCTAGAATGACACCCGTGACTTATCGTCCTACTCATGATAGGACTCTTCCGCCACCAAA
TCAAGTTATTAGTTCAGAAGCCAAAAATATACTTCTGAGACACTTAGAGCAGCGTGCTGAAGAGAAGTTG
AGACCGAAACGTGCTGCGACTGAAAATCTTACACCAGAGCATGGATCTAAGCATCTTAAGGCATCCATCT
GA
Nicotiana tabacum, cDNA
SEQ ID NO: 125
GGCTTTGCCAACATCAATATTTTGTCCAACCCACCATATATTTTGCAGCTTCTATTTACCTCCGGTGTCT
AAAACAGTGGCATTATTCTCTCGATTCTTCCCGTTAATAATTCAATGGGGTCAATGCTAGGTGATTGGCC
TTCTTTTGACCCTCACAATTTCAGCCAGCTTCGCCCTTTCGATCCCTCCACCCCTTCTAGAATGACACCC
GTGACTTATCGTCCTACTCATGATAGGACTCTTCCGCCACCAAATCAAGTTATTAGTTCAGAAGCCAAAA
ATATACTTCTGAGACACTTAGAGCAGCGTGCTGAAGAGAAGTTGAGACCGAAACGTGCTGCGACTGAAAA
TCTTACACCAGAGCATGGATCTAAGCATCTTAAGGCATCCATCTGAGTTGCTTCTCTTTTTGTGCTACTC
CTGGGGCGGGAAGAAGATGAGAAAATGCCAAGTGTGACAGTTTCAAGTCGGATGGTACACAACTTGGTTT
TGAGAAATGACTTCTAAATAGGTTTGACGTCTTCGGGTTTTCTTCATATTTCTGTAAATATTGTTTTAAT
GGCAGAGATGCATGTTGTTGTAAAATTGA
Nicotiana tabacum
SEQ ID NO: 126
MGSMLGDWPSFDPHNFSQLRPFDPSTPSRMTPVTYRPTHDRTLPPPNQVISSEAKNILLRHLEQRAEEKL
RPKRAATENLTPEHGSKHLKASI
Eucaliptus grandis, CDS
SEQ ID NO: 127
ATGGGTTCTATCCTGGGCGACTTGCCGTCGTTCGATCCTCACAACTTCAGCCATTTCAGGCCCTCCGATC
CCTCCAACCCTTCCAAAATGACGCCAACAACCTATCATCCCACCCACAGCAGGACTATTCCACCACCTGA
TCAAGTGATAACTACTGAATCCAAAAATATTCTGATAAGAAATTTCTATCGGCGTGCTGAAGAAAAGATG
AGACCAAAACGGGCTGCCTCTGAATTTCTTGCACAAGAACCAGGATGCAAGCAACCAAGGGCTTCCATGA
CCACCTCAGATACCCCATAA
Eucaliptus grandis, cDNA
SEQ ID NO: 128
GGAGTTTTCGGTGGCATTAAGGCTTCATGTTTTCACGACGGATTATTTCTTTCGTCCATAGATTTGTGTC
TATACTTCGGAGCGTCTCGTGTCGGGGGAGTATTAATGAGCTTTCGTCGTAAGGTCAGACACGACCGTCC
TGTCCTGTTTCCAGGCAACTCCAGCACCAGCAGCGAGGCTGATTCTAGAATTTAAGGCCATCGTCTCTCT
CTCTCTCTCTCTCTGGATTCGAGGGGGGAACACTGTGCAGAGGTTCTGCATTCACTCTTTCATGGGTTCT
ATCCTGGGCGACTTGCCGTCGTTCGATCCTCACAACTTCAGCCATTTCAGGCCCTCCGATCCCTCCAACC
CTTCCAAAATGACGCCAACAACCTATCATCCCACCCACAGCAGGACTATTCCACCACCTGATCAAGTGAT
AACTACTGAATCCAAAAATATTCTGATAAGAAATTTCTATCGGCGTGCTGAAGAAAAGATGAGACCAAAA
CGGGCTGCCTCTGAATTTCTTGCACAAGAACCAGGATGCAAGCAACCAAGGGCTTCCATGACCACCTCAG
ATACCCCATAATGAGCTTCTGCATCGGGGTTTGCGACATGAGAAGTTCAGCAGTCTGCACTCATTGAGTG
TATATATACTGCTGTAATATCAGACTGGTCTGTAGGCTTGGCATCTGCCTATTTTAATGGTATGTGGTTG
CTGACCTTGTGTCTGTTATTTGCTGATGCTGGTTGGTTTCTGTGAAGAAAGCGCCTTTTGGGGGATGCAC
AGCCTCTCCCACCGTGTACATTGGAAATAATCAATCCTTGATTTTCACCATCTCAATAAA
Eucaliptus grandis, gDNA
SEQ ID NO: 129
GGAGTTTTCGGTGGCATTAAGGCTTCATGTTTTCACGACGGATTATTTCTTTCGTCCATAGATTTGTGTC
TATACTTCGGAGCGTCTCGTGTCGGGGGAGTATTAATGAGCTTTCGTCGTAAGGTCAGACACGACCGTCC
TGTCCTGTTTCCAGGCAACTCCAGCACCAGCAGCGAGGCTGATTCTAGAATTTAAGGCCATCGTCTCTCT
CTCTCTCTCTCTCTGGATTCGAGGGGGGAACACTGTGCAGAGGTTCTGCATTCACTCTTTCATGGGTTCT
ATCCTGGGCGACTTGCCGTCGTTCGATCCTCACAACTTCAGCCATTTCAGGCCCTCCGATCCCTCCAACC
CTTCCGTGAGTTCCCCGTCTTTCTCTTGGCCCTCCGCCTTGTTCCCTTTTTATTTTTTGGGTGGCGTGTG
CTTCGTTTAGTTTCCTCAATTTCTGCTGCTGCTACCGATGCGTGTGATTCTTTTTCTTGTCGCTGTCCTT
AGTCTAATTTTTCGTGGTGGAAGATGATGAAGAATTTGCATAGGAGAAGTGAGGTTTCCTCAGCTTCCAT
GTCCGAACTGAGGGGTTTTTAGCAGCAGAGATCATTTGATGGAGCTGGGTTCACCCGTTTTGCGGCTTAA
CTGGGGCAAAATCACAACTTTGTTCCGGCTGAATGAGTTCTTGGCTTATATCTTAATTCTTCTAGTTGAT
TTTAGCCGGGICTAGGATTCGGACTGATTGGGACGGTAATGCTTGTTCTTAGAGATAGTGTTTTTATACA
TTTTGGACGCATTGAGTTCCTCAATTTCTGCTACCGATGAGAATGATTCTTTTCTTGTCTCTGTTGTTAG
TCATTTTTTTGGTGGTGAAAGATGATGAGGAATTGCATAGGAAAAGTGAAGTGTCCTCGGCTTCCATGTC
TGAACTGAGGGGTATTTATCAGTAGAGATCGTTTGATAGAGTTGGGTTCTGCACATTTTGTGGCTTAAAT
GGGGCAAAATCACAACTTTATTCAAGCTGAATGAGTTCTTAGCTGATGTCTTAATTCTTCTAGTTGATTT
CAGCCAGGGCTAGGATTCGGACTGGTTGGGACAGCAATGCTTGTTCTTAGAGAAGTGTCTTTATACATAT
TCGACACATTGAGTTCCTCAATTTCTGCTATTGATGCGTGCGATTCTTTCATTGTCTCTGTTCTTAGTCT
ATTTTTCAGTGGCCAAAGATGATGAAGAATTGCATAGGAGAAGTGAAGTGTCCTCGGCTTCCATGTCCAG
ACTGAGGGGTTTTTATCAGTAGAGGTCATTTGATAGAGTTGGCTTCTGCGCGTTTTGTGGCTTGAATGGG
GGCAAAATCACAAGTTTATTCCATCTGAATTAGTTCTTAGCTTATGTCTTGATTCTTCTAGTCGATTCTC
GCTGGGGACCAGTTGGGACAGCGATGCTTGTTCTTAAAAAAGTGTTTTTTTATACTTCTTCGACGCATTG
AGAATATGTTGCCTTGCCATGGCTTACCACATCTTATCTATCTTGTGATGAGATTTTTCTGGTCTTCTTG
GCTCTGCCCTGCTCAAGTGCTAGTTCTAAAACCTGAGAAACACTGGGAATTAGGGCGGTCACGAGAAGTC
TTTAGCACAAATTTGGCTGCTCATGTCATGTCCITTGTAACAGAGCCATAAGCTTTAAGCCAACGAGCCA
TTGTAGTTTGGCCAGATGGGCACACAGTGCTGCATCTTTGTGACTGGTTTGGAACATGGTGTTTTATAGT
TTGTAGGGATGATTTGATCTTGAGGAGATGTAGAGTAACTTCTCCTTTGAAATTCCGGCAAATTAGTTGT
ACTTGTGCAGAATTCCTGAAGTACTTTAAGAAACTATAATCACATAACAAGTTTCTGGTCTTTTGAATAA
GTTTCCTTCTTGGTTTAGAGAATTCTTATGGTAGTTCTTTTCAAGATTAGGAGGTTATATCAGGACATCA
ACTGATCTAAGCTACACCTATCAAAATTAAAGGGCCATACATTGTGTTCAAATTTAATTTTAAGTTATTA
ATGACCGGACGGAACCAAGAAATAACACCTCATGTGCAGTTTGCTGTTGGCAATTCAGCTGTCCAACAAA
TGACACAGCTTGTGTCTGGTGATTGGTGAATAAAGTGAGCTCTCTTCAATTCCTTCTAGGTCGAGGAAGC
TAGATTTGATTAAATCACCTTGTGGATGACAAAGATCTCTTTGCTTTATAACTGATGTCTATTGCAGGAA
CGCTGTTTTGTTACTTTTTGATTGGAACAAGAAATAGCATGGACCCGAGATAAGATGATTTGAACATAGC
TTTACTTACAATTTTCAAATGTTATATGTAGAAAATGACGCCAACAACCTATCATCCCACCCACAGCAGG
ACTATTCCACCACCTGATCAAGGTAATAATGAATTAGATATGCATCTCCAGCACTTTTTGCTCATTGATT
TAATGTCGATGTTGAGCTCTTTAGCTGATTGAGAAGAGAATGTCACTTTTATGTGGAAGATAAGCATTTT
TTCCATTTCAGTTTTTGGAGATGTGGTTCTCATCCTCTTATTGCGATGACCCTGACTTGGTTGCAACTCC
TTCCAGTGATAACTACTGAATCCAAAAATATTCTGATAAGAAATTTCTATCGGCGTGCTGAAGAAAAGGT
TAGTCATTCATATGAAACCAGAATTTTGATAACCTACATAGCACGTGCCTCTCTCATGCATCCTTTGCAT
TTCGCATCCATAGACTACGCATGCTACGTGGATGAATGTTCGCATGCATTTGCACTCTTCATTGGTGGAT
GTCTGCCTTACACACACAAAAACACACAGATGCTTTCCATTGCTGATTAGTTTTTGTCAATATCAGATGA
GACCAAAACGGGCTGCCTCTGAATTTCTTGCACAAGAACCAGGATGCAAGCAACCAAGGGCTTCCATGAC
CACCTCAGATACCCCATAATGAGCTTCTGCATCGGGGTTTGCGACATGAGAAGTTCAGCAGTCTGCACTC
ATTGAGTGTATATATACTGCTGTAATATCAGACTGGTCTGTAGGCTTGGCATCTGCCTATTTTAATGGTA
TGTGGTTGCTGACCTTGTGTCTGTTATTTGCTGATGCTGGTTGGTTTCTGTGAAGAAAGCGCCTTTTGGG
GGATGCACAGCCTCTCCCACCGTGTACATTGGAAATAATCAATCCTTGATTTTCACCATCTCAATAAA
Eucaliptus grandis
SEQ ID NO: 130
MGSILGDLPSFDPHNFSHFRPSDPSNPSKMTPTTYHPTHSRTIPPPDQVITTESKNILIRNFYRRAEEKM
RPKRAASEFLAQEPGCKQPRASMTTSDTP
Lactuca serviola, CDS
SEQ ID NO: 131
ATGGGGTCGTGGATTGTTGGTAATTGGCCTTCCTTCGACCCCCACAACTTCAGCCAACTTCGCCCCAACG
ATCCGTCTGCTCCTTCCAAGAAGACACCAATTACATATCATCCAACTCATGAACGAACTCTTCCACCACC
TGACCAAGTAATATCTTCGGATGCCAAAAACATACTTCTGAGGCAATTCTATGAGCGTGGTGATGAAAAG
TTGAGACCAAAGAGAGCTGCCCCTGAGAATCTGGCACCCGAGCAAGAATGCAAGCATCCAAGAGGTTCTT
CTTCAGATCCTCTATCATG
Lactuca serviola, cDNA
SEQ ID NO: 132
CGGGGGAGCCACTGTTCCAAAAATGTGTCAAGTATCCCTAAACAATCAATTTCCTCATCTGTCCATCTCC
AGAAACTGCGATTATCGGGGCTGAGAAATCTGTGGGACTGTAATCGATTTTAATGGGGTCGTGGATTGTT
GGTAATTGGCCTTCCTTCGACCCCCACAACTTCAGCCAACTTCGCCCCAACGATCCGTCTGCTCCTTCCA
AGAAGACACCAATTACATATCATCCAACTCATGAACGAACTCTTCCACCACCTGACCAAGTAATATCTTC
GGATGCCAAAAACATACTTCTGAGGCAATTCTATGAGCGTGGTGATGAAAAGTTGAGACCAAAGAGAGCT
GCCCCTGAGAATCTGGCACCCGAGCAAGAATGCAAGCATCCAAGAGGTTCTTCTTCAGATCCTCTATCAT
GATGGATTCATGAGTAATCGAGTATGCATGCATAAATCATAATGCATTGCACATTGATGTAAATATTATT
TGGTTGTCGATGCTATATGTGTGTTGTATGTTTTTGGGAAGCTATGAGATAAAGCCAATTGTTA
Lactuca serviola
SEQ ID NO: 133
MGSWIVGNWPSFDPHNFSQLRPHDPSAPSKKTPITYHPTHERTLPPPDQVISSDAKNILLRQFYERGDEK
LRPKRAAPENLAPEQECKHPRGSSSDPLS
Helianthus exilis CDS
SEQ ID NO: 134
ATGGGGTCGTCGTGGGATGTTGGTAATTGGCCTTCTTTCGACCCCCACAACTTCAGCCAACTTCGCCCCA
ACGATCCTTCCGCCCCTTCCAAGAAGACACCAATTACTTATCATCCAACTCATGAACGGACTCTTCCACC
CCCCGACCAAGTAATATCTTCGGAAGCCAAAAACATATTGCTGAGGCAATTCTATCAGCGTGGTGATGAG
AAGTTGAGACCAAAGAGAGCTGCTCCCGAGAATCTTTCACCGGAGCAAGAATGCAAGCACCCTAGAGCTT
CATTTGCTTCATCTTCCGAGCCTCCAAAATGA
Helianthus exilis cDNA
SEQ ID NO: 135
GAATTCGTATGCGTATGCACATCATCAATCTATCTTCCGATCTGCTGCTGCTGCTGCGAATCTAATAATC
GGGGCTGAATGGGGTCGTCGTGGGATGTTGGTAATTGGCCTTCTTTCGACCCCCACAACTTCAGCCAACT
TCGCCCCAACGATCCTTCCGCCCCTTCCAAGAAGACACCAATTACTTATCATCCAACTCATGAACGGACT
CTTCCACCCCCCGACCAAGTAATATCTTCGGAAGCCAAAAACATATTGCTGAGGCAATTCTATCAGCGTG
GTGATGAGAAGTTGAGACCAAAGAGAGCTGCTCCCGAGAATCTTTCACCGGAGCAAGAATGCAAGCACCC
TAGAGCTTCATTTGCTTCATCTTCCGAGCCTCCAAAATGAGGCATACTCACCTTTCTGCACAATGATGTA
AATAGTCTTCACTGCTGAGTGTTGATACCATATGTTGTGTGTGTTTTAGTGAGGTATGATACGAGTGAAT
CGTTTGCATCTTGGTGTGTACTTTCAGCTATACAGACTTGTACATTTCTATATTTATAAACAGGCAGATA
ACTAAATATGCAAAACAACATCCTTGGCAT
Helianthus exilis
SEQ ID NO: 136
MGSSWDVGNWPSFDPHNFSQLRPNDPSAPSKKTPITYHPTHERTLPPPDQVISSEAKHILLRQFYQRGDE
KLRPKRAAPENLSPEQECKHPRASFASSSEPPK
Helianthus annuus CDS
SEQ ID NO: 137
ATGGGGTCGTCGTGGGATGTTGGTAATTGGCCTTCTTTCGACCCCCACAACTTCAGCCAACTTCGCCCCA
ACGATCCTTCCGCCCCTTCCAAGAAGACACCAATTACTTATCATCCAACTCATGAACGGACTCTTCCACC
CCCCGACCAAGTAATATCTTCGGAAGCCAAAAACATATTGCTGAGGCAATTCTATCAGCGTGGTGATGAG
AAGTTGAGACCAAAGAGAGCTGCTCCCGAGAATCTTTCACCGGAGCAAGAATGCAAGCACCCTAGAGCTT
CATTTGCTTCATCTTCCGAGCCTCCAAAATGA
Helianthus annuus CDS
SEQ ID NO: 138
CGTATGCACATCATCAATCCATCTTCCGATCTGCTGCGATTATATCACCGGGGCTGAATGGGGTCGTCGT
GGGATGTTGGTAATTGGCCTTCTTTCGACCCCCACAACTTCAGCCAACTTCGCCCCAACGATCCTTCTGC
CCCTTCCAAGAAGACACCAATTACTTATCATCCAACTCATGAACGGACTCTTCCACCCCCCGACCAAGTA
ATATCTTCGGAAGCCAAAAACATATTGCTGAGGCAATTCTATCAGCGCGGTGATGAGAAGTTGAGACCAA
AGAGAGCTGCCCCCGAGAATCTTTCACCGGAGCAAGAATGCAAGCACCCTAGAGCTTCATTCGCTTCATC
TTCCGAGCCTCCAAAATGAGGCATACTCACCTTTCTGCACAATGATGTAAATAGTCTTCACTGTCGAGTG
TTGATACCATATGTTGTGTGTGTGTTTTAGTAAGGTATGATACGAGTGAATCGTTTGCATCTTGGTGTGT
ACTTTCAGCTATACAGACTTGTACATTTCTATATTTATAAACAGGCAGATAACTAAATATGAC
Helianthus annuus
SEQ ID NO: 139
MGSSWDVGNWPSFDPHNFSQLRPNDPSAPSKKTPITYHPTHERTLPPPDQVISSEAKHILLRQFYQRGDE
KLRPKRAAPENLSPEQECKHPRASFASSSEPPK
Zea mays 1 CDS
SEQ ID NO: 140
ATGGGGAGCCCTCTGGGCGGGTGGCCGTCATACAACCCGCACAACTTCAGCCAGTTGGTCCCTGCCGACC
CCTCCGCGCAGCCCTCGAATGTCACACCAGCCACTTATGTTGCGACCCACAGGACAGATCCGCCACCCAA
TCAAGTGATAACCACGGAGGCCAGGAACATCCTGCTGAGGCACTTGTACCAGAAATCCGAGGAGAAGCTG
AGGCCAAAGAGAGCTGCGGCGGACAACCTCGCTCCGGAGAACAACAACAAGAAGCAGCCCAGGGGACCTG
TGGGCGACGTCGGGGGCCAGTCGAGCGCAAGAAGCTGA
Zea mays 1 cDNA
SEQ ID NO: 141
CTTTTTCCCCGAAACCAAAACAGAAAAAAAGTAAAGTCCTGCTGGCAGCTGTCAACCACCCGTGGTCCCG
TGGAAGAGAAGAGAGCATCGCCGGACCCGGGGACGGCGCGCCGAGAAGGAACAAAAGAAGACGGCGGCGG
GGCGGAGATGGGGAGCCCTCTGGGCGGGTGGCCGTCATACAACCCGCACAACTTCAGCCAGTTGGTCCCT
GCCGACCCCTCCGCGCAGCCCTCGAATGTCACACCAGCCACTTATGTTGCGACCCACAGGACAGATCCGC
CACCCAATCAAGGGCGTGTTATTTGTGAGCAACTGGACAATTCAAAACATCTGAATGGGTACTTCAGCCA
CAGACTTCTGGTGAGGTGCAGTGATAACAGCAGAGATATCCCAATTTGTATAGCAGATAAATTGATAACC
ACGGAGGCCAGGAACATCCTGCTGAGGCACTTGTACCAGAAATCCGAGGAGAAGCTGAGGCCAAAGAGAG
CTGCGGCGGACAACCTCGCTCCGGAGAACAACAACAAGAAGCAGCCCAGGGGACCTGTGGGCGACGTCGG
GGGCCAGTCGAGCGCAAGAAGCTGAAGACGCACAGCTGGTGGCCGTCCTCCCCTGCTTCTCATCTATCGG
TGTCATGCAGCCTGCATCTCTCACTCACAGCTGAGCTGGTAGCTGGTGGTGGTTGCCCTCCCCTCCCCTG
TGCGTCCTCTTCGCCTCTCACGTCTCGTATGTACGTATGGTATGACCAGGAGAGCTAGTTTGCATACAAT
GGATATACTGGATGTGCATAGCCACCTGAGACGAGACGAGACGGGACTGGACGAGGTCGGTGCGTGCCAT
TTCACACGGCACTACCGCACTAGTCTGTGCGGCAGCCTCTCTCTCTCTCTCTCTCTCTCTCTCTCTCTCT
CTCTCTCTCTCTCTCTCATCCTCTGCAATGCAAAAATATGGATGCGCCCATGCTG
Zea mays 1 gDNA
SEQ ID NO: 142
CTTTTTCCCCGAAACCAAAACAGAAAAAAAGTAAAGTCCTGCTGGCAGCTGTCAACCACCCGTGGTCCCG
TGGAAGAGAAGAGAGCATCGCCGGACCCGGGGACGGCGCGCCGAGAAGGAACAAAAGAAGACGGCGGCGG
GGCGGAGATGGGGAGCCCTCTGGGCGGGTGGCCGTCATACAACCCGCACAACTTCAGCCAGTTGGTCCCT
GCCGACCCCTCCGCGCAGCCCTCGGTCGGTCAGCCGTCAGCAACTTGCCTTCCTGGCGATCTGGCCTCTA
GTATCATGATGTACTGCTAGGCTCCGTACTTCCTGCTAAGCTTACACAACCATGGATATGTCTAATTGAC
CGTGCTCGGCTGACCTGTTTCTTTTCTCTGCTGTCTGGTGTCGTCGCGAGAAAAAAAAAACTCTCTTTTT
TTATCCCGCAGTATCACTTTCGGGCAGGAGGCAGTAATCGGTGCCCGTATTCAGGGGCGGACCCAAGTAG
GGTCGAGTGAGGGCAATCGCTCGTTTCTTTGTTCTAAATACTGAATCTAGATTTTCGCTATAAGGTTCTC
TACTCAGTCATATTCTGTCTTGGGTCCGCCCCAGTGACGGATCTACACCCCGGGCCATTCGGTCCGTGGC
CCGGGGTTTGATCCATGTAGCTATATATATGTCTCTATTTAATATGGTATAAGATATTTAAAATAAAATG
AAGAGAAGATAATTTGGTAGATTTGGTCTGGGTCATAGAAAAATTCTGGATCTGGTCCGCGTCTACCCGT
ATTGTTAGTTTTTGCTGGAATTTTGGTATGTATGGATGGAGAAATGGGGTCTCACCGTTTGATTTTAGTT
CAACTGCCAAAGACCTGTTGAATTTGAGGGGACTGTCTGGCGAATTTCCAAACGCATGGTCTGGTTTTCT
CATGGTCATGTCTACCCTGGGCAGATTCAGTTGATGTGGTACTGATGAACTAACTGTAGTTCAGTTCATG
GGCTGCTAATGCTACCCGCTACCGGTTGATTTTTATAACGTCAGAAATTCATGCTAGCAATTGACATTAT
GAATGATATATCCATTTCACCTGGTGGTAATGTTAGTTCTTTTTCCTTTCCCTGTGTGTTTCTCATCAGA
ATGTCACACCAGCCACTTATGTTGCGACCCACAGGACAGATCCGCCACCCAATCAAGGTAAACCCTTTCC
ATGTCCTAAGCCAATGATGTTCTGCTGCATCCATCAGCAAATTTGTCCCATCTGATTTCCTAGTTTTGCA
TTTGCACGTAATTCATATCGTACAATTCCTTTCACATTAAGCCAAGAATCCCGCTATTTTGTTTATCGTA
GTGTTTTTTACATTACATTGCAAAATAGGTTTCATGAGCTGTTTCGTCTGAACTGACGTTCTTCAGTTAG
ATGACTCTCTTTGCTTCAATGGAGCATGATTAGCCATAAGCTTTTGTGCATGGGGTTGAAATGTAGAACG
TGTCTGCCAGTCACAGATGGTGGTATCAGCCATAGCGACAGACTGACAGAAGCTCTGAATACCTTATCCT
ACACAAATGACGCCTCTGCAACTCTTCTGTGTTTAGTGTTTAGCTGAACCCAGCAGCTCTGAACTTCCTC
GTTGTTGCTAGTAATCAGAATTCAGAAGTTAATACTCCCTCTGTTTCAAAATATAATTTGTTTTAGACTA
AACATACATTCATAAATTAACCTATGAATGTGGTTTGTATGTATGTCTACATTCATTATTTTTCATTCGA
ACGTGGACAGAAAAAAAAGAGGGCTAAAAAGAAATATATTTTGGGACAGATGGAGTATATTTTGGGACAG
ATGGAGTAGATATGATCGATAACTCAGCAGTTGCTGTCTTAGCCATGTACTCCAATACAATAAATACACA
ACGTTGCAAGTACTAACAGTTCCAGGCTACCAGCTTTGACTATGCGATTCCATATAACATTCTTTCTTTG
TTGCAAAATCCTTCAGCAATTATAGTGTTATGCCTTCAAAGAACGCAGCTGGGAAACATTGCCTGTTGTA
TTTAGGAAGTTCTAGATTCTGAAGGTCAGCTTTCTTATTTTACTGAAAGTCTGAAACACACTGACTATTA
ACACATTAATATTGATTCATCTAGTCACATCAAATGGTAAATTGATTTGTGACACTAATCCAGATTAATG
CATAGTAAGTATTCACCTCGAATACAGTAGATCAACAGAGGTGAGATTATACCATAGCCTATAATCTCTC
TTGATATCTCAGCATTTGGCATGGCTTATACATATTATGGCTGAGTTGTTTTGTGCCTTTTGTACCGTTT
TTGTCTGGAATATGCAGGAGGACTGCATATCGTTGCATTCATAGAATAAAGAGAGGGGAAAGACCCCCCC
CCCCGTACAACACACCCAAGCCACCCAAAGATCTCACCAGGAAAAAAACAGCAAGCAGACGACCGACCCA
CACAGATCTACCTAAACCGGCAGACCCAGAGGGCGGATAGATTTTTTGGCCCCAGCCATAGAACAAAACA
GCACCTCATCCTTTTGTTCCGTTGTGTAGGTTCCTTAGGAGATTTGCAGTGTTTTACATACTTAATCTCC
AAAACACTTTCTTATAGCACGAGAACGAGGAAGAAAAGTTTGGGTTAATTACTGCTTTATGGATCAGGGA
TGCTGGCGTTCAAAATCAACCCAGACACCAGTTAAATGCATCCATCATAATAATAGACCTTAAGAGTGGA
TTCTCTGGACTTTTTCAGTAAAGTTCGGAGCCTTTCGATCATATGAAAATGCTATCCACATGGAGCTCTA
GAACTGAGATGACCTTGAGAGAAGTTAGGTTAATTTACTACTGAATGATAGCCTAGTAATCACACCGACT
AGGATTTTTTGGCCTGAACACTCTAGTTGTCAGTTTCTGTAACATATGTCGCTCTTTGCTGCGGTCATTC
TCTGGTCCCGGACAATTTACCAGGTTAAGTGAAACCACCGGAAGCCCTTATTGAATTCGTGCCTTTTGGC
GCGGCTGATTCCACATCCCGTCGGTGGAAAATATAGTCGTGTGCTGCCTACCAACTGCATAAAAAGGTCC
CGAAAGAAACAAACAGGCTATGATTGTGCGTTTATATGGAGCTCATGACATATTTTCAGGGCGTGTTATT
TGTGAGCAACTGGACAATTCAAAACATCTGAATGGGTACTTCAGCCACAGACTTCTGGTGAGGTGCAGTG
ATAACAGCAGAGATATCCCAATTTGTATAGCAGATAAATGTACTGAACAAACCGTGGGCATTCTTTTAAC
TATATACATGCATGACAATTCTTCTCTCTCTCTCTCTCTCTCTCTCTCTCTCTCTCTTCCAAATTTCCTT
GTACCTATCATACCACTGCATTATTTTATATATGTATATAGGTCCATAGTTCACAGTACTTGAGGATCAA
TGACTGCTTCCTACAGACTACTGCGGACTGAATCGCGCCCTGGAATTGCAGTGATAACCACGGAGGCCAG
GAACATCCTGCTGAGGCACTTGTACCAGAAATCCGAGGAGAAGGTGAGCAGCTACTGCTACTGCTAGTAA
GACTTCACTATCACGCACGGCTACATAAAACCACATCACCGATAAAGGTTAAAACCCTGTCCTGAACTGT
AGCTGAGGCCAAAGAGAGCTGCGGCGGACAACCTCGCTCCGGAGAACAACAACAAGAAGCAGCCCAGGGG
ACCTGTGGGCGACGTCGGGGGCCAGTCGAGCGCAAGAAGCTGAAGACGCACAGCTGGTGGCCGTCCTCCC
CTGCTTCTCATCTATCGGTGTCATGCAGCCTGCATCTCTCACTCACAGCTGAGCTGGTAGCTGGTGGTGG
TTGCCCTCCCCTCCCCTGTGCGTCCTCTTCGCCTCTCACGTCTCGTATGTACGTATGGTATGACCAGGAG
AGCTAGTTTGCATACAATGGATATACTGGATGTGCATAGCCACCTGAGACGAGACGAGACGGGACTGGAC
GAGGTCGGTGCGTGCCATTTCACACGGCACTACCGCACTAGTCTGTGCGGCAGCCTCTCTCTCTCTCTCT
CTCTCTCTCTCTCTCTCTCTCTCTCTCTCTCTCTCATCCTCTGCAATGCAAAAATATGGATGCGCCCATG
CTG
Zea mays 1
SEQ ID NO: 143
MGSPLGGWPSYNPHNFSQLVPADPSAQPSNVTPATYVATHRTDPPPNQVITTEARNILLRHLYQKSEEKL
RPKRAAADNLAPENNNKKQPRGPVGDVGGQSSARS
Zea mays 2 CDS
SEQ ID NO: 144
ATGGGGAGCCCTCTGGGCGGGTGGCCGTCATACAACCCGCACAACTTCAGCCAGTTGGTCCCTGCCGACC
CCTCCGCGCAGCCCTCGAATGTCACACCAGCCACTTATGTTGCGACCCACAGGACAGATCCGCCACCCAA
TCAAGTGATAACCACGGAGGCCAGGAACATCCTGCTGAGGCACTTGTACCAGAAATCCGAGGAGAAGCTG
AGGCCAAAGAGAGCTGCGGCGGACAACCTCGCTCCGGAGAACAACAACAAGAAGCAGCCCAGGGGACCTG
TGGGCGACGTCGGGGGCCAGTCGAGCGCAAGAAGCTGA
Zea mays 2 cDNA
SEQ ID NO: 145
GTAACCATCCTTTTCCCAGACCAAAACAGAAGAAAGGAAAGTGCTACTGGGCATTGGCTACTGGCCTACT
GCCAACTACCCGTGGGGTCCCGTGGAAGAGAAGAGAGCATCGCCGGAGTTGGGGTCGGCGCGCCGACGAG
GAACAAAAGAAGACGGCGGTGGGGCGGAGATGGGGAGTCCTCTGGGCGGGTGGCCGTCTTACAACCCGCA
CAACTTCAGCCAGCTCGTCCCGGCCGACCCATCCGCACAGCCCTCGAATGTCACACCAGCCACTTATGTT
GCGACCCACAGGACTGATCCGCCACCCAATCAAGTGATAACAACGGAGTCGAGGAACATCCTGCTGAGGC
ACTTCTACCAGAAATCCGAGAAGCTGAGGCCCAAGAGACCTGCCCCGGACAACCTCGCTCCGGAGAACAA
CAACAGCAACAACAAGCAGCCCAGGGGACCGGTCGGCGACGTCGGTGGGCAGTCGTCGAGCGCGAGAAGC
TGAAGCCACAGCTGGTGGCCGTCCACTCCTCCCCCTGCCTGCTTGTTTCTCATCTCTGGGTTTCGTCATG
CAGAGGCAGAGGCAGATGCAGCCCTGTGTTGTCCTCTTCGCTCCTCACGTCTGTACGTACGACCCAAAGA
GCTACTAACCTAATCTGAAGTTAGGGTTACATACATGGATATCGGATAGATGGGTGTACATAAGCACCTG
AGAGCAGTGTTGTGTTATCCTAAATGCTAAACGGTAAACAGTCGTTCAACATCCTCTGTGTTTAGCGTAT
AA
Zea mays 2 gDNA
SEQ ID NO: 146
GTAACCATCCTTTTCCCAGACCAAAACAGAAGAAAGGAAAGTGCTACTGGGCATTGGCTACTGGCCTACT
GCCAACTACCCGTGGGGTCCCGTGGAAGAGAAGAGAGCATCGCCGGAGTTGGGGTCGGCGCGCCGACGAG
GAACAAAAGAAGACGGCGGTGGGGCGGAGATGGGGAGTCCTCTGGGCGGGTGGCCGTCTTACAACCCGCA
CAACTTCAGCCAGCTCGTCCCGGCCGACCCATCCGCACAGCCCTCGGTCAGCAACTTGCCCTTCCTGGCG
ATCTGGCCTCTAATATCATGCTGTGCTGCTTGGCTCCGTACTTCCTGATGAGCTTACACAAGCTTGAGTA
TGTTTAATTGGCCGTGCTCATCTGCGCTTGGCTTATTTCTTTTCTCTGGTTTATGGCGTCGTTGAGGAAA
ATATCTTTAAAAAAAAATCCGTAGTATCGTTTTCGGGCAGGAGGCAGTAATCGGTGATTCGGTGCCCGTG
TTGTTAGTTTTTGCTTGGAATTTTAGTATGAATGGCTAGAGAAATGGAGCCTCGCTGTTTGATTTTAGTT
CAATTGCCAGAGACCTGTTCAATTTGAGGGGACTGTCGGCCCAATGTCCAAATATCTGGTCTGGTTTTCT
CATGGTCATGTCATGTCTACCTGAGTATATTCAGCTGATGTAGTGCTGATGGACTAACGGTAGTTCAGTA
TTTCAGTTCATGGGATGCTAATGCTACCAGTTAATTTTTATAACGTCAGAAATTCGTGCTAGCAACTTGT
ATTATGAGTGGTATATTCATTTAACATGGTGCTAATGTTGGTTCTTTTTTCTTTCCCTGTGCATTTCTCA
TCAGAATGTCACACCAGCCACTTATGTTGCGACCCACAGGACTGATCCGCCACCCAATCAAGGTAACCCC
TTTCCATGTCCTTAAGCGGAGCCAACGATATTCTGCTCCACCCATCAGCGAGTTTGCCCCATCTGATTTT
CTAGGTATCATTTGCATAAAATCCATGCCGTGCAATTGCTTATTCGCATTAAGCTAAGAATCCCTTTTTT
ATTATTCTAGTGCTTTGCGTGTTACATTGCAAAATAGGTTTCCTGGGCTGTTTCATCTAAGGCAACGACT
GCTATGCAAGCAGTCCTTCTTTGCAAGCGTGCAAGCAAATCATCTGATCCATTAGATTACGATTCAACCA
CAGATACAACCATGATTTGTTAGGATTTTTTTATAAAGATTATTGAGCTGGTGGTGGAAGGGTTTAAGTG
TTAAATGTGACCTACGGTTGGATCACGATCTAATGGATCAGATGGTTTGCTTGCACGTTTGCACAGAAGG
ACTGCTTGCATAGCAGTCGATGCCTTCATCTAAACCGACGTTCTTCAGGTAGATGACTCTCTTTGCCTTC
AACGGAGCATTATTAGCCATAAGCTTTTGTGCATGGGGTTGAAATGTAGAACGTGTCTGCAAGTCACAGA
TTGTGGTATCAGCCATAGTGACAGAAGCTCTGAATACCGTATCCTACATAAACTGTGCCTCTGCAACTCT
TCTGTGTTTAGTGTTTAACTGAACCCAGTAGCTCTAAACTTCCTCGTTGTTGTCAGTAACCAGAACTCAA
AAGCTAATAGATATGGTAGATAATTCAGCAGTTGCTGTCTTTAGACATGTACTCCAGACAATAAATGCAT
AACCTTCCTTGCTGGCAAATTTACCAGTTCCAAGAGCTAACAGTTCCAGGCTACCAGCTTTGACTATATG
GTTCTTATAACCTTCTCTCTTTGTTGCAAAATCCTGTACTAATTCTAGCCTATGCCTTCAAAACCACAAC
TAGAAAACATGGTCTGCTGTATTCAGAAAATTTTAGATTCTGAAAGCTAGCTTTCTTATTTTACTGAAAG
TCTTCCGAAACACTGAATATTTAATCACAGCCTAAAATTCGCTCCCATGGGGAGTCGAACCCAGGACCTG
AGGAGCGTTACTCAGACCACCTAACCAACTCGGCTAGATCCCCTTTCGCTTTTCAATGCCTCTCTAACCT
TAGATTCTTGTATCACACGCACAAAGTACCTATTGGTATCATCAAAAGAGTCACAACTAAAAGGTCGTGT
CCTCATCCTCCCCGTAACCCCATTGACCAGTTTGTCAAAATACTCTTGTCATATATATCTGGTCTCATCT
GCCTTCACTAAGATGTGCTCTATTTTATCCTTAATGCACTTAACTTTGTTGAAGTCTCTTGTCTTTCTCT
CACGGACCCTAGCCATCCTATATATGTCTTACTCACAAGTTGGTAAAGGTTATCGTATGCCCTATTCTTT
GCCACACTTACAAGTCGTTTTGCGGTCTTCCTTGCTAGCTTATACTTCTCTATGTTGTCCACACTCTTGA
CATGGTACAAACGTTTGTAGCATTATTTATTCTCCTTAATAGCCCTTTGGTTTTCCTCATTCCAACTTCA
AGCGTCTTTAGCTTCACCTCCACTCTCTTTGGTTGCTCCACACACCTCTGAGGCCACCTTCCGAATACAG
GTTACCATCTTCTCCAATATGTTGTTTCTGTCATCTTCTTGCTTTCAAGTGTCCTCTTTGATGGCTCTTT
CCTTGGAGACTCCTGATGTCTCTTATTTCAGTTTTTACCACTTTGTTCTCACAATCTTGGTTTGTTTATC
CATATGTGCACACACCTAAAAAATGGAAGACCGCAACCACAAACTTATGTTGGTAGACATCACACTCCTT
TGGTATCACCTTGCAGTCTAAGCATGCTCGTTTATCCTCTCTTCTTGTGAGGATAAAGTCAATCTGACTA
GTGTTCTGGGCTTTTGGCCACTATTTTAGGTCAATAAATGGGATTCTCTCTTCTTAAAGAAACTAGTGGT
TATCTGTCGAGAATCTTTCAACAGTTAATTTGTGTGGTGGTGTTTGCGAGATCAAAGAACTAAAAGTAAA
AGCACAAGAGACAATATTTAGACTGGTTCGGGCTCTCGTCGTGAGATAATACCTCACGTCCAGTATTATG
GTGGCTATGCTTACGATCTGTTGTGCTCTGCCCTCTTGGGGCGCCTCGCCCCTCCTTTTATAGTTGGAGG
GGTGCGGTTACAAGAAAACGTGTGGTCGTACTAGACAAGGACTCGGACCTAAAAGTCCTCGGTTACAAGG
ATCCCTATCATCGCTATAATGGCTTCCCTTATAAATCGGGTATTACCGTTACAATGAAGATATGACCCAT
ATATCGTACAAACCCTACAATCTTCCTTATTTGCTCGACCACGTAGACTTTATCCCGACATGTGGATTAG
GTTCTCCTGAAAGTCTGAGACCGAGTCCAAGTCCTAGACTGAGTCCGAGTTGGGCCCACAAGCCAACACC
TTTAGGATCAACGTACCTATGGTACCCCTTGTATATATTGTTTACACTATCAATAGGTCAAAAGCTACTA
CAAAGTTTAAGATTTCCTCTCCCTCTTGGTTTGTACTAGTATACCTAAAACCTCCACCTACATGTTCATT
GTGATCTCCTATGCAAAGCTCCTCACTAATATGTAGAGCTCTAAACATGCCATCGAAGTCTTCCTACCAC
TCTCATCGTCGTCTACTTGAGAAGTATACTCGCTAATTACATTTAAGACCAAATCATCCATGGTAAGCTT
GACTAAGATAATTGTATTTCCTTCCCTTTTCACATCCATCACACCGTTCTAGATGCTCTTATCAATTAAA
ACTCCCTACTTCATTTCTATTTGCAGCTATCCATATGTACCAAAGCTTAAAACCGGTATTGTCCACCCCC
TTCGTCTTCTGACCCTTCCATTTAGTCTTATGGACACACAAGATATTTACATGTCTCTTAGTCATTATCT
CAACTAACTCCTTTATAGCTCATTACCTGTAAGCGACCCTATATTCCAGCTGCCTAAAAAGATTATAGTT
TGTTCCATCCACTAGCTTTCTTCCCCTTTGCACCTACGATGATGTGAAGACCCTTACATATTTTTTTACT
ATATCTGGGCACTTATCATAATCTTCCTTTGCCATGGTTTGGGACCTGCTATATTGAGACAACATAGGCG
GATTTTATATACTTAATCTCCAAAAGACTTTGAATCTTAGAACCTGAGAAAGAGGAAGAAAAGTTTGAGT
TAATTGGTTCTTTATGGGTCAGGGATGTTGGTGTTCAAAATGAACCCAAGGACCAGATAATGCATCCATC
AAAGAGTGGAGCATCTAAGGCTTCGTCATAATGATCACACTGACTAGAAATTTTGCTTGAGCACTCTGGT
TGTCGATCTCCGTAACATATGTCGCTGTTTGTTGCGGTCGTTTCCCTGGTCCTGAACAATTTACCAGGTT
AGGCGAAGCCACAGGAAGTCCTTATTGAATTTGTGCCTTTTGGCACAGTCGATTCCACATCCTGTCGGTG
GAAATATTATTGTGTGTGCTGCTGCCTACCATGTGCCAGCTGCATAAAAGGCCCCGAAAGGAACAAACTC
TGATTGTGCCTTTTATCTGCGGCTGATGACATGTTTTTGGAAGATGTTATTTGTGAACAATTGAGTATAT
CCAAAACATCTGAAGGGATACTTAGGGGCTGTTTGGTTCGTGGCTAAATGTGCCACACTTTGTCTAAGAT
TAGTCGTTCGAATTGAAGAACTAACCTTAGGCAGAAAAGTTAGTTAAAGTGTGGCAAGTTAGCTATCAAA
CCAAACAGACCTTTAATCCTGGACTACCGCCGGCACTTGAGCCACAGACTTCTGGCGAATTGCAGGGATA
TCCCAATTTGTAGCAGAAAAATGAACTGAACAGATCGTTGGGTCCTTCAACTATATCCCTGAAAATTCTC
TCTCTTTTAAATTTTCTTGTACCTATCAGTTATCACGCCACTGCATTGTTTTGTTTATATAGGCCCGTAG
TTCACAGTAGTTCATGCTCAATAACTGGTTCCTACCAATTACTGTGGGGGCACTAATCCGTTCCTGTGGA
ATTGCAGTGATAACAACGGAGTCGAGGAACATCCTGCTGAGGCACTTCTACCAGAAATCCGAGAAGGTGA
GCTGGTACTGCTAGCAAGTACCATGAAACCAGATGACCGAAACGAATCTAAGCTTGAAATCCTGTCCTGA
ACTGTAGCTGAGGCCCAAGAGACCTGCCCCGGACAACCTCGCTCCGGAGAACAACAACAGCAACAACAAG
CAGCCCAGGGGACCGGTCGGCGACGTCGGTGGGCAGTCGTCGAGCGCGAGAAGCTGAAGCCACAGCTGGT
GGCCGTCCACTCCTCCCCCTGCCTGCTTGTTTCTCATCTCTGGGTTTCGTCATGCAGAGGCAGAGGCAGA
TGCAGCCCTGTGTTGTCCTCTTCGCTCCTCACGTCTGTACGTACGACCCAAAGAGCTACTAACCTAATCT
GAAGTTAGGGTTACATACATGGATATCGGATAGATGGGTGTACATAAGCACCTGAGAGCAGTGTTGTGTT
ATCCTAAATGCTAAACGGTAAACAGTCGTTCAACATCCTCTGTGTTTAGCGTATAA
Zea mays 2
SEQ ID NO: 147
MGSPLGGWPSYNPHNFSQLVPADPSAQPSNVTPATYVATHRTDPPPNQVITTEARNILLRHLYQKSEEKL
RPKRAAADNLAPENNNKKQPRGPVGDVGGQSSARS
Brachypodium distachyon, CDS
SEQ ID NO: 148
ATGGGAAGCCCACTGGGCGGCTGGCCGTGCTACAGCCCGCAGAACTTCAGCCAGCTCGCCCCGGCCGACC
CCTCCGCGCAGCCATCGAATATCACACCAGCCACTTACATAGCGTCACATAGGACAGATCCACCTCCCAA
TCAAGTAATTACAACCGATCCCAAGAACATCCTGCTGAGGCATTTTTACCAACAGTCAGAGAGCAAGGTG
AGGCAGAAGAGAGCTGCGCCGGACAATCTCGCCCGGCATAACGACAAGCAGCCGAGGGGCCCCTTCGCCA
ACGGTGGAAGCCTGGCGAGCACAAGAAGCTGA
Brachypodium distachyon, cDNA
SEQ ID NO: 149
ATGGGAAGCCCACTGGGCGGCTGGCCGTGCTACAGCCCGCAGAACTTCAGCCAGCTCGCCCCGGCCGACC
CCTCCGCGCAGCCATCGAATATCACACCAGCCACTTACATAGCGTCACATAGGACAGATCCACCTCCCAA
TCAAGTAATTACAACCGATCCCAAGAACATCCTGCTGAGGCATTTTTACCAACAGTCAGAGAGCAAGGTG
AGGCAGAAGAGAGCTGCGCCGGACAATCTCGCCCGGCATAACGACAAGCAGCCGAGGGGCCCCTTCGCCA
ACGGTGGAAGCCTGGCGAGCACAAGAAGCTGAGGATCAGAGCTGGTGGTTCTCCTGATCTCCTCTGCATT
GCAGTGTCCTTTGCTGCCGCATGCCACACTGCAGCCCTCATGCCATAAGCGTTGCCAGTCTCTCATTTAA
CATGGTACGCGTGATCAAAACAACGGGGAGCCTTTACATGCAGGCAATGTACTGATGTACATAGACAGGC
ATATTCTTGGTCTTATTCTCACCCACTCGGTGCGGTTGGTTATCTTTGACAGGGCACTATAATTGCAGAC
TTTTTCTGTAGATAATGTGCCCACACCACCACCATGGGACGACGCTCCCCCCAACTTGTACTTTTGGTGA
AATAATTTATCATCATCATCATCATCATCTCATTGCCTCTGTAATTGATCTATGTACACTTTAGAT
Brachypodium distachyon, gDNA
SEQ ID NO: 150
ATGGGAAGCCCACTGGGCGGCTGGCCGTGCTACAGCCCGCAGAACTTCAGCCAGCTCGCCCCGGCCGACC
CCTCCGCGCAGCCATCGGTCAGTCAGCGCCTCCTTTTCGCTGTTGCGCTACCACATATCATACTTGTCGT
GGATCCCATGTGTGCTTGAATTGGAGACTACTCCATCGATTCCACAAGTTGTATTATGCTTGCGCAAATT
TGTTTGTGCCTAGAAGGAGAGGAATTTCCTCGCGACAGAAAGGAGAGGGTTCGTGTCTGCTGTACCTGAT
GCAGTAACAGGAGTCGCTCCTACTGTGCTTCTTTGATGAAATTGTAGTACACATAGGTGGCTAAATACTG
TAGTTTTTACATTGAAGTCCTGATTGCCGAACTATTGGATCCTATTCAAGTATTCAGAGGGCTTTGGCAC
AATGACTAAGCATTTAGGTTTAGGTTTTAGCTTCTCTCATTTGTTAGTACATTGAGTATATTCATTTGGT
GTAGTCCTAACTATTAACGGGTTTATTGCGGTTGCTATTTTTATGCATACTACAGGTCAGATATTCAGTT
GCCATGGTGCTAATGATACTAGTTATTCCTGTGTTTTCTGTAATGCCTTGTAGGCTTATAGTCAGAAAAT
AAAAGGCAGAACACAAAAATAAAAATAAAAATATATCATGCTAGCAACTTATACTGTGGGCAATAGTAGA
TATATTATTTGGTGACCTGGTGCTAATGTTAATTCTCATTTCTTTGTGTTTCTCGCCAGAATATCACACC
AGCCACTTACATAGCGTCACATAGGACAGATCCACCTCCCAATCAAGGTAACTCCATCCCCCGCTTCTTC
TACCAATGTCTCTGGTCTGTGCTGCTTCTGCTTCTCCCTTTTACAAATCTAGTCTGACTGATTTCCTCGT
TAGGCCTTTGTGCATAACTTAATGTAATTGCTCCTTCGCATTACGTCAAATTATCTTTACGATGGTATTT
CACAGGGCAAAAAATATGTCCCATAAACTGTTTGATCAACTAAGCCTTATGTTTAGCCATGAGAATCCGT
AAACCATATTATGTTCTCAATAATGTAGAAGTAGACGCGCCTTGGTTCTGCTAGGCCAAGCGCCATAGGA
TTTGGGAGGGCGTTCCTAAATTCTGCAATTTTATGATGTTGATTAGCAACAGTACTGTCTATGATGTGGG
ATCTGTGCTGGAGATATTATGCTGCCACAGCCCACATGTCCAAAGGTGATGTGGACAAGTGTTTAGATTC
CCAACCATCACCGTGCCACATTAACTGCAGTATTGTATCGAAGGTAGTATTTTCTGCATATCCTAACAAA
TGGTAACAATCTTATGTTGCCCATAGTGACTTAACACCATGTTCTACACTAATAGTGGCTTCCGCCTTCC
TCACTCGAACTTCTGATTCTTTTAAACACAGTTAACATTGGTAAGGCGGTAATTGCTATGGATAATGATT
CAACTAGACCTGTTTTCAACTTAACTACTCGCTTTGCTGACCCAACAGCAGCTCTCTATATTTCATCTAC
TGTATTTTTGTCGTGCAAATGTCCAGGTACTAGCCTTTTCCCAATGTTAAATATATCTTTACAAAATCTC
ACTTTGTGAAAAATATTTGTTCTATCATGCATTTCAAGATTAGGAAAACATGATCCCAAATTTTACTGAG
ACACATAGGCTAACAGCACATTTGTAAGGTCGAAACAACTACATAGTTACTGTTCATCTCAATTGCAATA
GTGCAACAGAGTTGAGAGACATGTCACATATGGCCATCGATGTTGGTGATCAAACTTCCTTGCAAACACT
CAAATGCTTGTAACAGTTAAATGTCTCTGGGAGAATCCCTTGTGGAGATGCAGAGGTGTTTTCTGTTATG
GGTGATGCATTCAAGTGTAGACCAGTTAGAGCATTAAATATTCCTTCACAATCGTTCCTTTTTCCATTTC
CTTGACTATCTCAGGGTTTGGCATGTAGTATGTAGGTTATGTACTTAATGGCCCATGGCTATATTGCCGG
ACCTTTCATACTTTAATGTAGGTACGCAGGGATGTCCACATGATCTTTGCACTGATATATCGTCTAAGTC
TCCCAAGTTAACTGTTCTCTCGGAGCATGAGGAAGAGGAAGACLAGGTGTAGTTAAAAAATACTTAAATG
TGTCAGCAGGGATGTCAACCTTGAAAGTAGAAATGGTAGGATCTTCAAGGTTCCCTTCAAGTTGAATTGT
GGCGTACTCCCAATGAAAATTCCTTCAACGTGGAGTGCTTGTAGTGAGAGGTCGTTGAGAGGAATTGCTT
GGGTTGAGTTGAAGGGGCAATCGGTTGCCTATGATGAATCAACAAAACTGGTTGGATATTAGGCTCAGAG
GTTTCGTAGAGCACACCATGGTACTTTCTATGACATTTCATGGGCCATTTACCGGGGTCTTTTTTTCTGG
GGCCTAATCAATTTATGATGCCAGGCTGAACTGCCTAAGTCTCTGTCTGCTTTCAAGAAAAGATATATTT
ACAATACATCAAACTAAGATATTTTTAGGCAGAGAAATATGCTGACGAACCGAACACTCTAACGACATTA
TGTGCCTTTTTAGGGTGCCACCAGCATATTTGTGAAGGAGATATTTGTTAACAAACTGAGAAATATGCAA
GAACAAAAATCATTTAAGTAGACACTTAACCCTAGACTGTAGTTCCGAGTTTCTGGCATATCCTTCAAAG
ATGTTCTTTTTGGTATATTGTAGGGATCGAGCAGGTCCTCTATTGCCAGCTTCTTTCAATATTTATAATA
ACTAATTATGTTTCTGGTAGAAACACTCGCCAAACAAATTGCTAATGGAACTAACCGCCAGTTATATGTT
TTGCATATCTTTTGAATGCATTAGTTTATACATATGTTCAGAGTAGCTCAGACTCAATGGCTGCCCCTTG
TTTTCTTCTTCTTTTTTGCCTTTTCGTTAATTTATATTCGTTGGAGGCACTCATCCACTCTCACCGTAAT
TGTTGCAAATCTTCTATGTCCATTTTCTTATGCTCTATGAAAACCACCTTTGCGGTGTCTCGACTGTTTA
TGCTGATAATCTGTCCCCTGGAAACTGCAGTAATTACAACCGATCCCAAGAACATCCTGCTGAGGCATTT
TTACCAACAGTCAGAGAGCAAGGTGAGCTAAGCCACCCAAGACACTGATGAAGAACAGATAGATTAAAAA
TACCGTCGAATAATAAAATCTTAATCTCAACATTATATATTTCTTCGTATCTTCATCCATAGGTGAGGCA
GAAGAGAGCTGCGCCGGACAATCTCGCCCGGCATAACGACAAGCAGCCGAGGGGCCCCTTCGCCAACGGT
GGAAGCCTGGCGAGCACAAGAAGCTGAGGATCAGAGCTGGTGGTTCTCCTGATCTCCTCTGCATTGCAGT
GTCCTTTGCTGCCGCATGCCACACTGCAGCCCTCATGCCATAAGCGTTGCCAGTCTCTCATTTAACATGG
TACGCGTGATCAAAACAACGGGGAGCCTTTACATGCAGGCAATGTACTGATGTACATAGACAGGCATATT
CTTGGTCTTATTCTCACCCACTCGGTGCGGTTGGTTATCTTTGACAGGGCACTATAATTGCAGACTTTTT
CTGTAGATAATGTGCCCACACCACCACCATGGGACGACGCTCCCCCCAACTTGTACTTTTGGTGAAATAA
TTTATCATCATCATCATCATCATCTCATTGCCTCTGTAATTGATCTATGTACACTTTAGAT
Brachypodium distachyon
SEQ ID NO: 151
MGSPLGGWPCYSPQHFSQLAPADPSAQPSNITPATYIASHRTDPPPHQVITTDPKNILLRHFYQQSESKV
RQKRAAPDHLARHNDKQPRGPFANGGSLASTRS
Oryza sativa ssp. Japonica CDS
SEQ ID NO: 152
ATGGAGAGCTCCCTGGGCGGCTGGCCGTCCTACAACCCGCAAAACTTCAGCCAGGTCGTCCCCGCCGACC
CCTCCGCGCAGCCCTTGAATGTCGTACCAGCCACTTACATTGCAACACACAGGACGGATCCACCTCCCGG
TCAAGTGATAACAACAGACCCCAAGAACATCCTGTTGAGGCATTTTTATCAAAAATCCGAGGAAAAGTTG
AGGCCAAAGAGAGCTGCACCAGACAACCTGACCCCACAGLACAACGGCAAACAACCAAGAGGCCCTCTCT
CTGATGGTGGTGGTAGCCAGGCAACTGCAAGTGGTAGAAGCTAA
Oryza sativa ssp. Japonica cDNA
SEQ ID NO: 153
GGAAAAGGGTGGAGAAACCAAGAGGGGGCGTCGCCGGAGTCGGAGTCGGAGACGTCACGGCGAGCTCCGC
GGCGGCGATGGAGAGCTCCCTGGGCGGCTGGCCGTCCTACAACCCGCAAAACTTCAGCCAGGTCGTCCCC
GCCGACCCCTCCGCGCAGCCCTTGAATGTCGTACCAGCCACTTACATTGCAACACACAGGACGGATCCAC
CTCCCGGTCAAGTGATAACLACAGACCCCAAGAACATCCTGTTGAGGCATTTTTATCAAAAATCCGAGGA
AAAGTTGAGGCCAAAGAGAGCTGCACCAGACAACCTGACCCCACAGAACAACGGCAAACAACCAAGAGGC
CCTCTCTCTGATGGTGGTGGTAGCCAGGCAACTGCAAGTGGTAGAAGCTAAAACGCAGCTGTTGTTCTCT
CCGGCATCTCTTGTGCTGCTGAACTGACAGCATGCATGTCATACTACCTGTATGTATGTGTGTGTGCTTG
TTCAGGCATATGCTTACTAGTAGTGTCATCATCTCTCTTGTGTGATTGATCAAAAGAGCTCCCCATGCAT
GTACATACACCCTCATCCTCAGTGTCAGTGCGGCACCTTTGATACGGAACTAGCACTATTGCAGTCTTTT
ATGCCGACACTAGCACAACTTGATGAAACCATTTTTCCCTACATAATTGCCTCAGCGTCAGCTTTCCAAA
GGCTGAAAGTGATCATTGCCTCTCTTA
Oryza sativa ssp. Japonica gDNA
SEQ ID NO: 154
GGAAAAGGGTGGAGAAACCAAGAGGGGGCGTCGCCGGAGTCGGAGTCGGAGACGTCACGGCGAGCTCCGC
GGCGGCGATGGAGAGCTCCCTGGGCGGCTGGCCGTCCTACAACCCGCAAAACTTCAGCCAGGTCGTCCCC
GCCGACCCCTCCGCGCAGCCCTTGGTCGGTCAGCCCCTCTGTGACTCTGTCCCCCCTCACTCGCGATCGA
TCTCTGTGCTTAGCTGTCGTGATCTCATGCCTCCGACCGCCAAGTACAATTCAGAATTAGGTTGATTGTG
TTGATGATGCCATGAATTGTGTGCCTGATCCCTCACGCCGGAGCTTTACCTCTAGTCAGTCCTATTGTGT
GTCAGAGCGTGTGTCTGAAATTCCAGTGCGCATGATTAACAAAAGGTGGTTAGTACTATTTGTTTCTAAG
TCCCAATGCCAAATAGTTGGATCATAATATCTCATTTGGGAGGTATTTGTGAATCATGGATTATACATTT
AGGATTTGGTTTCTCTCATGGCCAACGCCCTTTAAGGTTTCAGTTGTGGTGATGAATTACTAGTCGGTTT
TTGCTCCGAATGTCATTCCAGGCAGCCACTTGCATTGTAATTTGCAAAGCATATATTCAGTTTGTCATAT
TTGTGTTTCTCGTCAGAGTATCAAGCTGGTAACTTATTCTCTGGAGTCCGGACATGTTGCATTGGTGCGA
AACTGCCAATACTTATCCCTATTTTTTTTGTGTTTCAGAATGTCGTACCAGCCACTTACATTGCAACACA
CAGGACGGATCCACCTCCCGGTCAAGGTATCTTGCTCCCTTGTTTCTTTTACCAATGCTGATTTCCCTGT
GCTGCTCCTCACTTCTGAGTGTGTGGTTCCTAAACGTTCAAATGTGTATAGTTGAATGTGACTGCTCATT
CACATTGCATCAAATTCCTTTTACAGTAGTACTCTACATGGCATAGCAGATCCTCTGAGTTGTTTGGTCT
GAGCCAAAAAGTGACTATCTTGCCTTTCATGGCTGGCATATTTAACTCCAATCATCTATACTGTTTTCAG
TAATGCAGCCTCTTTAGTATCTTCTGAGTGATAGATCCATGAATAGATTCTGTTGGACTGTTAGATATGT
CTTCAAAGTCAATATTTTTTGCATTCCCTAGGACTGTAGTTTTGGCTTCTTGATATGTAACTCACATACA
TTATGGCTAATTTGCCCAGCTTACTTTCAAGTAGTTCCCTTACGACCTTTGTTCTGGTACATTTTTTATA
GCTTCAAAGCAAACTTTTCTCTTGGAGTATGACTTTATGAGGGAGAGCAGAAAAGGTTGAGAGTCAGAGA
GCCAGGGCCTAGCCGGCAAACTGGAAAGACCAACTAAACCTGATCTTACACCTTCATAGTTGAGTCCCCT
TGAATTGATTTGATAAGTTAAATGCTTTTATAATTTTAGCAATCAAATTTTGGAATAACACATGCTAGAG
CAACATCTATTTGGACTAGAGGTTGGAGTTTCCTAATGATGAATCAACATAATTTAGTATTTTCGCCTCA
GAGACTTTGCTCTGACCCACTATGTGTACCTATTACAATTTTGTATGAGCATTCTGTTAAGTCCTTTTTT
TCCTCCTAGCCTAATCTTTTGGCAGGCCAGGCCGAACCACCAAACATCCTGGTTGCTTTCAGGCCTGTCC
ACTGAACTATCTCCAGATTTCAGGTACAGGTATTCAGTAAAAAATTGATTGCATGCTGCTTACCATCTCC
ACAAAAGAAAAATGAAAGAAAACTATAGGCAAGCTGGGCAATTTATTAGTAAGGAGCAAATGAACTGGCC
AACCAAAGCAACCTCTCATATTAGTCATCTTCCTCTAGTGCTGCCAACATAGTTTTATTGAAGTGTTCTG
TTTATGCCATATTGCCAATATGATTTGGATGGTTACATTTATAGAACACAGTTTTTCTTCTCGACTCATT
GCCCGTGTGCCTGTTGGCAGAGGCCAAAGATGTAAACCATTTCCATTATCTAAAAAAAAAGAACATTGTT
TTTAGATATTCTATGTAAACATTGTACTTCATTGGTCCGTTGCAGGCATGGAGCTAGTTCCTTAGTTTCC
AGTTTATTCAGGGGCTGTTCAGATTGATGCCATTTTCAACCATATCATTTTTTGGCAAAGTTGCCAAAAA
TGTGCCTACATTTGGTTTGTTGCCAAATTTTGGTAAATACATAAGAAATCCTGCCAAAATTTTGGTAATA
TTGTCAACTTGCTAAAATTTTAGGTAAGGTTTATTTTGGCAACAATCTGAACAGCCCCTCCGTATTTTTG
CTGCCTATTCCCTCTTGAATTTCCTGTGCATGGGATATTGCTTCTGATAGTGGTGAATTCTTGAGTGCTT
TAGTTCATTAGAGCAAGTTTAATAGTATAGCCCACTGCTAACTCCAATTCATCTATAGCCAATCTAATAG
CCAATTCATACAATAGTTGCTTACTATACTATTAATATATGGTCACACCTGTCATACATACATTGCGTCT
TAGAGTCCGCGATGCAGCTGGCTACAGATCTATAGCCCGCTGCTCTTCTCTCTCATCCTTTCTCTCATTA
AAATATGTTTACAGCTGGCTAATAGCCTGCTATTGTACCTGCTCTTAGATATTTTCGTAGTCGATCAGAC
TCGATAGTTGCTCGCTCTCTTTACCTCGTTTTGGTTCTTTCTGGGCTCTCGTCCATTCCTACCAGAACTA
CCCCAACTATTCCAAGTTTTCTTTTTTACTGTACAGAAATCACCTCTTTTTTTTTTGTTGCTATTTTCAC
TATTTCCCTGACCGTTTGTGTCTGGAATCGCAGTGATAACAACAGACCCCAAGAACATCCTGTTGAGGCA
TTTTTATCAAAAATCCGAGGAAAAGGTGCGCTGCTAAGAGCCTAAGACTCTCACAAAGGTTACATAAATC
AGTATGGAACATCTATTTATCAACGCTTTATCTTGACTGTAGTTGAGGCCAAAGAGAGCTGCACCAGACA
ACCTGACCCCACAGAACAACGGCAAACAACCAAGAGGCCCTCTCTCTGATGGTGGTGGTAGCCAGGCAAC
TGCAAGTGGTAGAAGCTAAAACGCAGCTGTTGTTCTCTCCGGCATCTCTTGTGCTGCTGAACTGACAGCA
TGCATGTCATACTACCTGTATGTATGTGTGTGTGCTTGTTCAGGCATATGCTTACTAGTAGTGTCATCAT
CTCTCTTGTGTGATTGATCAAAAGAGCTCCCCATGCATGTACATACACCCTCATCCTCAGTGTCAGTGCG
GCACCTTTGATACGGAACTAGCACTATTGCAGTCTTTTATGCCGACACTAGCACAACTTGATGAAACCAT
TTTTCCCTACATAATTGCCTCAGCGTCAGCTTTCCAAAGGCTGAAAGTGATCATTGCCTCTCTTA
Oryza sativa ssp. japonica
SEQ ID NO: 155
MESSLGGWPSYNPQNFSQVVPADPSAQPLHVVPATYIATHRTDPPPGQVITTDPKNILLRHFYQKSEEKL
RPKRAAPDNLTPQNNGKQPRGPLSDGGGSQATASGRS
Oryza sativa ssp. Indica CDS
SEQ ID NO: 156
ATGGAGAGCTCCCTGGGCGGCTGGCCGTCCTACAACCCGCAAAACTTCAGCCAGGTCGTCCCCGCCGACC
CCTCCGCGCAGCCCTTGAATGTCGTACCAGCCACTTACATTGCAACACACAGGACGGATCCACCTCCCGG
TCAAGTGATAACAACAGACCCCAAGAACATCCTGTTGAGGCATTTTTATCAAAAATCCGAGGAAAAGTTG
AGGCCAAAGAGAGCTGCACCAGACAACCTGACCCCACAGAACAACGGCAAACAACCAAGAGGCCCTCTCT
CTGATGGTGGTGGTAGCCAGGCAACTGCAAGTGGTAGAAGCTAA
Oryza sativa ssp. Indica A cDNA
SEQ ID NO: 157
TCCGGGTCACCACGCGTCGCGGACGCGTGGGGGGGGCGTCGCCGGAGTCGGAGTCGGAGACGTCACGGCG
AGCTCCGCGGCGGCGATGGAGAGCTCCCTGGGCGGCTGGCCGTCCTACAACCCGCAAAACTTCAGCCAGG
TCGTCCCCGCCGACCCCTCCGCGCAGCCCTTGAATGTCGTACCAGCCACTTACATTGCAACACACAGGAC
GGATCCACCTCCCGGTCAAGTGATAACAACAGACCCCAAGAACATCCTGTTGAGGCATTTTTATCAAAAA
TCCGAGGAAAAGTTGAGGCCAAAGAGAGCTGCACCAGACAACCTGACCCCACAGAACAACGGCAAACAAC
CAAGAGGCCCTCTCTCTGATGGTGGTGGTAGCCAGGCAACTGCAAGTGGTAGAAGCTAAAACGCAGCTGT
TGTTCTCTCCGGCATCTCTTGTGCTGCTGAACTGACAGCATGCATGTCATACTACCTGTATGTATGTGTG
TGTGCTTGTTCAGGCATATGCTTACTAGTAGTGTCATCATCTCTCTTGTGTGATTGATCAAAAGAGCTCC
CCATGCATGTACATACACCCTCATCCTCAGTGTCAGTGCGG
Oryza sativa ssp. Indica A gDNA
SEQ ID NO: 158
ATGGACGTCAGCCAAGCCACCGAAGAGCAACTTCCTTCACACGGCCAGCACCAGAGCTCCTTGGAAGAGA
CTGCAACATGTCATCATTGCCGAGCGTCGCCGCACCCCCATCAGCAAGCAGCTTCGACTTCATTAGCAGA
AACAACCGAAGAGCGTGTTACCCACCACAAGAAAGAAGCGGACCCAAGAAGGCGAAGGCCTCACCGAACA
AAAGCTTACCTTGATATACCACTCCATTTGGACAAATTGTGGAGAATCCGAACTCTCCCACGACTGTCCT
CCACGAGTCCATGGTCGCTGGAAAAGGGTGGAGAAACCAAGAAAAGAGGGGGCGTCGCCGGAGTCGGAGT
CGGAAACGTCACGGCGAGCTCCGCGGCGGCGATGGAGAGCTCCCTGGGCGGCTGGCCGTCCTACAACCCG
CAAAACTTCAGCCAGGTCGTCCCCGCCGACCCCTCCGCGCAGCCCTTGGTCGAATGTCGTACCAGCCACT
TACATTGCAACACACAGGACGGGTCCACCTCCCGGTCAAGGCCAGGCCGAACCACCAGACATCCTGGTTG
CTTTCAGGCCTGTCCACTGAAATATCTCCAGATTTCAGTGATAACAACAGACCCCAAGAACATCCTGTTG
AGGCATTTTTATCAAAAATCCGAGGAAAAGTTGAGGCCAAAGAGAGCTGCACCAGACAACCTGACCCCAC
AGAACAACGGCAAACAACCAAGAGGCCCTCTCTCTGATGGTGGTGGTAGCCAGGCAACTGCAAGTGGTAG
AAGCTAA
Oryza sativa ssp. indica
SEQ ID NO: 159
MESSLGGWPSYNPQNFSQVVPADPSAQPLNVVPATYIATHRTDPPPGQVITTDPKNILLRHFYQKSEEKL
RPKRAAPDNLTPQNNGKQPRGPLSDGGGSQATASGRS
Hordeum vuigare CDS
SEQ ID NO: 160
ATGGGAAGCCTGCTGGGCGGCTGGCCGAGCCACAACCCTCAGAACTTCAGCCAGCTCGTCCCGGCCGACC
CCTCCGCCCAGCCCACGAATATCACACCAACAACTTACATTGCAACACATAGGACAGATCCACCTCCAAA
TCAAGTGATCACGACGGAGCCCAGGAACATCCTGCTGAGGCATTTCTACCAGAACTCTGAGAACAAGCCG
CGGCCGAAGAGGGCCGCCCCGGAGAGCGTTGCCCTGCGCAACGGCAAGCAGGCGAGGAGCCTCGCCGACG
GCGGAAGCCAGTCGAGCACGAGAAGCTAA
Hordeum vuigare cDNA
SEQ ID NO: 161
GCACGAGGACCAACCGTCGGCAAAAAAAGGGCAGAGCTTGGCCGGAGCGAGAGACGGCGCAGACGTCGCG
GGCGGCGGCAGCGGCGATGGGAAGCCTGCTGGGCGGCTGGCCGAGCCACAACCCTCAGAACTTCAGCCAG
CTCGTCCCGGCCGACCCCTCCGCCCAGCCCACGAATATCACACCAACAACTTACATTGCAACACATAGGA
CAGATCCACCTCCAAATCAAGTGATCACGACGGAGCCCAGGAACATCCTGCTGAGGCATTTCTACCAGAA
CTCTGAGAACAAGCCGCGGCCGAAGAGGGCCGCCCCGGAGAGCGTTGCCCTGCGCAACGGCAAGCAGGCG
AGGAGCCTCGCCGACGGCGGAAGCCAGTCGAGCACGAGAAGCTAAACAAGCAGGCGAGGAGC
Hordeum vuigare gDNA
SEQ ID NO: 162
TCGGCAAAAAAAGGGCAGAGCTTGGCCGGAGCGAGAGACGGCGCAGACGTCGCGGGCGGCGGCAGCGGCG
ATGGGAAGCCTGCTGGGCGGCTGGCCGAGCCACAACCCTCAGAACTTCAGCCAGCTCGTCCCGGCCGACC
CCTCCGCCCAGCCCACGGTCGGTCAGCCCCCCTTCCCTTCCCTCCCCTCCCTCTCCGTGGCAGTTCCGTG
GTCTCTTCGTCCTGTCCTGCCCCGTACCACACTGCTAGGGTATGCTCCAGTCGGAGGGCGCCTGATTCCA
CAGGTTTCAGGTGCCATGCATGCTTGCTTGCTTGCACAAGTGCGGAGTTCATCGGTGCCCAAAAGGGGAG
CGGGGTCTGCTGCCTGAGCCAGGAATTAGGAGTTACTCGCACTGTGCGTGTGTGCGTCTCCACTGGAATT
CTGGTATAGATGGCCAATGATTGTAGCTTTCTGTTGACTCAAGCCCAACTGTCGAGCGATTGGGTCGTAT
TCAGAGGGATCTTGCACAGTGAATAAGCATTTAGGTTTTGGTTTTGGTTTTGGTTGTGGTGGTTGTTGCT
ATGTGTACTGCAGGTCATATGTTCAGTTGATCTGGTGTTATGTATGCTAGTTTTCCTGTGTTTCTTGTCA
GAATGACTAGAATTCAGAAATAGAAAAGGCAGGCCGAAAGAAAGAAAAAAAAAACTATCATGCTAGCACT
TACAATGTTGCAAGTGTATTGGTATTTGGTTGACATGGCGCTAATGCTGATCCTCATTGTTTGTGTTTGT
TTCTCACCAGAATATCACACCAACAACTTACATTGCAGCACATAGGACAGATCCACCTCCAAATCAAGGT
AAACTCTTCCCATATTTATTCAACCAATGTCTCTTTGCTGCTTATACGTTCTGCGAATTTAACCTGCCTG
GCTTCCCCTTTGCTTTTTGCGCGTGATTGAATGTACGCCCCTCTGCACTGCGTCAAATTCTGTATTCCCC
ATGGCAAAATAGGTTTCAGGAACTGATTGATTTGAACTAAAAACTGTATAGGTAGATGGGTATCTTTTCA
TTCAGGAGGGCATCATATTTAGCCATGAGAGTCCATATTATGTTCCCAATAATGTCGAAAGGGCATGGGT
TGATTCTGCTAGGCCAAACACCATATCATTTCGCAGGATGTTCCTAAATTATAATTTGGCTCCTTTTTTT
TATGATGTTATTTTGGCAATGGTACTGGCGATGTCATGGGATTCTGTACTCAAGAAATTATGGTGCCACA
TGTCCAGATGTGATATGTACTCAACCACTTCTAATTCCCCATTGACATGCTACTTTAATCTCCATAATAT
ATTGTATCCAAGGTGCTATGTTCTGCATGTCCTACACAAATGGTGGCAATCTTATTTGCCAACAGTGACT
GGACACATAATACGCTAATAATAGTGGTCTCCTCCTGCCCTCACTTGAGCCCCTGGTCTGTAAGAATCAC
GGTTTGTGCCGGTAAGTGGTGATTACCAGCAAATAGCTATGGATGATAACTCGGTTACACCTGTTTAAAC
ACTGTATAAGACTGAACTGCTCACCTCGATTACGTAGCAGTAGTTCGATTTATTTCATCTCCTACAGCTT
TTCATTCGAATGTCCAGTTATTGGACCGTGTCCTATATTAGGTCTTCACAAAATATCAAAATATCACTTT
GAAAATATTGTCTATTCCATTGAGACATTCCAAGATTACGAAAGCATGCTCCCCAATTATTCTGAGTCAC
ATTAGTTAACGGCACATTAGTAAGGTTCAGACAGCTAAATACTTTAGTTTCATCTAAAATACAGTAGCTT
AATGAGATTGTGATTCATGTCATTGGCCATCGTTGTTGGTAATTGAACTTCTTCAGCAAACTCTGAAGTT
CTTGCAACAGTTAAATGTCACTGATTTACATGATTTGGGTGATTTGGAAGGAACGGACTGTCGTGTCTTT
CATAACAAGGCCCTGCAGCCTTTGGATGTGGCCGTAATAATTCAAGAGGAGGTAGTGCAGTGGCCATGGG
CATATGCCTTCTCCCATGATACCAGGCATAGTTGCAATTCCCTTGCTTTGGCTTCTTTGCTACCCTAGCG
TGTTTGGGTTTTGCCTTTTGCCCTTTATTATATATTTTGTATATTACTTGTTTTAATTTCAATCTATATG
AATGGGAAGAGCACCTCCCTTTTCCAAAATTAGTCGTACATTTCATTATAGGTCTGCAGGTTGCACTGTT
ACAAATTCTTAATCCACAAGTTAGTTTTTCTCTTGGGAGCATGAGGAAGAGGAGGACATAGCAGTGATGT
CAGACTTACATTTTTTACACCTTAAAGGAGATTCTTTAATGTTCCCTTCAAGTTGAATTCTTAGAGAACT
CATACTGAGAGTGCCTTCGACAAGGAGATATAGCAGCACTCGGTCCTTGAGAGGAATTGGTTGAGTTGAG
TGAAAGAGGCAAGCAGTTGCCTAATGATTAATCATGAATATAGGTGGGTTCTCGGTTCATAGGCTTATGG
CCCATTGTCTGGTGCCTAATAAATTTATTAGGCTAGGCTGAACTGCCAAAAATCCTGGCACATCGGCCCA
GCTGGCGGTCAGTGAAATTTTTATTGCATACTCCTAACGAACTCTACAAAGAAAGGTTAATTTCAATAAT
AAGAACTATATGATTGATTTCCTTTAAAAAATCGACATAATATTTTTAAGAGCAGAGTGCACTGACAGAG
CAAGCACCTGCTACATTATGTGCCATTTTTCGGCGTTGCGAGCATAAATTGTGAAGAAGTTATCAGTTGC
TAACATTGAGAAGTAGACAAGAACTAAAATCATCTGAATAGACCCTTAACACTGAACTTGCAAGTTTCAG
AGCTGTGCCACAGATTTTGGTTAGCTGAAATGCACTGAACACTCGATTTGGTTCATTGCAGGGATCGAGT
TGCTTCCTTTTATTGCCAGCTTGTGTCAACATTTCGTGATATACACATTTTCTTATGAAATTCCTGTGTG
GCAAACTATTCTTCCGGCAGATAGATTGGCTAAACAAGCTGCTAATGAAACGGACTGCTAGTTATGTGTT
GACAGAACCTCCTTTTCTAAAAAAAAGTTATGTGTTGAGTGTGTCGGTGTTGCGTGCCTCGTTGAGTGAT
TAGTTTTGCATATATGTATGTTCCAAGTAGATAAGCCTCTGGCTGCTCCCGTCGAGTTAATCTGTACTCC
GCTGACAATCTGAGCCCTGCGACTGCAGTGATCACGACGGAGCCCAGGAACATCCTGCTGAGGCATTTCT
ACCAGAACTCTGAGAACAAGGTGAGCTACCAGCACTCTGACGAAGAAGAGTAGGTAGACCGGCAACCGTT
GTTCTCkACGCCGTATACTCATGTCTGCAGCCGCGGCCGAAGAGGGCCGCCCCGGAGAGCGTTGCCCTGC
GCAACGGCAAGCAGGCGAGGAGCCTCGCCGACGGCGGAAGCCAGTCGAGCACGAGAAGCTAAACAAGCAG
GCGAGGAGCCACCGCTGCAGTCACTTTTCTGCTTTGTGTG
Hordeum vuigare
SEQ ID NO: 163
MGSLLGGWPSHNPQNFSQLVPADPSAQPTNITPTTYIATHRTDPPPHQVITTEPRNILLRHFYQNSENKP
RPKRAAPESVALRNGKQARSLADGGSQSSTRS
Triticum aestivum CDS
SEQ ID NO: 164
ATGGGAAGCCCGCTGGGCGGCTGGCCGAGCCACAACCCGCACAACTTCAGCCAGCTCGTCCCGGCTGACC
CCTCCGCCCAGCCCACGAATGTCACACCAACAACTTACATTGCAGCACATAGGACAGATCCACCTCCAAA
TCAAGTGATCACGACGGAGCCCAGGAACATCCTGTTGAGGCATTTCTACCAGAACTCGGAGAACAAGCCG
AGGCCGAAGAGGGCCGCCCCGGAGAGTGCCTCCGTGCGCAACGGCAAGCAGGCGAGGAGCCCCGCCGAGG
ACGGAAGCCAGTCGAGCACGAGAAGCTGA
Triticum aestivum cDNA
SEQ ID NO: 165
CCACGCGTCCGCAACTGTCGGCAAAGAGAGCCTGGCCGGAGCGAGAGACGGCACACACATCGCGGTCGCG
GACGGCGGCAGCGGCGATGGGAAGCCCGCTGGGCGGCTGGCCGAGCCACAACCCGCACAACTTCAGCCAG
CTCGTCCCGGCTGACCCCTCCGCCCAGCCCACGAATGTCACACCAACAACTTACATTGCAGCACATAGGA
CAGATCCACCTCCAAATCAAGTGATCACGACGGAGCCCAGGAACATCCTGTTGAGGCATTTCTACCAGAA
CTCGGAGAACLAGCCGAGGCCGAAGAGGGCCGCCCCGGAGAGTGCCTCCGTGCGCAACGGCAAGCAGGCG
AGGAGCCCCGCCGAGGACGGAAGCCAGTCGAGCACGAGAAGCTGAACCAGGGCTGGTGTTCTTGTCTTGC
GCCGCCGCCGTGGCATCTCTGAATCTCTGAATCTCCTCATATGTTTGATTTTGAGAAAGGTGTTGCACGC
ATGCACGCATGCACTGCACATACATGTGGGCCAGGCCTAGT
Triticum aestivum
SEQ ID NO: 166
MGSPLGGWPSHNPHNFSQLVPADPSAQPTNVTPTTYIAAHRTDPPPNQVITTEPRNILLRHFYQNSENKP
RPKRAAPESASVRNGKQARSPAEDGSQSSTRS
Triticum turgidum ssp. Durum CDS
SEQ ID NO: 167
ATGGGAAGCCCGCTGGGCGGCTGGCCGAGCCACAACCCGCACAACTTCAGCCAGCTCGTCCCGGCTGACC
CCTCCGCCCAGCCCACGAATGTCACACCAACAACTTACATTGCAGCACATAGGACAGATCCACCTCCAAA
TCAAGTGATCACGACGGAGCCCAGGAACATCCTGTTGAGGCATTTCTACCAGAACTCGGAGAACAAGCCG
AGGCCGAAGAGGGCCGCCCCGGAGAGTGCCTCCGTGCGCAACGGCAAGCAGGCGAGGAGCCCCGCCGAGG
ACGGAAGCCAGTCGAGCACGAGAAGCTGA
Triticum turgidum ssp. Durum cDNA
SEQ ID NO: 168
GAATTCGGCACGAGGGGACGGCGGCAGCGGCGATGGGAAGCCCGCTGGGCGGCTGGCCGAGCCACAACCC
GCACAACTTCAGCCAGCTCGTCCCGGCTGACCCCTCCGCCCAGCCCACGAATGTCACACCAACAACTTAC
ATTGCAGCACATAGGACAGATCCACCTCCAAATCAAGTGATCACGACGGAGCCCAGGAACATCCTGTTGA
GGCATTTCTACCAGAACTCGGAGAACAAGCCGAGGCCGAAGAGGGCCGCCCCGGAGAGTGCCTCCGTGCG
CAACGGCAAGCAGGCGAGGAGCCCCGCCGAGGACGGAAGCCAGTCGAGCACGAGAAGCTGAACCAGGGCT
GGTGTTCTTGTCTTGCGCCGCCGCCGTGGCATCTCTGAATCTCTGAATCTCCTCATATGTTTGATTTTGA
GAAAGGTGTTTGCACGCATGCACGCATGCACTGCACATACATGTTGGCCAGGCCTAGTGCGGGGTGACAC
CGGCATGGCACCACTGCAGTCTCTTTCTCCTTTGTGTAGATAATAATAATGTGGCCGCACCAGCACACCA
ACATGTACTACTGTAGTAGCGCTTTTGTATAATATTTGGATGATTTCCCT
Triticum turgidum ssp. durum
SEQ ID NO: 169
MGSPLGGWPSHNPHNFSQLVPADPSAQPTNVTPTTYIAAHRTDPPPNQVITTEPRNILLRHFYQNSENKP
RPKRAAPESASVRHGKQARSPAEDGSQSSTRS
Sorghum bicolor CDS
SEQ ID NO: 170
ATGGGGAGCCCCCTGGGCGGGTGGCCGTCGTACAACCCGCACAACTTCAGCCAGCTCGTCCCTGCCGACC
CCTCCGCGCAGCCCTCGAATGTCACACCAGCCACTTATGTTGCGACCCACAGGACAGACCCGCCACCCAA
TCAAGTGATAACAACGGAGGCCAGGAACATCCTGCTGAGGCACTTCTACCAGAAATCTGAGGAGAAGCTG
AGGCCAAAGAGAGCTGCTCCGGACAACCTCGCTCCGGAGAACAACAACAAGCAGCCCAGGGGACCTGTGG
GCGACGTCGGGGGCCAGTCAAGCGCAAGAGGCTGA
Sorghum bicolor cDNA
SEQ ID NO: 171
TGTAACCATCACTCTTTTTCCCCGAAACCAAAACGAAAAAAAAAAGAAAGTGCTGCTGGCTGCTGCCAAC
CACCCGTGGTCCCATGAAGAGAGCATCGCCGGAGTCGGGGACGGTGCGCCGAGAAGGAACAAAAGAAGAC
GGCGGCGGGGCGGAGATGGGGAGCCCCCTGGGCGGGTGGCCGTCGTACAACCCGCACAACTTCAGCCAGC
TCGTCCCTGCCGACCCCTCCGCGCAGCCCTCGAATGTCACACCAGCCACTTATGTTGCGACCCACAGGAC
AGACCCGCCACCCAATCAAGTGATAACAACGGAGGCCAGGAACATCCTGCTGAGGCACTTCTACCAGAAA
TCTGAGGAGAAGCTGAGGCCAAAGAGAGCTGCTCCGGACAACCTCGCTCCGGAGAACAACAACAAGCAGC
CCAGGGGACCTGTGGGCGACGTCGGGGGCCAGTCAAGCGCAAGAGGCTGAAGCCACACAGCTGGTGCTGG
TGCCCGTCCTCCCCTGCCTCTCATCTCTCGGTGTCATGCAGATGCAGCCTGCATCTCTCGCTCACATGTC
ACAGCTGGTGGTTGTTTCTCCCCTGTGCGTCCTCTTCGCCTCTCACGTATGTACGTATGACCCAAAGAGC
TGAGGTATACATACCTGGATGGTTGGATGGATGTACATAACCACCTGAGACGAGACAAAGCTCGGTGCGT
GCCATTTCACATGGCACTAGGTGTGCTGCAGCCTCTCCTTTTCATCCTCTACAATGCAAAAATATGGATG
TGCCCATGCTGCTATGCTAGCTAGCCCTACTCCCCCTGTGCTTTGGATCGTGCACCGCGTCAGCAGCTTT
TTGAAAGGCTGGTGGTGATGATTGCACTCTGAAAATCCCCGTCTTCTGCTGTCAGATTATACTATACGCT
GCTGCCGTGCAGCTGCTGCTGCGCCAGCCAGGGGCAGCCA
Sorghum bicolor gDNA
SEQ ID NO: 172
TGTAACCATCACTCTTTTTCCCCGAAACCAAAACGAAAAAAAAAAGAAAGTGCTGCTGGCTGCTGCCAAC
CACCCGTGGTCCCATGAAGAGAGCATCGCCGGAGTCGGGGACGGTGCGCCGAGAAGGAACAAAAGAAGAC
GGCGGCGGGGCGGAGATGGGGAGCCCCCTGGGCGGGTGGCCGTCGTACAACCCGCACAACTTCAGCCAGC
TCGTCCCTGCCGACCCCTCCGCGCAGCCCTCGGTCGGTCAGCAACTTGCCCTTCCTGGCGATCTGGCCTC
TAGTATCATGCTGTAATGCTAGGCTCCGTACTTCCTGACGAGCTTAGATAAGCTCGAATATGTTTAATTG
ACCGGGTTCATCTGCGCTTGGCCTGTTTCTTTTCTCTGGTTTCAGGTGCCGTCGAGAAAAAAAAATCTCT
CTTTTTTTTAATCCCGTAGTATCACTTTCGGGCAGGAGACAGTAATCGGTGCCGGTATTGTTAGTTTTGG
CTGAAATTTTGGTATGGATGGCTGGAGAAATGGGGTCTCACTGTTTGATTTTAGTTCAGCTGCCAAAGAC
CTGTTCAATTTGAGGGGACTGTCTGGCCAATTTCTGAACATCTGGTCTGGTTTTCTCATGGTCATGTCTA
CCCTGGGTAGATTCAGTTGATGTGGTACTGATGGGCTAATGGTAGTTCAGTTCATGGTTGCTAATGCTAC
CGGTTGATTTTCTACAACGTCAGAAATTCGTGCTGTCAACTTATATTATGAATTATATATGTCCATTTCA
CCTGGTGCTAATGTTAGTTCTTTTTTCTTTCCATGCGTGTTTGTCATCAGAATGTCACACCAGCCACTTA
TGTTGCGACCCACAGGACAGACCCGCCACCCAATCAAGGTAACCCCTTTCCATGTCCTTAAGCCAATGGT
ATTCTGCTGCATGTAATTGATGCCGTACAATTGCTTATTCGCAATAAGCCAACAAGCCCCTTTTTTTTGT
TTATTGTAGTGCTTTGTATGTTAATTGCAAAATAGGTTTCATGAGCTGTTTCGTCTGAACTGACATTCTT
CAGGTAGATGACTCTCTTTGCTTCAATGGAGCATGATTAGCCATAAGCTCCTGTGCATGTGTAGAATGTG
TCTGCAAGTCACAGATGGTGGTATCAGCCACAGTGAACAGAAGCTCTGAACGCCTTAATCCTTATCCTAC
ACAAACGACGCCCTCTGTAGCTTTGCTGTGTTTACCTGAACCCAGGTGCTCTGAACCTCGATCTTGTTGC
TGGTAATCTGAACTCAGAATAGTAGATATGGTAGATAATTAGCAGTCGCTGTCTTAGCCATATACTCCAA
TACAATACAATACATTACCTTCCTTGCTGGCTAATTTACCAGTTGCAAGTACTAACAGTACCAGGCTACC
AGCTTTGATTATGCGATTTCATATAATTTTCTTTCTCTGTTGCAAAATCTTATAGCAATTCTAGCGTTAA
GCCTTAAAAAAACACAACTGGGAAACATTGCCTGTTGTATTTTGGAAATTTTAGATTCTTAAGGCTAGCT
TTCTTATTTTACTGAAAGTCTGAAACACACTGACAACTATCAACAAATTAATATTGATTCATCTAGTCAC
AGCAAATGGTAAATTTGTTTTTGACGGTAATTCAGACTACTGCATAGTTAGTGTTCACCTCGAATACAGT
AGTCCAGCAGAGTTGAGATTTATACCATAGCTGATCATCGCTCTAGATATCTCAACATTTGGCATGCTAA
TGGCCTTCTGTGGCTTATACTATTCTATGCCTGAGTTGGTTTGCCTTTGTTCCATCGTGTAGGTTGCTTG
GGAGATTTACACTGTTTTTCCTTCAAATATTTTCTTAGAGCATGAGAAAGCGGAAGAAAAGTTTGAGTTA
ATTGGTGCTTTATGGATCAGGGATGTTGGTGTTCAAAATGAACCCATAGACTAGTTAAATGCATCCATCA
TAATATACCTTAAGAGTGGAGTCTCTAAGACTTCAGTAAAGTTGGAGCCTTCCATTTGAAAATGCAATCC
ACAGAGTTCTAGAACTGGGATGACCTTGAGAGAAGTTAGGTTAATTTACTACTAAATGGTAGCCTAATGG
TCACATTGACTAGGAATTTGGCTTGAACACTCTGGTTGTCAATTTGTATAACAATATGTCGCTCTTTGTT
GTGGTCATTCTCTGGTCCTGAACAATTTACCAGGTTAGGCGAATCCACAAGAGGTCCTTATTGAATTCGT
GCCTTTTTGGCACAGCTGATTCCACATCCTGTCGGTGAAAATATAATTATGTGCTGTCTACCAGCTGCAT
AAAAGGTCCCAAAAGGAACAAGCTATGATTGTGCCTTCATCTGGGGGCTAATGACATATTTTTCGGAGAT
GTTATTTGTGAACAATCGAACAATCCATAACATTTTAAAGGATACTTAATCCTGAACTATTGCCAGCACT
TCAACCACAGACTTCTGGTGAATTGCAGTGATAACAGGCTAACAGCAGAGATATCCCAATTTGTGGCAGA
TAAATTACTAACAAATCATGGGCATTCTTTAACTACATGCCTGACAATTCTCTCCTTTTAGTTTCCTTTT
ACTTATCATACTGCTGCATTATTTTATATATATGTCCATAGTTCACACTAATTTAGGCTCAATAACTGCT
TCCTACCATATCAGTGTATTTACTTTCAATTCTTGTGGGGACACTGATATGTTCCCGCTAAAATTGTCAC
AAACCCCCCAATTCCTTTCTCAACTTTGCTGCATGAAAACCAACCTTGTTATATTTTTACCTCTTACTGC
GGACTGAATCGCACCCTGGAATTGCAGTGATAACAACGGAGGCCAGGAACATCCTGCTGAGGCACTTCTA
CCAGAAATCTGAGGAGAAGGTGAGCTGCTACTGCTAGTAAGACTTCACCATCAAGGCTACATAAAACCAC
ATCACTATAGAATCTAAGCTTGAAATCCTATCCTGAACTGTAGCTGAGGCCAAAGAGAGCTGCTCCGGAC
AACCTCGCTCCGGAGAACAACAACAAGCAGCCCAGGGGACCTGTGGGCGACGTCGGGGGCCAGTCAAGCG
CAAGAGGCTGAAGCCACACAGCTGGTGCTGGTGCCCGTCCTCCCCTGCCTCTCATCTCTCGGTGTCATGC
AGATGCAGCCTGCATCTCTCGCTCACATGTCACAGCTGGTGGTTGTTTCTCCCCTGTGCGTCCTCTTCGC
CTCTCACGTATGTACGTATGACCCAAAGAGCTGAGGTATACATACCTGGATGGTTGGATGGATGTACATA
ACCACCTGAGACGAGACAAAGCTCGGTGCGTGCCATTTCACATGGCACTAGGTGTGCTGCAGCCTCTCCT
TTTCATCCTCTACAATGCAAAAATATGGATGTGCCCATGCTGCTATGCTAGCTAGCCCTACTCCCCCTGT
GCTTTGGATCGTGCACCGCGTCAGCAGCTTTTTGAAAGGCTGGTGGTGATGATTGCACTCTGAAAATCCC
CGTCTTCTGCTGTCAGATTATACTATACGCTGCTGCCGTGCAGCTGCTGCTGCGCCAGCCAGGGGCAGCC
A
Sorghum bicolor
SEQ ID NO: 173
MGSPLGGWPSYNPHNESQLVPADPSAQPSNVTPATYVATHRTDPPPNQVITTEARNILLRHEYQKSEEKL
RPKRAAPDNLAPENNNKQPRGPVGDVGGQSSARG
Setaria italica CDS
SEQ ID NO: 174
ATGGGGAGCCCTCTCGGTGGGTGGCCGTCGTACAATCCGCGCAACTTCAGCCAGCTCGTCCCGGCCGACC
CCTCCTCTCAGCCCTCGAATGTCACACCAGCCACTTACATTGCAACTCACAGGACAGATCCGCCTCCCAA
TCAAGTGATAACAACAGAGCCCAGGAACATCCTGTTGAGGCACTTCTACCAGAAATCCGAGGAGAAGCTG
AGGCCAAAGAGAGCAGCTCCTGACAATCTCGCTCCAGAGAACAACAACAAACAGCCCAGGGGCCCTGTCG
CCGATGTTGGAAGCCAGTCAAACGCAAGAAGCTGA
Setaria italica cDNA
SEQ ID NO: 175
ATGGCCTGTTCGGTAGTGCTGGCTGCTGCGCTGCTGCTGCTACAGTCAGCGTTCAACACAGCGAGTGGCT
GGCTCGCTGGGCTGCTGCAGCTGCCGCAGCCGGCCGAAAAAAGTGCAGCCGAATATCTGCCAAGGAACAC
GCACAGGTCCTTCACGGATCTTGTTTTTCGGTTTTACAGGCAAGTAGGCAACCATCGCCCGTTCTTTGAC
CCCGTCGGAGTTCAGATGATCGTGGCCGTGGCCGTTCAGCGATCAGGAGCTGGAAGACGATGTGAGGGGA
GCTTCGCCGGAGTTAGAGACGGCGCGGCGATTCCGGCTCAACAAACCACCAGGGGAACAAGAGGGGCGGC
GGCGTGGAGATGGGGAGCCCTCTCGGTGGGTGGCCGTCGTACAATCCGCGCAACTTCAGCCAGCTCGTCC
CGGCCGACCCCTCCTCTCAGCCCTCGGTCGAATGTCACACCAGCCACTTACATTGCAACTCACAGGACAG
ATCCGCCTCCCAATCAAGTGATAACAACAGAGCCCAGGAACATCCTGTTGAGGCACTTCTACCAGAAATC
CGAGGAGAAGCTGAGGCCAAAGAGAGCAGCTCCTGACAATCTCGCTCCAGAGAACAACAACAAACAGCCC
AGGGGCCCTGTCGCCGATGTTGGAAGCCAGTCAAACGCAAGAAGCTGAATACAGCTGGTGCTTGTCCTCC
CCTGCGTCTCTCAATGCCGTGTGCAACCTGCATGCTGCATGCCAGCTGAAGCCCTGGTCCTCTTGATCCA
AAGAGCTACGCTCATTACATGCATGAATGTACATAACAACCTCCCCCCTTTCCCTCCAACATTGGTTTGT
TATTTGTTAGCGACTGGTGGCTGCATTTTAGTGACAGATTTTAGTAAAGAAAAAGGATGGTTCGGCATGA
AAAGATAGCCGCTTTTCTCTTGCTTATGCAATACTCCGTACAATTTAGTAAAATATAGACACTATTTGTA
Setaria Italica cDNA
SEQ ID NO: 176
ATGGCCTGTTCGGTAGTGCTGGCTGCTGCGCTGCTGCTGCTACAGTCAGCGTTCAACACAGCGAGTGGCT
GGCTCGCTGGGCTGCTGCAGCTGCCGCAGCCGGCCGAAAAAAGTGCAGCCGAATATCTGCCAAGGAACAC
GCACAGGTCCTTCACGGATCTTGTTTTTCGGTTTTACAGGCAAGTAGGCAACCATCGCCCGTTCTTTGAC
CCCGTCGGAGTTCAGATGATCGTGGCCGTGGCCGTTCAGCGATCAGGAGCGTGGGCCTGCTAGTCCAAGT
TGGGCCACGACCACGCACTGACGAGCGTATGGCCGGTCTGGGCCAGATAGCGCTATGGGCCGCAACAAGA
TTCTTTTTTTCTCCCAAAAAGGGAGGTGGAAAAAAAAAGAAAACGAAAAGTGCTAACCACCAGTGGAAGA
CGATGTGAGGGGAGCTTCGCCGGAGTTAGAGACGGCGCGGCGATTCCGGCTCAACAAACCACCAGGGGAA
CAAGAGGGGCGGCGGCGTGGAGATGGGGAGCCCTCTCGGTGGGTGGCCGTCGTACAATCCGCGCAACTTC
AGCCAGCTCGTCCCGGCCGACCCCTCCTCTCAGCCCTCGGTCGGTCAGCACTTCCCCCTCTTTGGCGATC
TCGTCTCCAATACACCGCACTGACTCTCTCCATAGTTCCTGATGATCTTGCATAAGCTTGAATATTTAGT
TAGGAGGTATTGGTTGGTGCTTGGTCAGTGACATCTGTGGACTCTTGTATCCACAATAAAAAACTTCCTT
TTGTACTGCTTTCAGGCAGGGAGCATGAATCAATGGTCGTATGGTTCGATTCTGCTGAAACCACAGTATG
GATGGTTTGAGAAAGAGGGTATCGATGTTTGTTTTTAGTTCATCTGCCAGAGACCCGGCTCAGTTTCAGT
GAATTTTCTGCATACTGTCCAAACAGTTAGGTCTTGGTTTTGTCATGGCTGCCCTGATAAGATTCAGTTG
ATGTTGTACTTATCGATTGATGGTGGTTCTTTTTGTGTTTCTAATCGTACTCCATCACCAGCAGCTCATA
TGGCACATATATATATCCATTTAGAGTGGCCCTAATGCTATTATTTAGTTTTTGTATTCTTGACTGAGCT
TGATGCTGACAAGTGAGAACTTATACTATGAAAGATATATTCGGTTGACCTCATGCTGACTGTTTTTTCC
CCTTTCCATGTGTATCTCATCAGAATGTCACACCAGCCACTTACATTGCAACTCACAGGACAGATCCGCC
TCCCAATCAAGGTAACACCTTTCCATGTTCTCGAACCAACGTTCGTGTGCTGCTCCCTTAAATGTATCCT
ATTTTATTTGCTCATTGTCCACTTGCATGCAATTGAGGCCATGTAATTACTGTTCATAACTTCATATTAT
GCCGACAATTTCTTTTCTACGTATTGTCGTACTATGTAAGCTCCGTGTTGCAAAATAGGTCTCCTGAACT
GTTTGGTCTGAACTGAAAGTTCTGTAGGTAGATGGCTCTCTTATCTTTCAGGAACGGAGCATGTTTCTCC
AAACATTTTGCGCCTCAGGGTAGAGTGCTGCAAGTCGCAAAGAGTGATACCAGCCATAATGACTGAACTA
CTGAACACCTTATCCTACACAAATGATGACCTCTGCTACGTTTCCCCAACCTATAGTCCAAAAGTTCCTC
GCTGCCACTAGTATCAGCAGCTGAATGCTATGATTGTTTTTTCAATAGTAGCTGTCTTAACCATGGCGCA
GTATAGCACTACTTGCCTCACTTGCCAATTTACCAGCTGCAACTGTCAGTAGTACCAGGCTTTATGTGAT
TTATTAAATGCTGTTTGTTTCTTGCAAAAAGCCCAGGTACTGCCCCTTCTCACCAAAGTTTACATTCAAA
CTTAATTAGGAAACCTTTTTTTGTTACATTAGGAAATTCTGAGAACTGATTGATACTCGCTTGGTACAGT
CCCCGTTCGAGGAGGCCACCCACCTAGGCTTGAAACTGGGTGCTTGCAAAATGTTTGTGTGTGTACCTCT
GCTTTCATCGAGTTTCCCGGTCAGCCACAGTTTTGCACTCCCGTTCTTGAAACAAAGCATGGGGGATTTC
ATCTTCCCATGGTCAAGTTTTTTTTAAGTAGGAAATTCCAATTTATGAAGGTATGTTTCTTAGTTGTACT
AAAACACATTGGCTAACAGCATATTAGTATTTCTCTTGGGTTTGATCTCCATATGAGTGGCTGGGTTTTT
ATGCTGGCTCGCCAAGCCTATCACAACCCCCCTCCTCCTTTATCCGGGCTATGTTGAAACAACACAGGCT
GACAGGCAGAGTTCTTGGTATGTCCATGTGTAGCTTATCTATATTGCTGCCAAGTTGGTTTGCCTTCTGT
TCAGACATCTAGATCCCTCAGAACCATTGCCCTGATCTATATGGTAAGTGCCCGAAAGAATATTGATATT
GGAGCATGAGGAAGATGAATAAAGGTTGGGTAAGTAGCGTTCAAAGGCGAGGGTGGAGCAAGGGTGTTGG
TGTTTAAGATGGAACAACATGCCAGTTAAACGCCATTGGAGCCGTCCAGGTGAAATGCCATCCACATAGA
GTGCTAGAACTGCGATATCCTTGAGAGTAGTAGTAAACTAATGGTCAAACTGATTAGGTTGTTGGCTTAA
ACACTCTGGTTTGTCAATTTCTATTACATCTCACCCTAAACATTTACCAGGCGACACAAAACCAGAAGAA
GTCCTTATTGCATCCAGGCCTTTTGGCTCAGCCATGTCCTCTGCATGTTGGTGAAAACTTGATGGTGCAC
TGCTGACCAAGTGTACCAAAAAAAAAGATCAAAGATAGAAACAAACTATTCTTTTTAAGGAGCAAAAGGT
ACTGATAGATAAAAGCATACTAGGTGCCCATTCGGCCATTCCTGGGGCTGGTGAGGCGATGAAATATTAT
TGGAGAAGTTTTGTGTGAACAATTTAAATCATTTGGAGGGATACTTAACTCTGAACTATTTTATTAAGTA
CCATGCCTAGATGTTACAGTCATGGAGTTGATTCCTTAAAAGTTGTTGGCAGATTCTATTTCTCTGTCTA
TTTCATTGTGGAAGTTGTATGTGGCCAAGTTAATTTCCAAGTTTTGGCAGATACACTCAATAAAAAAATC
GTTGGTGTTTCTTTAACCAATATATCTGCCAGTTATGTGTGTTTTCTGTTTATGTGACTGCATTCGTTTA
TAGATATGCTACACTTCATATTTGATCAGGCTGACCAAGTGCTTTCTATTATTTCATTTTATTGAATTTT
AATTCCTCTGAGGCACTCATGAACTTCCTCTAAAATTGTCATAGATTGCCCAATTCCTTTCTCTTCTCCA
CTGCACCAAAACCATCTACCTGGTGTTGTATTTTTCTGCTTCCTGAATTCAGTAATTGATGAATCGTGCC
CTGGGATTGCAGTGATAACAACAGAGCCCAGGAACATCCTGTTGAGGCACTTCTACCAGAAATCCGAGGA
GAAGGTAAGCTGTTCCATCAAGGCTGCATATACGCCACATCACAATGGAATCTAATCATCTATGCTTAAA
TCCTGTCCTGGACTCTAGCTGAGGCCAAAGAGAGCAGCTCCTGACAATCTCGCTCCAGAGAACAACAACA
AACAGCCCAGGGGCCCTGTCGCCGATGTTGGAAGCCAGTCAAACGCAAGAAGCTGAATACAGCTGGTGCT
TGTCCTCCCCTGCGTCTCTCAATGCCGTGTGCAACCTGCATGCTGCATGCCAGCTGAAGCCCTGGTCCTC
TTGATCCAAAGAGCTACGCTCATTACATGCATGAATGTACATAACAACCTCCCCCCTTTCCCTCCAACAT
TGGTTTGTTATTTGTTAGCGACTGGTGGCTGCATTTTAGTGACAGATTTTAGTAAAGAAAAAGGATGGTT
CGGCATGAAAAGATAGCCGCTTTTCTCTTGCTTATGCAATACTCCGTACAATTTAGTAAAATATAGACAC
TATTTGTA
Setaria italica
SEQ ID NO: 177
MGSPLGGWPSYNPRNFSQLVPADPSSQPSNVTPATYIATHRTDPPPNQVITTEPRNILLRHFYQKSEEKL
RPKRAAPDNLAPENNNKQPRGPVADVGSQSNARS
Panicum virgatum CDS
SEQ ID NO: 178
ATGGGGAGCCCACTCGGCGGGTGGCCGTCGTACAACCCGCACAACTTCAGCCAGCTCGTCCCGGCCGACC
CCTCCGCTCAGCCCTCGAATGTCACACCAGCCACTTACATTGCAGCTCACAGGACAGATCCACCTCCCAA
TCAAGTGATAACAACAGAGCCCAGGAACATCCTGCTGAGGCACTTCTATCAGAAATCTGAGGAGAAGCTG
AGGCCAAAGAGAGCAGCTCCAGACAATCTCGCTCCGGAGAACAACAACAAACAGCCCAGGGGTCCCGTCG
CCGATGTTGGAAGCCAGTCAAACGCTAGAAGCTGA
Panicum virgatum cDNA
SEQ ID NO: 179
AAGGAAAGCGCTAACCACCAGCGGCAGACGAAGTGAGGGGAGCATCGCCGGACGCCGGAGTCAGAGACGG
CGCGGCGATTCCGGCTCAACGAACCACCAGGGGAACAAGACGGGCGGTGGCGGCGCGGAGATGGGGAGCC
CACTCGGCGGGTGGCCGTCGTACAACCCGCACAACTTCAGCCAGCTCGTCCCGGCCGACCCCTCCGCTCA
GCCCTCGAATGTCACACCAGCCACTTACATTGCAGCTCACAGGACAGATCCACCTCCCAATCAAGTGATA
ACAACAGAGCCCAGGAACATCCTGCTGAGGCACTTCTATCAGAAATCTGAGGAGAAGCTGAGGCCAAAGA
GAGCAGCTCCAGACAATCTCGCTCCGGAGAACAACAACAAACAGCCCAGGGGTCCATGGAATACAAAACC
GCTCGATAATCGCGATTATCGGTGAAATTTACCGTTACCGATGTTGACTGATATCGGTTTTCAATTGATT
TTTCGATGGATTTCGATCCAAATTTCAAAAATTCAAAGAAATTTATAACTAGTGTGGAAAAAATTCTATA
AAAAACTAGAGCCTCTCTATAGTCTAGAATGATGTCACATATTAAAAACAACCACCGTTTGTTTAGACAA
AAAAATGTTTCCAATACTAAAGCCTGATAATTGATGCAAATCCATCGATAATCAATGCAAATCAGTTGAT
ATTCAACAATTTTGGTTGATTTTCTATTTCCTTTCACCAACTTGACCAAATATGCATGGGGTATTTACTA
TATTGTTGTATATTATGCTACAAATGGATGGTTATACTGATAATTTCCAATGTAGATTAGTGTTAAATAT
TAGTGGTGGGAAGAAAGACTTCAATGTTGACTTGTTGTTAAATCAGTTAGGATACAATAGGCTTCAATGT
TGACTATAATATGTATGCTTATACTAAAAAAAACTATGTCTAACTGGT
Panicum virgatum gDNA
SEQ ID NO: 180
AAGGAAAGCGCTAACCACCAGCGGCAGACGAAGTGAGGGGAGCATCGCCGGACGCCGGAGTCAGAGACGG
CGCGGCGATTCCGGCTCAACGAACCACCAGGGGAACAAGACGGGCGGTGGCGGCGCGGAGATGGGGAGCC
CACTCGGCGGGTGGCCGTCGTACAACCCGCACAACTTCAGCCAGCTCGTCCCGGCCGACCCCTCCGCTCA
GCCCTCGGTCGGTCAGCACTTCCCCTCTTTGGCGATCTCGTCTCTAATATACAGTAATTGACTGTCTCCA
TACTTCCTGATGATGCTGCATAAGCTTGAATAGGTTAGCTAGGACGTATTAGTGGGTGCTTGGCCTGTGC
TGTGACAACTGCGGCCTCTTGTTATCTTGTATCTGCAATAAAAACTTCTTTAGTACTGTACTGCCTTTAT
GCAGCCAGGAAGCAGGATGATCGTATTGTTCGATTCTGCTGAAGTCGCGGTATGGATGGCTGGAAAAGGA
GGGTATAAATGTTTGTTTTTAGCTCATCAGCCAGATACTCGGCTCAATTTTAGTGAATTTTCTGCATGAT
GTCCAAAAATTATTAGGTCTTGGTTTCTCATGGCTGCCCTGACAAGATTCAGTTGATGTAGTGTTGTCCT
AATCGATTAATGCTAGTTCTTTTAGTGTTTCTGATCACACTGTACCGCCAGCGGCTCACATGGCAAAGCA
CATATATATCCATTTAGAGTGACTCTAATGTTATTAGGTAGTTTTTATGTTCTTAACAGAACTTCATGCT
GACAAGTGATAATTTAGGCTATGAAAGATATACTCTGTTGACCTCATGCTGATGCTGATGGTATGTTTCC
TTTCCTTGTGTATCCCATCAGAATGTCACACCAGCCACTTACATTGCAGCTCACAGGACAGATCCACCTC
CCAATCAAGGTAACTCCTTCCCATGTTCTTGAAAAAATGTTCTGTGTGCTGCTACCTGCTGTAAATGTAT
CTTATTTTATTTTCTCACTGTGCATTTTCCCGCAATTGAGATCATGCAATTACTTGTTCGCAATAAGCCG
AGAATTTCTTTTCTGTCTATTTTAGTACTATGTAAGCTCAATATTGCAAAGTAGATCTTGTGAACCCGTT
TGGCAAAAGTTCTTAGCATGTTTCTCCATAAGATTCTTTGCTCATGGTATATTGTGTCTGCAAGTCACAG
AGGTGATATATTAGCCATGATGACTGAACTACTGAACACCTTATCCTACACAAATGATGGTCTCCCCTCT
GCTATGTCTCCCCAATCTATAGACCATAATTTTCCTTGCTGCCACTGGTAATCAGCAGCTGAAAGCTATG
ATTGATTGTTGGCTGTCTTAACCATGTGCAGTATAATACTAATTGTCTTACTGCCCATTTACCTGCTGTA
AGTGTCAGTAGTACCAGGTACTGCCCCTTTTTCAATATCAAAGTTTTACCAGGTAATGCATGCAGTGCAA
TTTTTCTTTGATCTACATGGACAACAATTCAATTTGCTAAATACTGCACATATAGTACTGATTCCAAAAT
CTGAGGATGCTACTGATCTCTCACATTATAGACCTATCAGCTTGACAAGTAGTATTCCAAAATTATTCTC
AAAGCTGCTTGCACTCAGATTGGCCAAGAGTTTGGACACACTAATCTCAAGGAATCAAAGTGCTTTTATT
CGAAGGAGTATCCATGATAACTTCTTATACACACAAAATCTCATTCGAGCTCTACATAAAGATGGCAGGC
CCTCCCTTTTTATTAAGCTGGACATTGCAAAGGCTTTTGACACTGTGCGATGGAATTATCTGATGGAGGT
GTTAGAGAAACTTGGGTTTGGTCACAAATGGAGGGGCTGGATTTCTTTACTGCTATCAACTGCCACTTCC
TCGGTCTTAGTCAATGGAGCACAAACTCCAAAATTTAAGCACATGATCAGGTTAAGGCAGGGAGACCCTT
TGTCTCCAATGCTTTTCATCCTGGCACTTGAACCTTTGCAACACTTGCTGGCTTTAGAAGAAGCTTCGGG
CAACCTATCACCAATACACACAAATATGGCAACGTTAAGAATAAGTTTATTTGCCGATGATGCTGCAGTT
TTTCTAAACCCAGTGAAAGAAGAGATTGATGTGATCAAAGAGGTATTTCAGGCATTTGGAAATGCTTCTG
GACTGAAGGTGAACTTAAGTAAAAGTGCTATCTATCCTATTAGATGTGAGGGCATTGATCTTGAAGAAGT
ACTGCAGAATTTCCCATGCCAAATAAAAGCCTTCCCCTGCAAGTACCTGGGACTACCAGTGAGTACAAGG
TGTCTAAGAAGAATTGAGGTGCAACCTTTATTTGACAAAATTGCAGCTAGGCTGCCAGCATGGAAGGGGA
AGCTTTTGAATAGAGCAGGCTGGTTGACTTTGGTAAAGTCTGTACTCGCCGCAGTGCCAATTTATTTCCT
CACGGTGTTTCCTCTTAAGAAATGGGCCTTAAAGAAAATTGATAGACTGAGAAGAGCCTTTCTTTGGAGA
GGAACTGAGGAGGCCCGTGGTGATTACTGCTTGGTCAATTGGAAGAAGGTAATGCTACCAAAGGAGATGG
GAGGGCTCGAGTATTGGATCTAAGTTGTTTTGGGAGAGCTCTAAGATTGCGTTGGTTGTGGTACGCTTGG
ACAGAGCCTGACAGACCTTGGGTGGGATCGGCACCACCATGTGATGAGGTGGATAAACAACTTTTCAGAG
CAAGCACAATTGTTCAGTTGGGGGATGGTAACAAAGCTTCTTTCTGGAAATGTAGCTGGTTAAATGGAAG
GGCCCCTAGGGACATTGCACCTGGGCTGTTTAAGTTGGCTTGGAGAAAGAATAGAACTGTAAGAGAAGAC
ATCATAAATCAGCAATGGACAAGGGGGCTCTGTAGAATGGATTCAGTTGAGTTAATGTCACAGTTTGTGG
TTCTTTGGGATGCAGTACAGCAGGTTCAGTTGACGGATAGGCCGGATGAGATAGTCTGGAGATGGACAGC
TAATGGGGCTTATACTTCAAAGTCTGCTTATCTTGCTCAACTCAAGGGAACTTTTTGTACATTTGATGCC
CAATCAATCTGGCATGCACATGCTGAAGGGAAACACCGCTTCTTTGCTTGGCTTCTAGTGCAAAGCAAAA
TATTAACGGCCGACAAGCTGGTCGCTAGGAATTGGCTGTGTGACACTAATTGTGCTTTGTGTGACCAAGT
TCATGAAACAGCTGCACATCTGTTTGCATTGCTCTTATGCTAAGCAGGTCTGGCTCGCGATGAGCAACTG
GACATCAGGCGCCATACACATACTGGCGGTTCAAGACGAGGGGGTCGAGGATTGGTGGAACAGAAGCTTA
GCGTTGCTACCGGTGGCACAGAAACGCTCAGTTGCGGCCATCTTGATGTACACTTGCTGGAATTTGTGGA
AAGAAAGGAACAGGAGAGTGTTTGACCAAAAATGTTTGCAGCCACATGAAGTTGTCCAGCTGATCAAGGA
AGAAGTCAACCTGAGAAGGGTGGCTTGTGGCACACCCATGGTGTTCTAGTTGGTTTTCATGTTTAGAGGA
TTCTTGTTTAGAGGAGGGTTAATGTTTTTATGTAAATTAAACTCTTATTGAACTCGCTTGCTTCCTTCTT
AAATGCATCGGCAGCGCTCCTGCCAAACTTTCAAAAAAAAAAGTTTTACCTTAAAAAACTAATTAGGAAA
CCTTCTCTGTTACATTAGGGAATTCCAAAAAGCAATCATACTTGCTTTCTACAGTCTCCTTCGAGGAGGT
CACCCACCTAGCCTCAAACCTGGGTGCTTGCAAAATGTGTGTACCTCTCTGAGAACTGAAAGAACAAGTT
TCCTGGTCAGCCACGGCCGGGTCCTCCCCTTCTTGAAACAAAGCCAGGGGGAATTCATCTTGCCATGGTC
AAGTTCTTTCTAATAACTTTGCATTAGGAAATTCCAATTTATGAAGGCATGCTTCATAGTTTTACTGAAA
CATATTGGCTAACAGCACATTAGTATTTCTCTTGGGTAGCTCGGTTTCATCTCCATATGAAACCACAAGA
AATCCTTGTTGCATTCAGGCCTTTTGGCCCAGTCATGTCCTCCGTGTGTTGGTGAAAACTTGATAGTGCG
CTGCTGACCAAGTGTACCAAAAGACAAACGAACGAAAGAAAGAAAGAAACAAGCTATTCTTGTTAAGGAG
CGAGAGGAGGTGGTAGAAGAAAAGCATGTGCCTTATTCTGGGGCTGATGAGGCAATGAGATACTATTGGA
TTAGTTTTTATGTGAACAATTCAAATCATTTGGAGGCATACTTGAATCTGAACTATACCTCAGACTTCAG
GCACAAACTTCTGGTGGTGAATATTTATTAAATACCATGCCTAGATGTTACAGGCATGGAGTTGAATCCT
TAAAAGCTGTTGACAGATTCTATTTCTGCTGTCTACTTTCCTTAAGGAAGTTGTATGCGGACATGTTTAT
TTCCAAGTTTTAGCAGATACATTCAATGAATAATTCGTTGGTGTTTTGTTAACCAATATATCTTCTTTTC
ATTATGTGAGTGCATTCGTCTATAGATATGCTACACTCATGTTAGATCAGACTCAAGAAGCGCTTTATAT
AAAAGTCATCCATGTTGTATTTTTACTGCTTCCTTAATTCATTGATTGACAAATCGTGCCATTGGAATTG
CAGTGATAACAACAGAGCCCAGGAACATCCTGCTGAGGCACTTCTATCAGAAATCTGAGGAGAAGGTAAG
CTGTTCCATCAAGGCTGTACAGATCACATGACTATGGAATCTAACCATCTATACCTTAATCCTGTCCTGA
ACTTTAGCTGAGGCCAAAGAGAGCAGCTCCAGACAATCTCGCTCCGGAGAACAACAACAAACAGCCCAGG
GGTCCATGGAATACAAAACCGCTCGATAATCGCGATTATCGGTGAAATTTACCGTTACCGATGTTGACTG
ATATCGGTTTTCAATTGATTTTTCGATGGATTTCGATCCAAATTTCAAAAATTCAAAGAAATTTATAACT
AGTGTGGAAAAAATTCTATAAAAAACTAGAGCCTCTCTATAGTCTAGAATGATGTCACATATTAAAAACA
ACCACCGTTTGTTTAGACAAAAAAATGTTTCCAATACTAAAGCCTGATAATTGATGCAAATCCATCGATA
ATCAATGCAAATCAGTTGATATTCAACAATTTTGGTTGATTTTCTATTTCCTTTCACCAACTTGACCAAA
TATGCATGGGGTATTTACTATATTGTTGTATATTATGCTACAAATGGATGGTTATACTGATAATTTCCAA
TGTAGATTAGTGTTAAATATTAGTGGTGGGAAGAAAGACTTCAATGTTGACTTGTTGTTAAATCAGTTAG
GATACAATAGGCTTCAATGTTGACTATAATATGTATGCTTATACTAAAAAAAACTATGTCTAACTGGT
Panicum virgatum
SEQ ID NO: 181
MGSPLGGWPSYNPHNFSQLVPADPSAQPSNVTPATYIAAHRTDPPPNQVITTEPRNILLRHFYQKSEEKL
RPKRAAPDNLAPENNNKQPRGPVADVGSQSHARS
Phyllostachys edulis CDS
SEQ ID NO: 182
ATGGGGAGCCCCCTGGGTGACTGGCCGTCCTACAACCCGCACAACTTCAGCCAGCTCGTCCCGGCCGACC
CCTCCGCCCAGCCCTCGAATGTCACACCAGCCACGTACATTGCGACGCATAGGACAGATCCACCTCCCAA
TCAAGTGATAACAACTGACTCTAGGAACATCCTGTTGAGGCATTTTTATCAAAAATCCGAGGAGAAGTTG
AGGCCAAAGAGAGCCGCACCGGACAATCTTACCCTGCAGAACAATTGCAAACAGCCAAGGGGCCCTGTTG
CCGATGGTGGAAGCCAGTCAAGTAGTAGAAGCTAA
Phyllostachys edulis cDNA
SEQ ID NO: 183
GAAGAGGAAGAAGAAGAAGAAGAAGAAGGAAGCATCGGCGGTGGCGTCGCGGCGATGGGGAGCCCCCTGG
GTGACTGGCCGTCCTACAACCCGCACAACTTCAGCCAGCTCGTCCCGGCCGACCCCTCCGCCCAGCCCTC
GAATGTCACACCAGCCACGTACATTGCGACGCATAGGACAGATCCACCTCCCAATCAAGTGATAACAACT
GACTCTAGGAACATCCTGTTGAGGCATTTTTATCAAAAATCCGAGGAGAAGTTGAGGCCAAAGAGAGCCG
CACCGGACAATCTTACCCTGCAGAACAATTGCAAACAGCCAAGGGGCCCTGTTGCCGATGGTGGAAGCCA
GTCAAGTAGTAGAAGCTAAATCACCGCCAGTGTTCTCCTCTCCTGCATCTCTTACGGTCGTTGCGGCTGC
TGCTGATGCATGTCATGCTACCTGTGTGGCTGTGTGCTTGTTCAAGCATGCGAAGCCCTCTCATTTCTCA
TGTATTATCAAAAGAGCTTGGATGCATGTACATACCCTTCAGCGAGCCCCTCAGTGCGGTACCTTTCACA
TGGCACTACTGCAGTCTCTTCTGAATATAATGTGCCCACACTAGCCAACTTGTGCTTTTGATTGAAACAA
AACCATGGCTCCATAATTGCGTTGCTTC
Phyllostachys edulis
SEQ ID NO: 184
MGSPLGDWPSYNPHNFSQLVPADPSAQPSNVTPATYIATHRTDPPPNQVITTDSRNILLRHFYQKSEEKL
RPKRAAPDNLTLQNNCKQPRGPVADGGSQSSSRS
Picea glauca CDS
SEQ ID NO: 185
ATGGGGTCATTGCTTGGAGATTGGCCCTCCTATAATCCGCACAATTTCAGTCAGTTGAGGCCGTCGGATC
CCTCGCATCCCTCGCAATTGACACCGGTCACTTACTATCCTACTCATAATAGAACAGCACCCCCAGCACA
CCAAGTAATTTCAACTGAGGCTACAAATATCCTTTTAAGGCAGTTTTATCAGCGAGCAGAAGAGAAGTTG
AAGGCAAAGAGGCCGGCCTCTGATGCTCTTGTACAAGAACACATGAACAAGCACCCCAAGAGCTGA
Picea glauca cDNA
SEQ ID NO: 186
AAGACACATGGATCGGTTCTGCACATGCAGCCGCGAGGATCTGCGTCCAGGCAGTGGCTGGAGACGGCCC
CTCCACCTGTTATTCGCGTCAAGAAACGGACTCTCCCTGCGCAGAAACTGGAGACCATAGCAGAAGAATC
CTGCTGTTTCGAAGACCCTGAAAGCATCGAGCCTGATTCCCCGTCACAGACACGGGCGTCAGCTTTGAGA
TTTGGGCAGAGCGGCTACGAAATCATCGAGCCCGATTCCCCGTCACAGACACGGGCGTCAGCGTTGAGAT
TTGGGCAGAGCGGTTATGAAAGCTTCGAGCCCGATTTCCCGTCACAGATACGGGCGTCGGCGTTGAGATC
TGGGTAATGACGGGTTCTGTTTTTCTGCTGTATTGGTTGAGTGGGTTGCCGTCAAGTGACGATTCTAGAC
TGACGGGGGGTTAAGCGTGTTTCGGGCTCAAATGGGTTTTTTTATTTTATGTAATTTGTCAGAAATTTTC
TCCATCGGCGATCGTATGGATCAAGATGGCAGTTATCTCCTCGTGTACAGTGGAATTTTCTGTTGTCAAT
CTCATGTACATAATTTGGAATTTTCTGTTGTCAATCTCATGTACATAATTCGTGGATATAGTGGAATCGG
AATTTTCTGTACGTC
Picea glauca
SEQ ID NO: 187
MGSLLGDWPSYNPHNFSQLRPSDPSHPSQLTPVTYYPTHNRTAPPAHQVISTEATNILLRQFYQRAEEKL
KAKRPASDALVQEHMNKHPKS
Selaginella moellendorffii CDS
SEQ ID NO: 188
ATGGGTTCCTTGCTGGGCGATCTTCCTTCGTACAACCCGCACAATTTCAGCCAGTTGAGACCATCGGATC
CTTCTCATCGCTCCCAACTCACACCGCTCACTTATCACGCTACTCACGACCGGACGATGCCTCCGGCGGA
TCAAGTCATCTCCACTGAAGCTACCAACATTTTGCTGAGGCACTTCTATCAAAAAGCCGATCACAAGCTC
AAGTTGAAGCGCTCGGCCACCGATTCGCCTCTCGGGGATCACAAGCGTCCCAAGAGCACAACTTGCGCTC
CAGAGAAGAGATGA
Selaginella moellendorffii cDNA
SEQ ID NO: 189
GGCTCTTTTCCATGTCATAGGAGGAGGAGAGAAGGGACATTCTTTTAGCTGCGGGGTTGCGATCGATCGA
GCGAGAGGGAATCGGTGTGCGCCTTAAAATCCTGGTCGCTCTATCGGATAGAAGCGAGCGATCGTGTCGC
TTGCGCTCGAAGGGTAGGGTTTTTGGTTCTCCCAGAGTGTAGGTAGGGCTTTGCAATGCCGCTGCGCCTC
CTCCTCTAGAAGCGCGCAGATCTATCGTCTTCGTCGAGTAGCAACGCAAAGCGAAAAAAGAGGTTTTCTT
TTCGCGAGGATCACAATGGGTTCCTTGCTGGGCGATCTTCCTTCGTACAACCCGCACAATTTCAGCCAGT
TGAGACCATCGGATCCTTCTCATCGCTCCCAACTCACACCGCTCACTTATCACGCTACTCACGACCGGAC
GATGCCTCCGGCGGATCAAGTCATCTCCACTGAAGCTACCAACATTTTGCTGAGGCACTTCTATCAAAAA
GCCGATCACAAGCTCAAGTTGAAGCGCTCGGCCACCGATTCGCCTCTCGGGGATCACAAGCGTCCCAAGA
GCACAACTTGCGCTCCAGAGAAGAGATGATCGCGAGTTCTCCCTGTACTTAACAAGCCCGCGATGGAAAA
AAAAACAGAGGTTGGCTACACAGGTTTGATGAGCAGAATCCATTTTCTCGATCTCTAAGCTTGTGAATAT
CTAGATCGACAATGGTAACTTTCTTTTAGAAA
Selaginella moellendorffii gDNA
SEQ ID NO: 190
GGCTCTTTTCCATGTCATAGGAGGAGGAGAGAAGGGACATTCTTTTAGCTGCGGGGTTGCGATCGATCGA
GCGAGAGGGAATCGGTGTGCGCCTTAAAATCCTGGTCGCTCTATCGGATAGAAGCGAGCGATCGTGTCGC
TTGCGCTCGAAGGGTAGGGTTTTTGGTTCTCCCAGAGTGTAGGTAGGGCTTTGCAATGCCGCTGCGCCTC
CTCCTCTAGAAGCGCGCAGATCTATCGTCTTCGTCGAGGTATGTGGAGTAATCTCTCCTTGTTCTTCCCC
TCTTCTCATTAGCTCTTTTCATTCATCAGTAGCAACGCAAAGCGAAAAAAGAGGTTTTCTTTTCGCGAGG
ATCACAATGGGTTCCTTGCTGGGCGATCTTCCTTCGTACAACCCGCACAATTTCAGCCAGTTGAGACCAT
CGGATCCTTCTCATCGCTCCGTAAGAGATCGACGAGCATTTTCTCTTCGGTTTTTCTTCTCTTCGTGTTT
TCTTCGTTGTTCTTGCTTGACTGACCACCATTTCTTTTTTTTTTTTCTTTTTTTTTTTGCAGCAACTCAC
ACCGCTCACTTATCACGCTACTCACGACCGGACGATGCCTCCGGCGGATCAAGGTAACCATCACCATAGC
TTCGCGAATTTGAGCTAACTTTGCTTTCTTTGCAGTCATCTCCACTGAAGCTACCAACATTTTGCTGAGG
CACTTCTATCAAAAAGCCGATCACAAGGTAAGTTCTTCCCGATCAATGCTATGATTCATTCATCACTCAC
TCGAGTGTATGCAAGCAGCTCAAGTTGAAGCGCTCGGCCACCGATTCGCCTCTCGGGGATCACAAGCGTC
CCAAGAGCACAACTTGCGCTCCAGAGAAGAGATGATCGCGAGTTCTCCCTGTACTTAACAAGCCCGCGAT
GGAAAAAAAAACAGAGGTTGGCTACACAGGTTTGATGAGCAGAATCCATTTTCTCGATCTCTAAGCTTGT
GAATATCTAGATCGACAATGGTAACTTTCTTTTAGAAA
Selaginella moellendorffii
SEQ ID NO: 191
MGSLLGDLPSYNPHNFSQLRPSDPSHRSQLTPLTYHATHDRTMPPADQVISTEATNILLRHFYQKADHKL
KLKRSATDSPLGDHKRPKSTTCAPEKR
DDA1 consensus sequence
SEQ ID No: 192
MGSSS[LM]LGDWPSFDPHNESQLRPSDPSSNPSKMTPATYHPTHSRTLPPPDQVITTEAKNILLRHEYQ
RAEEKLRPKRAASENLLAPEHGCKQPRGPVAS[ST]SDTQSSASGRS

1. A method for increasing yield and/or growth of a plant under stress conditions or mitigating the impact of a plant under stress, said method comprising introducing and expressing in said plant a nucleic acid construct comprising a plant DDA I nucleic acid sequence.

2. A method for reducing a plant response to abscisic acid (ABA), or modulating the interaction of the PYL8 receptor with the same, said method comprising introducing and expressing in said plant a nucleic acid construct comprising a plant DDA1 nucleic acid sequence.

3. (canceled)

4. A method for reducing seed dormancy said method comprising introducing and expressing in said plant a nucleic acid construct comprising a plant DDA1 nucleic acid sequence.

5. (canceled)

6. A method for producing a transgenic plant with improved yield/growth under stress conditions said method comprising introducing and expressing in said plant a nucleic acid construct comprising a plant DDA1 nucleic acid sequence.

7. A method according to claim 1 wherein said plant DDA1 nucleic acid sequence is a monocot or dicot plant DDA1 nucleic acid sequence.

8. A method according to claim 1, wherein said plant DDA1 nucleic acid sequence comprises SEQ ID NO: 1, 2 or 3 or a functional variant or homolog thereof.

9. A method according to claim 8 wherein said homolog has at least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the nucleic acid represented by SEQ ID NO: 1, 2 or 3 or wherein the peptide encoded by a homolog of SEQ II) NO: 1 has at least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 4.

10. A method according to claim 9 wherein said plant DDA1 nucleic acid sequence encodes a protein which comprises SEQ ID No: 4, 8, 11, 14, 18, 22, 26, 30, 34, 38, 42, 45, 49, 52, 56, 60, 64, 68, 71, 75, 79, 83, 87, 90, 94, 98, 102, 106, 109, 112, 115, 119, 123, 126, 130, 133, 136, 139, 143, 147, 151, 155, 159, 163, 166, 169, 173, 177, 181, 184, 187, 191 or a functional variant thereof.

11. A method according to claim wherein said stress is abiotic stress.

12. A method according to claim 11 wherein said stress is moderate stress.

13. A method according to claim 11 wherein said stress is drought or salinity.

14. A method according to claim 1, wherein said plant is a monocot or dicot plant

15. A method according to claim 1, wherein said plant is a crop plant or biofuel plant.

16. A method according to claim 15 wherein said crop plant is selected from maize, rice, wheat, oilseed rape, sorghum, soybean, potato, tomato, grape, barley, pea, bean, field bean, lettuce, cotton, sugar cane, sugar beet, broccoli or other vegetable brassicas or poplar.

17. A method according to claim 1, wherein said construct further comprises a regulatory sequence.

18. A method according to claim 17 wherein said regulatory sequence is a constitutive promoter, a strong promoter, an inducible promoter, a stress inducible promoter or a tissue specific promoter.

19. A method according to claim 18 wherein said regulatory sequence is the CaMV35S promoter.

20. A transgenic plant with an altered response to ABA wherein said plant expresses a nucleic acid construct comprising a plant DDA 1 nucleic acid sequence.

21. A plant according to claim 20 wherein said plant DDA I nucleic acid sequence comprises SEQ ID No: 1, 2 or 3 or a functional variant or homolog thereof.

22. A plant according to claim 21 wherein said plant DDA1 nucleic acid sequence encodes a polypeptide comprising SEQ ID NO: 4, a functional variant or homolog thereof.

23. A plant according to claim 22 wherein said homolog has at least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 4.

24. A plant according to claim 21 wherein said plant DDA1 nucleic acid encodes a protein which comprises SEQ ID No: 4, 8, 11, 14, 18, 22, 26, 30, 34, 38, 42, 45, 49, 52, 56, 60, 64, 68, 71, 75, 79, 83, 87, 90, 94, 98, 102, 106, 109, 112, 115, 119, 123, 126, 130, 133, 136, 139, 143, 147, 151, 155, 159, 1.63, 166, 169, 173, 1.77, 181, 184, 187, 191 or a functional variant thereof.

25. A plant according to claim 20 wherein said construct further comprises a regulatory sequence.

26. A plant according to claim 20 wherein said regulatory sequence is a constitutive promoter, a strong promoter, an inducible promoter, a stress inducible promoter or a tissue specific promoter.

27. A plant according to claim 26 wherein said regulatory sequence is the CaMV35S promoter.

28. A plant according to claim 27 wherein said regulatory sequence is a stress inducible promoter.

29. A plant according to claim 28 wherein said stress inducible promoter is selected from Hahh1, RD29A or rabl7, P5CS1 or ABA- and drought-inducible promoters of Arabidopsis clade A PP2Cs, tbr example ABI1, ABI2, HAB1, PP2CA, HAI1, HAI2 and HAI3 or their corresponding crop orthologs.

30. A plant according to claim 20 wherein said plant is a monocot or dicot plant.

31. A plant according to claims 20 wherein said plant is a crop plant or biofuel plant.

32. A plant according to claim 31 wherein said crop plant is selected from maize, rice, wheat, oilseed rape, sorghum, soybean, potato, tomato, grape, barley, pea, bean, field bean, lettuce, cotton, sugar cane, sugar beet, broccoli or other vegetable brassicas or poplar.

33. Plant according to claim 20 wherein said plant has increased stress resistance.

34. A product derived from a plant as defined in claim or from a part thereof.

35. A vector comprising a plant DDA1 nucleic acid sequence.

36. A vector according to claim 35 wherein said plant DDA1 nucleic acid sequence is a nucleic acid corresponding to SEQ D NO: 1, 2 or 3 or a functional variant or homolog thereof.

37. A vector according to claim 35 further comprising a regulatory sequence which directs expression of the nucleic acid.

38. A vector according to claim 37 wherein said regulatory sequence is a constitutive promoter, a strong promoter, an inducible promoter, a stress inducible promoter or a tissue specific promoter.

39. A vector according to claim 38 wherein said regulatory sequence is the CaMV35S promoter,

40. A vector according to claim 38 wherein said regulatory sequence is a stress inducible promoter.

41. A vector according to claim 40 wherein said stress inducible promoter is selected from a Hahb1, RD29A or rabl7, P5CS1 or ABA- and drought-inducible promoters of Arabidopsis clade A PP2Cs, for example ABI1, ABI2, HAB1, PP2CA, HAI1, HAI2 and HAI3 or their corresponding crop orthologs.

42. A host cell comprising a vector according to claim 34.

43. A host cell according to claim 42 wherein said host cell is a bacterial or a plant cell.

44. The use of a DDAI plant nucleic acid sequence in reducing a plant response to ABA.

45. The use of a DDAI plant nucleic acid sequence in reducing seed dormancy.

46. The use of a DDA 1 plant nucleic acid sequence in increasing yield/growth of a plant under stress conditions.

47. The use according to any of claims 44 to 46 wherein said plant DDA1 nucleic acid comprises SEQ ID NO: 1, 2 or 3 or a functional variant or homolog thereof.

48. A plant with increased expression of an endogenous DDA1 plant nucleic acid sequence wherein said endogenous DDA1 promoter carries a mutation introduced by mutagenesis or genome editing which. results in increased expression of the DDA1 gene.

49. A plant with increased stability of the endogenous DDA1 polypeptide wherein said endogenous DDA1 nucleic acid sequence carries a mutation introduced by mutagenesis or genome editing and which results in increased stability of the DDA1 protein,

50. A method for increasing expression of a DDA1 plant nucleic acid sequence or improving stability of a DDA1 protein in a plant, producing plants, a method for mitigating the impacts of stress conditions on plant growth and yield and a method for producing plants with improved yield/growth under stress conditions comprising the steps of mutagenising a plant population, identifying and selecting plants with an improved yield/growth under stress conditions and identifying a variant DDA1 promoter or gene sequence.

51. A method according to claim 2 wherein said plant DDA1 nucleic acid sequence is a monocot or dicot plant DDA1 nucleic acid sequence.

52. A method according to claim 2, *herein said plant DDA1 nucleic acid sequence comprises SEQ ID NO 1, 2 or 3 or a functional variant or homolog thereof.

53. A method according to claim 51 wherein said homolog has at least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 9.4%, 95%, 96%, 97%, 98%, or 99% overall sequence identity to the nucleic acid represented by SEQ ID NO: 1, 2 or 3 or wherein the peptide encoded by a homolog of. SEQ ID NO: 1 has at least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ. ID NO: 4.

54. A method according to claim 52 wherein said plant DDA1 nucleic acid sequence encodes a protein which comprises SEQ ID No: 4, 8, 11, 14, 18, 22, 26, 30, 34, 38, 42, 45, 49, 52, 56, 60, 64, 68, 71, 75, 79, 83, 87, 90, 94, 98, 102, 106, 109, 112, 115, 119, 123, 126, 130, 133, 136, 139, 143, 147, 151, 155, 159, 163, 166, 169, 173, 177, 181, 184, 187, 191 or a functional variant thereof.

55. A method according to claim 50, wherein said plant is a monocot or dicot plant.

56. A method according to claim 54, wherein said plant is a crop plant or biofuel plant.

57. A method according to claim 55 wherein said crop plant is selected from maize, rice, wheat., oilseed rape, sorghum, soybean, potato, tomato, grape, barley, pea, bean, field bean, lettuce, cotton, sugar cane, sugar beet, broccoli or other vegetable brassicas or poplar.

58. A method according to claim 50, wherein said construct further comprises a regulatory sequence.

59. A method according to claim 57 wherein said regulatory sequence is a constitutive promoter, a strong promoter, an inducible promoter, a stress inducible promoter or a tissue specific promoter.

60. A method according to claim 58 wherein said regulatory sequence is the CaMV35S promoter.

61. A method according to claim 4 wherein said plant DDA1 nucleic acid. sequence is a monocot or dicot plant DDA1 nucleic acid sequence.

62. A method according to claim 4, wherein said plant DDA1 nucleic acid sequence comprises SEQ ID NO: 1, 2 or 3 or a functional variant or homolog thereof.

63. A method according to claim 7 wherein said homolog has at. least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the nucleic acid represented by SEQ ID NO: 1, 2 or 3 or wherein the peptide encoded by a homolog of SEQ ID NO: 1 has at least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 4.

64. A method according to claim 62 wherein said plant DDA1 nucleic acid sequence encodes a protein which comprises SEQ ID No: 4, 8, 11, 14, 18, 22, 26, 30, 34, 38, 42, 45, 49, 52, 56, 60, 64, 68, 71, 75, 79, 83, 87, 90, 94, 98, 102, 106, 109, 112, 115, 119, 123, 126, 130, 133, 136, 139, 143, 147, 151, 155, 159, 163, 166, 169, 173, 177, 181, 184, 187, 191 or a functional variant thereof.

65. A method according to claim 4, wherein said plant is a monocot or dicot plant.

66. A method according to claim 4, wherein said plant is a. crop plant or biofuel plant.

67. A method according to claim 65 wherein said crop plant is selected from maize, rice, wheat, oilseed rape, sorghum, soybean, potato, tomato, grape, barley, pea, bean, field bean, lettuce, cotton, sugar cane, sugar beet, broccoli or other vegetable brassicas or poplar.

68. A method according to claim 4, wherein said construct further comprises a regulatory sequence.

69. A method according to claim 67 wherein said regulatory sequence is a constitutive promoter, a strong promoter, an inducible promoter, a stress inducible promoter or a tissue specific promoter.

70. A method according to claim 68 wherein said regulatory sequence is the CaMV35S promoter.

71. A method according to claim 6 wherein said plant DDA1 nucleic acid sequence is a monocot or dicot plant DDA1 nucleic acid sequence.

72. A method according to claim 6, wherein said plant DDA1 nucleic acid sequence comprises SEQ ID NO: 1, 2 or 3 or a functional variant or homolog thereof.

73. A method according to claim 71 wherein said homolog has at least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the nucleic acid represented by SEQ ID NO: 1, 2 or 3 or wherein the peptide encoded by a homolog of SEQ ID NO: 1 has at least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 4.

74. A method according to claim 72 wherein said plant DDA1 nucleic acid sequence encodes a protein which comprises SEQ ID No: 4, 8, 11, 14, 18, 22, 26, 30, 34, 38, 42, 45, 49, 52, 56, 60, 64, 68, 71, 75, 79, 83, 87, 90, 94, 98, 102, 106, 109, 112, 115, 119, 123, 126, 130, 133, 136, 139, 143, 147, 151, 155, 159, 163, 166, 169, 173, 177, 181, 184, 187, 191 or a functional variant thereof.

75. A method according to claim 6, wherein said plant is a monocot or dicot plant.

76. A method according to claim 6, wherein said plant is a crop plant or biofuel plant.

77. A method according to claim 75 wherein said crop plant is selected from maize, rice, wheat, oilseed rape, sorghum, soybean, potato, tomato, grape, barley, pea, bean, field bean, lettuce, cotton, sugar cane, sugar beet, broccoli or other vegetable brassicas or poplar.

78. A method according to claim 6, wherein said construct further comprises a regulatory sequence.

79. A method according to claim 77 wherein said regulatory sequence is a constitutive promoter, a strong promoter, an inducible promoter, a stress inducible promoter or a tissue specific promoter.

80. A method according to claim 79 wherein said regulatory sequence is the CaMV35S promoter.
